SEQUENCE LISTING (99 sequences)

SEQ ID NO: 1 - protein, 631 aa, Artificial Sequence
  protein sequence of fusion protein #1 N1-ATD-Fc
MSTMRLLTLALLFSCSVARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSFLRTVPPYSHQSSVWFEMMRVYSWNHIILLVSDDHEGRAAQKRLETLLEERESKAEKVLQFDPGTKNVTALLMEAKELEARVIILSASEDDAATVYRAAAMLNMTGSGYVWLVGEREISGNALRYAPDGILGLQLINGKNESAHISDAVGVVAQAVHELLEKENITDPPRGCVGNTNIWKTGPLFKRVLMSSKYADGVTGRVEFNEDGDRKFANYSIMNLQNRKLVQVGIYNGTHVIPNDRKIIWPGGETEKPRGYQMSTRLKIGSSTMVRSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 2 - protein, 400 aa, Homo sapiens
  Amino terminal domain (ATD) of GluN1 including the signal sequence as in fusion protein #1
MSTMRLLTLALLFSCSVARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSFLRTVPPYSHQSSVWFEMMRVYSWNHIILLVSDDHEGRAAQKRLETLLEERESKAEKVLQFDPGTKNVTALLMEAKELEARVIILSASEDDAATVYRAAAMLNMTGSGYVWLVGEREISGNALRYAPDGILGLQLINGKNESAHISDAVGVVAQAVHELLEKENITDPPRGCVGNTNIWKTGPLFKRVLMSSKYADGVTGRVEFNEDGDRKFANYSIMNLQNRKLVQVGIYNGTHVIPNDRKIIWPGGETEKPRGYQMSTRLKI

SEQ ID NO: 3 - protein, 8 aa, Artificial Sequence
  Linker between NMDAR domain and rabbit Fc to be used in fusion proteins of NMDAR protein constructs of the invention
GSSTMVRS

SEQ ID NO: 4 - protein, 223 aa, Artificial Sequence
  Rabbit Fc from Vector pFuse-rIgG-Fc1
SKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 5 - protein, 376 aa, Homo sapiens
  Amino terminal domain of GluN1 in NM_007327 with domain borders as in cd06379 PBP1_iGluR_NMDA_NR1 (NCBI)
TLALLFSCSVARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSFLRTVPPYSHQSSVWFEMMRVYSWNHIILLVSDDHEGRAAQKRLETLLEERESKAEKVLQFDPGTKNVTALLMEAKELEARVIILSASEDDAATVYRAAAMLNMTGSGYVWLVGEREISGNALRYAPDGILGLQLINGKNESAHISDAVGVVAQAVHELLEKENITDPPRGCVGNTNIWKTGPLFKRVLMSSKYADGVTGRVEFNEDGDRKFANYSIMNLQNRKLVQVGIYNGTHVIPNDRKIIWPG

SEQ ID NO: 6 - protein, 915 aa, Artificial Sequence
  protein seq of fusion protein #2 N1ecd-Fc
MSTMRLLTLALLFSCSVARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSFLRTVPPYSHQSSVWFEMMRVYSWNHIILLVSDDHEGRAAQKRLETLLEERESKAEKVLQFDPGTKNVTALLMEAKELEARVIILSASEDDAATVYRAAAMLNMTGSGYVWLVGEREISGNALRYAPDGILGLQLINGKNESAHISDAVGVVAQAVHELLEKENITDPPRGCVGNTNIWKTGPLFKRVLMSSKYADGVTGRVEFNEDGDRKFANYSIMNLQNRKLVQVGIYNGTHVIPNDRKIIWPGGETEKPRGYQMSTRLKIVTIHQEPFVYVKPTLSDGTCKEEFTVNGDPVKKVICTGPNDTSPGSPRHTVPQCCYGFCIDLLIKLARTMNFTYEVHLVADGKFGTQERVNNSNKKEWNGMMGELLSGQADMIVAPLTINNERAQYIEFSKPFKYQGLTILVKKGTRITGINDPRLRNPSDKFIYATVKQSSVDIYFRRQVELSTMYRHMEKHNYESAAEAIQAVRDNKLHAFIWDSAVLEFEASQKCDLVTTGELFFRSGFGIGMRKDSPWKQNVSLSILKSHENGFMEDLDKTWVRYQECDSGSSTMVRSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 7 - protein, 394 aa, Homo sapiens
  Amino terminal domain of GluN1
MSTMRLLTLALLFSCSVARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSFLRTVPPYSHQSSVWFEMMRVYSWNHIILLVSDDHEGRAAQKRLETLLEERESKAEKVLQFDPGTKNVTALLMEAKELEARVIILSASEDDAATVYRAAAMLNMTGSGYVWLVGEREISGNALRYAPDGILGLQLINGKNESAHISDAVGVVAQAVHELLEKENITDPPRGCVGNTNIWKTGPLFKRVLMSSKYADGVTGRVEFNEDGDRKFANYSIMNLQNRKLVQVGIYNGTHVIPNDRKIIWPGGETEKPRGYQM

SEQ ID NO: 8 - protein, 150 aa, Homo sapiens
  Ligand-binding domain of GluN1 S1
STRLKIVTIHQEPFVYVKPTLSDGTCKEEFTVNGDPVKKVICTGPNDTSPGSPRHTVPQCCYGFCIDLLIKLARTMNFTYEVHLVADGKFGTQERVNNSNKKEWNGMMGELLSGQADMIVAPLTINNERAQYIEFSKPFKYQGLTILVKK

SEQ ID NO: 9 - protein, 138 aa, Homo sapiens
  Ligand-binding domain of GluN1 S2
RITGINDPRLRNPSDKFIYATVKQSSVDIYFRRQVELSTMYRHMEKHNYESAAEAIQAVRDNKLHAFIWDSAVLEFEASQKCDLVTTGELFFRSGFGIGMRKDSPWKQNVSLSILKSHENGFMEDLDKTWVRYQECDS

SEQ ID NO: 10 - protein, 137 aa, Homo sapiens
  Ligand-binding domain of GluN1 S2 in NM_007327
RITGINDPRLRNPSDKFIYATVKQSSVDIYFRRQVELSTMYRHMEKHNYESAAEAIQAVRDNKLHAFIWDSAVLEFEASQKCDLVTTGELFFRSGFGIGMRKDSPWKQNVSLSILKSHENGFMEDLDKTWVRYQECD

SEQ ID NO: 11 - protein, 915 aa, Artificial Sequence
  protein seq of fusion protein #3 N2Becd-Fc
MKPRAECCSPKFWLVLAVLAVSGSRARSQKSPPSIGIAVILVGTSDEVAIKDAHEKDDFHHLSVVPRVELVAMNETDPKSIITRICDLMSDRKIQGVVFADDTDQEAIAQILDFISAQTLTPILGIHGGSSMIMADKDESSMFFQFGPSIEQQASVMLNIMEEYDWYIFSIVTTYFPGYQDFVNKIRSTIENSFVGWELEEVLLLDMSLDDGDSKIQNQLKKLQSPIILLYCTKEEATYIFEVANSVGLTGYGYTWIVPSLVAGDTDTVPAEFPTGLISVSYDEWDYGLPARVRDGIAIITTAASDMLSEHSFIPEPKSSCYNTHEKRIYQSNMLNRYLINVTFEGRNLSFSEDGYQMHPKLVIILLNKERKWERVGKWKDKSLQMKYYVWPRMCPETEEQEDDHLSIVTLEEAPFVIVESVDPLSGTCMRNTVPCQKRIVTENKTDEEPGYIKKCCKGFCIDILKKISKSVKFTYDLYLVTNGKHGKKINGTWNGMIGEVVMKRAYMAVGSLTINEERSEVVDFSVPFIETGISVMVSRGTQVSGLSDKKFQRPNDFSPPFRFGTVPNGSTERNIRNNYAEMHAYMGKFNQRGVDDALLSLKTGKLDAFIYDAAVLNYMAGRDEGCKLVTIGSGKVFASTGYGIAIQKDSGWKRQVDLAILQLFGDGEMEELEALWLTGICHNGSSTMVRSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 12 - protein, 401 aa, Homo sapiens
  Amino terminal domain of GluN2B (including signal sequence)
MKPRAECCSPKFWLVLAVLAVSGSRARSQKSPPSIGIAVILVGTSDEVAIKDAHEKDDFHHLSVVPRVELVAMNETDPKSIITRICDLMSDRKIQGVVFADDTDQEAIAQILDFISAQTLTPILGIHGGSSMIMADKDESSMFFQFGPSIEQQASVMLNIMEEYDWYIFSIVTTYFPGYQDFVNKIRSTIENSFVGWELEEVLLLDMSLDDGDSKIQNQLKKLQSPIILLYCTKEEATYIFEVANSVGLTGYGYTWIVPSLVAGDTDTVPAEFPTGLISVSYDEWDYGLPARVRDGIAIITTAASDMLSEHSFIPEPKSSCYNTHEKRIYQSNMLNRYLINVTFEGRNLSFSEDGYQMHPKLVIILLNKERKWERVGKWKDKSLQMKYYVWPRMCPETEEQ

SEQ ID NO: 13 - protein, 139 aa, Homo sapiens
  Ligand-binding domain of GluN2B S1
EDDHLSIVTLEEAPFVIVESVDPLSGTCMRNTVPCQKRIVTENKTDEEPGYIKKCCKGFCIDILKKISKSVKFTYDLYLVTNGKHGKKINGTWNGMIGEVVMKRAYMAVGSLTINEERSEVVDFSVPFIETGISVMVSR

SEQ ID NO: 14 - protein, 142 aa, Homo sapiens
  Ligand-binding domain of GluN2B S2
QVSGLSDKKFQRPNDFSPPFRFGTVPNGSTERNIRNNYAEMHAYMGKFNQRGVDDALLSLKTGKLDAFIYDAAVLNYMAGRDEGCKLVTIGSGKVFASTGYGIAIQKDSGWKRQVDLAILQLFGDGEMEELEALWLTGICHN

SEQ ID NO: 15 - protein, 361 aa, Homo sapiens
  Amino terminal domain of GluN2B in NM_000834
PSIGIAVILVGTSDEVAIKDAHEKDDFHHLSVVPRVELVAMNETDPKSIITRICDLMSDRKIQGVVFADDTDQEAIAQILDFISAQTLTPILGIHGGSSMIMADKDESSMFFQFGPSIEQQASVMLNIMEEYDWYIFSIVTTYFPGYQDFVNKIRSTIENSFVGWELEEVLLLDMSLDDGDSKIQNQLKKLQSPIILLYCTKEEATYIFEVANSVGLTGYGYTWIVPSLVAGDTDTVPAEFPTGLISVSYDEWDYGLPARVRDGIAIITTAASDMLSEHSFIPEPKSSCYNTHEKRIYQSNMLNRYLINVTFEGRNLSFSEDGYQMHPKLVIILLNKERKWERVGKWKDKSLQMKYYVWPR

SEQ ID NO: 16 - protein, 138 aa, Homo sapiens
  Ligand-binding domain of GluN2B S1 in NM_000834
DDHLSIVTLEEAPFVIVESVDPLSGTCMRNTVPCQKRIVTENKTDEEPGYIKKCCKGFCIDILKKISKSVKFTYDLYLVTNGKHGKKINGTWNGMIGEVVMKRAYMAVGSLTINEERSEVVDFSVPFIETGISVMVSR

SEQ ID NO: 17 - protein, 137 aa, Homo sapiens; identical to SEQ ID NO: 10
  Ligand-binding domain of GluN2B S2 in NM_000834

SEQ ID NO: 18 - protein, 1594 aa, Artificial Sequence
  protein seq of fusion protein #4 N1ecd-N2Becd- Fc
MSTMRLLTLALLFSCSVARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSFLRTVPPYSHQSSVWFEMMRVYSWNHIILLVSDDHEGRAAQKRLETLLEERESKAEKVLQFDPGTKNVTALLMEAKELEARVIILSASEDDAATVYRAAAMLNMTGSGYVWLVGEREISGNALRYAPDGILGLQLINGKNESAHISDAVGVVAQAVHELLEKENITDPPRGCVGNTNIWKTGPLFKRVLMSSKYADGVTGRVEFNEDGDRKFANYSIMNLQNRKLVQVGIYNGTHVIPNDRKIIWPGGETEKPRGYQMSTRLKIVTIHQEPFVYVKPTLSDGTCKEEFTVNGDPVKKVICTGPNDTSPGSPRHTVPQCCYGFCIDLLIKLARTMNFTYEVHLVADGKFGTQERVNNSNKKEWNGMMGELLSGQADMIVAPLTINNERAQYIEFSKPFKYQGLTILVKKGTRITGINDPRLRNPSDKFIYATVKQSSVDIYFRRQVELSTMYRHMEKHNYESAAEAIQAVRDNKLHAFIWDSAVLEFEASQKCDLVTTGELFFRSGFGIGMRKDSPWKQNVSLSILKSHENGFMEDLDKTWVRYQECDSGSTGGGGSGGGGSGGGGSGAASRQKSPPSIGIAVILVGTSDEVAIKDAHEKDDFHHLSVVPRVELVAMNETDPKSIITRICDLMSDRKIQGVVFADDTDQEAIAQILDFISAQTLTPILGIHGGSSMIMADKDESSMFFQFGPSIEQQASVMLNIMEEYDWYIFSIVTTYFPGYQDFVNKIRSTIENSFVGWELEEVLLLDMSLDDGDSKIQNQLKKLQSPIILLYCTKEEATYIFEVANSVGLTGYGYTWIVPSLVAGDTDTVPAEFPTGLISVSYDEWDYGLPARVRDGIAIITTAASDMLSEHSFIPEPKSSCYNTHEKRIYQSNMLNRYLINVTFEGRNLSFSEDGYQMHPKLVIILLNKERKWERVGKWKDKSLQMKYYVWPRMCPETEEQEDDHLSIVTLEEAPFVIVESVDPLSGTCMRNTVPCQKRIVTENKTDEEPGYIKKCCKGFCIDILKKISKSVKFTYDLYLVTNGKHGKKINGTWNGMIGEVVMKRAYMAVGSLTINEERSEVVDFSVPFIETGISVMVSRGTQVSGLSDKKFQRPNDFSPPFRFGTVPNGSTERNIRNNYAEMHAYMGKFNQRGVDDALLSLKTGKLDAFIYDAAVLNYMAGRDEGCKLVTIGSGKVFASTGYGIAIQKDSGWKRQVDLAILQLFGDGEMEELEALWLTGICHNGSSTMVRSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 19 - protein, 23 aa, Artificial Sequence
  Linker between a GluN1-domain and GluN2B-domain in a fusion protein of NMDAR construct of the invention
GSTGGGGSGGGGSGGGGSGAASR

SEQ ID NO: 20 - protein, 373 aa, Homo sapiens
  Amino terminal domain of GluN2B
QKSPPSIGIAVILVGTSDEVAIKDAHEKDDFHHLSVVPRVELVAMNETDPKSIITRICDLMSDRKIQGVVFADDTDQEAIAQILDFISAQTLTPILGIHGGSSMIMADKDESSMFFQFGPSIEQQASVMLNIMEEYDWYIFSIVTTYFPGYQDFVNKIRSTIENSFVGWELEEVLLLDMSLDDGDSKIQNQLKKLQSPIILLYCTKEEATYIFEVANSVGLTGYGYTWIVPSLVAGDTDTVPAEFPTGLISVSYDEWDYGLPARVRDGIAIITTAASDMLSEHSFIPEPKSSCYNTHEKRIYQSNMLNRYLINVTFEGRNLSFSEDGYQMHPKLVIILLNKERKWERVGKWKDKSLQMKYYVWPRMCPETEEQ

SEQ ID NO: 21 - protein, 639 aa, Artificial Sequence
  protein seq of fusion protein #5 N2A-ATD-Fc
MGRVGYWTLLVLPALLVWRGPAPSAAAEKGPPALNIAVMLGHSHDVTERELRTLWGPEQAAGLPLDVNVVALLMNRTDPKSLITHVCDLMSGARIHGLVFGDDTDQEAVAQMLDFISSHTFVPILGIHGGASMIMADKDPTSTFFQFGASIQQQATVMLKIMQDYDWHVFSLVTTIFPGYREFISFVKTTVDNSFVGWDMQNVITLDTSFEDAKTQVQLKKIHSSVILLYCSKDEAVLILSEARSLGLTGYDFFWIVPSLVSGNTELIPKEFPSGLISVSYDDWDYSLEARVRDGIGILTTAASSMLEKFSYIPEAKASCYGQMERPEVPMHTLHPFMVNVTWDGKDLSFTEEGYQVHPRLVVIVLNKDREWEKVGKWENHTLSLRHAVWPRYKSFSDCEPDDNHLSIGSSTMVRSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 22 - protein, 408 aa, Homo sapiens
  Amino terminal domain of GluN2A (including signal sequence)
MGRVGYWTLLVLPALLVWRGPAPSAAAEKGPPALNIAVMLGHSHDVTERELRTLWGPEQAAGLPLDVNVVALLMNRTDPKSLITHVCDLMSGARIHGLVFGDDTDQEAVAQMLDFISSHTFVPILGIHGGASMIMADKDPTSTFFQFGASIQQQATVMLKIMQDYDWHVFSLVTTIFPGYREFISFVKTTVDNSFVGWDMQNVITLDTSFEDAKTQVQLKKIHSSVILLYCSKDEAVLILSEARSLGLTGYDFFWIVPSLVSGNTELIPKEFPSGLISVSYDDWDYSLEARVRDGIGILTTAASSMLEKFSYIPEAKASCYGQMERPEVPMHTLHPFMVNVTWDGKDLSFTEEGYQVHPRLVVIVLNKDREWEKVGKWENHTLSLRHAVWPRYKSFSDCEPDDNHLSI

SEQ ID NO: 23 - protein, 361 aa, Homo sapiens
  Amino terminal domain of GluN2A in NM_001134407
PALNIAVMLGHSHDVTERELRTLWGPEQAAGLPLDVNVVALLMNRTDPKSLITHVCDLMSGARIHGLVFGDDTDQEAVAQMLDFISSHTFVPILGIHGGASMIMADKDPTSTFFQFGASIQQQATVMLKIMQDYDWHVFSLVTTIFPGYREFISFVKTTVDNSFVGWDMQNVITLDTSFEDAKTQVQLKKIHSSVILLYCSKDEAVLILSEARSLGLTGYDFFWIVPSLVSGNTELIPKEFPSGLISVSYDDWDYSLEARVRDGIGILTTAASSMLEKFSYIPEAKASCYGQMERPEVPMHTLHPFMVNVTWDGKDLSFTEEGYQVHPRLVVIVLNKDREWEKVGKWENHTLSLRHAVWPR

SEQ ID NO: 24 - protein, 639 aa, Artificial Sequence
  protein seq of fusion protein #6 N2B-ATD-Fc
MKPRAECCSPKFWLVLAVLAVSGSRARSQKSPPSIGIAVILVGTSDEVAIKDAHEKDDFHHLSVVPRVELVAMNETDPKSIITRICDLMSDRKIQGVVFADDTDQEAIAQILDFISAQTLTPILGIHGGSSMIMADKDESSMFFQFGPSIEQQASVMLNIMEEYDWYIFSIVTTYFPGYQDFVNKIRSTIENSFVGWELEEVLLLDMSLDDGDSKIQNQLKKLQSPIILLYCTKEEATYIFEVANSVGLTGYGYTWIVPSLVAGDTDTVPAEFPTGLISVSYDEWDYGLPARVRDGIAIITTAASDMLSEHSFIPEPKSSCYNTHEKRIYQSNMLNRYLINVTFEGRNLSFSEDGYQMHPKLVIILLNKERKWERVGKWKDKSLQMKYYVWPRMCPETEEQEDDHLSIGSSTMVRSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 25 - protein, 408 aa, Homo sapiens
  Amino terminal domain of GluN2B (including signal sequence)
MKPRAECCSPKFWLVLAVLAVSGSRARSQKSPPSIGIAVILVGTSDEVAIKDAHEKDDFHHLSVVPRVELVAMNETDPKSIITRICDLMSDRKIQGVVFADDTDQEAIAQILDFISAQTLTPILGIHGGSSMIMADKDESSMFFQFGPSIEQQASVMLNIMEEYDWYIFSIVTTYFPGYQDFVNKIRSTIENSFVGWELEEVLLLDMSLDDGDSKIQNQLKKLQSPIILLYCTKEEATYIFEVANSVGLTGYGYTWIVPSLVAGDTDTVPAEFPTGLISVSYDEWDYGLPARVRDGIAIITTAASDMLSEHSFIPEPKSSCYNTHEKRIYQSNMLNRYLINVTFEGRNLSFSEDGYQMHPKLVIILLNKERKWERVGKWKDKSLQMKYYVWPRMCPETEEQEDDHLSI

SEQ ID NO: 26 - protein, 1035 aa, Artificial Sequence
  protein seq of fusion protein #7 N1-ATD-N2A- ATD-Fc
MSTMRLLTLALLFSCSVARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSFLRTVPPYSHQSSVWFEMMRVYSWNHIILLVSDDHEGRAAQKRLETLLEERESKAEKVLQFDPGTKNVTALLMEAKELEARVIILSASEDDAATVYRAAAMLNMTGSGYVWLVGEREISGNALRYAPDGILGLQLINGKNESAHISDAVGVVAQAVHELLEKENITDPPRGCVGNTNIWKTGPLFKRVLMSSKYADGVTGRVEFNEDGDRKFANYSIMNLQNRKLVQVGIYNGTHVIPNDRKIIWPGGETEKPRGYQMSTRLKIGSTGGGGSGGGGSGGGGSGAASREKGPPALNIAVMLGHSHDVTERELRTLWGPEQAAGLPLDVNVVALLMNRTDPKSLITHVCDLMSGARIHGLVFGDDTDQEAVAQMLDFISSHTFVPILGIHGGASMIMADKDPTSTFFQFGASIQQQATVMLKIMQDYDWHVFSLVTTIFPGYREFISFVKTTVDNSFVGWDMQNVITLDTSFEDAKTQVQLKKIHSSVILLYCSKDEAVLILSEARSLGLTGYDFFWIVPSLVSGNTELIPKEFPSGLISVSYDDWDYSLEARVRDGIGILTTAASSMLEKFSYIPEAKASCYGQMERPEVPMHTLHPFMVNVTWDGKDLSFTEEGYQVHPRLVVIVLNKDREWEKVGKWENHTLSLRHAVWPRYKSFSDCEPDDNHLSIGSSTMVRSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 27 - protein, 400 aa, Homo sapiens; identical to SEQ ID NO: 2
  Amino terminal domain of GluN1 (including signal sequence)

SEQ ID NO: 28 - protein, 381 aa, Homo sapiens
  Amino terminal domain of GluN2A
EKGPPALNIAVMLGHSHDVTERELRTLWGPEQAAGLPLDVNVVALLMNRTDPKSLITHVCDLMSGARIHGLVFGDDTDQEAVAQMLDFISSHTFVPILGIHGGASMIMADKDPTSTFFQFGASIQQQATVMLKIMQDYDWHVFSLVTTIFPGYREFISFVKTTVDNSFVGWDMQNVITLDTSFEDAKTQVQLKKIHSSVILLYCSKDEAVLILSEARSLGLTGYDFFWIVPSLVSGNTELIPKEFPSGLISVSYDDWDYSLEARVRDGIGILTTAASSMLEKFSYIPEAKASCYGQMERPEVPMHTLHPFMVNVTWDGKDLSFTEEGYQVHPRLVVIVLNKDREWEKVGKWENHTLSLRHAVWPRYKSFSDCEPDDNHLSI

SEQ ID NO: 29 - protein, 1034 aa, Artificial Sequence
  protein seq of fusion protein #8 N1-ATD-N2B- ATD-Fc
MSTMRLLTLALLFSCSVARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSFLRTVPPYSHQSSVWFEMMRVYSWNHIILLVSDDHEGRAAQKRLETLLEERESKAEKVLQFDPGTKNVTALLMEAKELEARVIILSASEDDAATVYRAAAMLNMTGSGYVWLVGEREISGNALRYAPDGILGLQLINGKNESAHISDAVGVVAQAVHELLEKENITDPPRGCVGNTNIWKTGPLFKRVLMSSKYADGVTGRVEFNEDGDRKFANYSIMNLQNRKLVQVGIYNGTHVIPNDRKIIWPGGETEKPRGYQMSTRLKIGSTGGGGSGGGGSGGGGSGAASRQKSPPSIGIAVILVGTSDEVAIKDAHEKDDFHHLSVVPRVELVAMNETDPKSIITRICDLMSDRKIQGVVFADDTDQEAIAQILDFISAQTLTPILGIHGGSSMIMADKDESSMFFQFGPSIEQQASVMLNIMEEYDWYIFSIVTTYFPGYQDFVNKIRSTIENSFVGWELEEVLLLDMSLDDGDSKIQNQLKKLQSPIILLYCTKEEATYIFEVANSVGLTGYGYTWIVPSLVAGDTDTVPAEFPTGLISVSYDEWDYGLPARVRDGIAIITTAASDMLSEHSFIPEPKSSCYNTHEKRIYQSNMLNRYLINVTFEGRNLSFSEDGYQMHPKLVIILLNKERKWERVGKWKDKSLQMKYYVWPRMCPETEEQEDDHLSIGSSTMVRSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 30 - protein, 400 aa, Homo sapiens; identical to SEQ ID NO: 2
  Amino terminal domain of GluN1 (including signal sequence)

SEQ ID NO: 31 - protein, 380 aa, Homo sapiens
  Amino terminal domain of GluN2B
QKSPPSIGIAVILVGTSDEVAIKDAHEKDDFHHLSVVPRVELVAMNETDPKSIITRICDLMSDRKIQGVVFADDTDQEAIAQILDFISAQTLTPILGIHGGSSMIMADKDESSMFFQFGPSIEQQASVMLNIMEEYDWYIFSIVTTYFPGYQDFVNKIRSTIENSFVGWELEEVLLLDMSLDDGDSKIQNQLKKLQSPIILLYCTKEEATYIFEVANSVGLTGYGYTWIVPSLVAGDTDTVPAEFPTGLISVSYDEWDYGLPARVRDGIAIITTAASDMLSEHSFIPEPKSSCYNTHEKRIYQSNMLNRYLINVTFEGRNLSFSEDGYQMHPKLVIILLNKERKWERVGKWKDKSLQMKYYVWPRMCPETEEQEDDHLSI

SEQ ID NO: 32 - protein, 1233 aa, Homo sapiens
  Human GluN2C NM_000835
MGGALGPALLLTSLFGAWAGLGPGQGEQGMTVAVVFSSSGPPQAQFRARLTPQSFLDLPLEIQPLTVGVNTTNPSSLLTQICGLLGAAHVHGIVFEDNVDTEAVAQILDFISSQTHVPILSISGGSAVVLTPKEPGSAFLQLGVSLEQQLQVLFKVLEEYDWSAFAVITSLHPGHALFLEGVRAVADASHVSWRLLDVVTLELGPGGPRARTQRLLRQLDAPVFVAYCSREEAEVLFAEAAQAGLVGPGHVWLVPNLALGSTDAPPATFPVGLISVVTESWRLSLRQKVRDGVAILALGAHSYWRQHGTLPAPAGDCRVHPGPVSPAREAFYRHLLNVTWEGRDFSFSPGGYLVQPTMVVIALNRHRLWEMVGRWEHGVLYMKYPVWPRYSASLQPVVDSRHLTVATLEERPFVIVESPDPGTGGCVPNTVPCRRQSNHTFSSGDVAPYTKLCCKGFCIDILKKLARVVKFSYDLYLVTNGKHGKRVRGVWNGMIGEVYYKRADMAIGSLTINEERSEIVDFSVPFVETGISVMVARSNGTVSPSAFLEPYSPAVWVMMFVMCLTVVAITVFMFEYFSPVSYNQNLTRGKKSGGPAFTIGKSVWLLWALVFNNSVPIENPRGTTSKIMVLVWAFFAVIFLASYTANLAAFMIQEQYIDTVSGLSDKKFQRPQDQYPPFRFGTVPNGSTERNIRSNYRDMHTHMVKFNQRSVEDALTSLKMGKLDAFIYDAAVLNYMAGKDEGCKLVTIGSGKVFATTGYGIAMQKDSHWKRAIDLALLQFLGDGETQKLETVWLSGICQNEKNEVMSSKLDIDNMAGVFYMLLVAMGLALLVFAWEHLVYWKLRHSVPNSSQLDFLLAFSRGIYSCFSGVQSLASPPRQASPDLTASSAQASVLKMLQAARDMVTTAGVSSSLDRATRTIENWGGGRRAPPPSPCPTPRSGPSPCLPTPDPPPEPSPTGWGPPDGGRAALVRRAPQPPGRPPTPGPPLSDVSRVSRRPAWEARWPVRTGHCGRHLSASERPLSPARCHYSSFPRADRSGRPFLPLFPELEDLPLLGPEQLARREALLHAAWARGSRPRHASLPSSVAEAFARPSSLPAGCTGPACARPDGHSACRRLAQAQSMCLPIYREACQEGEQAGAPAWQHRQHVCLHAHAHLPFCWGAVCPHLPPCASHGSWLSGAWGPLGHRGRTLGLGTGYRDSGGLDEISRVARGTQGFPGPCTWRRISSLESEV

SEQ ID NO: 33 - protein, 362 aa, Homo sapiens
  GluN2C ATD
QGMTVAVVFSSSGPPQAQFRARLTPQSFLDLPLEIQPLTVGVNTTNPSSLLTQICGLLGAAHVHGIVFEDNVDTEAVAQILDFISSQTHVPILSISGGSAVVLTPKEPGSAFLQLGVSLEQQLQVLFKVLEEYDWSAFAVITSLHPGHALFLEGVRAVADASHVSWRLLDVVTLELGPGGPRARTQRLLRQLDAPVFVAYCSREEAEVLFAEAAQAGLVGPGHVWLVPNLALGSTDAPPATFPVGLISVVTESWRLSLRQKVRDGVAILALGAHSYWRQHGTLPAPAGDCRVHPGPVSPAREAFYRHLLNVTWEGRDFSFSPGGYLVQPTMVVIALNRHRLWEMVGRWEHGVLYMKYPVWPR

SEQ ID NO: 34 - protein, 138 aa, Homo sapiens
  GluN2C S1
SRHLTVATLEERPFVIVESPDPGTGGCVPNTVPCRRQSNHTFSSGDVAPYTKLCCKGFCIDILKKLARVVKFSYDLYLVTNGKHGKRVRGVWNGMIGEVYYKRADMAIGSLTINEERSEIVDFSVPFVETGISVMVAR

SEQ ID NO: 35 - protein, 142 aa, Homo sapiens
  GluN2C S2
TVSGLSDKKFQRPQDQYPPFRFGTVPNGSTERNIRSNYRDMHTHMVKFNQRSVEDALTSLKMGKLDAFIYDAAVLNYMAGKDEGCKLVTIGSGKVFATTGYGIAMQKDSHWKRAIDLALLQFLGDGETQKLETVWLSGICQN

SEQ ID NO: 36 - protein, 681 aa, Artificial Sequence
  GluN2C ecd incl. signal sequence and GT-linker between S1 and S2
MGGALGPALLLTSLFGAWAGLGPGQGEQGMTVAVVFSSSGPPQAQFRARLTPQSFLDLPLEIQPLTVGVNTTNPSSLLTQICGLLGAAHVHGIVFEDNVDTEAVAQILDFISSQTHVPILSISGGSAVVLTPKEPGSAFLQLGVSLEQQLQVLFKVLEEYDWSAFAVITSLHPGHALFLEGVRAVADASHVSWRLLDVVTLELGPGGPRARTQRLLRQLDAPVFVAYCSREEAEVLFAEAAQAGLVGPGHVWLVPNLALGSTDAPPATFPVGLISVVTESWRLSLRQKVRDGVAILALGAHSYWRQHGTLPAPAGDCRVHPGPVSPAREAFYRHLLNVTWEGRDFSFSPGGYLVQPTMVVIALNRHRLWEMVGRWEHGVLYMKYPVWPRYSASLQPVVDSRHLTVATLEERPFVIVESPDPGTGGCVPNTVPCRRQSNHTFSSGDVAPYTKLCCKGFCIDILKKLARVVKFSYDLYLVTNGKHGKRVRGVWNGMIGEVYYKRADMAIGSLTINEERSEIVDFSVPFVETGISVMVARGTTVSGLSDKKFQRPQDQYPPFRFGTVPNGSTERNIRSNYRDMHTHMVKFNQRSVEDALTSLKMGKLDAFIYDAAVLNYMAGKDEGCKLVTIGSGKVFATTGYGIAMQKDSHWKRAIDLALLQFLGDGETQKLETVWLSGICQN

SEQ ID NO: 37 - protein, 1336 aa, Homo sapiens
  Human GluN2D NP_000827
MRGAGGPRGPRGPAKMLLLLALACASPFPEEAPGPGGAGGPGGGLGGARPLNVALVFSGPAYAAEAARLGPAVAAAVRSPGLDVRPVALVLNGSDPRSLVLQLCDLLSGLRVHGVVFEDDSRAPAVAPILDFLSAQTSLPIVAVHGGAALVLTPKEKGSTFLQLGSSTEQQLQVIFEVLEEYDWTSFVAVTTRAPGHRAFLSYIEVLTDGSLVGWEHRGALTLDPGAGEAVLSAQLRSVSAQIRLLFCAREEAEPVFRAAEEAGLTGSGYVWFMVGPQLAGGGGSGAPGEPPLLPGGAPLPAGLFAVRSAGWRDDLARRVAAGVAVVARGAQALLRDYGFLPELGHDCRAQNRTHRGESLHRYFMNITWDNRDYSFNEDGFLVNPSLVVISLTRDRTWEVVGSWEQQTLRLKYPLWSRYGRFLQPVDDTQHLTVATLEERPFVIVEPADPISGTCIRDSVPCRSQLNRTHSPPPDAPRPEKRCCKGFCIDILKRLAHTIGFSYDLYLVTNGKHGKKIDGVWNGMIGEVFYQRADMAIGSLTINEERSEIVDFSVPFVETGISVMVARSNGTVSPSAFLEPYSPAVWVMMFVMCLTVVAVTVFIFEYLSPVGYNRSLATGKRPGGSTFTIGKSIWLLWALVFNNSVPVENPRGTTSKIMVLVWAFFAVIFLASYTANLAAFMIQEEYVDTVSGLSDRKFQRPQEQYPPLKFGTVPNGSTEKNIRSNYPDMHSYMVRYNQPRVEEALTQLKAGKLDAFIYDAAVLNYMARKDEGCKLVTIGSGKVFATTGYGIALHKGSRWKRPIDLALLQFLGDDEIEMLERLWLSGICHNDKIEVMSSKLDIDNMAGVFYMLLVAMGLSLLVFAWEHLVYWRLRHCLGPTHRMDFLLAFSRGMYSCCSAEAAPPPAKPPPPPQPLPSPAYPAPRPAPGPAPFVPRERASVDRWRRTKGAGPPGGAGLADGFHRYYGPIEPQGLGLGLGEARAAPRGAAGRPLSPPAAQPPQKPPPSYFAIVRDKEPAEPPAGAFPGFPSPPAPPAAAATAVGPPLCRLAFEDESPPAPARWPRSDPESQPLLGPGAGGAGGTGGAGGGAPAAPPPCRAAPPPCPYLDLEPSPSDSEDSESLGGASLGGLEPWWFADFPYPYAERLGPPPGRYWSVDKLGGWRAGSWDYLPPRSGPAAWHCRHCASLELLPPPRHLSCSHDGLDGGWWAPPPPPWAAGPLPRRRARCGCPRSHPHRPRASHRTPAAAAPHHHRHRRAAGGWDLPPPAPTSRSLEDLSSCPRAAPARRLTGPSRHARRCPHAAHWGPPLPTASHRRHRGGDLGTRRGSAHFSSLESEV

SEQ ID NO: 38 - protein, 1336 aa, Homo sapiens
  Human GluN2D Protein seq U77783
MRGAGGPRGPRGPAKMLLLLALACASPFPEEAPGPGGAGGPGGGLGGARPLNVALVFSGPAYAAEAARLGPAVAAAVRSPGLDVRPVALVLNGSDPRSLVLQLCDLLSGLRVHGVVFEDDSRAPAVAPILDFLSAQTSLPIVAVHGGAALVLTPKEKGSTFLQLGSSTEQQLQVIFEVLEEYDWTSFVAVTTRAPGHRAFLSYIEVLTDGSLVGWEHRGALTLDPGAGEAVLSAQLRSVSAQIRLLFCAREEAEPVFRAAEEAGLTGSGYVWFMVGPQLAGGGGSGAPGEPPLLPGGAPLPAGLFAVRSAGWRDDLARRVAAGVAVVARGAQALLRDYGFLPELGHDCRAQNRTHRGESLHRYFMNITWDNRDYSFNEDGFLVNPSLVVISLTRDRTWEVVGSWEQQTLRLKYPLWSRYGRFLQPVDDTQHLTVATLEERPFVIVEPADPISGTCIRDSVPCRSQLNRTHSPPPDAPRPEKRCCKGFCIDILKRLAHTIGFSYDLYLVTNGKHGKKIDGVWNGMIGEVFYQRADMAIGSLTINEERSEIVDFSVPFVETGISVMVARSNGTVSPSAFLEPYSPAVWVMMFVMCLTVVAVTVFIFEYLSPVGYNRSLATGKRPGGSTFTIGKSIWLLWALVFNNSVPVENPRGTTSKIMVLVWAFFAVIFLASYTANLAAFMIQEEYVDTVSGLSDRKFQRPQEQYPPLKFGTVPNGSTEKNIRSNYPDMHSYMVRYNQPRVEEALTQLKAGKLDAFIYDAAVLNYMARKDEGCKLVTIGSGKVFATTGYGIALHKGSRWKRPIDLALLQFLGDDEIEMLERLWLSGICHNDKIEVMSSKLDIDNMAGVFYMLLVAMGLSLLVFAWEHLVYWRLRHCLGPTHRMDFLLAFSRGMYSCCSAEAAPPPAKPPPPPQPLPSPAYPAPGPAPGPAPFVPRERASVDRWRRTKGAGPPGGAGLADGFHRYYGPIEPQGLGLGLGEARAAPRGAAGRPLSPPAAQPPQKPPASYFAIVRDKEPAEPPAGAFPGFPSPPAPPAAAATAVGPPLCRLAFEDESPPAPARWPRSDPESQPLLGPGAGGAGGTGGAGGGAPAAPPPCCAAPPPCPYLDLEPSPSDSEDSESLGGASLGGLDPWWFADFPYPYAERLGPPPGRYWSVDKLGGWRAGSWDYLPPRSGPAAWHCRHCASLELLPPPRHLSCSHDGLDGGWWAPPPPPWAAGPLPRRRARCGCPRSHPHRPRASHRTPAAAAPHHHRHRRAAGGWDLPPPAPTSRSLEDLSSCPRAAPARRLTGPSRHARRCPHAAHWGPPLPTASHRRHRGGDLGTRRGSAHFSSLESEV

SEQ ID NO: 39 - protein, 370 aa, Homo sapiens
  GluN2D ATD
RPLNVALVFSGPAYAAEAARLGPAVAAAVRSPGLDVRPVALVLNGSDPRSLVLQLCDLLSGLRVHGVVFEDDSRAPAVAPILDFLSAQTSLPIVAVHGGAALVLTPKEKGSTFLQLGSSTEQQLQVIFEVLEEYDWTSFVAVTTRAPGHRAFLSYIEVLTDGSLVGWEHRGALTLDPGAGEAVLSAQLRSVSAQIRLLFCAREEAEPVFRAAEEAGLTGSGYVWFMVGPQLAGGGGSGAPGEPPLLPGGAPLPAGLFAVRSAGWRDDLARRVAAGVAVVARGAQALLRDYGFLPELGHDCRAQNRTHRGESLHRYFMNITWDNRDYSFNEDGFLVNPSLVVISLTRDRTWEVVGSWEQQTLRLKYPLWSR

SEQ ID NO: 40 - protein, 139 aa, Homo sapiens
  GluN2D S1
TQHLTVATLEERPFVIVEPADPISGTCIRDSVPCRSQLNRTHSPPPDAPRPEKRCCKGFCIDILKRLAHTIGFSYDLYLVTNGKHGKKIDGVWNGMIGEVFYQRADMAIGSLTINEERSEIVDFSVPFVETGISVMVAR

SEQ ID NO: 41 - protein, 142 aa, Homo sapiens
  GluN2D S2
TVSGLSDRKFQRPQEQYPPLKFGTVPNGSTEKNIRSNYPDMHSYMVRYNQPRVEEALTQLKAGKLDAFIYDAAVLNYMARKDEGCKLVTIGSGKVFATTGYGIALHKGSRWKRPIDLALLQFLGDDEIEMLERLWLSGICHN

SEQ ID NO: 42 - protein, 711 aa, Artificial Sequence
  GluN2D ecd incl. signal sequence and GT-linker between S1 and S2
MRGAGGPRGPRGPAKMLLLLALACASPFPEEAPGPGGAGGPGGGLGGARPLNVALVFSGPAYAAEAARLGPAVAAAVRSPGLDVRPVALVLNGSDPRSLVLQLCDLLSGLRVHGVVFEDDSRAPAVAPILDFLSAQTSLPIVAVHGGAALVLTPKEKGSTFLQLGSSTEQQLQVIFEVLEEYDWTSFVAVTTRAPGHRAFLSYIEVLTDGSLVGWEHRGALTLDPGAGEAVLSAQLRSVSAQIRLLFCAREEAEPVFRAAEEAGLTGSGYVWFMVGPQLAGGGGSGAPGEPPLLPGGAPLPAGLFAVRSAGWRDDLARRVAAGVAVVARGAQALLRDYGFLPELGHDCRAQNRTHRGESLHRYFMNITWDNRDYSFNEDGFLVNPSLVVISLTRDRTWEVVGSWEQQTLRLKYPLWSRYGRFLQPVDDTQHLTVATLEERPFVIVEPADPISGTCIRDSVPCRSQLNRTHSPPPDAPRPEKRCCKGFCIDILKRLAHTIGFSYDLYLVTNGKHGKKIDGVWNGMIGEVFYQRADMAIGSLTINEERSEIVDFSVPFVETGISVMVARGTTVSGLSDRKFQRPQEQYPPLKFGTVPNGSTEKNIRSNYPDMHSYMVRYNQPRVEEALTQLKAGKLDAFIYDAAVLNYMARKDEGCKLVTIGSGKVFATTGYGIALHKGSRWKRPIDLALLQFLGDDEIEMLERLWLSGICHN

SEQ ID NO: 43 - protein, 1464 aa, Homo sapiens [sequence omitted]

SEQ ID NO: 44 - protein, 137 aa, Homo sapiens
  GluN2A S1
DNHLSIVTLEEAPFVIVEDIDPLTETCVRNTVPCRKFVKINNSTNEGMNVKKCCKGFCIDILKKLSRTVKFTYDLYLVTNGKHGKKVNNVWNGMIGEVVYQRAVMAVGSLTINEERSEVVDFSVPFVETGISVMVSR

SEQ ID NO: 45 - protein, 142 aa, Homo sapiens
  GluN2A S2
QVTGLSDKKFQRPHDYSPPFRFGTVPNGSTERNIRNNYPYMHQYMTKFNQKGVEDALVSLKTGKLDAFIYDAAVLNYKAGRDEGCKLVTIGSGYIFATTGYGIALQKGSPWKRQIDLALLQFVGDGEMEELETLWLTGICHN

SEQ ID NO: 46 - protein, 683 aa, Artificial Sequence
  GluN2A ecd incl. signal sequence and GT-linker between S1 and S2
MGRVGYWTLLVLPALLVWRGPAPSAAAEKGPPALNIAVMLGHSHDVTERELRTLWGPEQAAGLPLDVNVVALLMNRTDPKSLITHVCDLMSGARIHGLVFGDDTDQEAVAQMLDFISSHTFVPILGIHGGASMIMADKDPTSTFFQFGASIQQQATVMLKIMQDYDWHVFSLVTTIFPGYREFISFVKTTVDNSFVGWDMQNVITLDTSFEDAKTQVQLKKIHSSVILLYCSKDEAVLILSEARSLGLTGYDFFWIVPSLVSGNTELIPKEFPSGLISVSYDDWDYSLEARVRDGIGILTTAASSMLEKFSYIPEAKASCYGQMERPEVPMHTLHPFMVNVTWDGKDLSFTEEGYQVHPRLVVIVLNKDREWEKVGKWENHTLSLRHAVWPRYKSFSDCEPDDNHLSIVTLEEAPFVIVEDIDPLTETCVRNTVPCRKFVKINNSTNEGMNVKKCCKGFCIDILKKLSRTVKFTYDLYLVTNGKHGKKVNNVWNGMIGEVVYQRAVMAVGSLTINEERSEVVDFSVPFVETGISVMVSRGTQVTGLSDKKFQRPHDYSPPFRFGTVPNGSTERNIRNNYPYMHQYMTKFNQKGVEDALVSLKTGKLDAFIYDAAVLNYKAGRDEGCKLVTIGSGYIFATTGYGIALQKGSPWKRQIDLALLQFVGDGEMEELETLWLTGICHN

SEQ ID NO: 47 - protein, 1484 aa, Homo sapiens [sequence omitted]

SEQ ID NO: 48 - protein, 684 aa, Artificial Sequence
  GluN2B ecd incl. signal sequence and GT-linker between S1 and S2
MKPRAECCSPKFWLVLAVLAVSGSRARSQKSPPSIGIAVILVGTSDEVAIKDAHEKDDFHHLSVVPRVELVAMNETDPKSIITRICDLMSDRKIQGVVFADDTDQEAIAQILDFISAQTLTPILGIHGGSSMIMADKDESSMFFQFGPSIEQQASVMLNIMEEYDWYIFSIVTTYFPGYQDFVNKIRSTIENSFVGWELEEVLLLDMSLDDGDSKIQNQLKKLQSPIILLYCTKEEATYIFEVANSVGLTGYGYTWIVPSLVAGDTDTVPAEFPTGLISVSYDEWDYGLPARVRDGIAIITTAASDMLSEHSFIPEPKSSCYNTHEKRIYQSNMLNRYLINVTFEGRNLSFSEDGYQMHPKLVIILLNKERKWERVGKWKDKSLQMKYYVWPRMCPETEEQEDDHLSIVTLEEAPFVIVESVDPLSGTCMRNTVPCQKRIVTENKTDEEPGYIKKCCKGFCIDILKKISKSVKFTYDLYLVTNGKHGKKINGTWNGMIGEVVMKRAYMAVGSLTINEERSEVVDFSVPFIETGISVMVSRGTQVSGLSDKKFQRPNDFSPPFRFGTVPNGSTERNIRNNYAEMHAYMGKFNQRGVDDALLSLKTGKLDAFIYDAAVLNYMAGRDEGCKLVTIGSGKVFASTGYGIAIQKDSGWKRQVDLAILQLFGDGEMEELEALWLTGICHN

SEQ ID NO: 49 - DNA, 1896 bp, Artificial Sequence
  DNA sequence of fusion protein #1 N1-ATD-Fc
atgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccggacggcatcctcgggctgcagctcatcaacggcaagaacgagtcggcccacatcagcgacgccgtaggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagattgggtcgagcaccatggttagatctagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 50 - DNA, 1200 bp, Homo sapiens
  Amino terminal domain (ATD) of GluN1 including the signal sequence
atgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccggacggcatcctcgggctgcagctcatcaacggcaagaacgagtcggcccacatcagcgacgccgtaggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagatt

SEQ ID NO: 51 - DNA, 24 bp, Homo sapiens
  Linker between NMDAR domain and rabbit Fc to be used in fusion proteins of NMDAR protein constructs of invention
gggtcgagcaccatggttagatct

SEQ ID NO: 52 - DNA, 672 bp, Artificial Sequence
  Rabbit Fc from Vector pFuse-rIgG-Fc1
agcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 53 - DNA, 1128 bp, Homo sapiens
  Amino terminal domain of GluN1 in NM_007327
acgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccagacggcatcctcgggctgcagctcatcaacggcaagaacgagtcggcccacatcagcgacgccgtgggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggc

SEQ ID NO: 54 - DNA, 2748 bp, Artificial Sequence
  DNA seq of fusion protein #2 N1ecd-Fc
atgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccggacggcatcctcgggctgcagctcatcaacggcaagaacgagtcggcccacatcagcgacgccgtaggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagattgtgacgatccaccaggagcccttcgtgtacgtcaagcccacgctgagtgatgggacatgcaaggaggagttcacagtcaacggcgacccagtcaagaaggtgatctgcaccgggcccaacgacacgtcgccgggcagcccccgccacacggtgcctcagtgttgctacggcttttgcatcgacctgctcatcaagctggcacggaccatgaacttcacctacgaggtgcacctggtggcagatggcaagttcggcacacaggagcgggtgaacaacagcaacaagaaggagtggaatgggatgatgggcgagctgctcagcgggcaggcagacatgatcgtggcgccgctaaccataaacaacgagcgcgcgcagtacatcgagttttccaagcccttcaagtaccagggcctgactattctggtcaagaagggcacccgcatcacgggcatcaacgacccccggctgaggaacccctcggacaagtttatctacgccacggtgaagcagagctccgtggatatctacttccggcgccaggtggagctgagcaccatgtaccggcatatggagaagcacaactacgagagtgcggcggaggccatccaggccgtgagagacaacaagctgcatgccttcatctgggactcggcggtgctggagttcgaggcctcgcagaagtgcgacctggtgacgactggagagctgtttttccgctcgggcttcggcataggcatgcgcaaagacagcccctggaagcagaacgtctccctgtccatcctcaagtcccacgagaatggcttcatggaagacctggacaagacgtgggttcggtatcaggaatgtgactcggggtcgagcaccatggttagatctagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 55 - DNA, 1200 bp, Homo sapiens; identical to SEQ ID NO: 50
  Amino terminal domain of GluN1 (including signal sequence)

SEQ ID NO: 56 - DNA, 450 bp, Homo sapiens
  Ligand-binding domain of GluN1 S1
tccaccagactgaagattgtgacgatccaccaggagcccttcgtgtacgtcaagcccacgctgagtgatgggacatgcaaggaggagttcacagtcaacggcgacccagtcaagaaggtgatctgcaccgggcccaacgacacgtcgccgggcagcccccgccacacggtgcctcagtgttgctacggcttttgcatcgacctgctcatcaagctggcacggaccatgaacttcacctacgaggtgcacctggtggcagatggcaagttcggcacacaggagcgggtgaacaacagcaacaagaaggagtggaatgggatgatgggcgagctgctcagcgggcaggcagacatgatcgtggcgccgctaaccataaacaacgagcgcgcgcagtacatcgagttttccaagcccttcaagtaccagggcctgactattctggtcaagaag

SEQ ID NO: 57 - DNA, 414 bp, Homo sapiens
  Ligand-binding domain of GluN1 S2
cgcatcacgggcatcaacgacccccggctgaggaacccctcggacaagtttatctacgccacggtgaagcagagctccgtggatatctacttccggcgccaggtggagctgagcaccatgtaccggcatatggagaagcacaactacgagagtgcggcggaggccatccaggccgtgagagacaacaagctgcatgccttcatctgggactcggcggtgctggagttcgaggcctcgcagaagtgcgacctggtgacgactggagagctgtttttccgctcgggcttcggcataggcatgcgcaaagacagcccctggaagcagaacgtctccctgtccatcctcaagtcccacgagaatggcttcatggaagacctggacaagacgtgggttcggtatcaggaatgtgactcg

SEQ ID NO: 58 - DNA, 411 bp, Homo sapiens
  Ligand-binding domain of GluN1 S2 in NM_007327
cgcatcacgggcatcaacgaccctcggctgaggaacccctcggacaagtttatctacgccacggtgaagcagagctccgtggatatctacttccggcgccaggtggagctgagcaccatgtaccggcatatggagaagcacaactacgagagtgcggcggaggccatccaggccgtgagagacaacaagctgcatgccttcatctgggactcggcggtgctggagttcgaggcctcgcagaagtgcgacctggtgacgactggagagctgtttttccgctcgggcttcggcataggcatgcgcaaagacagcccctggaagcagaacgtctccctgtccatcctcaagtcccacgagaatggcttcatggaagacctggacaagacgtgggttcggtatcaggaatgtgac

SEQ ID NO: 59 - DNA, 2748 bp, Artificial Sequence
  DNA seq of fusion protein #3 N2Becd-Fc
atgaagcccagagcggagtgctgttctcccaagttctggttggtgttggccgtcctggccgtgtcaggcagcagagctcgttctcagaagagcccccccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcaccccgatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccgaatgtgtccagagactgaagagcaggaggatgaccatctgagcattgtgaccctggaggaggcaccatttgtcattgtggaaagtgtggaccctctgagtggaacctgcatgaggaacacagtcccctgccaaaaacgcatagtcactgagaataaaacagacgaggagccgggttacatcaaaaaatgctgcaaggggttctgtattgacatccttaagaaaatttctaaatctgtgaagttcacctatgacctttacctggttaccaatggcaagcatgggaagaaaatcaatggaacctggaatggtatgattggagaggtggtcatgaagagggcctacatggcagtgggctcactcaccatcaatgaggaacgatcggaggtggtcgacttctctgtgcccttcatagagacaggcatcagtgtcatggtgtcacgcggcacccaggtttctggcctgagcgacaaaaagttccagagacctaatgacttctcaccccctttccgctttgggaccgtgcccaacggcagcacagagagaaatattcgcaataactatgcagaaatgcatgcctacatgggaaagttcaaccagaggggtgtagatgatgcattgctctccctgaaaacagggaaactggatgccttcatctatgatgcagcagtgctgaactatatggcaggcagagatgaaggctgcaagctggtgaccattggcagtgggaaggtctttgcttccactggctatggcattgccatccaaaaagattctgggtggaagcgccaggtggaccttgctatcctgcagctctttggagatggggagatggaagaactggaagctctctggctcactggcatttgtcacaatggctcgagcaccatggttagatctagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 60 - DNA, 1203 bp, Homo sapiens
  Amino terminal domain of GluN2B (including signal sequence)
atgaagcccagagcggagtgctgttctcccaagttctggttggtgttggccgtcctggccgtgtcaggcagcagagctcgttctcagaagagcccccccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcaccccgatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccgaatgtgtccagagactgaagagcag

SEQ ID NO: 61 - DNA, 417 bp, Homo sapiens
  Ligand-binding domain of GluN2B S1
gaggatgaccatctgagcattgtgaccctggaggaggcaccatttgtcattgtggaaagtgtggaccctctgagtggaacctgcatgaggaacacagtcccctgccaaaaacgcatagtcactgagaataaaacagacgaggagccgggttacatcaaaaaatgctgcaaggggttctgtattgacatccttaagaaaatttctaaatctgtgaagttcacctatgacctttacctggttaccaatggcaagcatgggaagaaaatcaatggaacctggaatggtatgattggagaggtggtcatgaagagggcctacatggcagtgggctcactcaccatcaatgaggaacgatcggaggtggtcgacttctctgtgcccttcatagagacaggcatcagtgtcatggtgtcacgc

SEQ ID NO: 62 - DNA, 425 bp, Homo sapiens
  Ligand-binding domain of GluN2B S2
aggtttctggcctgagcgacaaaaagttccagagacctaatgacttctcaccccctttccgctttgggaccgtgcccaacggcagcacagagagaaatattcgcaataactatgcagaaatgcatgcctacatgggaaagttcaaccagaggggtgtagatgatgcattgctctccctgaaaacagggaaactggatgccttcatctatgatgcagcagtgctgaactatatggcaggcagagatgaaggctgcaagctggtgaccattggcagtgggaaggtctttgcttccactggctatggcattgccatccaaaaagattctgggtggaagcgccaggtggaccttgctatcctgcagctctttggagatggggagatggaagaactggaagctctctggctcactggcatttgtcacaat

SEQ ID NO: 63 - DNA, 1083 bp, Homo sapiens
  Amino terminal domain of GluN2B in NM_000834
cccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcacccccatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccga

SEQ ID NO: 64 - DNA, 414 bp, Homo sapiens
  Ligand-binding domain of GluN2B S1 in NM_000834
gatgaccatctgagcattgtgaccctggaggaggcaccatttgtcattgtggaaagtgtggaccctctgagtggaacctgcatgaggaacacagtcccctgccaaaaacgcatagtcactgagaataaaacagacgaggagccgggttacatcaaaaaatgctgcaaggggttctgtattgacatccttaagaaaatttctaaatctgtgaagttcacctatgacctttacctggttaccaatggcaagcatgggaagaaaatcaatggaacctggaatggtatgattggagaggtggtcatgaagagggcctacatggcagtgggctcactcaccatcaatgaggaacgatcggaggtggtcgacttctctgtgcccttcatagagacaggcatcagtgtcatggtgtcacgc

SEQ ID NO: 65 - DNA, 426 bp, Homo sapiens
  Ligand-binding domain of GluN2B S2 in NM_000834
caggtttctggcctgagcgacaaaaagttccagagacctaatgacttctcaccccctttccgctttgggaccgtgcccaacggcagcacagagagaaatattcgcaataactatgcagaaatgcatgcctacatgggaaagttcaaccagaggggtgtagatgatgcattgctctccctgaaaacagggaaactggatgccttcatctatgatgcagcagtgctgaactatatggcaggcagagatgaaggctgcaagctggtgaccattggcagtgggaaggtctttgcttccactggctatggcattgccatccaaaaagattctgggtggaagcgccaggtggaccttgctatcctgcagctctttggagatggggagatggaagaactggaagctctctggctcactggcatttgtcacaat

SEQ ID NO: 66 - DNA, 4785 bp, Artificial Sequence
  DNA seq of fusion protein #4 N1ecd-N2Becd-Fc
atgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccggacggcatcctcgggctgcagctcatcaacggcaagaacgagtcggcccacatcagcgacgccgtaggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagattgtgacgatccaccaggagcccttcgtgtacgtcaagcccacgctgagtgatgggacatgcaaggaggagttcacagtcaacggcgacccagtcaagaaggtgatctgcaccgggcccaacgacacgtcgccgggcagcccccgccacacggtgcctcagtgttgctacggcttttgcatcgacctgctcatcaagctggcacggaccatgaacttcacctacgaggtgcacctggtggcagatggcaagttcggcacacaggagcgggtgaacaacagcaacaagaaggagtggaatgggatgatgggcgagctgctcagcgggcaggcagacatgatcgtggcgccgctaaccataaacaacgagcgcgcgcagtacatcgagttttccaagcccttcaagtaccagggcctgactattctggtcaagaagggcacccgcatcacgggcatcaacgacccccggctgaggaacccctcggacaagtttatctacgccacggtgaagcagagctccgtggatatctacttccggcgccaggtggagctgagcaccatgtaccggcatatggagaagcacaactacgagagtgcggcggaggccatccaggccgtgagagacaacaagctgcatgccttcatctgggactcggcggtgctggagttcgaggcctcgcagaagtgcgacctggtgacgactggagagctgtttttccgctcgggcttcggcataggcatgcgcaaagacagcccctggaagcagaacgtctccctgtccatcctcaagtcccacgagaatggcttcatggaagacctggacaagacgtgggttcggtatcaggaatgtgactcggggtcgactggtggaggagggtctggcggaggtggttccgggggcggaggatctggggccgcttctagacagaagagcccccccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcaccccgatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccgaatgtgtccagagactgaagagcaggaggatgaccatctgagcattgtgaccctggaggaggcaccatttgtcattgtggaaagtgtggaccctctgagtggaacctgcatgaggaacacagtcccctgccaaaaacgcatagtcactgagaataaaacagacgaggagccgggttacatcaaaaaatgctgcaaggggttctgtattgacatccttaagaaaatttctaaatctgtgaagttcacctatgacctttacctggttaccaatggcaagcatgggaagaaaatcaatggaacctggaatggtatgattggagaggtggtcatgaagagggcctacatggcagtgggctcactcaccatcaatgaggaacgatcggaggtggtcgacttctctgtgcccttcatagagacaggcatcagtgtcatggtgtcacgcggcacccaggtttctggcctgagcgacaaaaagttccagagacctaatgacttctcaccccctttccgctttgggaccgtgcccaacggcagcacagagagaaatattcgcaataactatgcagaaatgcatgcctacatgggaaagttcaaccagaggggtgtagatgatgcattgctctccctgaaaacagggaaactggatgccttcatctatgatgcagcagtgctgaactatatggcaggcagagatgaaggctgcaagctggtgaccattggcagtgggaaggtctttgcttccactggctatggcattgccatccaaaaagattctgggtggaagcgccaggtggaccttgctatcctgcagctctttggagatggggagatggaagaactggaagctctctggctcactggcatttgtcacaatggctcgagcaccatggttagatctagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 67 - DNA, 69 bp, Artificial Sequence
  Linker between a GluN1-domain and GluN2B-domain in a NMDAR construct of the invention
gggtcgactggtggaggagggtctggcggaggtggttccgggggcggaggatctggggccgcttctaga

SEQ ID NO: 68 - DNA, 1119 bp, Homo sapiens
  Amino terminal domain of GluN2B
cagaagagcccccccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcaccccgatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccgaatgtgtccagagactgaagagcag

SEQ ID NO: 69 - DNA, 1920 bp, Artificial Sequence
  DNA seq of fusion protein #5 N2A-ATD-Fc
atgggcagagtgggctattggaccctgctggtgctgccggcccttctggtctggcgcggtccggcgccgagcgcggcggcggagaagggtccccccgcgctaaatattgcggtgatgctgggtcacagccacgacgtgacagagcgcgaacttcgaacactgtggggccccgagcaggcggcggggctgcccctggacgtgaacgtggtagctctgctgatgaaccgcaccgaccccaagagcctcatcacgcacgtgtgcgacctcatgtccggggcacgcatccacggcctcgtgtttggggacgacacggaccaggaggccgtagcccagatgctggattttatctcctcccacaccttcgtccccatcttgggcattcatgggggcgcatctatgatcatggctgacaaggatccgacgtctaccttcttccagtttggagcgtccatccagcagcaagccacggtcatgctgaagatcatgcaggattatgactggcatgtcttctccctggtgaccactatcttccctggctacagggaattcatcagcttcgtcaagaccacagtggacaacagctttgtgggctgggacatgcagaatgtgatcacactggacacttcctttgaggatgcaaagacacaagtccagctgaagaagatccactcttctgtcatcttgctctactgttccaaagacgaggctgttctcattctgagtgaggcccgctcccttggcctcaccgggtatgatttcttctggattgtccccagcttggtctctgggaacacggagctcatcccaaaagagtttccatcgggactcatttctgtctcctacgatgactgggactacagcctggaggcgagagtgagggacggcattggcatcctaaccaccgctgcatcttctatgctggagaagttctcctacatccccgaggccaaggccagctgctacgggcagatggagaggccagaggtcccgatgcacaccttgcacccatttatggtcaatgttacatgggatggcaaagacttatccttcactgaggaaggctaccaggtgcaccccaggctggtggtgattgtgctgaacaaagaccgggaatgggaaaaggtgggcaagtgggagaaccatacgctgagcctgaggcacgccgtgtggcccaggtacaagtccttctccgactgtgagccggatgacaaccatctcagcatcggctcgagcaccatggttagatctagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 70 - DNA, 1224 bp, Homo sapiens
  Amino terminal domain of GluN2A (including signal sequence)
atgggcagagtgggctattggaccctgctggtgctgccggcccttctggtctggcgcggtccggcgccgagcgcggcggcggagaagggtccccccgcgctaaatattgcggtgatgctgggtcacagccacgacgtgacagagcgcgaacttcgaacactgtggggccccgagcaggcggcggggctgcccctggacgtgaacgtggtagctctgctgatgaaccgcaccgaccccaagagcctcatcacgcacgtgtgcgacctcatgtccggggcacgcatccacggcctcgtgtttggggacgacacggaccaggaggccgtagcccagatgctggattttatctcctcccacaccttcgtccccatcttgggcattcatgggggcgcatctatgatcatggctgacaaggatccgacgtctaccttcttccagtttggagcgtccatccagcagcaagccacggtcatgctgaagatcatgcaggattatgactggcatgtcttctccctggtgaccactatcttccctggctacagggaattcatcagcttcgtcaagaccacagtggacaacagctttgtgggctgggacatgcagaatgtgatcacactggacacttcctttgaggatgcaaagacacaagtccagctgaagaagatccactcttctgtcatcttgctctactgttccaaagacgaggctgttctcattctgagtgaggcccgctcccttggcctcaccgggtatgatttcttctggattgtccccagcttggtctctgggaacacggagctcatcccaaaagagtttccatcgggactcatttctgtctcctacgatgactgggactacagcctggaggcgagagtgagggacggcattggcatcctaaccaccgctgcatcttctatgctggagaagttctcctacatccccgaggccaaggccagctgctacgggcagatggagaggccagaggtcccgatgcacaccttgcacccatttatggtcaatgttacatgggatggcaaagacttatccttcactgaggaaggctaccaggtgcaccccaggctggtggtgattgtgctgaacaaagaccgggaatgggaaaaggtgggcaagtgggagaaccatacgctgagcctgaggcacgccgtgtggcccaggtacaagtccttctccgactgtgagccggatgacaaccatctcagcatc

SEQ ID NO: 71 - DNA, 2211 bp, Homo sapiens
  Amino terminal domain of GluN2A in NM_001134407
cccgcgctaaatattgcggtgatgctgggtcacagccacgacgtgacagagcgcgaacttcgaacactgtggggccccgagcaggcggcggggctgcccctggacgtgaacgtggtagctctgctgatgaaccgcaccgaccccaagagcctcatcacgcacgtgtgcgacctcatgtccggggcacgcatccacggcctcgtgtttggggacgacacggaccaggaggccgtagcccagatgctggattttatctcctcccacaccttcgtccccatcttgggcattcatgggggcgcatctatgatcatggctgacaaggatccgacgtctaccttcttccagtttggagcgtccatccagcagcaagccacggtcatgctgaagatcatgcaggattatgactggcatgtcttctccctggtgaccactatcttccctggctacagggaattcatcagcttcgtcaagaccacagtggacaacagctttgtgggctgggacatgcagaatgtgatcacactggacacttcctttgaggatgcaaagacacaagtccagctgaagaagatccactcttctgtcatcttgctctactgttccaaagacgaggctgttctcattctgagtgaggcccgctcccttggcctcaccgggtatgatttcttctggattgtccccagcttggtctctgggaacacggagctcatcccaaaagagtttccatcgggactcatttctgtctcctacgatgactgggactacagcctggaggcgagagtgagggacggcattggcatcctaaccaccgctgcatcttctatgctggagaagttctcctacatccccgaggccaaggccagctgctacgggcagatggagaggccagaggtcccgatgcacaccttgcacccatttatggtcaatgttacatgggatggcaaagacttatccttcactgaggaaggctaccaggtgcaccccaggctggtggtgattgtgctgaacaaagaccgggaatgggaaaaggtgggcaagtgggagaaccatacgctgagcctgaggcacgccgtgtggcccaggacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccagacggcatcctcgggctgcagctcatcaacggcaagaacgagtcggcccacatcagcgacgccgtgggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggc

SEQ ID NO: 72 - DNA, 1920 bp, Artificial Sequence
  DNA seq of fusion protein #6 N2B-ATD-Fc
atgaagcccagagcggagtgctgttctcccaagttctggttggtgttggccgtcctggccgtgtcaggcagcagagctcgttctcagaagagcccccccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcaccccgatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccgaatgtgtccagagactgaagagcaggaggatgaccatctgagcattggctcgagcaccatggttagatctagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 73 - DNA, 1224 bp, Homo sapiens
  Amino terminal domain of GluN2B (including signal sequence)
atgaagcccagagcggagtgctgttctcccaagttctggttggtgttggccgtcctggccgtgtcaggcagcagagctcgttctcagaagagcccccccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcaccccgatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccgaatgtgtccagagactgaagagcaggaggatgaccatctgagcatt

SEQ ID NO: 74 - DNA, 3108 bp, Artificial Sequence
  DNA seq of fusion protein #7 N1-ATD-N2A-ATD-Fc
atgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccggacggcatcctcgggctgcagctcatcaacggcaagaacgagtcggcccacatcagcgacgccgtaggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagattgggtcgactggtggaggagggtctggcggaggtggttccgggggcggaggatctggggccgcttctagagagaagggtccccccgcgctaaatattgcggtgatgctgggtcacagccacgacgtgacagagcgcgaacttcgaacactgtggggccccgagcaggcggcggggctgcccctggacgtgaacgtggtagctctgctgatgaaccgcaccgaccccaagagcctcatcacgcacgtgtgcgacctcatgtccggggcacgcatccacggcctcgtgtttggggacgacacggaccaggaggccgtagcccagatgctggattttatctcctcccacaccttcgtccccatcttgggcattcatgggggcgcatctatgatcatggctgacaaggatccgacgtctaccttcttccagtttggagcgtccatccagcagcaagccacggtcatgctgaagatcatgcaggattatgactggcatgtcttctccctggtgaccactatcttccctggctacagggaattcatcagcttcgtcaagaccacagtggacaacagctttgtgggctgggacatgcagaatgtgatcacactggacacttcctttgaggatgcaaagacacaagtccagctgaagaagatccactcttctgtcatcttgctctactgttccaaagacgaggctgttctcattctgagtgaggcccgctcccttggcctcaccgggtatgatttcttctggattgtccccagcttggtctctgggaacacggagctcatcccaaaagagtttccatcgggactcatttctgtctcctacgatgactgggactacagcctggaggcgagagtgagggacggcattggcatcctaaccaccgctgcatcttctatgctggagaagttctcctacatccccgaggccaaggccagctgctacgggcagatggagaggccagaggtcccgatgcacaccttgcacccatttatggtcaatgttacatgggatggcaaagacttatccttcactgaggaaggctaccaggtgcaccccaggctggtggtgattgtgctgaacaaagaccgggaatgggaaaaggtgggcaagtgggagaaccatacgctgagcctgaggcacgccgtgtggcccaggtacaagtccttctccgactgtgagccggatgacaaccatctcagcatcggctcgagcaccatggttagatctagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 75 - DNA, 1200 bp, Homo sapiens; identical to SEQ ID NO: 50
  Amino terminal domain of GluN1 (including signal sequence)

SEQ ID NO: 76 - DNA, 1143 bp, Homo sapiens
  Amino terminal domain of GluN2A
gagaagggtccccccgcgctaaatattgcggtgatgctgggtcacagccacgacgtgacagagcgcgaacttcgaacactgtggggccccgagcaggcggcggggctgcccctggacgtgaacgtggtagctctgctgatgaaccgcaccgaccccaagagcctcatcacgcacgtgtgcgacctcatgtccggggcacgcatccacggcctcgtgtttggggacgacacggaccaggaggccgtagcccagatgctggattttatctcctcccacaccttcgtccccatcttgggcattcatgggggcgcatctatgatcatggctgacaaggatccgacgtctaccttcttccagtttggagcgtccatccagcagcaagccacggtcatgctgaagatcatgcaggattatgactggcatgtcttctccctggtgaccactatcttccctggctacagggaattcatcagcttcgtcaagaccacagtggacaacagctttgtgggctgggacatgcagaatgtgatcacactggacacttcctttgaggatgcaaagacacaagtccagctgaagaagatccactcttctgtcatcttgctctactgttccaaagacgaggctgttctcattctgagtgaggcccgctcccttggcctcaccgggtatgatttcttctggattgtccccagcttggtctctgggaacacggagctcatcccaaaagagtttccatcgggactcatttctgtctcctacgatgactgggactacagcctggaggcgagagtgagggacggcattggcatcctaaccaccgctgcatcttctatgctggagaagttctcctacatccccgaggccaaggccagctgctacgggcagatggagaggccagaggtcccgatgcacaccttgcacccatttatggtcaatgttacatgggatggcaaagacttatccttcactgaggaaggctaccaggtgcaccccaggctggtggtgattgtgctgaacaaagaccgggaatgggaaaaggtgggcaagtgggagaaccatacgctgagcctgaggcacgccgtgtggcccaggtacaagtccttctccgactgtgagccggatgacaaccatctcagcatc

SEQ ID NO: 77 - DNA, 3105 bp, Artificial Sequence
  DNA seq of fusion protein #8 N1-ATD-N2B-ATD-Fc
atgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccggacggcatcctcgggctgcagctcatcaacggcaagaacgagtcggcccacatcagcgacgccgtaggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagattgggtcgactggtggaggagggtctggcggaggtggttccgggggcggaggatctggggccgcttctagacagaagagcccccccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcaccccgatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccgaatgtgtccagagactgaagagcaggaggatgaccatctgagcattggctcgagcaccatggttagatctagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 78 - DNA, 1200 bp, Homo sapiens; identical to SEQ ID NO: 50
  Amino terminal domain of GluN1 (including signal sequence)

SEQ ID NO: 79 - DNA, 1140 bp, Homo sapiens
  Amino terminal domain of GluN2B
cagaagagcccccccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcaccccgatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccgaatgtgtccagagactgaagagcaggaggatgaccatctgagcatt

SEQ ID NO: 80 - DNA, 3702 bp, Homo sapiens
  Human GluN2C NM_000835
atgggtggggccctggggccggccctgttgctcacctcgctcttcggtgcctgggcagggctgggtccggggcagggcgagcagggcatgacggtggccgtggtgtttagcagctcagggccgccccaggcccagttccgtgcccgcctcaccccccagagcttcctggacctacccctggagatccagccgctcacagttggggtcaacaccaccaaccccagcagcctcctcacccagatctgcggcctcctgggtgctgcccacgtccacggcattgtctttgaggacaacgtggacaccgaggcggtggcccagatccttgacttcatctcctcccagacccatgtgcccatcctcagcatcagcggaggctctgctgtggtcctcacccccaaggagccgggctccgccttcctgcagctgggcgtgtccctggagcagcagctgcaggtgctgttcaaggtgctggaagagtacgactggagcgccttcgccgtcatcaccagcctgcacccgggccacgcgctcttcctggagggcgtgcgcgccgtcgccgacgccagccacgtgagttggcggctgctggacgtggtcacgctggagctgggcccgggagggccgcgcgcgcgcacgcagcgcctgctgcgccagctcgacgcgcccgtgtttgtggcctactgctcgcgcgaggaggccgaggtgctcttcgccgaggcggcgcaggccggtctggtggggcccggccacgtgtggctggtgcccaacctggcgctgggcagcaccgatgcgccccccgccaccttccccgtgggcctcatcagcgtcgtcaccgagagctggcgcctcagcctgcgccagaaggtgcgcgacggcgtggccattctggccctgggcgcccacagctactggcgccagcatggaaccctgccagccccggccggggactgccgtgttcaccctgggcccgtcagccctgcccgggaggccttctacaggcacctactgaatgtcacctgggagggccgagacttctccttcagccctggtgggtacctggtccagcccaccatggtggtgatcgccctcaaccggcaccgcctctgggagatggtggggcgctgggagcatggcgtcctatacatgaagtaccccgtgtggcctcgctacagtgcctctctgcagcctgtggtggacagtcggcacctgacggtggccacgctggaagagcggccctttgtcatcgtggagagccctgaccctggcacaggaggctgtgtccccaacaccgtgccctgccgcaggcagagcaaccacaccttcagcagcggggacgtggccccctacaccaagctctgctgtaagggattctgcatcgacatcctcaagaagctggccagagtggtcaaattctcctacgacctgtacctggtgaccaacggcaagcatggcaagcgggtgcgcggcgtatggaacggcatgattggggaggtgtactacaagcgggcagacatggccatcggctccctcaccatcaatgaggaacgctccgagatcgtagacttctctgtaccctttgtggagacgggcatcagtgtgatggtggctcgcagcaatggcaccgtctccccctcggccttcttggagccatatagccctgcagtgtgggtgatgatgtttgtcatgtgcctcactgtggtggccatcaccgtcttcatgttcgagtacttcagccctgtcagctacaaccagaacctcaccagaggcaagaagtccgggggcccagctttcactatcggcaagtccgtgtggctgctgtgggcgctggtcttcaacaactcagtgcccatcgagaacccgcggggcaccaccagcaagatcatggttctggtctgggccttctttgctgtcatcttcctcgccagctacacggccaacctggccgccttcatgatccaagagcaatacatcgacactgtgtcgggcctcagtgacaagaagtttcagcggcctcaagatcagtacccacctttccgcttcggcacggtgcccaacggcagcacggagcggaacatccgcagtaactaccgtgacatgcacacccacatggtcaagttcaaccagcgctcggtggaggacgcgctcaccagcctcaagatggggaagctggatgccttcatctatgatgctgctgtcctcaactacatggcaggcaaggacgagggctgcaagctggtcaccattgggtctggcaaggtctttgctaccactggctacggcatcgccatgcagaaggactcccactggaagcgggccatagacctggcgctcttgcagttcctgggggacggagagacacagaaactggagacagtgtggctctcagggatctgccagaatgagaagaacgaggtgatgagcagcaagctggacatcgacaacatggcaggcgtcttctacatgctgctggtggccatggggctggccctgctggtcttcgcctgggagcacctggtctactggaagctgcgccactcggtgcccaactcatcccagctggacttcctgctggctttcagcaggggcatctacagctgcttcagcggggtgcagagcctcgccagcccaccgcggcaggccagcccggacctcacggccagctcggcccaggccagcgtgctcaagatgctgcaggcagcccgcgacatggtgaccacggcgggcgtaagcagctccctggaccgcgccactcgcaccatcgagaattggggtggcggccgccgtgcgcccccaccgtccccctgcccgaccccgcggtctggccccagcccatgcctgcccacccccgacccgcccccagagccgagccccacgggctggggaccgccagacgggggtcgcgcggcgcttgtgcgcagggctccgcagcccccgggccgccccccgacgccggggccgcccctgtccgacgtctcccgagtgtcgcgccgcccagcctgggaggcgcggtggccggtgcggaccgggcactgcgggaggcacctctcggcctccgagcggcccctgtcgcccgcgcgctgtcactacagctcctttcctcgagccgaccgatccggccgccccttcctcccgctcttcccggagctggaggacctgccgctgctcggtccggagcagctggcccggcgggaggccctgctgcacgcggcctgggcccggggctcgcgcccgcgtcacgcttccctgcccagctccgtggccgaggccttcgctcggcccagctcgctgcccgctgggtgcaccggccccgcctgcgcccgccccgacggccactcggcctgcaggcgcttggcgcaggcgcagtcgatgtgcttgccgatctaccgggaggcctgccaggagggcgagcaggcaggggcccccgcctggcagcacagacagcacgtctgcctgcacgcccacgcccacctgccattttgctggggggctgtctgtcctcaccttccaccctgtgccagccacggctcctggctctccggggcctgggggcctctggggcacaggggcaggactctggggctgggcacaggctacagagacagtgggggactggacgagatcagcagggtagcccgtgggacgcaaggcttcccgggaccctgcacctggagacggatctccagtctggagtcagaagtgtga

SEQ ID NO: 81 - DNA, 1086 bp, Homo sapiens
  GluN2C ATD
cagggcatgacggtggccgtggtgtttagcagctcagggccgccccaggcccagttccgtgcccgcctcaccccccagagcttcctggacctacccctggagatccagccgctcacagttggggtcaacaccaccaaccccagcagcctcctcacccagatctgcggcctcctgggtgctgcccacgtccacggcattgtctttgaggacaacgtggacaccgaggcggtggcccagatccttgacttcatctcctcccagacccatgtgcccatcctcagcatcagcggaggctctgctgtggtcctcacccccaaggagccgggctccgccttcctgcagctgggcgtgtccctggagcagcagctgcaggtgctgttcaaggtgctggaagagtacgactggagcgccttcgccgtcatcaccagcctgcacccgggccacgcgctcttcctggagggcgtgcgcgccgtcgccgacgccagccacgtgagttggcggctgctggacgtggtcacgctggagctgggcccgggagggccgcgcgcgcgcacgcagcgcctgctgcgccagctcgacgcgcccgtgtttgtggcctactgctcgcgcgaggaggccgaggtgctcttcgccgaggcggcgcaggccggtctggtggggcccggccacgtgtggctggtgcccaacctggcgctgggcagcaccgatgcgccccccgccaccttccccgtgggcctcatcagcgtcgtcaccgagagctggcgcctcagcctgcgccagaaggtgcgcgacggcgtggccattctggccctgggcgcccacagctactggcgccagcatggaaccctgccagccccggccggggactgccgtgttcaccctgggcccgtcagccctgcccgggaggccttctacaggcacctactgaatgtcacctgggagggccgagacttctccttcagccctggtgggtacctggtccagcccaccatggtggtgatcgccctcaaccggcaccgcctctgggagatggtggggcgctgggagcatggcgtcctatacatgaagtaccccgtgtggcctcgc

SEQ ID NO: 82 - DNA, 414 bp, Homo sapiens
  GluN2C S1
agtcggcacctgacggtggccacgctggaagagcggccctttgtcatcgtggagagccctgaccctggcacaggaggctgtgtccccaacaccgtgccctgccgcaggcagagcaaccacaccttcagcagcggggacgtggccccctacaccaagctctgctgtaagggattctgcatcgacatcctcaagaagctggccagagtggtcaaattctcctacgacctgtacctggtgaccaacggcaagcatggcaagcgggtgcgcggcgtatggaacggcatgattggggaggtgtactacaagcgggcagacatggccatcggctccctcaccatcaatgaggaacgctccgagatcgtagacttctctgtaccctttgtggagacgggcatcagtgtgatggtggctcgc

SEQ ID NO: 83 - DNA, 426 bp, Homo sapiens
  GluN2C S2
actgtgtcgggcctcagtgacaagaagtttcagcggcctcaagatcagtacccacctttccgcttcggcacggtgcccaacggcagcacggagcggaacatccgcagtaactaccgtgacatgcacacccacatggtcaagttcaaccagcgctcggtggaggacgcgctcaccagcctcaagatggggaagctggatgccttcatctatgatgctgctgtcctcaactacatggcaggcaaggacgagggctgcaagctggtcaccattgggtctggcaaggtctttgctaccactggctacggcatcgccatgcagaaggactcccactggaagcgggccatagacctggcgctcttgcagttcctgggggacggagagacacagaaactggagacagtgtggctctcagggatctgccagaat

SEQ ID NO: 84 - DNA, 2043 bp, Artificial Sequence
  GluN2C ecd incl. signal sequence and GT-linker between S1 and S2
atgggtggggccctggggccggccctgttgctcacctcgctcttcggtgcctgggcagggctgggtccggggcagggcgagcagggcatgacggtggccgtggtgtttagcagctcagggccgccccaggcccagttccgtgcccgcctcaccccccagagcttcctggacctacccctggagatccagccgctcacagttggggtcaacaccaccaaccccagcagcctcctcacccagatctgcggcctcctgggtgctgcccacgtccacggcattgtctttgaggacaacgtggacaccgaggcggtggcccagatccttgacttcatctcctcccagacccatgtgcccatcctcagcatcagcggaggctctgctgtggtcctcacccccaaggagccgggctccgccttcctgcagctgggcgtgtccctggagcagcagctgcaggtgctgttcaaggtgctggaagagtacgactggagcgccttcgccgtcatcaccagcctgcacccgggccacgcgctcttcctggagggcgtgcgcgccgtcgccgacgccagccacgtgagttggcggctgctggacgtggtcacgctggagctgggcccgggagggccgcgcgcgcgcacgcagcgcctgctgcgccagctcgacgcgcccgtgtttgtggcctactgctcgcgcgaggaggccgaggtgctcttcgccgaggcggcgcaggccggtctggtggggcccggccacgtgtggctggtgcccaacctggcgctgggcagcaccgatgcgccccccgccaccttccccgtgggcctcatcagcgtcgtcaccgagagctggcgcctcagcctgcgccagaaggtgcgcgacggcgtggccattctggccctgggcgcccacagctactggcgccagcatggaaccctgccagccccggccggggactgccgtgttcaccctgggcccgtcagccctgcccgggaggccttctacaggcacctactgaatgtcacctgggagggccgagacttctccttcagccctggtgggtacctggtccagcccaccatggtggtgatcgccctcaaccggcaccgcctctgggagatggtggggcgctgggagcatggcgtcctatacatgaagtaccccgtgtggcctcgctacagtgcctctctgcagcctgtggtggacagtcggcacctgacggtggccacgctggaagagcggccctttgtcatcgtggagagccctgaccctggcacaggaggctgtgtccccaacaccgtgccctgccgcaggcagagcaaccacaccttcagcagcggggacgtggccccctacaccaagctctgctgtaagggattctgcatcgacatcctcaagaagctggccagagtggtcaaattctcctacgacctgtacctggtgaccaacggcaagcatggcaagcgggtgcgcggcgtatggaacggcatgattggggaggtgtactacaagcgggcagacatggccatcggctccctcaccatcaatgaggaacgctccgagatcgtagacttctctgtaccctttgtggagacgggcatcagtgtgatggtggctcgcggcaccactgtgtcgggcctcagtgacaagaagtttcagcggcctcaagatcagtacccacctttccgcttcggcacggtgcccaacggcagcacggagcggaacatccgcagtaactaccgtgacatgcacacccacatggtcaagttcaaccagcgctcggtggaggacgcgctcaccagcctcaagatggggaagctggatgccttcatctatgatgctgctgtcctcaactacatggcaggcaaggacgagggctgcaagctggtcaccattgggtctggcaaggtctttgctaccactggctacggcatcgccatgcagaaggactcccactggaagcgggccatagacctggcgctcttgcagttcctgggggacggagagacacagaaactggagacagtgtggctctcagggatctgccagaat

SEQ ID NO: 85 - DNA, 4011 bp, Homo sapiens
  Human GluN2D U77783 (DNA)
atgcgcggcgccggtggcccccgcggccctcggggccccgctaagatgctgctgctgctggcgctggcctgcgccagcccgttcccggaggaggcgccggggccgggcggggccggtgggcccggcggcggcctcggcggggcgcggccgctcaacgtggcgctcgtgttctcggggcccgcgtacgcggccgaggcggcacgcctgggcccggccgtggcggcggcggtgcgcagcccgggcctagacgtgcggcccgtggcgctggtgctcaacggctcggacccgcgcagcctcgtgctgcagctctgcgacctgctgtcggggttgcgcgtgcacggcgtggtcttcgaagacgactcgcgcgcgcccgccgtcgcgcccatcctcgacttcctgtcggcgcagacctcgctgcccatcgtggccgtgcacggcggcgccgcgctcgtgctcacgcccaaggagaagggctccaccttcctgcagctgggctcttccaccgagcaacagcttcaggtcatctttgaggtgctggaggagtatgactggacgtcctttgtagccgtgaccactcgtgcccctggccaccgggccttcctgtcctacattgaggtgctgactgacggtagtctggtgggctgggagcaccgcggagcgctgacgctggaccctggggcgggcgaggccgtgctcagtgcccagctccgcagtgtcagcgcgcagatccgcctgctcttctgcgcccgagaggaggccgagcccgtgttccgcgcagctgaggaggctggcctcactggatctggctacgtctggttcatggtggggccccagctggctggaggcgggggctctggggcccctggtgagccccctcttctgccaggaggcgcccccctgcctgccgggctgtttgcagtgcgctcggctggctggcgggatgacctggctcggcgagtggcagctggcgtggccgtagtggccagaggtgcccaggccctgctgcgtgattatggtttccttcctgagctcggccacgactgtcgcgcccagaaccgcacccaccgcggcgagagtctgcataggtacttcatgaacatcacgtgggataaccgggattactccttcaatgaggacggcttcctagtgaacccctccctggtggtcatctccctcaccagagacaggacgtgggaggtggtgggcagctgggagcagcagacgctccgcctcaagtacccgctgtggtcccgctatggtcgcttcctgcagccagtggacgacacgcagcacctcacggtggccacgctggaggaaaggccgtttgtcatcgtggagcctgcagaccctatcagcggcacctgcatccgagactccgtcccctgccggagccagctcaaccgaacccacagccctccaccggatgccccccgcccggaaaagcgctgctgcaagggtttctgcatcgacattctgaagcggctggcgcataccatcggcttcagctacgacctctacctggtcaccaatggcaagcacggaaagaagatcgatggcgtctggaacggcatgatcggggaggtgttctaccagcgcgcagacatggccatcggctccctcaccatcaacgaggagcgctccgagatcgtggacttctccgtccccttcgtggagaccggcatcagcgtcatggtggcgcgcagcaatggcacggtgtccccctcggccttcctcgagccctacagccccgccgtgtgggtgatgatgttcgtcatgtgcctcactgtggtcgccgtcactgttttcatcttcgagtacctcagtcctgttggttacaaccgcagcctggccacgggcaagcgccctggcggttcaaccttcaccattgggaaatccatctggctgctctgggccctggtgttcaataattcggtgcccgtggagaacccccggggaaccaccagcaaaatcatggtgctggtgtgggccttcttcgccgtcatcttcctcgccagctacacagccaacctggccgccttcatgatccaggaggagtacgtggatactgtgtctgggctcagtgaccgcaagttccagaggccccaggagcagtacccgcccctgaagtttgggaccgtgcccaacggctccacggagaagaacatccgcagcaactatcccgacatgcacagctacatggtgcgctacaaccagccccgcgtagaggaagcgctcactcagctcaaggcagggaagctggacgccttcatctacgatgctgcagtgctcaattacatggcccgcaaggacgagggctgcaagcttgtcaccatcggctccggcaaggtcttcgccacgacaggctatggcatcgccctgcacaagggctcccgctggaagcggcccatcgacctggcgttgctgcagttcctgggggatgatgagatcgagatgctggagcggctgtggctctctgggatctgccacaatgacaaaatcgaggtgatgagcagcaagctggacatcgacaacatggcgggcgtcttctacatgctcctggtggccatgggcctgtccctgctggtcttcgcctgggagcacctggtgtactggcgcctgcggcactgcctggggcccacccaccgcatggacttcctgctggccttctccaggggcatgtacagctgctgcagcgctgaggccgccccaccgcccgccaagcccccgccgccgccacagcccctgcccagccccgcgtaccccgcgccggggccggctcccgggcccgcacctttcgtgccccgcgagcgcgcctcagtggaccgctggcgccggaccaagggcgcggggccgccggggggcgcgggcctggccgacggcttccaccgctactacggccccatcgagccgcagggcctaggcctcggcctgggcgaagcgcgcgcggcaccgcggggcgcagccgggcgcccgctgtccccgccggccgctcagcccccgcagaagccgccggcctcctatttcgccatcgtacgcgacaaggagccagccgagccccccgccggcgccttccccggcttcccgtcgccgcccgcgccccccgccgccgcggccaccgccgtcgggccgccactctgccgcttggccttcgaggacgagagcccgccggcgcccgcgcggtggccgcgctcggaccccgagagccaacccctgctggggccaggcgcgggcggcgcggggggcacggggggcgcaggcggaggagccccggccgctccgcccccgtgctgcgccgcgccgcccccgtgcccttacctcgatctcgagccgtcgccgtcggactcggaggactcggagagcctgggcggcgcgtcgctgggcggcctggatccctggtggttcgccgacttcccttacccgtatgccgagcgcctcgggccgccgcccggccgctactggtcggtcgacaagctcgggggctggcgcgccgggagctgggactacctgcccccgcgcagcggtccggccgcctggcactgtcggcactgcgccagcctggagctgctgccgccgccgcgccatctcagctgctcgcacgatggcctggacggcggctggtgggcgccaccgcctccaccctgggccgccgggcccctgccccgacgccgggcccgctgcgggtgcccgcggtcgcacccgcaccgcccgcgggcctcgcaccgcacgcccgccgctgccgcgccccaccaccacaggcaccggcgcgccgctgggggctgggacctcccgccgcccgcgcccacctcgcgctcgctcgaggacctcagctcgtgccctcgcgccgcccctgcgcgcaggcttaccgggccctcccgccacgctcgcaggtgtccgcacgccgcgcactgggggccgccgctgcccacagcttcccaccggagacaccggggcggggacctgggcacccgcaggggctcggcgcacttctctagcctcgagtccgaggtatga

SEQ ID NO: 86 - DNA, 1110 bp, Homo sapiens
  GluN2D ATD
cggccgctcaacgtggcgctcgtgttctcggggcccgcgtacgcggccgaggcggcacgcctgggcccggccgtggcggcggcggtgcgcagcccgggcctagacgtgcggcccgtggcgctggtgctcaacggctcggacccgcgcagcctcgtgctgcagctctgcgacctgctgtcggggttgcgcgtgcacggcgtggtcttcgaagacgactcgcgcgcgcccgccgtcgcgcccatcctcgacttcctgtcggcgcagacctcgctgcccatcgtggccgtgcacggcggcgccgcgctcgtgctcacgcccaaggagaagggctccaccttcctgcagctgggctcttccaccgagcaacagcttcaggtcatctttgaggtgctggaggagtatgactggacgtcctttgtagccgtgaccactcgtgcccctggccaccgggccttcctgtcctacattgaggtgctgactgacggtagtctggtgggctgggagcaccgcggagcgctgacgctggaccctggggcgggcgaggccgtgctcagtgcccagctccgcagtgtcagcgcgcagatccgcctgctcttctgcgcccgagaggaggccgagcccgtgttccgcgcagctgaggaggctggcctcactggatctggctacgtctggttcatggtggggccccagctggctggaggcgggggctctggggcccctggtgagccccctcttctgccaggaggcgcccccctgcctgccgggctgtttgcagtgcgctcggctggctggcgggatgacctggctcggcgagtggcagctggcgtggccgtagtggccagaggtgcccaggccctgctgcgtgattatggtttccttcctgagctcggccacgactgtcgcgcccagaaccgcacccaccgcggcgagagtctgcataggtacttcatgaacatcacgtgggataaccgggattactccttcaatgaggacggcttcctagtgaacccctccctggtggtcatctccctcaccagagacaggacgtgggaggtggtgggcagctgggagcagcagacgctccgcctcaagtacccgctgtggtcccgc

SEQ ID NO: 87 - DNA, 417 bp, Homo sapiens
  GluN2D S1
acgcagcacctcacggtggccacgctggaggaaaggccgtttgtcatcgtggagcctgcagaccctatcagcggcacctgcatccgagactccgtcccctgccggagccagctcaaccgaacccacagccctccaccggatgccccccgcccggaaaagcgctgctgcaagggtttctgcatcgacattctgaagcggctggcgcataccatcggcttcagctacgacctctacctggtcaccaatggcaagcacggaaagaagatcgatggcgtctggaacggcatgatcggggaggtgttctaccagcgcgcagacatggccatcggctccctcaccatcaacgaggagcgctccgagatcgtggacttctccgtccccttcgtggagaccggcatcagcgtcatggtggcgcgc

SEQ ID NO: 88 - DNA, 426 bp, Homo sapiens
  GluN2D S2
actgtgtctgggctcagtgaccgcaagttccagaggccccaggagcagtacccgcccctgaagtttgggaccgtgcccaacggctccacggagaagaacatccgcagcaactatcccgacatgcacagctacatggtgcgctacaaccagccccgcgtagaggaagcgctcactcagctcaaggcagggaagctggacgccttcatctacgatgctgcagtgctcaattacatggcccgcaaggacgagggctgcaagcttgtcaccatcggctccggcaaggtcttcgccacgacaggctatggcatcgccctgcacaagggctcccgctggaagcggcccatcgacctggcgttgctgcagttcctgggggatgatgagatcgagatgctggagcggctgtggctctctgggatctgccacaat

SEQ ID NO: 89 - DNA, 2133 bp, Artificial Sequence
  GluN2D ecd incl. signal sequence and GT-linker between S1 and S2
atgcgcggcgccggtggcccccgcggccctcggggccccgctaagatgctgctgctgctggcgctggcctgcgccagcccgttcccggaggaggcgccggggccgggcggggccggtgggcccggcggcggcctcggcggggcgcggccgctcaacgtggcgctcgtgttctcggggcccgcgtacgcggccgaggcggcacgcctgggcccggccgtggcggcggcggtgcgcagcccgggcctagacgtgcggcccgtggcgctggtgctcaacggctcggacccgcgcagcctcgtgctgcagctctgcgacctgctgtcggggttgcgcgtgcacggcgtggtcttcgaagacgactcgcgcgcgcccgccgtcgcgcccatcctcgacttcctgtcggcgcagacctcgctgcccatcgtggccgtgcacggcggcgccgcgctcgtgctcacgcccaaggagaagggctccaccttcctgcagctgggctcttccaccgagcaacagcttcaggtcatctttgaggtgctggaggagtatgactggacgtcctttgtagccgtgaccactcgtgcccctggccaccgggccttcctgtcctacattgaggtgctgactgacggtagtctggtgggctgggagcaccgcggagcgctgacgctggaccctggggcgggcgaggccgtgctcagtgcccagctccgcagtgtcagcgcgcagatccgcctgctcttctgcgcccgagaggaggccgagcccgtgttccgcgcagctgaggaggctggcctcactggatctggctacgtctggttcatggtggggccccagctggctggaggcgggggctctggggcccctggtgagccccctcttctgccaggaggcgcccccctgcctgccgggctgtttgcagtgcgctcggctggctggcgggatgacctggctcggcgagtggcagctggcgtggccgtagtggccagaggtgcccaggccctgctgcgtgattatggtttccttcctgagctcggccacgactgtcgcgcccagaaccgcacccaccgcggcgagagtctgcataggtacttcatgaacatcacgtgggataaccgggattactccttcaatgaggacggcttcctagtgaacccctccctggtggtcatctccctcaccagagacaggacgtgggaggtggtgggcagctgggagcagcagacgctccgcctcaagtacccgctgtggtcccgctatggtcgcttcctgcagccagtggacgacacgcagcacctcacggtggccacgctggaggaaaggccgtttgtcatcgtggagcctgcagaccctatcagcggcacctgcatccgagactccgtcccctgccggagccagctcaaccgaacccacagccctccaccggatgccccccgcccggaaaagcgctgctgcaagggtttctgcatcgacattctgaagcggctggcgcataccatcggcttcagctacgacctctacctggtcaccaatggcaagcacggaaagaagatcgatggcgtctggaacggcatgatcggggaggtgttctaccagcgcgcagacatggccatcggctccctcaccatcaacgaggagcgctccgagatcgtggacttctccgtccccttcgtggagaccggcatcagcgtcatggtggcgcgcggcaccactgtgtctgggctcagtgaccgcaagttccagaggccccaggagcagtacccgcccctgaagtttgggaccgtgcccaacggctccacggagaagaacatccgcagcaactatcccgacatgcacagctacatggtgcgctacaaccagccccgcgtagaggaagcgctcactcagctcaaggcagggaagctggacgccttcatctacgatgctgcagtgctcaattacatggcccgcaaggacgagggctgcaagcttgtcaccatcggctccggcaaggtcttcgccacgacaggctatggcatcgccctgcacaagggctcccgctggaagcggcccatcgacctggcgttgctgcagttcctgggggatgatgagatcgagatgctggagcggctgtggctctctgggatctgccacaat

SEQ ID NO: 90 - DNA, 4395 bp, Homo sapiens
  Human GluN2A NM_001134407
atgggcagagtgggctattggaccctgctggtgctgccggcccttctggtctggcgcggtccggcgccgagcgcggcggcggagaagggtccccccgcgctaaatattgcggtgatgctgggtcacagccacgacgtgacagagcgcgaacttcgaacactgtggggccccgagcaggcggcggggctgcccctggacgtgaacgtggtagctctgctgatgaaccgcaccgaccccaagagcctcatcacgcacgtgtgcgacctcatgtccggggcacgcatccacggcctcgtgtttggggacgacacggaccaggaggccgtagcccagatgctggattttatctcctcccacaccttcgtccccatcttgggcattcatgggggcgcatctatgatcatggctgacaaggatccgacgtctaccttcttccagtttggagcgtccatccagcagcaagccacggtcatgctgaagatcatgcaggattatgactggcatgtcttctccctggtgaccactatcttccctggctacagggaattcatcagcttcgtcaagaccacagtggacaacagctttgtgggctgggacatgcagaatgtgatcacactggacacttcctttgaggatgcaaagacacaagtccagctgaagaagatccactcttctgtcatcttgctctactgttccaaagacgaggctgttctcattctgagtgaggcccgctcccttggcctcaccgggtatgatttcttctggattgtccccagcttggtctctgggaacacggagctcatcccaaaagagtttccatcgggactcatttctgtctcctacgatgactgggactacagcctggaggcgagagtgagggacggcattggcatcctaaccaccgctgcatcttctatgctggagaagttctcctacatccccgaggccaaggccagctgctacgggcagatggagaggccagaggtcccgatgcacaccttgcacccatttatggtcaatgttacatgggatggcaaagacttatccttcactgaggaaggctaccaggtgcaccccaggctggtggtgattgtgctgaacaaagaccgggaatgggaaaaggtgggcaagtgggagaaccatacgctgagcctgaggcacgccgtgtggcccaggtacaagtccttctccgactgtgagccggatgacaaccatctcagcatcgtcaccctggaggaggccccattcgtcatcgtggaagacatagaccccctgaccgagacgtgtgtgaggaacaccgtgccatgtcggaagttcgtcaaaatcaacaattcaaccaatgaggggatgaatgtgaagaaatgctgcaaggggttctgcattgatattctgaagaagctttccagaactgtgaagtttacttacgacctctatctggtgaccaatgggaagcatggcaagaaagttaacaatgtgtggaatggaatgatcggtgaagtggtctatcaacgggcagtcatggcagttggctcgctcaccatcaatgaggaacgttctgaagtggtggacttctctgtgccctttgtggaaacgggaatcagtgtcatggtttcaagaagtaatggcaccgtctcaccttctgcttttctagaaccattcagcgcctctgtctgggtgatgatgtttgtgatgctgctcattgtttctgccatagctgtttttgtctttgaatacttcagccctgttggatacaacagaaacttagccaaagggaaagcaccccatgggccttcttttacaattggaaaagctatatggcttctttggggcctggtgttcaataactccgtgcctgtccagaatcctaaagggaccaccagcaagatcatggtatctgtatgggccttcttcgctgtcatattcctggctagctacacagccaatctggctgccttcatgatccaagaggaatttgtggaccaagtgaccggcctcagtgacaaaaagtttcagagacctcatgactattccccaccttttcgatttgggacagtgcctaatggaagcacggagagaaacattcggaataactatccctacatgcatcagtacatgaccaaatttaatcagaaaggagtagaggacgccttggtcagcctgaaaacggggaagctggacgctttcatctacgatgccgcagtcttgaattacaaggctgggagggatgaaggctgcaagctggtgaccatcgggagtgggtacatctttgccaccaccggttatggaattgcccttcagaaaggctctccttggaagaggcagatcgacctggccttgcttcagtttgtgggtgatggtgagatggaggagctggagaccctgtggctcactgggatctgccacaacgagaagaacgaggtgatgagcagccagctggacattgacaacatggcgggcgtattctacatgctggctgccgccatggcccttagcctcatcaccttcatctgggagcacctcttctactggaagctgcgcttctgtttcacgggcgtgtgctccgaccggcctgggttgctcttctccatcagcaggggcatctacagctgcattcatggagtgcacattgaagaaaagaagaagtctccagacttcaatctgacgggatcccagagcaacatgttaaaactcctccggtcagccaaaaacatttccagcatgtccaacatgaactcctcaagaatggactcacccaaaagagctgctgacttcatccaaagaggttccctcatcatggacatggtttcagataaggggaatttgatgtactcagacaacaggtcctttcaggggaaagagagcatttttggagacaacatgaacgaactccaaacatttgtggccaaccggcagaaggataacctcaataactatgtattccagggacaacatcctcttactctcaatgagtccaaccctaacacggtggaggtggccgtgagcacagaatccaaagcgaactctagaccccggcagctgtggaagaaatccgtggattccatacgccaggattcactatcccagaatccagtctcccagagggatgaggcaacagcagagaataggacccactccctaaagagccctaggtatcttccagaagagatggcccactctgacatttcagaaacgtcaaatcgggccacgtgccacagggaacctgacaacagtaagaaccacaaaaccaaggacaactttaaaaggtcagtggcctccaaataccccaaggactgtagtgaggtcgagcgcacctacctgaaaaccaaatcaagctcccctagagacaagatctacactatagatggtgagaaggagcctggtttccacttagatccaccccagtttgttgaaaatgtgaccctgcccgagaacgtggacttcccggacccctaccaggatcccagtgaaaacttccgcaagggggactccacgctgccaatgaaccggaaccccttgcataatgaagaggggctttccaacaacgaccagtataaactctactccaagcacttcaccttgaaagacaagggttccccgcacagtgagaccagcgagcgataccggcagaactccacgcactgcagaagctgcctttccaacatgcccacctattcaggccacttcaccatgaggtcccccttcaagtgcgatgcctgcctgcggatggggaacctctatgacatcgatgaagaccagatgcttcaggagacaggtaacccagccaccggggagcaggtctaccagcaggactgggcacagaacaatgcccttcaattacaaaagaacaagctaaggattagccgtcagcattcctacgataacattgtcgacaaacctagggagctagaccttagcaggccctcccggagcataagcctcaaggacagggaacggcttctggagggaaatttttacggcagcctgtttagtgtcccctcaagcaaactctcggggaaaaaaagctcccttttcccccaaggtctggaggacagcaagaggagcaagtctctcttgccagaccacacctccgataaccctttcctccactcccacagggatgaccaacgcttggttattgggagatgcccctcggacccttacaaacactcgttgccatcccaggcggtgaatgacagctatcttcggtcgtccttgaggtcaacggcatcgtactgttccagggacagtcggggccacaatgatgtgtatatttcggagcatgttatgccttatgctgcaaataagaataatatgtactctacccccagggttttaaattcctgcagcaatagacgcgtgtacaagaaaatgcctagtatcgaatctgatgtttaa

SEQ ID NO: 91 - DNA, 411 bp, Homo sapiens
  GluN2A S1
gacaaccatctcagcatcgtcaccctggaggaggccccattcgtcatcgtggaagacatagaccccctgaccgagacgtgtgtgaggaacaccgtgccatgtcggaagttcgtcaaaatcaacaattcaaccaatgaggggatgaatgtgaagaaatgctgcaaggggttctgcattgatattctgaagaagctttccagaactgtgaagtttacttacgacctctatctggtgaccaatgggaagcatggcaagaaagttaacaatgtgtggaatggaatgatcggtgaagtggtctatcaacgggcagtcatggcagttggctcgctcaccatcaatgaggaacgttctgaagtggtggacttctctgtgccctttgtggaaacgggaatcagtgtcatggtttcaaga

SEQ ID NO: 92 - DNA, 426 bp, Homo sapiens
  GluN2A S2
caagtgaccggcctcagtgacaaaaagtttcagagacctcatgactattccccaccttttcgatttgggacagtgcctaatggaagcacggagagaaacattcggaataactatccctacatgcatcagtacatgaccaaatttaatcagaaaggagtagaggacgccttggtcagcctgaaaacggggaagctggacgctttcatctacgatgccgcagtcttgaattacaaggctgggagggatgaaggctgcaagctggtgaccatcgggagtgggtacatctttgccaccaccggttatggaattgcccttcagaaaggctctccttggaagaggcagatcgacctggccttgcttcagtttgtgggtgatggtgagatggaggagctggagaccctgtggctcactgggatctgccacaac

SEQ ID NO: 93 - DNA, 2049 bp, Artificial Sequence
  GluN2A ecd incl. signal sequence and GT-linker between S1 and S2
atgggcagagtgggctattggaccctgctggtgctgccggcccttctggtctggcgcggtccggcgccgagcgcggcggcggagaagggtccccccgcgctaaatattgcggtgatgctgggtcacagccacgacgtgacagagcgcgaacttcgaacactgtggggccccgagcaggcggcggggctgcccctggacgtgaacgtggtagctctgctgatgaaccgcaccgaccccaagagcctcatcacgcacgtgtgcgacctcatgtccggggcacgcatccacggcctcgtgtttggggacgacacggaccaggaggccgtagcccagatgctggattttatctcctcccacaccttcgtccccatcttgggcattcatgggggcgcatctatgatcatggctgacaaggatccgacgtctaccttcttccagtttggagcgtccatccagcagcaagccacggtcatgctgaagatcatgcaggattatgactggcatgtcttctccctggtgaccactatcttccctggctacagggaattcatcagcttcgtcaagaccacagtggacaacagctttgtgggctgggacatgcagaatgtgatcacactggacacttcctttgaggatgcaaagacacaagtccagctgaagaagatccactcttctgtcatcttgctctactgttccaaagacgaggctgttctcattctgagtgaggcccgctcccttggcctcaccgggtatgatttcttctggattgtccccagcttggtctctgggaacacggagctcatcccaaaagagtttccatcgggactcatttctgtctcctacgatgactgggactacagcctggaggcgagagtgagggacggcattggcatcctaaccaccgctgcatcttctatgctggagaagttctcctacatccccgaggccaaggccagctgctacgggcagatggagaggccagaggtcccgatgcacaccttgcacccatttatggtcaatgttacatgggatggcaaagacttatccttcactgaggaaggctaccaggtgcaccccaggctggtggtgattgtgctgaacaaagaccgggaatgggaaaaggtgggcaagtgggagaaccatacgctgagcctgaggcacgccgtgtggcccaggtacaagtccttctccgactgtgagccggatgacaaccatctcagcatcgtcaccctggaggaggccccattcgtcatcgtggaagacatagaccccctgaccgagacgtgtgtgaggaacaccgtgccatgtcggaagttcgtcaaaatcaacaattcaaccaatgaggggatgaatgtgaagaaatgctgcaaggggttctgcattgatattctgaagaagctttccagaactgtgaagtttacttacgacctctatctggtgaccaatgggaagcatggcaagaaagttaacaatgtgtggaatggaatgatcggtgaagtggtctatcaacgggcagtcatggcagttggctcgctcaccatcaatgaggaacgttctgaagtggtggacttctctgtgccctttgtggaaacgggaatcagtgtcatggtttcaagaggcacccaagtgaccggcctcagtgacaaaaagtttcagagacctcatgactattccccaccttttcgatttgggacagtgcctaatggaagcacggagagaaacattcggaataactatccctacatgcatcagtacatgaccaaatttaatcagaaaggagtagaggacgccttggtcagcctgaaaacggggaagctggacgctttcatctacgatgccgcagtcttgaattacaaggctgggagggatgaaggctgcaagctggtgaccatcgggagtgggtacatctttgccaccaccggttatggaattgcccttcagaaaggctctccttggaagaggcagatcgacctggccttgcttcagtttgtgggtgatggtgagatggaggagctggagaccctgtggctcactgggatctgccacaac

SEQ ID NO: 94 - DNA, 4455 bp, Homo sapiens
  Human GluN2B NM_000834
atgaagcccagagcggagtgctgttctcccaagttctggttggtgttggccgtcctggccgtgtcaggcagcagagctcgttctcagaagagcccccccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcacccccatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccgaatgtgtccagagactgaagagcaggaggatgaccatctgagcattgtgaccctggaggaggcaccatttgtcattgtggaaagtgtggaccctctgagtggaacctgcatgaggaacacagtcccctgccaaaaacgcatagtcactgagaataaaacagacgaggagccgggttacatcaaaaaatgctgcaaggggttctgtattgacatccttaagaaaatttctaaatctgtgaagttcacctatgacctttacctggttaccaatggcaagcatgggaagaaaatcaatggaacctggaatggtatgattggagaggtggtcatgaagagggcctacatggcagtgggctcactcaccatcaatgaggaacgatcggaggtggtcgacttctctgtgcccttcatagagacaggcatcagtgtcatggtgtcacgcagcaatgggactgtctcaccttctgccttcttagagccattcagcgctgacgtatgggtgatgatgtttgtgatgctgctcatcgtctcagccgtggctgtctttgtctttgagtacttcagccctgtgggttataacaggtgcctcgctgatggcagagagcctggtggaccctctttcaccatcggcaaagctatttggttgctctggggtctggtgtttaacaactccgtacctgtgcagaacccaaaggggaccacctccaagatcatggtgtcagtgtgggccttctttgctgtcatcttcctggccagctacactgccaacttagctgccttcatgatccaagaggaatatgtggaccaggtttctggcctgagcgacaaaaagttccagagacctaatgacttctcaccccctttccgctttgggaccgtgcccaacggcagcacagagagaaatattcgcaataactatgcagaaatgcatgcctacatgggaaagttcaaccagaggggtgtagatgatgcattgctctccctgaaaacagggaaactggatgccttcatctatgatgcagcagtgctgaactatatggcaggcagagatgaaggctgcaagctggtgaccattggcagtgggaaggtctttgcttccactggctatggcattgccatccaaaaagattctgggtggaagcgccaggtggaccttgctatcctgcagctctttggagatggggagatggaagaactggaagctctctggctcactggcatttgtcacaatgagaagaatgaggtcatgagcagccagctggacattgacaacatggcaggggtcttctacatgttgggggcggccatggctctcagcctcatcaccttcatctgcgaacaccttttctattggcagttccgacattgctttatgggtgtctgttctggcaagcctggcatggtcttctccatcagcagaggtatctacagctgcatccatggggtggcgatcgaggagcgccagtctgtaatgaactcccccaccgcaaccatgaacaacacacactccaacatcctgcgcctgctgcgcacggccaagaacatggctaacctgtctggtgtgaatggctcaccgcagagcgccctggacttcatccgacgggagtcatccgtctatgacatctcagagcaccgccgcagcttcacgcattctgactgcaaatcctacaacaacccgccctgtgaggagaacctcttcagtgactacatcagtgaggtagagagaacgttcgggaacctgcagctgaaggacagcaacgtgtaccaagatcactaccaccatcaccaccggccccatagtattggcagtgccagctccatcgatgggctctacgactgtgacaacccacccttcaccacccagtccaggtccatcagcaagaagcccctggacatcggcctcccctcctccaagcacagccagctcagtgacctgtacggcaaattctccttcaagagcgaccgctacagtggccacgacgacttgatccgctccgatgtctctgacatctcaacccacaccgtcacctatgggaacatcgagggcaatgccgccaagaggcgtaagcagcaatataaggacagcctgaagaagcggcctgcctcggccaagtcccgcagggagtttgacgagatcgagctggcctaccgtcgccgaccgccccgctcccctgaccacaagcgctacttcagggacaaggaagggctacgggacttctacctggaccagttccgaacaaaggagaactcaccccactgggagcacgtagacctgaccgacatctacaaggagcggagtgatgactttaagcgcgactccgtcagcggaggagggccctgtaccaacaggtctcacatcaagcacgggacgggcgacaaacacggcgtggtcagcggggtacctgcaccttgggagaagaacctgaccaacgtggagtgggaggaccggtccgggggcaacttctgccgcagctgtccctccaagctgcacaactactccacgacggtgacgggtcagaactcgggcaggcaggcgtgcatccggtgtgaggcttgcaagaaagcaggcaacctgtatgacatcagtgaggacaactccctgcaggaactggaccagccggctgccccagtggcggtgacgtcaaacgcctccaccactaagtaccctcagagcccgactaattccaaggcccagaagaagaaccggaacaaactgcgccggcagcactcctacgacaccttcgtggacctgcagaaggaagaagccgccctggccccgcgcagcgtaagcctgaaagacaagggccgattcatggatgggagcccctacgcccacatgtttgagatgtcagctggcgagagcacctttgccaacaacaagtcctcagtgcccactgccggacatcaccaccacaacaaccccggcggcgggtacatgctcagcaagtcgctctaccctgaccgggtcacgcaaaaccctttcatccccacttttggggacgaccagtgcttgctccatggcagcaaatcctacttcttcaggcagcccacggtggcgggggcgtcgaaagccaggccggacttccgggcccttgtcaccaacaagccggtggtctcggcccttcatggggccgtgccagcccgtttccagaaggacatctgtatagggaaccagtccaacccctgtgtgcctaacaacaaaaaccccagggctttcaatggctccagcaatgggcatgtttatgagaaactttctagtattgagtctgatgtctga

SEQ ID NO: 95 - DNA, 2052 bp, Artificial Sequence
  GluN2B ecd incl. signal sequence and GT-linker between S1 and S2
atgaagcccagagcggagtgctgttctcccaagttctggttggtgttggccgtcctggccgtgtcaggcagcagagctcgttctcagaagagcccccccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcacccccatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccgaatgtgtccagagactgaagagcaggaggatgaccatctgagcattgtgaccctggaggaggcaccatttgtcattgtggaaagtgtggaccctctgagtggaacctgcatgaggaacacagtcccctgccaaaaacgcatagtcactgagaataaaacagacgaggagccgggttacatcaaaaaatgctgcaaggggttctgtattgacatccttaagaaaatttctaaatctgtgaagttcacctatgacctttacctggttaccaatggcaagcatgggaagaaaatcaatggaacctggaatggtatgattggagaggtggtcatgaagagggcctacatggcagtgggctcactcaccatcaatgaggaacgatcggaggtggtcgacttctctgtgcccttcatagagacaggcatcagtgtcatggtgtcacgcggcacccaggtttctggcctgagcgacaaaaagttccagagacctaatgacttctcaccccctttccgctttgggaccgtgcccaacggcagcacagagagaaatattcgcaataactatgcagaaatgcatgcctacatgggaaagttcaaccagaggggtgtagatgatgcattgctctccctgaaaacagggaaactggatgccttcatctatgatgcagcagtgctgaactatatggcaggcagagatgaaggctgcaagctggtgaccattggcagtgggaaggtctttgcttccactggctatggcattgccatccaaaaagattctgggtggaagcgccaggtggaccttgctatcctgcagctctttggagatggggagatggaagaactggaagctctctggctcactggcatttgtcacaat

SEQ ID NO: 96 - protein, 636 aa, Artificial Sequence
  protein seq of fusion protein #9 N2C-ATD-Fc
MGGALGPALLLTSLFGAWAGLGPGQGEQGMTVAVVFSSSGPPQAQFRARLTPQSFLDLPLEIQPLTVGVNTTNPSSLLTQICGLLGAAHVHGIVFEDNVDTEAVAQILDFISSQTHVPILSISGGSAVVLTPKEPGSAFLQLGVSLEQQLQVLFKVLEEYDWSAFAVITSLHPGHALFLEGVRAVADASHVSWRLLDVVTLELGPGGPRARTQRLLRQLDAPVFVAYCSREEAEVLFAEAAQAGLVGPGHVWLVPNLALGSTDAPPATFPVGLISVVTESWRLSLRQKVRDGVAILALGAHSYWRQHGTLPAPAGDCRVHPGPVSPAREAFYRHLLNVTWEGRDFSFSPGGYLVQPTMVVIALNRHRLWEMVGRWEHGVLYMKYPVWPRYSASLQPVVDSRHLTVGSSTMVRSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 97 - protein, 405 aa, Homo sapiens
  Amino terminal domain of GluN2C (including signal sequence)
MGGALGPALLLTSLFGAWAGLGPGQGEQGMTVAVVFSSSGPPQAQFRARLTPQSFLDLPLEIQPLTVGVNTTNPSSLLTQICGLLGAAHVHGIVFEDNVDTEAVAQILDFISSQTHVPILSISGGSAVVLTPKEPGSAFLQLGVSLEQQLQVLFKVLEEYDWSAFAVITSLHPGHALFLEGVRAVADASHVSWRLLDVVTLELGPGGPRARTQRLLRQLDAPVFVAYCSREEAEVLFAEAAQAGLVGPGHVWLVPNLALGSTDAPPATFPVGLISVVTESWRLSLRQKVRDGVAILALGAHSYWRQHGTLPAPAGDCRVHPGPVSPAREAFYRHLLNVTWEGRDFSFSPGGYLVQPTMVVIALNRHRLWEMVGRWEHGVLYMKYPVWPRYSASLQPVVDSRHLTV

SEQ ID NO: 98 - DNA, 1911 bp, Artificial Sequence
  DNA seq of fusion protein #9 N2C-ATD-Fc
atgggtggcgcacttggccctgctttgctgctcactagcctgtttggagcatgggctggtcttgggccaggtcaaggcgagcaagggatgaccgtggccgtggtgttctcctcaagtggaccacctcaggcacagttccgtgccagactgacaccgcagagcttcctggatctcccactggagatacagcctctcactgtgggcgtgaacaccaccaatccctcatccctgctgacacagatctgcggacttctgggtgcagcccatgtccacgggatcgtgttcgaggacaacgtggacacagaagccgtagcccagattctggacttcatcagctcacagacccacgttcccattctgagcattagtggcgggagcgctgtagtgctcactcccaaagagccggggtctgcatttctgcagctcggagtaagccttgagcagcagctccaggtcttgttcaaggtgctggaagagtacgactggtctgcgtttgccgtcatcacctctctgcatcctggccatgctctgtttctggaaggcgttagggctgtcgccgatgcgtcccacgtcagttggcggttgctggatgtggtcacgttggagcttggacctggaggccccagggccagaacacagcgactgctgcgccaactggatgctcccgtgtttgtggcctattgttcccgcgaggaggccgaggtgctcttcgccgaggcggcgcaggccggtctggtggggcccggccacgtgtggctggtgcccaacctggcgctgggcagcaccgatgcgccccccgccaccttccccgtgggcctcatcagcgtcgtcaccgagagctggcgcctcagcctgcgccagaaggtgcgcgacggcgtggccattctggccctgggcgcccacagctactggcgccagcatggaaccctgccagccccggccggggactgccgtgttcaccctgggcccgtcagccctgcccgggaggccttctacaggcacctactgaatgtcacctgggagggccgagacttctccttcagccctggtgggtacctggtccagcccaccatggtggtgatcgccctcaaccggcaccgcctctgggagatggtggggcgctgggagcatggcgtcctatacatgaagtaccccgtgtggcctcgctacagtgcctctctgcagcctgtggtggacagtcggcacctgacggtgggctcgagcaccatggttagatctagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 99 - DNA, 1215 bp, Homo sapiens
  Amino terminal domain of GluN2C (including signal sequence)
atgggtggcgcacttggccctgctttgctgctcactagcctgtttggagcatgggctggtcttgggccaggtcaaggcgagcaagggatgaccgtggccgtggtgttctcctcaagtggaccacctcaggcacagttccgtgccagactgacaccgcagagcttcctggatctcccactggagatacagcctctcactgtgggcgtgaacaccaccaatccctcatccctgctgacacagatctgcggacttctgggtgcagcccatgtccacgggatcgtgttcgaggacaacgtggacacagaagccgtagcccagattctggacttcatcagctcacagacccacgttcccattctgagcattagtggcgggagcgctgtagtgctcactcccaaagagccggggtctgcatttctgcagctcggagtaagccttgagcagcagctccaggtcttgttcaaggtgctggaagagtacgactggtctgcgtttgccgtcatcacctctctgcatcctggccatgctctgtttctggaaggcgttagggctgtcgccgatgcgtcccacgtcagttggcggttgctggatgtggtcacgttggagcttggacctggaggccccagggccagaacacagcgactgctgcgccaactggatgctcccgtgtttgtggcctattgttcccgcgaggaggccgaggtgctcttcgccgaggcggcgcaggccggtctggtggggcccggccacgtgtggctggtgcccaacctggcgctgggcagcaccgatgcgccccccgccaccttccccgtgggcctcatcagcgtcgtcaccgagagctggcgcctcagcctgcgccagaaggtgcgcgacggcgtggccattctggccctgggcgcccacagctactggcgccagcatggaaccctgccagccccggccggggactgccgtgttcaccctgggcccgtcagccctgcccgggaggccttctacaggcacctactgaatgtcacctgggagggccgagacttctccttcagccctggtgggtacctggtccagcccaccatggtggtgatcgccctcaaccggcaccgcctctgggagatggtggggcgctgggagcatggcgtcctatacatgaagtaccccgtgtggcctcgctacagtgcctctctgcagcctgtggtggacagtcggcacctgacggtg

(19) United States
(12) Patent Application Publication
(10) Pub. No.: US 2023/0280340 A1
(43) Pub. Date: Sep. 7, 2023

(54) NMDA RECEPTOR CONSTRUCTS TO DETECT AND ISOLATE NMDAR AUTOANTIBODIES

(71) Applicant: Deutsches Zentrum für Neurodegenerative Erkrankungen e.V. (DZNE), Bonn (DE)

(72) Inventors: Hans-Christian Kornau, Berlin (DE); Harald Prüss, Berlin (DE); Craig Curtis Garner, Berlin (DE); Tanita Frey, Tokyo (JP)

(21) Appl. No.: 17/627,077

(22) PCT Filed: Jul. 2, 2020
(86) PCT No.: PCT/EP2020/068732

§ 371 Date: Jan. 13, 2022

(30) Foreign Application Priority Data
Jul. 16, 2019 (EP) 19186524.5
Jul. 18, 2019 (LU) LU101308

(51) Int. Cl. G01N 33/564; C07K 14/705

(52) U.S. Cl. CPC G01N 33/564; C07K 14/70571; G01N 2333/70571; G01N 2800/24

(57) ABSTRACT

A soluble N-methyl-D-aspartate receptor (NMDAR) protein construct includes one or more NMDAR autoantibody epitopes. The construct includes an extracellular domain (ECD) of the NMDAR subunit GluN1 or a fragment of the subunit and an ECD of at least one of the NMDAR subunits GluN2A, GluN2B, GluN2C or GluN2D, or fragment of these subunits. An in vitro method for the detection of NMDAR autoantibodies in a sample includes providing a sample suspected of including NMDAR autoantibodies, providing the NMDAR protein construct as a capture molecule, contacting the sample with the NMDAR protein construct, thereby binding NMDAR autoantibodies from the sample to the NMDAR protein construct, and determining the presence and optionally the amount of bound NMDAR autoantibodies. The method is applied for the diagnosis, prognosis, disease monitoring, patient stratification and/or therapy monitoring of a medical condition associated with autoantibodies against the NMDAR, preferably anti-NMDAR encephalitis.

### REFERENCE TO SEQUENCE LISTING

A Sequence Listing submitted as an ASCII text file via EFS-Web is hereby incorporated by reference in accordance with 35 U.S.C. § 1.52(e). The name of the ASCII text file for the Sequence Listing is 56453321_1.txt, the date of creation of the ASCII text file is Oct. 12, 2022, and the size of the ASCII text file is 317 KB.

### DESCRIPTION

The invention relates to a soluble N-methyl-D-aspartate receptor (NMDAR) protein construct comprising one or more NMDAR autoantibody epitopes, wherein the construct comprises an extracellular domain (ECD) of the NMDAR subunit GluN1 or a fragment thereof and an ECD of at least one of the NMDAR subunits GluN2A, GluN2B, GluN2C or GluN2D, or fragment thereof. Further, the invention relates to an in vitro method for the detection of NMDAR autoantibodies in a sample, the method comprising, a.) providing a sample suspected of comprising NMDAR autoantibodies, b.) providing a NMDAR protein construct of the invention as a capture molecule, c.) contacting said sample with said NMDAR protein construct, thereby binding NMDAR autoantibodies from said sample to said NMDAR protein construct, and d.) determining the presence and optionally the amount of bound NMDAR autoantibodies. In embodiments, the method of the invention is applied for the diagnosis, prognosis, disease monitoring, patient stratification and/or therapy monitoring of a medical condition associated with autoantibodies against the NMDAR, preferably anti-NMDAR encephalitis.

### BACKGROUND

Anti-NMDA receptor encephalitis (NMDAR encephalitis) is the most common form of a growing number of autoimmune encephalitides (Dalmau et al., 2017; Dalmau and Graus, 2018). This disorder affects primarily young women, and patients present with psychiatric and neurological symptoms including memory loss, hallucinations, paranoia as well as seizures and dyskinesias. Current treatment options include glucocorticoids, plasma exchange as well as Rituximab and Cyclophosphamide. NMDAR encephalitis is caused by the generation of autoantibodies targeting the extracellular region of the principal NMDA receptor subunit GluN1 both in the blood and in the brain. The antibodies alter the surface dynamics, induce crosslinking and internalization of NMDA receptors, and the resulting depletion of NMDA receptors may explain several of the neurological symptoms observed in patients (Hughes et al., 2010; Jezequel et al., 2017a; Ladepeche et al., 2018). Single recombinant human antibodies to GluN1 derived from CSF B cells induced downregulation of NMDA receptor function (Kreye et al., 2016), suggesting they are the main pathogenic agents in this disease. This conjecture has been consolidated recently by mouse models using active and passive immunization (Hughes et al., 2010; Jones et al., 2018; Malviya et al., 2017).

In striking contrast to other forms of antibody-mediated encephalitides and to many autoimmune disorders, NMDAR encephalitis appears not to be linked to a specific HLA-II type (Kim et al., 2017; Mueller et al., 2018). A significant number of female NMDAR encephalitis patients have an ovarian teratoma. Interestingly, some antibody-secreting cells isolated from the brain of NMDAR encephalitis patients expressed unmutated/germline antibodies to NMDA receptors (Kreye et al., 2016, Wenke et al., 2019). While the origin of the autoimmune response is not fully clarified, it appears that a large fraction of the population may have NMDA receptor antibodies and the presence of NMDA receptor autoantibodies in the serum may constitute a general problem in pregnant women and in older people with an impaired blood-brain barrier. Furthermore, NMDA receptor autoantibodies have been found in patients with neuropsychiatric diseases other than NMDAR encephalitis and are likely contributing to the disease progress (Jezequel et al., 2017a).

Immunosuppression and plasma exchange or intravenous immunoglobulins are established treatments of NMDAR encephalitis. However, a selective removal of the disease-causing antibodies would be preferred since it is expected to show lower side effects. Depleting sera from NMDA receptor antibodies, for example using extracorporeal techniques similar to plasma exchange, may be applied to NMDAR encephalitis and to other disorders connected with NMDA receptor autoantibodies. A prerequisite for such a form of apheresis would be the generation of a stable protein that is capable of binding NMDA receptor autoantibodies from patients, i.e. a soluble antigen for NMDA receptor antibodies.

NMDA receptors assemble from the principal GluN1 subunit and modulatory GluN2/3 subunits (Paoletti et al., 2013). Heterologous expression of GluN1 and deletion mutants thereof revealed that human IgG isolated from NMDAR encephalitis patients bound to a specific extracellular region termed the amino terminal domain (ATD) of GluN1 (Gleichman et al., 2012). Mutations of an amino acid at the hinge region of this clam shell-like domain (N368/9) abrogated antibody binding suggesting that the closure of the clam shell or posttranslational modifications of these amino acids influence or constitute the epitope recognized by the antibodies. Furthermore, the ATD conformation is linked to channel opening and the antibodies preferentially bind to the receptor in the activated state (Gleichman et al., 2012). Native core NMDA receptors function as dimers of GluN1-GluN2 dimers. Within this structure the ATD of GluN1 interacts directly with the ATD of a GluN2 subunit (Lee and Gouaux, 2011) and their conformations are coordinately altered during receptor activation and inhibition (Lee et al., 2014; Tajima et al., 2016; Zhu et al., 2016). Furthermore, NMDA receptor antibodies may be directed to a specific NMDA receptor subtype defined by the GluN2 subunit in some patients. Antigens encompassing GluN2 extracellular domains may therefore be preferable over antigens containing only GluN1.

Autoantibodies to NMDA receptors are routinely detected using the Euroimmun cell-based assay (CBA) kit based on biochips containing acetone-fixed GluN1-expressing heterologous cells as described in WO2012/076000A2. However, NMDA receptor autoantibodies often recognize the native conformation of their antigens and live staining of NMDA receptor-expressing HEK293 cells proved more sensitive than the commercial assay using prefixed cells for detecting low titers (Jezequel et al., 2017b). These cell-based assays require a visual inspection of the results that may cause bias. Furthermore, even if such tests would be accessible to automated routine testing, the fact that there are many different antigens on the cell surfaces and not just the antigen of interest makes them prone to false positive results.

Therefore, a sensitive, quantifiable high throughput method to detect NMDA receptor autoantibodies is urgently needed. In this direction, a single nanoparticle imaging approach on primary hippocampal neurons detected NMDA receptor autoantibodies at low titers and would be automatable, but it is technically quite challenging (Jezequel et al., 2017b). An ELISA allowing the comparison of different titers of NMDA receptor autoantibodies based on lysed HEK293 cells expressing NMDA receptors has been described early-on (Dalmau et al., 2008). US2003/096331A1 discloses a method for the detection of antibodies against NR2A (GluN2A) and/or NR2B (GluN2B) in the context of a diagnostic method for stroke, for which an amino-terminal fragment of NR2A/NR2B is synthesized and purified, but not in the combination with an ECD of GluN1. In fact, the disclosed method does not intend to identify antibodies directed against GluN1, but antibodies directed specifically against GluN2A or GluN2B, independent of their association with GluN1, and is therefore intended for a completely different use as compared to the present invention. Importantly, using the method of US2003/096331A1 it is not possible to identify antibodies that bind GluN1 or parts thereof.

Recently, a cell line expressing the entire amino terminal region of GluN1 (amino acids 1-561, encompassing ATD and S1 domain) fused to a myc- and a polyhistidin-tag, a tobacco etch virus (TEV) cleavage site, and the transmembrane region of the PDGF receptor was presented (Sharma et al., 2018). TEV treatment of these cells released the amino terminal extracellular segment of GluN1. This fragment, attached to an ELISA plate via an anti-polyhistidin antibody, was able to detect monoclonal anti-GluN1 antibodies. However, the detection sensitivity of this antigen may be limited. In summary, there are no stable and soluble antigens of NMDA receptors available that maintain the native conformation and incorporate GluN1 as well as GluN2 segments for detecting autoantibodies present for example in serum or CSF, for example in an ELISA.

Accordingly, there remains a strong need in the art for the provision of NMDAR protein constructs comprising one or more NMDAR autoantibody epitopes that may be composed of or stabilized by the extracellular domains of GluN1 and GluN2A, GluN2B, GluN2C and/or GluN2D or fragments thereof.

### SUMMARY OF THE INVENTION

In light of the prior art the technical problem underlying the present invention is the provision of improved NMDAR protein constructs for the detection of NMDAR autoantibodies and for the treatment of autoimmune diseases associated with NMDAR autoantibodies.

This problem is solved by the features of the independent claims. Preferred embodiments of the present invention are provided by the dependent claims.

The NMDAR Protein Construct

The invention therefore relates to soluble N-methyl-D-aspartate receptor (NMDAR) protein construct comprising one or more NMDAR autoantibody epitopes, wherein the construct comprises an extracellular domain (ECD) of the NMDAR subunit GluN1 or a fragment thereof and an ECD at least one of the NMDAR subunits GluN2A, GluN2B, GluN2C or GluN2D, or fragment thereof. In embodiments, the NMDAR protein construct comprises an extracellular domain (ECD) of the NMDAR subunit GluN1 or a fragment thereof and an ECD of the NMDAR subunit GluN2A or fragment thereof and/or GluN2B or fragment thereof.

The invention also relates to a N-methyl-D-aspartate receptor (NMDAR) protein construct lacking a NMDAR transmembrane domain comprising one or more NMDAR autoantibody epitopes, wherein the construct comprises an extracellular domain (ECD) of the NMDAR subunit GluN1 or a fragment thereof and an ECD of at least one of the NMDAR subunits GluN2A, GluN2B, GluN2C or GluN2D, or fragment thereof. In embodiments, NMDAR protein construct lacking a NMDAR transmembrane domain comprises an extracellular domain (ECD) of the NMDAR subunit GluN1 or a fragment thereof and an ECD of the NMDAR subunit GluN2A or fragment thereof and/or GluN2B or fragment thereof.

The invention further relates to a N-methyl-D-aspartate receptor (NMDAR) protein construct comprising one or more NMDAR autoantibody epitopes, wherein the construct comprises an extracellular domain (ECD) of the NMDAR subunit GluN1 or a fragment thereof and an ECD of at least one of the NMDAR subunits GluN2A, GluN2B, GluN2C or GluN2D, or fragment thereof and a dimerization domain. In embodiments, the NMDAR protein construct comprises an extracellular domain (ECD) of the NMDAR subunit GluN1 or a fragment thereof and an ECD of the NMDAR subunit GluN2A or fragment thereof and/or GluN2B or fragment thereof and a dimerization domain.

Additionally, the invention relates to a N-methyl-D-aspartate receptor (NMDAR) protein construct comprising one or more NMDAR autoantibody epitopes, wherein the construct comprises an extracellular domain (ECD) of the NMDAR subunit GluN1 or a fragment thereof and an ECD at least one of the NMDAR subunits GluN2A, GluN2B, GluN2C or GluN2D, or fragment thereof and is not present in or on a cell. In embodiments, the NMDAR protein construct comprises an extracellular domain (ECD) of the NMDAR subunit GluN1 or a fragment thereof and an ECD of the NMDAR subunit GluN2A or fragment thereof and/or GluN2B or fragment thereof and is not present in or on a cell.

The NMDAR protein constructs of the present invention may be used for screening for NMDA receptor autoantibodies in the serum or CSF of patients.

The data disclosed herein show that soluble NMDAR protein constructs of the invention, in particular in embodiments as Fc fusion proteins, are able to detect antibodies in the serum of NR encephalitis patients. The soluble NMDAR-Fc protein constructs (srNR-Fc proteins) have several advantages over cells and cell-based assays of the state of the art detecting antibodies by using NMDAR expressed on the surface of in vitro cultured cells. The srNR-Fc proteins can be purified and stored. The purified srNR-Fc proteins constitute a clean antigen as compared to heterologous cells that contain a number of additional proteins. They allow generating a very high antigen concentration leading to an improved sensitivity in the antibody detection as compared to the cell-based assays (CBA).

In contrast to known NMDAR constructs of the state of the art the present invention relates to NMDAR constructs that comprise at least fragments of two NMDAR subunits, making it possible to identify autoantibodies that bind to epitopes that are formed by residues of two subunits or that are only formed or stabilized by the assembly of two subunits. In contrast, constructs of the state of the art only comprising one subunit or fragments therefore will not bind and remove such autoantibodies.

By using a protein construct of the invention for the recognition of NMDAR specific antibodies in a sample, it is possible to identify antibodies that only bind to NMDAR and GluN1 when GluN1 is in association with a GluN2 subunit, which leads to stabilization of the respective epitope of such an antibody. It is not possible to identify such antibodies by proteins or protein constructs that only comprise GluN1 or a GluN2 subunit, because the respective epitope is only stabilized or formed by association of GluN1 with the respective GluN2 subunit.

The NMDAR protein constructs disclosed herein, for example comprising a Fc fragment or other or additional tags may be useful in a variety of diagnostic assays. For example, a diagnostic assay in form of an ELISA-like assay as disclosed in the examples may be used as a companion diagnostic that allows a quantifiable high-throughput method to detect NMDA receptor autoantibodies and to detect autoantibodies in other autoimmune encephalopathies.

Furthermore, the NMDAR protein constructs may be used for distinguishing between autoimmune responses against different NMDA receptor compositions allowing patient classification.

As disclosed in the examples provided herein, the NMDAR protein constructs, such as various srNR-Fc fusion proteins and dimers formed by such fusion proteins, yield different signals in response to antibodies/sera derived from different patients, indicating patient-specific variant antibody profiles. Accordingly, a set comprising different NMDAR protein constructs of the invention comprising different combinations of ECD and ATD of GluN1, GluN2AGluN2B, GluN2C and GluN2D such as the set of srNR-Fc fusions described in the examples, can be used to classify the patient-specific antibody profile, i.e. distinguish between antibodies mainly directed to GluN1 or to a heteromeric GluN1/GluN2 structure and potentially to a GluN1/GluN2 structure of a specific composition. Subclassification of NMDAR encephalitis patients may ultimately lead to an improved therapy.

Accordingly, the present invention also relates to a set or kit providing two or more NMDAR protein constructs of the present invention providing different combinations of GluN1 and one or more of GluN2A-D, or fragments thereof.

In embodiments, the invention relates to two NMDAR protein constructs of the invention, wherein one construct comprises the ECDs of GluN1 and GluN2A or fragments thereof, and the other construct comprises ECDs of GluN1 and Glu2B or fragments thereof.

In embodiments, the invention relates to two NMDAR protein constructs of the invention, wherein one construct comprises the ECDs of GluN1 and GluN2A or fragments thereof, and the other construct comprises ECDs of GluN1 and Glu2C or fragments thereof.

In embodiments, the invention relates to two NMDAR protein constructs of the invention, wherein one construct comprises the ECDs of GluN1 and GluN2A or fragments thereof, and the other construct comprises ECDs of GluN1 and Glu2D or fragments thereof.

In embodiments, the invention relates to two NMDAR protein constructs of the invention, wherein one construct comprises the ECDs of GluN1 and GluN2C or fragments thereof, and the other construct comprises ECDs of GluN1 and Glu2B or fragments thereof.

In embodiments, the invention relates to two NMDAR protein constructs of the invention, wherein one construct comprises the ECDs of GluN1 and GluN2D or fragments thereof, and the other construct comprises ECDs of GluN1 and Glu2B or fragments thereof.

In embodiments, the invention relates to two NMDAR protein constructs of the invention, wherein one construct comprises the ECDs of GluN1 and GluN2C or fragments thereof, and the other construct comprises ECDs of GluN1 and Glu2D or fragments thereof.

The NMDAR protein constructs of the invention can comprise four ECD of GluN subunits mimicking the tetrameric assembly of the NMDAR. For example, a construct of the invention can comprise two GluN1 ECD or fragments thereof and two equal or different ECD of GluN2A, GluN2B, GluN2C and/or GluN2D.

Also, the constructs enable labelling of B cells expressing NMDA receptor autoantibodies, which can be used as a diagnostic tool and to facilitate cell isolation and IgG sequence analyses.

The NMADR protein constructs of the invention may be used to detect not only soluble NMDAR autoantibodies, but also NMDAR autoantibodies expressed on the surface of B cells that are present in the serum and CSF of patients. Labelling of B cells expressing NMDA receptor autoantibodies may also be used as a diagnostic tool.

Additionally, such constructs can be used for selective removal of antibodies to NMDA receptors from sera or CSF of patients.

Current apheresis protocols mostly deplete the serum from all IgG. The (soluble) NMDAR protein constructs of the invention may be non-covalently or covalently linked to agarose/sepharose beads or a different matrix material. The resulting matrix may be used to specifically immunodeplete NMDAR autoantibodies from sera of patient. This procedure will be efficient and avoids all side effects connected to the complete immuno-depletion, such as severe infections or impaired wound healing. The immune system will not be weakened by this method in contrast to current treatment options.

The following further and preferred embodiments of the invention all relate to each of the NMDAR protein constructs listed above.

In embodiments of the invention, the constructs of the invention lack a NMDAR transmembrane domain.

Furthermore, the constructs can comprise a dimerization domain and/or a capture domain.

Importantly, in particular constructs of the invention the dimerization domain is the capture domain.

In embodiments, the dimerization domain comprises a leucin-zipper and/or a coiled-coil-domain.

In specific embodiments of the invention, the dimerization domain and/or the capture domain comprises or consists of an antibody Fc-fragment. In embodiments, the Fc-fragment is a fragment of rabbit IgG Fc.

The presence of a dimerization and/or capture domain, for example a Fc-fragment, can be particularly advantageous since the soluble NMDAR protein construct is stabilized by these domains. In particular a Fc-moiety enables long-term storage of the construct with preserved conformation.

In particular, NMDAR protein constructs of the invention the ECD of GluN1 or fragment thereof comprises or consists of the amino-terminal domain (ATD) of GluN1 or a fragment thereof.

In the context of the constructs of the present invention, the ECD of at least one of the NMDAR subunits GluN2A, GluN2B, GluN2C or GluN2D, or fragment thereof can comprise or consist of the ATD of at least one of the NMDAR subunits GluN2A, GluN2B, GluN2C or GluN2D, or fragment thereof, respectively.

Furthermore, the ECD of GluN2A or fragment thereof and/or the ECD of GluN2B or fragment thereof can comprise or consist of the ATD of GluN2A or fragment thereof and/or the ATD of GluN2B or fragment thereof, respectively.

In certain NMDAR protein constructs of the invention, the ECD of GluN1 and the ECD of at least one of the NMDAR subunits GluN2A, GluN2B, GluN2C or GluN2D, or fragment thereof, are covalently linked, preferably as a fusion protein. Therein, the ECD of GluN1 and the ECD of at least one of the NMDAR subunits GluN2A, GluN2B, GluN2C or GluN2D, or fragment thereof, may be linked directly or through a protein linker as a fusion protein. In embodiments, the ECD of GluN1 and the ECD of GluN2A and/or the ECD of GluN2B, or fragments thereof, are covalently linked, preferably as a fusion protein. Therein, the ECD of GluN1 and the ECD of GluN2A and/or GluN2B, or fragments thereof, may be linked directly or through a protein linker as a fusion protein.

In embodiments, the ECD of GluN1 and the ECD of at least one of the NMDAR subunits GluN2A, GluN2B, GluN2C or GluN2D, or fragment thereof, are linked through a protein linker comprising or consisting of one or more repeats of the amino-acid sequence GGGGS.

In embodiments, the ECD of GluN1 and the ECD of GluN2A and/or GluN2B, or fragments thereof, are linked through a protein linker comprising or consisting of one or more repeats of the amino-acid sequence GGGGS.

In specific embodiments of the invention, the construct comprises one or more protease cleavage sites, such as a TEV cleavage site or any other cleavage site that is recognized by suitable proteases known to a person skilled in the art, between a part of the construct comprising the ECD of GluN1, the ECD of at least one of the NMDAR subunits GluN2A, GluN2B, GluN2C or GluN2D, or fragment thereof, and a part of the construct comprising the dimerization domain and/or the capture domain.

In embodiments, the construct comprises one or more protease cleavage sites, such as a TEV cleavage site or any other cleavage site that is recognized by suitable proteases known to a person skilled in the art, between a part of the construct comprising the ECD of GluN1, the ECD of GluN2A and/or the ECD of GluN2B, or fragments thereof, and a part of the construct comprising the dimerization domain and/or the capture domain.

In preferred embodiments of the NMDAR protein constructs of the invention, the construct is a protein dimer of non-covalently bound monomers, wherein the construct can be a homodimer or a heterodimer. In certain dimeric constructs of the invention, the construct may be a heterodimer formed from the ECD of GluN1 or fragment thereof (as one monomer) and the ECD of at least one of the NMDAR subunits GluN2A, GluN2B, GluN2C or GluN2D, or fragment thereof, (as one monomer). In embodiments of dimeric constructs, the construct may be a heterodimer formed from the ECD of GluN1 or fragment thereof (as one monomer) and the ECD of GluN2A or fragment thereof and/or the ECD of GluN2A or fragment thereof, (as one monomer). Preferably, the dimer is formed through a dimerization domain comprised by each of the monomers. In further embodiments, the NMDAR protein construct of the invention can be a dimer of two monomers, wherein each of the monomers comprises two ECD of fragments thereof of two NMDAR subunits, for example GluN1 and GluN2A, GluN1 and GluN2B, gluN1 and GluN2C or GluN1 and GluN2D. Accordingly, it is possible to provide a NMDAR protein construct that comprises 4 ECD of NMDAR subunits or fragments thereof (provided by two monomers) and therefore mimics the naturally occurring NMDAR receptor, which is a tetramer of 4 subunits, 2 GluN1 subunits and 2 GluN2 subunits. For example, dimerization of the fusion-protein N1-ATD-N2B-ATD-Fc disclosed herein leads to the formation of a NMDAR protein construct of the invention formed by two monomers of N1-ATD-N2B-ATD-Fc comprising 4 ATD domains.

The NMDAR protein constructs of the invention are advantageous in comparison to known NMDAR constructs since they enable assembly of ECD or ATD of GluN1, GluN2A, GluN2B, GluN2C and/or GluN2D subunits in a given construct. This potential for ligand combination represents a strong advantage, since autoantibodies that are specific to a certain subunit or subunit combination can be efficiently bound by the constructs of the invention and a set of autoantibodies present in a sample can be assessed for its differential binding profile. Furthermore, the possibility of combining the ECD of fragments of the ECD of GluN1, GluN2A, GluN2B, GluN2C and/or GluN2D subunits with a capture domain enables flexible use of the constructs in the context of for example an ELISA assay, which is advantageous in comparison to cell-based ELISA assays of the state of the art.

Subunit assemblies that contain domains both from GluN1 and GluN2, in particular GluN2A, GluN2B, GluN2C and/or GluN2D reconstitute the native situation more closely than presentation of only GluN1 subunits as in state of the art assays. By combining ligands/subunits, a more complete binding, detection and/or removal of pathogenic autoantibodies can be achieved. For example, as shown in the examples disclosed herein, preferred combinations of subunit in a NMDAR protein construct of the invention may contain GluN1-ATD and GluN2B-ATD or GluN1-ECD and GluN2B-ECD, either in a single fusion protein or in a construct comprising two proteins that form a heterodimer through assembly via a dimerization domain, such as a Fc-domain. In the examples, it was shown that the combination of fusion protein #1 (N1-ATD-Fc) and fusion protein #6 (N2B-ATD-Fc), the fusion protein #8 (N1-ATD-N2B-ATD-Fc) and the fusion protein #4 (N1ecd-N2Becd-Fc) are particularly advantageous for binding autoantibodies present in patient samples.

The In Vitro Method for Detection of NMDAR Autoantibodies in a Sample

The present invention further relates to an in vitro method for the detection of NMDAR autoantibodies in a sample, the method comprising,
providing a sample suspected of comprising NMDAR autoantibodies,
providing a NMDA protein construct according to any one of the preceding claim as a capture molecule,
contacting said sample with said NMDAR protein construct, thereby binding NMDAR autoantibodies from said sample to said NMDAR protein construct, and
determining the presence and optionally the amount of bound NMDAR autoantibodies.

In embodiments of the method for the detection of NMDAR autoantibodies of the invention, the NMDAR autoantibodies in said sample are present in solution or on a cell-membrane.

In embodiments, the method for the detection of NMDAR autoantibodies is carried out with multiple and different NMDAR protein constructs in the sense of the present invention. In the context of the method of the invention using multiple different constructs, the method may comprise additionally a step of determining against which NMDAR protein construct of said multiple constructs the NMDAR autoantibodies bind or preferably bind in the largest amounts and/or most efficiently bind. Accordingly, it may be possible to profile and categorize the patient providing the sample on the basis of the determined NMDAR autoantibodies and their binding properties with respect to the NMDAR protein constructs used in the method of the invention. In this context, the method of the invention may be carried out separately for each of the multiple constructs (parallel determining), or binding of NMDAR autoantibodies to more than one NMDAR construct is determined in a single assay (multiplexing).

The method of the invention can comprise a step of determining a NMDAR autoantibody-profile present in said sample.

In embodiments of the method of the invention, the presence and optionally the amount of cells displaying NMDAR autoantibodies on their cell surface present in said sample may be determined. It is a great advantage of the method of the invention that it is possible to detect soluble NMDAR autoantibodies as well as NMDAR autoantibodies on the cell surface, in particular on the surface of B-cells producing the NMDAR autoantibodies.

In embodiments, the method of the invention is applied for the diagnosis, prognosis, disease monitoring, patient stratification and/or therapy monitoring of a medical condition associated with autoantibodies against the NMDAR, preferably anti-NMDAR encephalitis, and the sample suspected of comprising NMDAR autoantibodies is a sample of a human subject exhibiting symptoms of having said medical disorder.

In embodiments of the method for the diagnosis, prognosis, disease monitoring, patient stratification and/or therapy monitoring of a medical condition associated with autoantibodies against the NMDAR, preferably anti-NMDAR encephalitis, the presence of bound NMDAR autoantibodies, preferably an amount of bound NMDAR autoantibodies above a suitable control, such as an amount from a healthy control population, indicates the presence or likelihood of the subject developing a medical condition associated with autoantibodies against the NMDAR, preferably anti-NMDAR encephalitis.

Current diagnostics are based on tissue profiling or CBAs expressing the GluN1 subunit of the NMDA receptor. In comparison to these, the method of the invention based on the NMADR protein constructs of the invention offers several advantages. For example, the method of the invention enables binding or capturing of autoantibodies that bind to GluN1 in the context of GluN2 subunits, therefore also capturing autoantibodies that bind to overlapping epitopes or conformationally stabilized epitopes. Further, the method of the invention enables differentiation for preferential binding of autoantibodies to for example GluN1 and GluN2A vs. GluN1 and GluN2B vs. individual subunits vs. GluN1 and GluN2C vs. GluN1 and GluN2D and further combinations of GluN1 with more than one isoform of the GluN2 subunit.

A further advantage of the detection method disclosed herein is the robustness of the assay and the fact that it is amenable to standardization and even full automation, such as sFIDA. Furthermore, the assay can be optimized to function as a single molecule level assay (e.g. SIMOA) thereby arriving at quantification of autoantibodies. Furthermore, the method of the invention is more sensitive than assays of the state of the art for detecting NMDAR autoantibodies due to a reduction of the cellular background from mammalian cells, preferably human cells such as HEK cells.

In the context of the method of the invention, the NMDAR protein construct can be immobilized on a solid phase before contacting with said sample.

In embodiments of the invention, the soluble NMDAR protein construct of the invention is provided in an immobilized form. In such embodiments, the soluble NMDAR protein construct of the invention may be immobilized on a solid phase after it has been purified in its soluble form from a suitable expression system. Accordingly, in the context of such embodiments of an immobilized NMDAR protein construct of the invention, “soluble” is relating to the previous state of the construct before it has been immobilized.

Furthermore, the method of the invention may be conducted as an Enzyme Linked Immunosorbent Assay (ELISA).

The determining of NMDAR autoantibodies in the context of the method of the invention can comprise the following steps:
immobilizing NMDAR autoantibodies from the sample through binding to the NMDAR protein construct that is immobilized on the solid surface,
treating said immobilized NMDAR autoantibodies with a labelled secondary affinity reagent directed to NMDAR autoantibodies,
detecting a signal emitted from said labelled secondary affinity reagent directed to NMDAR autoantibodies, and
comparing the signal obtained from said labelled secondary affinity reagent with the signal from one or more control samples of pre-determined NMDAR autoantibody concentration.

In embodiments, the signal is obtained from horseradish peroxidase conjugated to the secondary affinity reagent. In further embodiments, other labels of the secondary affinity reagent may be used, such as fluorescent or chemiluminescent labels and further labels known to a skilled person.

In specific embodiments, the method of the invention may be applied for therapy guidance of a subject suspected of having and/or developing a medical condition associated with NMDAR autoantibodies, the method comprising selecting one or more of the corresponding NMDAR protein constructs of the invention for subsequent treatment of said subject.

The Kit for Detecting NMDAR Autoantibodies

The present invention also relates to a kit for detecting NMDAR autoantibodies in a sample, the kit comprising
a NMDAR protein constructs of the present invention and optionally a solid surface for immobilization of said NMDAR protein construct, or a NMDAR protein constructs of the invention immobilized on a solid surface, and a labelled secondary affinity reagent directed to human NMDAR autoantibodies, such as a labelled anti-human IgG antibody, and optionally means for detecting the signal emitted from said label, or
a labelled NMDAR protein construct of the invention and optionally means for detecting the signal emitted from said label, and
optionally a control sample of pre-determined NMDAR autoantibody concentration.

The kit of the invention may be used to detect NMDAR autoantibody expressing cells, for example by FACS, using a fluorescently labeled construct of the invention or a fluorescently labeled secondary antibody directed against rabbit Fc. Furthermore, the kit may be used to carry out an ELISA for detecting the NMDAR autoantibodies present in a sample.

The invention also relates to a kit for the diagnosis of an autoimmune disease associated with NMDAR autoantibodies, such as NMDAR encephalitis, in a subject by detection of NMDAR autoantibodies, comprising:
a NMDAR protein construct of the invention and optionally a solid surface for immobilization of said NMDAR protein construct, or a NMDAR protein construct of the invention immobilized on a solid surface, and a labelled secondary affinity reagent directed to human NMDAR autoantibodies, such as a labelled anti-human IgG antibody, and optionally means for detecting the signal emitted from said label, or
a labelled NMDAR protein construct of the invention, and
optionally a control sample of pre-determined NMDAR autoantibody concentration.

The Blood Treatment Device Comprising NMDAR Protein Constructs

The present invention further relates to a blood treatment device configured to remove NMDAR autoantibodies from the blood or blood plasma of a person in need thereof in an extracorporeal blood circuit, wherein the device comprises a matrix having one or more NMDAR protein constructs of the invention immobilized thereon.

In embodiments, the blood treatment device of the invention is located in an extracorporeal blood circuit through which the blood of the patient passes, and which comprises means for transporting blood from the patient's vascular system to the blood treatment device at a defined flow rate and for returning the treated blood back to the patient.

Further Aspects of the Invention

Furthermore, the invention comprises NMDAR protein constructs as disclosed herein for use as a medicament. Also, the invention relates to the NMDAR protein constructs as disclosed herein for use as a medicament in the treatment of a subject suffering from an autoimmune disease associated with NMDAR autoantibodies, preferably NMDAR encephalitis.

The invention also relates to an in vitro method for producing a NMDAR protein construct of the invention, the method comprising expressing a nucleic acid sequence encoding a NMDAR protein construct of the invention in a mammalian cell, preferably a human cell, and subsequent isolation of said NMDAR protein construct. Preferably, the constructs are isolated from the cell supernatant after secretion of the protein constructs by the cells.

It is a great advantage of the present invention that the soluble NMDAR protein constructs can be isolated from cell culture supernatants of cells that have been modified to express a nucleic acid sequence encoding a NMDAR protein construct of the invention, in particular in comparison to methods where the antigen first needs to be isolated from cell membranes using either protease cleavage or detergent solubilization.

Additionally, the invention includes NMDAR protein construct as disclosed herein that are produced by the disclosed method for producing a NMDAR protein construct of the invention.

The various embodiments and features of the NMDAR protein constructs disclosed herein also apply to the various embodiments of the methods for detecting NMDAR autoantibodies in a sample, the kit for detecting NMDAR autoantibodies, the blood treatment device configured to remove NMDAR autoantibodies from the blood or blood plasma of a person in need thereof, and the method for producing a NMDAR protein construct of the invention presented herein, and vice versa.

### DETAILED DESCRIPTION OF THE INVENTION

All cited documents of the patent and non-patent literature are hereby incorporated by reference in their entirety.

The present invention is directed to a soluble NMDAR protein construct comprising one or more NMDAR autoantibody epitopes, wherein the construct comprises an extracellular domain (ECD) of the NMDAR subunit GluN1 or a fragment thereof and an ECD of at least one of the NMDAR subunits GluN2A, GluN2B, GluN2C or GluN2D, or fragment thereof.

In the context of the present invention the term “protein construct” can relate to an individual protein or peptide formed by a single amino acid chain. Furthermore, the term “protein construct” as used herein also comprises constructs or complexes of two or more proteins or peptides or amino acid chains that are covalently linked, for example by disulfide-bridges or other linkers between individual amino acid chains. Further, the term “protein construct” comprises protein complexes formed by more than one protein or peptide or amino acid chain through non-covalent bonding or non-covalent interactions, such as electrostatic interaction, van der Waals forces, hydrophobic interactions or others known to the skilled person, leading to the formation of for example protein dimers or protein multimers that can be assembled via a dimerization or multimerization domain, respectively.

In embodiments of the invention, the protein construct is one or more proteins comprising an extracellular domain (ECD) of the NMDAR subunit GluN1 or a fragment thereof and an ECD of at least one of the NMDAR subunits GluN2A, GluN2B, GluN2C or GluN2D, or fragment thereof. Therein, the one or more proteins can be a single protein that comprises both ECDs or fragments thereof in a single amino acid chain; or the one or more proteins can be, for example, two proteins, wherein each of the two proteins comprises one or more ECD of GluN1 or GluN2 (A, B, C or D) or fragment thereof and the two proteins are assembled in a protein complex. In embodiments comprising two (or more) proteins, the two (or more) proteins can be assembled in a complex in which the proteins are covalently linked, for example by disulfide-bridges or other linkers, or in which the proteins assemble through non-covalent bonding/interactions.

“Peptide”, “polypeptide”, “polypeptide fragment”, “amino acid chain” and “protein” are used interchangeably, unless specified to the contrary, and according to conventional meaning, i.e., as a sequence of amino acids. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring.

An “isolated peptide” or an “isolated polypeptide” or “isolated protein construct” and the like, as used herein, refer to in vitro isolation and/or purification of a peptide or polypeptide molecule or protein construct from a cellular environment or the cell culture supernatant, and from association with other components of the cell, i.e., it is not significantly associated with in vivo substances.

In the context of the present invention, a dimerization domain is any domain that can be comprised or integrated in a protein or peptide capable of binding to another domain, such as another dimerization domain, of another protein or peptide. Many examples of dimerization domains are known to the skilled person, including antibody Fc fragments of antibodies, leucin-zipper domains or coiled coil domains. Dimerization can lead to the formation of homo- and heterodimers, meaning assembly of two identical or two different proteins, respectively. In both cases, the dimerization domains can be identical and in case of a heterodimer also differential in the monomers forming the dimer.

In embodiments of the invention, the protein constructs comprise a dimerization domain. Such constructs can be composed of one or more proteins. It is obvious to a person skilled in the art that in case of a protein construct of the invention that is composed of a single protein, the presence of a dimerization domain can lead to formation of homodimers. Furthermore, it is immediately obvious to a skilled person that a protein construct, which comprises a dimerization domain and that is composed of two or more proteins, can comprise a dimerization domain in each of the proteins. In such embodiments, dimerization of the two proteins of the protein construct is preferentially mediated by the dimerization domain. In other words, if the protein construct of the invention is or forms a dimer of two proteins (either a homodimer or a heterodimer), dimerization can be brought about by dimerization domains comprised by each of the proteins. In preferred embodiments, the dimerization domain is a Fc domain.

As used herein the term capture domain refers to a domain or part of the protein constructs of the present invention that can be used for binding a construct of the present invention to a solid phase, either through non-covalent interaction or covalently. Typical examples of such capture domains are domains or amino acid sequences that are recognized by commonly available proteins that bind to the capture domains, such as preferably antibodies. For example, Fc fragments of antibodies can serve as capture domains since there are high affinity antibodies that specifically bind to these domains. Furthermore, protein-tags such as a Myc-Tag, HA-Tag, HIS-Tag or the like can be used as capture domains.

There is a huge variety of possible dimerization domains and capture domains that can be integrated into a protein construct of the present invention and a skilled person is capable of identifying suitable variants. Furthermore, in some cases it is possible that the dimerization domain also serves as a capture domain. This is for example the case for antibody Fc-fragments, which are capable of forming dimers and can also be easily bound by commonly available antibodies. Therefore, the Fc-fragment can serve at the same time as a capture- and dimerization domain. Further examples are known or can be identified by the skilled person without undue effort.

Embodiments of the present invention relate to recombinant proteins, such as recombinant fusion proteins, which are proteins created through genetic engineering of a fusion gene. This typically involves appending the cDNA sequence of a second protein fragment in frame with cDNA of a first protein (fragment) without a stop codon in between, for example through ligation or overlap extension PCR. That DNA sequence will then be expressed by a cell as a single protein. The protein can be engineered to include the full sequence of both original proteins, or only a portion of either. More than two proteins or fragments can be connected to form a complex fusion protein. In between the various parts of a fusion proteins, there are often so-called linker (or “spacer”) peptides, which make it more likely that the proteins fold independently and behave as expected. Especially in the case where the linkers enable protein purification, linkers in protein or peptide fusions are sometimes engineered with cleavage sites for proteases or chemical agents that enable the liberation of the two separate proteins. This technique is often used for identification and purification of proteins, by fusing a GST protein, FLAG peptide, or a hexa-his peptide (6xHis-tag), which can be isolated using affinity chromatography with nickel or cobalt resins. Di- or multimeric chimeric proteins can be manufactured through genetic engineering by fusion to the original proteins of peptide domains that induce artificial protein di- or multimerization (e.g., streptavidin or leucine zippers).

Protein linkers aid fusion protein design by providing appropriate spacing between domains, supporting correct protein folding in the case that N or C termini interactions are crucial to folding. Commonly, protein linkers permit important domain interactions, reinforce stability, and reduce steric hindrance, making them preferred for use in fusion protein design even when N and C termini can be fused. At least three major types of linkers are flexible, rigid, and (in vivo) cleavable. Flexible linkers may consist of many small glycine residues, giving them the ability curl into a dynamic, adaptable shape. Rigid linkers may be formed of large, cyclic proline residues, which can be helpful when highly specific spacing between domains must be maintained. (In vivo) cleavable linkers are unique in that they are designed to allow the release of one or more fused domains under certain reaction conditions, such as a specific pH gradient, or when coming in contact with another biomolecule in the cell. Selection and design of suitable linker sequences is a standard procedure known to a person skilled in the art. In case of cleavable linker sequence, the skilled person is also able to select a suitable enzyme or reagent for linker cleavage and to design or select a corresponding linker.

Preferred Sequences Comprised by NMDAR Constructs of the Invention

Preferred amino acid sequences comprised by embodiments of the NMDAR protein constructs of the invention or fusion proteins that can be used in NMDAR protein constructs of the invention are disclosed in Table 1. Preferred nucleic acid sequences comprised by nucleic acid molecules encoding for NMDAR protein constructs of the invention fusion proteins that can be used in NMDAR protein constructs of the invention are disclosed in Table 2.

TABLE 1
Preferred amino acid sequences of the present invention.

| SEQ ID NO. | Amino Acid Sequence | Description |
|---|---|---|
| SEQ ID NO 1 | MSTMRLLTLALLFSCSVARAACDPKIVNIGAVLSTR | Protien sequence of |
| | KHEQMFREAVNQANKRHGSWKIQLNATSVTHKPN | fusion protein #1 N1- |
| | AIQMALSVCEDLISSQVYAILVSHPPTPNDHFTPTPV | ATD-Fc |
| | SYTAGFYRIPVLGLTTRMSIYSDKSIHLSFLRTVPPY | |
| | SHQSSVWFEMMRVYSWNHIILLVSDDHEGRAAQK | |
| | RLETLLEERESKAEKVLQFDPGTKNVTALLMEAKEL | |
| | EARVIILSASEDDAATVYRAAAMLNMTGSGYVWLV | |
| | GEREISGNALRYAPDGILGLQLINGKNESAHISDAV | |
| | GVVAQAVHELLEKENITDPPRGCVGNTNIWKTGPL | |
| | FKRVLMSSKYADGVTGRVEFNEDGDRKFANYSIM | |
| | NLQNRKLVQVGIYNGTHVIPNDRKIIWPGGETEKPR | |
| | GYQMSTRLKIGSSTMVRSSKPTCPPPELLGGPSVFI | |
| | FPPKPKDTLMISRTPEVTCVVVDVSQDDPEVQFTW | |
| | YINNEQVRTARPPLREQQFNSTIRVVSTLPIAHQDW | |
| | LRGKEFKCKVHNKALPAPIEKTISKARGQPLEPKVY | |
| | TMGPPREELSSRSVSLTCMINGFYPSDISVEWEKN | |
| | GKAEDNYKTTPAVLDSDGSYFLYSKLSVPTSEWQR | |
| | GDVFTCSVMHEALHNHYTQKSISRSPGK | |
| SEQ ID NO 2 | MSTMRLLTLALLFSCSVARAACDPKIVNIGAVLSTR | Amino terminal domain |
| | KHEQMFREAVNQANKRHGSWKIQLNATSVTHKPN | (ATD) of GluN1 |
| | AIQMALSVCEDLISSQVYAILVSHPPTPNDHFTPTPV | including the signal |
| | SYTAGFYRIPVLGLTTRMSIYSDKSIHLSFLRTVPPY | sequence as in fusion |
| | SHQSSVWFEMMRVYSWNHIILLVSDDHEGRAAQK | protein #1 |
| | RLETLLEERESKAEKVLQFDPGTKNVTALLMEAKEL | |
| | EARVIILSASEDDAATVYRAAAMLNMTGSGYVWLV | |
| | GEREISGNALRYAPDGILGLQLINGKNESAHISDAV | |
| | GVVAQAVHELLEKENITDPPRGCVGNTNIWKTGPL | |
| | FKRVLMSSKYADGVTGRVEFNEDGDRKFANYSIM | |
| | NLQNRKLVQVGIYNGTHVIPNDRKIIWPGGETEKPR | |
| | GYQMSTRLKI | |
| SEQ ID NO 3 | GSSTMVRS | Linker between |
| | | NMDAR domain and |
| | | rabbit Fc to be used in |
| | | fusion proteins of |
| | | NMDAR protein |
| | | constructs of the |
| | | invention |
| SEQ ID NO 4 | SKPTCPPPELLGGPSVFIFPPKPKDTLMISRTPEVT | Rabbit Fc from Vector |
| | CVVVDVSQDDPEVQFTWYINNEQVRTARPPLREQ | pFuse-rIgG-Fc1 |
| | QFNSTIRVVSTLPIAHQDWLRGKEFKCKVHNKALPA | (InvivoGen) as used in |
| | PIEKTISKARGQPLEPKVYTMGPPREELSSRSVSLT | fusion proteins #1-#8 |
| | CMINGFYPSDISVEWEKNGKAEDNYKTTPAVLDSD | |
| | GSYFLYSKLSVPTSEWQRGDVFTCSVMHEALHNH | |
| | YTQKSISRSPGK | |
| SEQ ID NO 5 | TLALLFSCSVARAACDPKIVNIGAVLSTRKHEQMFR | Amino terminal domain |
| | EAVNQANKRHGSWKIQLNATSVTHKPNAIQMALSV | of GluN1 in |
| | CEDLISSQVYAILVSHPPTPNDHFTPTPVSYTAGFY | NM_007327 with |
| | RIPVLGLTTRMSIYSDKSIHLSFLRTVPPYSHQSSV | domain borders as in |
| | WFEMMRVYSWNHIILLVSDDHEGRAAQKRLETLLE | cd06379: |
| | ERESKAEKVLQFDPGTKNVTALLMEAKELEARVIIL | PBP1_iGluR_NMDA |
| | SASEDDAATVYRAAAMLNMTGSGYVWLVGEREIS | NR1 (NCBI) |
| | GNALRYAPDGILGLQLINGKNESAHISDAVGVVAQA | |
| | VHELLEKENITDPPRGCVGNTNIWKTGPLFKRVLM | |
| | SSKYADGVTGRVEFNEDGDRKFANYSIMNLQNRKL | |
| | VQVGIYNGTHVIPNDRKIIWPG | |
| SEQ ID NO 6 | MSTMRLLTLALLFSCSVARAACDPKIVNIGAVLSTR | protein seq of fusion |
| | KHEQMFREAVNQANKRHGSWKIQLNATSVTHKPN | protein #2 N1 ecd-Fc |
| | AIQMALSVCEDLISSQVYAILVSHPPTPNDHFTPTPV | |
| | SYTAGFYRIPVLGLTTRMSIYSDKSIHLSFLRTVPPY | |
| | SHQSSVWFEMMRVYSWNHIILLVSDDHEGRAAQK | |
| | RLETLLEERESKAEKVLQFDPGTKNVTALLMEAKEL | |
| | EARVIILSASEDDAATVYRAAAMLNMTGSGYVWLV | |
| | GEREISGNALRYAPDGILGLQLINGKNESAHISDAV | |
| | GVVAQAVHELLEKENITDPPRGCVGNTNIWKTGPL | |
| | FKRVLMSSKYADGVTGRVEFNEDGDRKFANYSIM | |
| | NLQNRKLVQVGIYNGTHVIPNDRKIIWPGGETEKPR | |
| | GYQMSTRLKIVTIHQEPFVYVKPTLSDGTCKEEFTV | |
| | NGDPVKKVICTGPNDTSPGSPRHTVPQCCYGFCID | |
| | LLIKLARTMNFTYEVHLVADGKFGTQERVNNSNKK | |
| | EWNGMMGELLSGQADMIVAPLTINNERAQYIEFSK | |
| | PFKYQGLTILVKKGTRITGINDPRLRNPSDKFIYATV | |
| | KQSSVDIYFRRQVELSTMYRHMEKHNYESAAEAIQ | |
| | AVRDNKLHAFIWDSAVLEFEASQKCDLVTTGELFF | |
| | RSGFGIGMRKDSPWKQNVSLSILKSHENGFMEDLD | |
| | KTWVRYQECDSGSSTMVRSSKPTCPPPELLGGPS | |
| | VFIFPPKPKDTLMISRTPEVTCVVVDVSQDDPEVQF | |
| | TWYINNEQVRTARPPLREQQFNSTIRVVSTLPIAHQ | |
| | DWLRGKEFKCKVHNKALPAPIEKTISKARGQPLEPK | |
| | VYTMGPPREELSSRSVSLTCMINGFYPSDISVEWE | |
| | KNGKAEDNYKTTPAVLDSDGSYFLYSKLSVPTSEW | |
| | QRGDVFTCSVMHEALHNHYTQKSISRSPGK | |
| SEQ ID NO 7 | MSTMRLLTLALLFSCSVARAACDPKIVNIGAVLSTR | Amino terminal domain |
| | KHEQMFREAVNQANKRHGSWKIQLNATSVTHKPN | of GluN1 (including |
| | AIQMALSVCEDLISSQVYAILVSHPPTPNDHFTPTPV | signal sequence) as |
| | SYTAGFYRIPVLGLTTRMSIYSDKSIHLSFLRTVPPY | included in fusion |
| | SHQSSVWFEMMRVYSWNHIILLVSDDHEGRAAQK | proteins #2, #4 |
| | RLETLLEERESKAEKVLQFDPGTKNVTALLMEAKEL | |
| | EARVIILSASEDDAATVYRAAAMLNMTGSGYVWLV | |
| | GEREISGNALRYAPDGILGLQLINGKNESAHISDAV | |
| | GVVAQAVHELLEKENITDPPRGCVGNTNIWKTGPL | |
| | FKRVLMSSKYADGVTGRVEFNEDGDRKFANYSIM | |
| | NLQNRKLVQVGIYNGTHVIPNDRKIIWPGGETEKPR | |
| | GYQM | |
| SEQ ID NO 8 | STRLKIVTIHQEPFVYVKPTLSDGTCKEEFTVNGDP | Ligand-binding domain |
| | VKKVICTGPNDTSPGSPRHTVPQCCYGFCIDLLIKL | of GluN1 S1, as for |
| | ARTMNFTYEVHLVADGKFGTQERVNNSNKKEWNG | example included in |
| | MMGELLSGQADMIVAPLTINNERAQYIEFSKPFKYQ | fusion proteins #2, #4; |
| | GLTILVKK | corresponds to Ligand- |
| | | binding domain of |
| | | GluN1 S1 in |
| | | NM_007327 with |
| | | domain borders as in |
| | | cd13719: |
| | | PBP2_iGluR_NMDA |
| | | Nr1 (NCBI); |
| | GT | Linker between S1 and |
| | | S2, as for example |
| | | included in fusion |
| | | proteins #2, #3, #4 |
| SEQ ID NO 9 | RITGINDPRLRNPSDKFIYATVKQSSVDIYFRRQVE | Ligand-binding domain |
| | LSTMYRHMEKHNYESAAEAIQAVRDNKLHAFIWDSA | of GluN1 S2, as for |
| | VLEFEASQKCDLVTTGELFFRSGFGIGMRKDSPWK | example included in |
| | QNVSLSILKSHENGFMEDLDKTWVRYQECDS | fusion proteins #2, #4 |
| SEQ ID NO | RITGINDPRLRNPSDKFIYATVKQSSVDIYFRRQVE | Ligand-binding domain |
| 10 | LSTMYRHMEKHNYESAAEAIQAVRDNKLHAFIWDSA | of GluN1 S2 in |
| | VLEFEASQKCDLVTTGELFFRSGFGIGMRKDSPWK | NM_007327 with |
| | QNVSLSILKSHENGFMEDLDKTWVRYQECD | domain borders as in |
| | | cd13719: |
| | | PBP2_iGluR_NMDA |
| | | Nr1 (NCBI) |
| SEQ ID NO | MKPRAECCSPKFWLVLAVLAVSGSRARSQKSPPSI | protein seq of fusion |
| 11 | GIAVILVGTSDEVAIKDAHEKDDFHHLSVVPRVELVA | protein #3 N2Becd-Fc |
| | MNETDPKSIITRICDLMSDRKIQGVVFADDTDQEAIA | |
| | QILDFISAQTLTPILGIHGGSSMIMADKDESSMFFQF | |
| | GPSIEQQASVMLNIMEEYDWYIFSIVTTYFPGYQDF | |
| | VNKIRSTIENSFVGWELEEVLLLDMSLDDGDSKIQN | |
| | QLKKLQSPIILLYCTKEEATYIFEVANSVGLTGYGYT | |
| | WIVPSLVAGDTDTVPAEFPTGLISVSYDEWDYGLP | |
| | ARVRDGIAIITTAASDMLSEHSFIPEPKSSCYNTHEK | |
| | RIYQSNMLNRYLINVTFEGRNLSFSEDGYQMHPKL | |
| | VIILLNKERKWERVGKWKDKSLQMKYYVWPRMCP | |
| | ETEEQEDDHLSIVTLEEAPFVIVESVDPLSGTCMRN | |
| | TVPCQKRIVTENKTDEEPGYIKKCCKGFCIDILKKIS | |
| | KSVKFTYDLYLVTNGKHGKKINGTWNGMIGEVVMK | |
| | RAYMAVGSLTINEERSEVVDFSVPFIETGISVMVSR | |
| | GTQVSGLSDKKFQRPNDFSPPFRFGTVPNGSTER | |
| | NIRNNYAEMHAYMGKFNQRGVDDALLSLKTGKLDA | |
| | FIYDAAVLNYMAGRDEGCKLVTIGSGKVFASTGYGI | |
| | AIQKDSGWKRQVDLAILQLFGDGEMEELEALWLTG | |
| | ICHNGSSTMVRSSKPTCPPPELLGGPSVFIFPPKPK | |
| | DTLMISRTPEVTCVVVDVSQDDPEVQFTWYINNEQ | |
| | VRTARPPLREQQFNSTIRVVSTLPIAHQDWLRGKE | |
| | FKCKVHNKALPAPIEKTISKARGQPLEPKVYTMGPP | |
| | REELSSRSVSLTCMINGFYPSDISVEWEKNGKAED | |
| | NYKTTPAVLDSDGSYFLYSKLSVPTSEWQRGDVFT | |
| | CSVMHEALHNHYTQKSISRSPGK | |
| SEQ ID NO | MKPRAECCSPKFWLVLAVLAVSGSRARSQKSPPSI | Amino terminal domain |
| 12 | GIAVILVGTSDEVAIKDAHEKDDFHHLSVVPRVELVA | of GluN2B (including |
| | MNETDPKSIITRICDLMSDRKIQGVVFADDTDQEAIA | signal sequence), as |
| | QILDFISAQTLTPILGIHGGSSMIMADKDESSMFFQF | for example included |
| | GPSIEQQASVMLNIMEEYDWYIFSIVTTYFPGYQDF | in fusion protein #3 |
| | VNKIRSTIENSFVGWELEEVLLLDMSLDDGDSKIQN | N2Becd-Fc |
| | QLKKLQSPIILLYCTKEEATYIFEVANSVGLTGYGYT | |
| | WIVPSLVAGDTDTVPAEFPTGLISVSYDEWDYGLP | |
| | ARVRDGIAIITTAASDMLSEHSFIPEPKSSCYNTHEK | |
| | RIYQSNMLNRYLINVTFEGRNLSFSEDGYQMHPKL | |
| | VIILLNKERKWERVGKWKDKSLQMKYYVWPRMCP | |
| | ETEEQ | |
| SEQ ID NO | EDDHLSIVTLEEAPFVIVESVDPLSGTCMRNTVPCQ | Ligand-binding domain |
| 13 | KRIVTENKTDEEPGYIKKCCKGFCIDILKKISKSVKFT | of GluN2B S1, as for |
| | YDLYLVTNGKHGKKINGTWNGMIGEVVMKRAYMA | example included in |
| | VGSLTINEERSEVVDFSVPFIETGISVMVSR | fusion proteins #3, #4 |
| SEQ ID NO | QVSGLSDKKFQRPNDFSPPFRFGTVPNGSTERNIR | Ligand-binding domain |
| 14 | NNYAEMHAYMGKFNQRGVDDALLSLKTGKLDAFIY | of GluN2B S2, as for |
| | DAAVLNYMAGRDEGCKLVTIGSGKVFASTGYGIAIQ | example included in |
| | KDSGWKRQVDLAILQLFGDGEMEELEALWLTGICH | fusion proteins #3, #4 |
| | N | |
| SEQ ID NO | PSIGIAVILVGTSDEVAIKDAHEKDDFHHLSVVPRVE | Amino terminal domain |
| 15 | LVAMNETDPKSIITRICDLMSDRKIQGVVFADDTDQ | of GluN2B in |
| | EAIAQILDFISAQTLTPILGIHGGSSMIMADKDESSMF | NM_000834 with |
| | FQFGPSIEQQASVMLNIMEEYDWYIFSIVTTYFPGY | domain borders as in |
| | QDFVNKIRSTIENSFVGWELEEVLLLDMSLDDGDSK | cd06378: |
| | IQNQLKKLQSPIILLYCTKEEATYIFEVANSVGLTGY | PBP1_iGluR_NMDA |
| | GYTWIVPSLVAGDTDTVPAEFPTGLISVSYDEWDY | NR2 (NCBI) |
| | GLPARVRDGIAIITTAASDMLSEHSFIPEPKSSCYNT | |
| | HEKRIYQSNMLNRYLINVTFEGRNLSFSEDGYQMH | |
| | PKLVIILLNKERKWERVGKWKDKSLQMKYYVWPR | |
| SEQ ID NO | DDHLSIVTLEEAPFVIVESVDPLSGTCMRNTVPCQK | Ligand-binding domain |
| 16 | RIVTENKTDEEPGYIKKCCKGFCIDILKKISKSVKFTY | of GluN2B S1 in |
| | DLYLVTNGKHGKKINGTWNGMIGEVVMKRAYMAV | NM_000834 with |
| | GSLTINEERSEVVDFSVPFIETGISVMVSR | domain borders as in |
| | | cd13718: |
| | | PBP2_iGluR_NMDA_ |
| | | Nr2 (NCBI) |
| SEQ ID NO | RITGINDPRLRNPSDKFIYATVKQSSVDIYFRRQVEL | Ligand-binding domain |
| 17 | STMYRHMEKHNYESAAEAIQAVRDNKLHAFIWDSA | of GluN2B S2 in |
| | VLEFEASQKCDLVTTGELFFRSGFGIGMRKDSPWK | NM_000834 with |
| | QNVSLSILKSHENGFMEDLDKTWVRYQECD | domain borders as in |
| | | cd13718: |
| | | PBP2_iGluR_NMDA |
| | | Nr2 (NCBI) |
| SEQ ID NO | MSTMRLLTLALLFSCSVARAACDPKIVNIGAVLSTR | protein seq of fusion |
| 18 | KHEQMFREAVNQANKRHGSWKIQLNATSVTHKPN | protein #4 N1 ecd- |
| | AIQMALSVCEDLISSQVYAILVSHPPTPNDHFTPTPV | N2Becd-Fc |
| | SYTAGFYRIPVLGLTTRMSIYSDKSIHLSFLRTVPPY | |
| | SHQSSVWFEMMRVYSWNHIILLVSDDHEGRAAQK | |
| | RLETLLEERESKAEKVLQFDPGTKNVTALLMEAKEL | |
| | EARVIILSASEDDAATVYRAAAMLNMTGSGYVWLV | |
| | GEREISGNALRYAPDGILGLQLINGKNESAHISDAV | |
| | GVVAQAVHELLEKENITDPPRGCVGNTNIWKTGPL | |
| | FKRVLMSSKYADGVTGRVEFNEDGDRKFANYSIM | |
| | NLQNRKLVQVGIYNGTHVIPNDRKIIWPGGETEKPR | |
| | GYQMSTRLKIVTIHQEPFVYVKPTLSDGTCKEEFTV | |
| | NGDPVKKVICTGPNDTSPGSPRHTVPQCCYGFCID | |
| | LLIKLARTMNFTYEVHLVADGKFGTQERVNNSNKK | |
| | EWNGMMGELLSGQADMIVAPLTINNERAQYIEFSK | |
| | PFKYQGLTILVKKGTRITGINDPRLRNPSDKFIYATV | |
| | KQSSVDIYFRRQVELSTMYRHMEKHNYESAAEAIQ | |
| | AVRDNKLHAFIWDSAVLEFEASQKCDLVTTGELFF | |
| | RSGFGIGMRKDSPWKQNVSLSILKSHENGFMEDLD | |
| | KTWVRYQECDSGSTGGGGSGGGGSGGGGSGAA | |
| | SRQKSPPSIGIAVILVGTSDEVAIKDAHEKDDFHHLS | |
| | VVPRVELVAMNETDPKSIITRICDLMSDRKIQGVVFA | |
| | DDTDQEAIAQILDFISAQTLTPILGIHGGSSMIMADK | |
| | DESSMFFQFGPSIEQQASVMLNIMEEYDWYIFSIVT | |
| | TYFPGYQDFVNKIRSTIENSFVGWELEEVLLLDMSL | |
| | DDGDSKIQNQLKKLQSPIILLYCTKEEATYIFEVANS | |
| | VGLTGYGYTWIVPSLVAGDTDTVPAEFPTGLISVSY | |
| | DEWDYGLPARVRDGIAIITTAASDMLSEHSFIPEPK | |
| | SSCYNTHEKRIYQSNMLNRYLINVTFEGRNLSFSED | |
| | GYQMHPKLVIILLNKERKWERVGKWKDKSLQMKYY | |
| | VWPRMCPETEEQEDDHLSIVTLEEAPFVIVESVDPL | |
| | SGTCMRNTVPCQKRIVTENKTDEEPGYIKKCCKGF | |
| | CIDILKKISKSVKFTYDLYLVTNGKHGKKINGTWNG | |
| | MIGEVVMKRAYMAVGSLTINEERSEVVDFSVPFIET | |
| | GISVMVSRGTQVSGLSDKKFQRPNDFSPPFRFGTV | |
| | PNGSTERNIRNNYAEMHAYMGKFNQRGVDDALLS | |
| | LKTGKLDAFIYDAAVLNYMAGRDEGCKLVTIGSGKV | |
| | FASTGYGIAIQKDSGWKRQVDLAILQLFGDGEMEE | |
| | LEALWLTGICHNGSSTMVRSSKPTCPPPELLGGPS | |
| | VFIFPPKPKDTLMISRTPEVTCVVVDVSQDDPEVQF | |
| | TWYINNEQVRTARPPLREQQFNSTIRVVSTLPIAHQ | |
| | DWLRGKEFKCKVHNKALPAPIEKTISKARGQPLEPK | |
| | VYTMGPPREELSSRSVSLTCMINGFYPSDISVEWE | |
| | KNGKAEDNYKTTPAVLDSDGSYFLYSKLSVPTSEW | |
| | QRGDVFTCSVMHEALHNHYTQKSISRSPGK | |
| SEQ ID NO | GSTGGGGSGGGGSGGGGSGAASR | Linker between a |
| 19 | | GluN1-domain and |
| | | GluN2B-domain in a |
| | | fusion protein of |
| | | NMDAR construct of |
| | | the invention, as for |
| | | example in fusion |
| | | protein #4 N1 ecd- |
| | | N2Becd-Fc or in fusion |
| | | proteins #7, #8 |
| SEQ ID NO | QKSPPSIGIAVILVGTSDEVAIKDAHEKDDFHHLSVV | Amino terminal domain |
| 20 | PRVELVAMNETDPKSIITRICDLMSDRKIQGVVFAD | of GluN2B |
| | DTDQEAIAQILDFISAQTLTPILGIHGGSSMIMADKD | |
| | ESSMFFQFGPSIEQQASVMLNIMEEYDWYIFSIVTT | |
| | YFPGYQDFVNKIRSTIENSFVGWELEEVLLLDMSLD | |
| | DGDSKIQNQLKKLQSPIILLYCTKEEATYIFEVANSV | |
| | GLTGYGYTWIVPSLVAGDTDTVPAEFPTGLISVSYD | |
| | EWDYGLPARVRDGIAIITTAASDMLSEHSFIPEPKSS | |
| | CYNTHEKRIYQSNMLNRYLINVTFEGRNLSFSEDG | |
| | YQMHPKLVIILLNKERKWERVGKWKDKSLQMKYYV | |
| | WPRMCPETEEQ | |
| SEQ ID NO | MGRVGYWTLLVLPALLVWRGPAPSAAAEKGPPAL | protein seq of fusion |
| 21 | NIAVMLGHSHDVTERELRTLWGPEQAAGLPLDVNV | protein #5 N2A-ATD- |
| | VALLMNRTDPKSLITHVCDLMSGARIHGLVFGDDTD | FC |
| | QEAVAQMLDFISSHTFVPILGIHGGASMIMADKDPT | |
| | STFFQFGASIQQQATVMLKIMQDYDWHVFSLVTTIF | |
| | PGYREFISFVKTTVDNSFVGWDMQNVITLDTSFED | |
| | AKTQVQLKKIHSSVILLYCSKDEAVLILSEARSLGLT | |
| | GYDFFWIVPSLVSGNTELIPKEFPSGLISVSYDDWD | |
| | YSLEARVRDGIGILTTAASSMLEKFSYIPEAKASCYG | |
| | QMERPEVPMHTLHPFMVNVTWDGKDLSFTEEGYQ | |
| | VHPRLVVIVLNKDREWEKVGKWENHTLSLRHAVW | |
| | PRYKSFSDCEPDDNHLSIGSSTMVRSSKPTCPPPE | |
| | LLGGPSVFIFPPKPKDTLMISRTPEVTCVVVDVSQD | |
| | DPEVQFTWYINNEQVRTARPPLREQQFNSTIRVVS | |
| | TLPIAHQDWLRGKEFKCKVHNKALPAPIEKTISKAR | |
| | GQPLEPKVYTMGPPREELSSRSVSLTCMINGFYPS | |
| | DISVEWEKNGKAEDNYKTTPAVLDSDGSYFLYSKL | |
| | SVPTSEWQRGDVFTCSVMHEALHNHYTQKSISRS | |
| | PGK | |
| SEQ ID NO | MGRVGYWTLLVLPALLVWRGPAPSAAAEKGPPAL | Amino terminal domain |
| 22 | NIAVMLGHSHDVTERELRTLWGPEQAAGLPLDVNV | of GluN2A (including |
| | VALLMNRTDPKSLITHVCDLMSGARIHGLVFGDDTD | signal sequence) |
| | QEAVAQMLDFISSHTFVPILGIHGGASMIMADKDPT | |
| | STFFQFGASIQQQATVMLKIMQDYDWHVFSLVTTIF | |
| | PGYREFISFVKTTVDNSFVGWDMQNVITLDTSFED | |
| | AKTQVQLKKIHSSVILLYCSKDEAVLILSEARSLGLT | |
| | GYDFFWIVPSLVSGNTELIPKEFPSGLISVSYDDWD | |
| | YSLEARVRDGIGILTTAASSMLEKFSYIPEAKASCYG | |
| | QMERPEVPMHTLHPFMVNVTWDGKDLSFTEEGYQ | |
| | VHPRLVVIVLNKDREWEKVGKWENHTLSLRHAVW | |
| | PRYKSFSDCEPDDNHLSI | |
| SEQ ID NO | PALNIAVMLGHSHDVTERELRTLWGPEQAAGLPLD | Amino terminal domain |
| 23 | VNVVALLMNRTDPKSLITHVCDLMSGARIHGLVFGD | of GluN2A in |
| | DTDQEAVAQMLDFISSHTFVPILGIHGGASMIMADK | NM_001134407 with |
| | DPTSTFFQFGASIQQQATVMLKIMQDYDWHVFSLV | domain borders as in |
| | TTIFPGYREFISFVKTTVDNSFVGWDMQNVITLDTS | cd06378: |
| | FEDAKTQVQLKKIHSSVILLYCSKDEAVLILSEARSL | PBP1_iGluR_NMDA |
| | GLTGYDFFWIVPSLVSGNTELIPKEFPSGLISVSYDD | NR2 (NCBI) |
| | WDYSLEARVRDGIGILTTAASSMLEKFSYIPEAKAS | |
| | CYGQMERPEVPMHTLHPFMVNVTWDGKDLSFTEE | |
| | GYQVHPRLVVIVLNKDREWEKVGKWENHTLSLRH | |
| | AVWPR | |
| SEQ ID NO | MKPRAECCSPKFWLVLAVLAVSGSRARSQKSPPSI | protein seq of fusion |
| 24 | GIAVILVGTSDEVAIKDAHEKDDFHHLSVVPRVELVA | protein #6 N2B-ATD- |
| | MNETDPKSIITRICDLMSDRKIQGVVFADDTDQEAIA | Fc |
| | QILDFISAQTLTPILGIHGGSSMIMADKDESSMFFQF | |
| | GPSIEQQASVMLNIMEEYDWYIFSIVTTYFPGYQDF | |
| | VNKIRSTIENSFVGWELEEVLLLDMSLDDGDSKIQN | |
| | QLKKLQSPIILLYCTKEEATYIFEVANSVGLTGYGYT | |
| | WIVPSLVAGDTDTVPAEFPTGLISVSYDEWDYGLP | |
| | ARVRDGIAIITTAASDMLSEHSFIPEPKSSCYNTHEK | |
| | RIYQSNMLNRYLINVTFEGRNLSFSEDGYQMHPKL | |
| | VIILLNKERKWERVGKWKDKSLQMKYYVWPRMCP | |
| | ETEEQEDDHLSIGSSTMVRSSKPTCPPPELLGGPS | |
| | VFIFPPKPKDTLMISRTPEVTCVVVDVSQDDPEVQF | |
| | TWYINNEQVRTARPPLREQQFNSTIRVVSTLPIAHQ | |
| | DWLRGKEFKCKVHNKALPAPIEKTISKARGQPLEPK | |
| | VYTMGPPREELSSRSVSLTCMINGFYPSDISVEWE | |
| | KNGKAEDNYKTTPAVLDSDGSYFLYSKLSVPTSEW | |
| | QRGDVFTCSVMHEALHNHYTQKSISRSPGK | |
| SEQ ID NO | MKPRAECCSPKFWLVLAVLAVSGSRARSQKSPPSI | Amino terminal domain |
| 25 | GIAVILVGTSDEVAIKDAHEKDDFHHLSVVPRVELVA | of GluN2B (including |
| | MNETDPKSIITRICDLMSDRKIQGVVFADDTDQEAIA | signal sequence) |
| | QILDFISAQTLTPILGIHGGSSMIMADKDESSMFFQF | |
| | GPSIEQQASVMLNIMEEYDWYIFSIVTTYFPGYQDF | |
| | VNKIRSTIENSFVGWELEEVLLLDMSLDDGDSKIQN | |
| | QLKKLQSPIILLYCTKEEATYIFEVANSVGLTGYGYT | |
| | WIVPSLVAGDTDTVPAEFPTGLISVSYDEWDYGLP | |
| | ARVRDGIAIITTAASDMLSEHSFIPEPKSSCYNTHEK | |
| | RIYQSNMLNRYLINVTFEGRNLSFSEDGYQMHPKL | |
| | VIILLNKERKWERVGKWKDKSLQMKYYVWPRMCP | |
| | ETEEQEDDHLSI | |
| SEQ ID NO | MSTMRLLTLALLFSCSVARAACDPKIVNIGAVLSTR | protein seq of fusion |
| 26 | KHEQMFREAVNQANKRHGSWKIQLNATSVTHKPN | protein #7 N1-ATD- |
| | AIQMALSVCEDLISSQVYAILVSHPPTPNDHFTPTPV | N2A-ATD-Fc |
| | SYTAGFYRIPVLGLTTRMSIYSDKSIHLSFLRTVPPY | |
| | SHQSSVWFEMMRVYSWNHIILLVSDDHEGRAAQK | |
| | RLETLLEERESKAEKVLQFDPGTKNVTALLMEAKEL | |
| | EARVIILSASEDDAATVYRAAAMLNMTGSGYVWLV | |
| | GEREISGNALRYAPDGILGLQLINGKNESAHISDAV | |
| | GVVAQAVHELLEKENITDPPRGCVGNTNIWKTGPL | |
| | FKRVLMSSKYADGVTGRVEFNEDGDRKFANYSIM | |
| | NLQNRKLVQVGIYNGTHVIPNDRKIIWPGGETEKPR | |
| | GYQMSTRLKIGSTGGGGSGGGGSGGGGSGAASR | |
| | EKGPPALNIAVMLGHSHDVTERELRTLWGPEQAAG | |
| | LPLDVNVVALLMNRTDPKSLITHVCDLMSGARIHGL | |
| | VFGDDTDQEAVAQMLDFISSHTFVPILGIHGGASMI | |
| | MADKDPTSTFFQFGASIQQQATVMLKIMQDYDWH | |
| | VFSLVTTIFPGYREFISFVKTTVDNSFVGWDMQNVI | |
| | TLDTSFEDAKTQVQLKKIHSSVILLYCSKDEAVLILS | |
| | EARSLGLTGYDFFWIVPSLVSGNTELIPKEFPSGLIS | |
| | VSYDDWDYSLEARVRDGIGILTTAASSMLEKFSYIP | |
| | EAKASCYGQMERPEVPMHTLHPFMVNVTWDGKD | |
| | LSFTEEGYQVHPRLVVIVLNKDREWEKVGKWENHT | |
| | LSLRHAVWPRYKSFSDCEPDDNHLSIGSSTMVRSS | |
| | KPTCPPPELLGGPSVFIFPPKPKDTLMISRTPEVTC | |
| | VVVDVSQDDPEVQFTWYINNEQVRTARPPLREQQ | |
| | FNSTIRVVSTLPIAHQDWLRGKEFKCKVHNKALPAP | |
| | IEKTISKARGQPLEPKVYTMGPPREELSSRSVSLTC | |
| | MINGFYPSDISVEWEKNGKAEDNYKTTPAVLDSDG | |
| | SYFLYSKLSVPTSEWQRGDVFTCSVMHEALHNHY | |
| | TQKSISRSPGK | |
| SEQ ID NO | MSTMRLLTLALLFSCSVARAACDPKIVNIGAVLSTR | Amino terminal domain |
| 27 | KHEQMFREAVNQANKRHGSWKIQLNATSVTHKPN | of GluN1 (including |
| | AIQMALSVCEDLISSQVYAILVSHPPTPNDHFTPTPV | signal sequence) as |
| | SYTAGFYRIPVLGLTTRMSIYSDKSIHLSFLRTVPPY | for example used in |
| | SHQSSVWFEMMRVYSWNHIILLVSDDHEGRAAQK | fusion protein #7 |
| | RLETLLEERESKAEKVLQFDPGTKNVTALLMEAKEL | |
| | EARVIILSASEDDAATVYRAAAMLNMTGSGYVWLV | |
| | GEREISGNALRYAPDGILGLQLINGKNESAHISDAV | |
| | GVVAQAVHELLEKENITDPPRGCVGNTNIWKTGPL | |
| | FKRVLMSSKYADGVTGRVEFNEDGDRKFANYSIM | |
| | NLQNRKLVQVGIYNGTHVIPNDRKIIWPGGETEKPR | |
| | GYQMSTRLKI | |
| SEQ ID NO | EKGPPALNIAVMLGHSHDVTERELRTLWGPEQAAG | Amino terminal domain |
| 28 | LPLDVNVVALLMNRTDPKSLITHVCDLMSGARIHGL | of GluN2A as for |
| | VFGDDTDQEAVAQMLDFISSHTFVPILGIHGGASMI | example used in fusion |
| | MADKDPTSTFFQFGASIQQQATVMLKIMQDYDWH | protein #7 |
| | VFSLVTTIFPGYREFISFVKTTVDNSFVGWDMQNVI | |
| | TLDTSFEDAKTQVQLKKIHSSVILLYCSKDEAVLILS | |
| | EARSLGLTGYDFFWIVPSLVSGNTELIPKEFPSGLIS | |
| | VSYDDWDYSLEARVRDGIGILTTAASSMLEKFSYIP | |
| | EAKASCYGQMERPEVPMHTLHPFMVNVTWDGKD | |
| | LSFTEEGYQVHPRLVVIVLNKDREWEKVGKWENHT | |
| | LSLRHAVWPRYKSFSDCEPDDNHLSI | |
| SEQ ID NO | MSTMRLLTLALLFSCSVARAACDPKIVNIGAVLSTR | protein seq of fusion |
| 29 | KHEQMFREAVNQANKRHGSWKIQLNATSVTHKPN | protein #8 N1-ATD- |
| | AIQMALSVCEDLISSQVYAILVSHPPTPNDHFTPTPV | N2B-ATD-Fc |
| | SYTAGFYRIPVLGLTTRMSIYSDKSIHLSFLRTVPPY | |
| | SHQSSVWFEMMRVYSWNHIILLVSDDHEGRAAQK | |
| | RLETLLEERESKAEKVLQFDPGTKNVTALLMEAKEL | |
| | EARVIILSASEDDAATVYRAAAMLNMTGSGYVWLV | |
| | GEREISGNALRYAPDGILGLQLINGKNESAHISDAV | |
| | GVVAQAVHELLEKENITDPPRGCVGNTNIWKTGPL | |
| | FKRVLMSSKYADGVTGRVEFNEDGDRKFANYSIM | |
| | NLQNRKLVQVGIYNGTHVIPNDRKIIWPGGETEKPR | |
| | GYQMSTRLKIGSTGGGGSGGGGSGGGGSGAASR | |
| | QKSPPSIGIAVILVGTSDEVAIKDAHEKDDFHHLSVV | |
| | PRVELVAMNETDPKSIITRICDLMSDRKIQGVVFAD | |
| | DTDQEAIAQILDFISAQTLTPILGIHGGSSMIMADKD | |
| | ESSMFFQFGPSIEQQASVMLNIMEEYDWYIFSIVTT | |
| | YFPGYQDFVNKIRSTIENSFVGWELEEVLLLDMSLD | |
| | DGDSKIQNQLKKLQSPIILLYCTKEEATYIFEVANSV | |
| | GLTGYGYTWIVPSLVAGDTDTVPAEFPTGLISVSYD | |
| | EWDYGLPARVRDGIAIITTAASDMLSEHSFIPEPKSS | |
| | CYNTHEKRIYQSNMLNRYLINVTFEGRNLSFSEDG | |
| | YQMHPKLVIILLNKERKWERVGKWKDKSLQMKYYV | |
| | WPRMCPETEEQEDDHLSIGSSTMVRSSKPTCPPP | |
| | ELLGGPSVFIFPPKPKDTLMISRTPEVTCVVVDVSQ | |
| | DDPEVQFTWYINNEQVRTARPPLREQQFNSTIRVV | |
| | STLPIAHQDWLRGKEFKCKVHNKALPAPIEKTISKA | |
| | RGQPLEPKVYTMGPPREELSSRSVSLTCMINGFYP | |
| | SDISVEWEKNGKAEDNYKTTPAVLDSDGSYFLYSK | |
| | LSVPTSEWQRGDVFTCSVMHEALHNHYTQKSISRS | |
| | PGK | |
| SEQ ID NO | MSTMRLLTLALLFSCSVARAACDPKIVNIGAVLSTR | Amino terminal domain |
| 30 | KHEQMFREAVNQANKRHGSWKIQLNATSVTHKPN | of GluN1 (including |
| | AIQMALSVCEDLISSQVYAILVSHPPTPNDHFTPTPV | signal sequence) as |
| | SYTAGFYRIPVLGLTTRMSIYSDKSIHLSFLRTVPPY | used for example in |
| | SHQSSVWFEMMRVYSWNHIILLVSDDHEGRAAQK | fusion protein #8 |
| | RLETLLEERESKAEKVLQFDPGTKNVTALLMEAKEL | |
| | EARVIILSASEDDAATVYRAAAMLNMTGSGYVWLV | |
| | GEREISGNALRYAPDGILGLQLINGKNESAHISDAV | |
| | GVVAQAVHELLEKENITDPPRGCVGNTNIWKTGPL | |
| | FKRVLMSSKYADGVTGRVEFNEDGDRKFANYSIM | |
| | NLQNRKLVQVGIYNGTHVIPNDRKIIWPGGETEKPR | |
| | GYQMSTRLKI | |
| SEQ ID NO | QKSPPSIGIAVILVGTSDEVAIKDAHEKDDFHHLSVV | Amino terminal domain |
| 31 | PRVELVAMNETDPKSIITRICDLMSDRKIQGVVFAD | of GluN2B as for |
| | DTDQEAIAQILDFISAQTLTPILGIHGGSSMIMADKD | example used in fusion |
| | ESSMFFQFGPSIEQQASVMLNIMEEYDWYIFSIVTT | protein #8 |
| | YFPGYQDFVNKIRSTIENSFVGWELEEVLLLDMSLD | |
| | DGDSKIQNQLKKLQSPIILLYCTKEEATYIFEVANSV | |
| | GLTGYGYTWIVPSLVAGDTDTVPAEFPTGLISVSYD | |
| | EWDYGLPARVRDGIAIITTAASDMLSEHSFIPEPKSS | |
| | CYNTHEKRIYQSNMLNRYLINVTFEGRNLSFSEDG | |
| | YQMHPKLVIILLNKERKWERVGKWKDKSLQMKYYV | |
| | WPRMCPETEEQEDDHLSI | |
| SEQ ID NO | MGGALGPALLLTSLFGAWAGLGPGQGEQGMTVAV | Human GluN2C |
| 32 | VFSSSGPPQAQFRARLTPQSFLDLPLEIQPLTVGVN | NM_000835 |
| | TTNPSSLLTQICGLLGAAHVHGIVFEDNVDTEAVAQI | |
| | LDFISSQTHVPILSISGGSAVVLTPKEPGSAFLQLGV | |
| | SLEQQLQVLFKVLEEYDWSAFAVITSLHPGHALFLE | |
| | GVRAVADASHVSWRLLDVVTLELGPGGPRARTQR | |
| | LLRQLDAPVFVAYCSREEAEVLFAEAAQAGLVGPG | |
| | HVWLVPNLALGSTDAPPATFPVGLISVVTESWRLSL | |
| | RQKVRDGVAILALGAHSYWRQHGTLPAPAGDCRV | |
| | HPGPVSPAREAFYRHLLNVTWEGRDFSFSPGGYL | |
| | VQPTMVVIALNRHRLWEMVGRWEHGVLYMKYPV | |
| | WPRYSASLQPVVDSRHLTVATLEERPFVIVESPDP | |
| | GTGGCVPNTVPCRRQSNHTFSSGDVAPYTKLCCK | |
| | GFCIDILKKLARVVKFSYDLYLVTNGKHGKRVRGV | |
| | WNGMIGEVYYKRADMAIGSLTINEERSEIVDFSVPF | |
| | VETGISVMVARSNGTVSPSAFLEPYSPAVWVMMFV | |
| | MCLTVVAITVFMFEYFSPVSYNQNLTRGKKSGGPA | |
| | FTIGKSVWLLWALVENNSVPIENPRGTTSKIMVLVW | |
| | AFFAVIFLASYTANLAAFMIQEQYIDTVSGLSDKKFQ | |
| | RPQDQYPPFRFGTVPNGSTERNIRSNYRDMHTHM | |
| | VKFNQRSVEDALTSLKMGKLDAFIYDAAVLNYMAG | |
| | KDEGCKLVTIGSGKVFATTGYGIAMQKDSHWKRAI | |
| | DLALLQFLGDGETQKLETVWLSGICQNEKNEVMSS | |
| | KLDIDNMAGVFYMLLVAMGLALLVFAWEHLVYWKL | |
| | RHSVPNSSQLDFLLAFSRGIYSCFSGVQSLASPPR | |
| | QASPDLTASSAQASVLKMLQAARDMVTTAGVSSSL | |
| | DRATRTIENWGGGRRAPPPSPCPTPRSGPSPCLP | |
| | TPDPPPEPSPTGWGPPDGGRAALVRRAPQPPGRP | |
| | PTPGPPLSDVSRVSRRPAWEARWPVRTGHCGRH | |
| | LSASERPLSPARCHYSSFPRADRSGRPFLPLFPEL | |
| | EDLPLLGPEQLARREALLHAAWARGSRPRHASLPS | |
| | SVAEAFARPSSLPAGCTGPACARPDGHSACRRLA | |
| | QAQSMCLPIYREACQEGEQAGAPAWQHRQHVCL | |
| | HAHAHLPFCWGAVCPHLPPCASHGSWLSGAWGP | |
| | LGHRGRTLGLGTGYRDSGGLDEISRVARGTQGFP | |
| | GPCTWRRISSLESEV | |
| SEQ ID NO | QGMTVAVVFSSSGPPQAQFRARLTPQSFLDLPLEI | GluN2C ATD |
| 33 | QPLTVGVNTTNPSSLLTQICGLLGAAHVHGIVFEDN | |
| | VDTEAVAQILDFISSQTHVPILSISGGSAVVLTPKEP | |
| | GSAFLQLGVSLEQQLQVLFKVLEEYDWSAFAVITSL | |
| | HPGHALFLEGVRAVADASHVSWRLLDVVTLELGPG | |
| | GPRARTQRLLRQLDAPVFVAYCSREEAEVLFAEAA | |
| | QAGLVGPGHVWLVPNLALGSTDAPPATFPVGLISV | |
| | VTESWRLSLRQKVRDGVAILALGAHSYWRQHGTL | |
| | PAPAGDCRVHPGPVSPAREAFYRHLLNVTWEGRD | |
| | FSFSPGGYLVQPTMVVIALNRHRLWEMVGRWEHG | |
| | VLYMKYPVWPR | |
| SEQ ID NO | SRHLTVATLEERPFVIVESPDPGTGGCVPNTVPCR | GluN2C S1 |
| 34 | RQSNHTFSSGDVAPYTKLCCKGFCIDILKKLARVVK | |
| | FSYDLYLVTNGKHGKRVRGVWNGMIGEVYYKRAD | |
| | MAIGSLTINEERSEIVDFSVPFVETGISVMVAR | |
| SEQ ID NO | TVSGLSDKKFQRPQDQYPPFRFGTVPNGSTERNIR | GluN2C S2 |
| 35 | SNYRDMHTHMVKFNQRSVEDALTSLKMGKLDAFIY | |
| | DAAVLNYMAGKDEGCKLVTIGSGKVFATTGYGIAM | |
| | QKDSHWKRAIDLALLQFLGDGETQKLETVWLSGIC | |
| | QN | |
| SEQ ID NO | MGGALGPALLLTSLFGAWAGLGPGQGEQGMTVAV | GluN2C ecd incl. |
| 36 | VFSSSGPPQAQFRARLTPQSFLDLPLEIQPLTVGVN | signal sequence and |
| | TTNPSSLLTQICGLLGAAHVHGIVFEDNVDTEAVAQI | GT-linker between S1 |
| | LDFISSQTHVPILSISGGSAVVLTPKEPGSAFLQLGV | and S2 |
| | SLEQQLQVLFKVLEEYDWSAFAVITSLHPGHALFLE | |
| | GVRAVADASHVSWRLLDVVTLELGPGGPRARTQR | |
| | LLRQLDAPVFVAYCSREEAEVLFAEAAQAGLVGPG | |
| | HVWLVPNLALGSTDAPPATFPVGLISVVTESWRLSL | |
| | RQKVRDGVAILALGAHSYWRQHGTLPAPAGDCRV | |
| | HPGPVSPAREAFYRHLLNVTWEGRDFSFSPGGYL | |
| | VQPTMVVIALNRHRLWEMVGRWEHGVLYMKYPV | |
| | WPRYSASLQPVVDSRHLTVATLEERPFVIVESPDP | |
| | GTGGCVPNTVPCRRQSNHTFSSGDVAPYTKLCCK | |
| | GFCIDILKKLARVVKFSYDLYLVTNGKHGKRVRGV | |
| | WNGMIGEVYYKRADMAIGSLTINEERSEIVDFSVPF | |
| | VETGISVMVARGTTVSGLSDKKFQRPQDQYPPFRF | |
| | GTVPNGSTERNIRSNYRDMHTHMVKFNQRSVEDA | |
| | LTSLKMGKLDAFIYDAAVLNYMAGKDEGCKLVTIGS | |
| | GKVFATTGYGIAMQKDSHWKRAIDLALLQFLGDGE | |
| | TQKLETVWLSGICQN | |
| SEQ ID NO | MRGAGGPRGPRGPAKMLLLLALACASPFPEEAPG | Human GluN2D |
| 37 | PGGAGGPGGGLGGARPLNVALVFSGPAYAAEAAR | NP_000827 (protein) |
| | LGPAVAAAVRSPGLDVRPVALVLNGSDPRSLVLQL | |
| | CDLLSGLRVHGVVFEDDSRAPAVAPILDFLSAQTSL | |
| | PIVAVHGGAALVLTPKEKGSTFLQLGSSTEQQLQVI | |
| | FEVLEEYDWTSFVAVTTRAPGHRAFLSYIEVLTDGS | |
| | LVGWEHRGALTLDPGAGEAVLSAQLRSVSAQIRLL | |
| | FCAREEAEPVFRAAEEAGLTGSGYVWFMVGPQLA | |
| | GGGGSGAPGEPPLLPGGAPLPAGLFAVRSAGWRD | |
| | DLARRVAAGVAVVARGAQALLRDYGFLPELGHDC | |
| | RAQNRTHRGESLHRYFMNITWDNRDYSFNEDGFL | |
| | VNPSLVVISLTRDRTWEVVGSWEQQTLRLKYPLWS | |
| | RYGRFLQPVDDTQHLTVATLEERPFVIVEPADPISG | |
| | TCIRDSVPCRSQLNRTHSPPPDAPRPEKRCCKGFC | |
| | IDILKRLAHTIGFSYDLYLVTNGKHGKKIDGVWNGMI | |
| | GEVFYQRADMAIGSLTINEERSEIVDFSVPFVETGIS | |
| | VMVARSNGTVSPSAFLEPYSPAVWVMMFVMCLTV | |
| | VAVTVFIFEYLSPVGYNRSLATGKRPGGSTFTIGKSI | |
| | WLLWALVFNNSVPVENPRGTTSKIMVLVWAFFAVI | |
| | FLASYTANLAAFMIQEEYVDTVSGLSDRKFQRPQE | |
| | QYPPLKFGTVPNGSTEKNIRSNYPDMHSYMVRYN | |
| | QPRVEEALTQLKAGKLDAFIYDAAVLNYMARKDEG | |
| | CKLVTIGSGKVFATTGYGIALHKGSRWKRPIDLALL | |
| | QFLGDDEIEMLERLWLSGICHNDKIEVMSSKLDIDN | |
| | MAGVFYMLLVAMGLSLLVFAWEHLVYWRLRHCLG | |
| | PTHRMDFLLAFSRGMYSCCSAEAAPPPAKPPPPP | |
| | QPLPSPAYPAPRPAPGPAPFVPRERASVDRWRRT | |
| | KGAGPPGGAGLADGFHRYYGPIEPQGLGLGLGEA | |
| | RAAPRGAAGRPLSPPAAQPPQKPPPSYFAIVRDKE | |
| | PAEPPAGAFPGFPSPPAPPAAAATAVGPPLCRLAF | |
| | EDESPPAPARWPRSDPESQPLLGPGAGGAGGTG | |
| | GAGGGAPAAPPPCRAAPPPCPYLDLEPSPSDSED | |
| | SESLGGASLGGLEPWWFADFPYPYAERLGPPPGR | |
| | YWSVDKLGGWRAGSWDYLPPRSGPAAWHCRHC | |
| | ASLELLPPPRHLSCSHDGLDGGWWAPPPPPWAAG | |
| | PLPRRRARCGCPRSHPHRPRASHRTPAAAAPHHH | |
| | RHRRAAGGWDLPPPAPTSRSLEDLSSCPRAAPAR | |
| | RLTGPSRHARRCPHAAHWGPPLPTASHRRHRGG | |
| | DLGTRRGSAHFSSLESEV | |
| SEQ ID NO | MRGAGGPRGPRGPAKMLLLLALACASPFPEEAPG | Human GluN2D |
| 38 | PGGAGGPGGGLGGARPLNVALVFSGPAYAAEAAR | Protein seq U77783 |
| | LGPAVAAAVRSPGLDVRPVALVLNGSDPRSLVLQL | |
| | CDLLSGLRVHGVVFEDDSRAPAVAPILDFLSAQTSL | |
| | PIVAVHGGAALVLTPKEKGSTFLQLGSSTEQQLQVI | |
| | FEVLEEYDWTSFVAVTTRAPGHRAFLSYIEVLTDGS | |
| | LVGWEHRGALTLDPGAGEAVLSAQLRSVSAQIRLL | |
| | FCAREEAEPVFRAAEEAGLTGSGYVWFMVGPQLA | |
| | GGGGSGAPGEPPLLPGGAPLPAGLFAVRSAGWRD | |
| | DLARRVAAGVAVVARGAQALLRDYGFLPELGHDC | |
| | RAQNRTHRGESLHRYFMNITWDNRDYSFNEDGFL | |
| | VNPSLVVISLTRDRTWEVVGSWEQQTLRLKYPLWS | |
| | RYGRFLQPVDDTQHLTVATLEERPFVIVEPADPISG | |
| | TCIRDSVPCRSQLNRTHSPPPDAPRPEKRCCKGFC | |
| | IDILKRLAHTIGFSYDLYLVTNGKHGKKIDGVWNGMI | |
| | GEVFYQRADMAIGSLTINEERSEIVDFSVPFVETGIS | |
| | VMVARSNGTVSPSAFLEPYSPAVWVMMFVMCLTV | |
| | VAVTVFIFEYLSPVGYNRSLATGKRPGGSTFTIGKSI | |
| | WLLWALVENNSVPVENPRGTTSKIMVLVWAFFAVI | |
| | FLASYTANLAAFMIQEEYVDTVSGLSDRKFQRPQE | |
| | QYPPLKFGTVPNGSTEKNIRSNYPDMHSYMVRYN | |
| | QPRVEEALTQLKAGKLDAFIYDAAVLNYMARKDEG | |
| | CKLVTIGSGKVFATTGYGIALHKGSRWKRPIDLALL | |
| | QFLGDDEIEMLERLWLSGICHNDKIEVMSSKLDIDN | |
| | MAGVFYMLLVAMGLSLLVFAWEHLVYWRLRHCLG | |
| | PTHRMDFLLAFSRGMYSCCSAEAAPPPAKPPPPP | |
| | QPLPSPAYPAPGPAPGPAPFVPRERASVDRWRRT | |
| | KGAGPPGGAGLADGFHRYYGPIEPQGLGLGLGEA | |
| | RAAPRGAAGRPLSPPAAQPPQKPPASYFAIVRDKE | |
| | PAEPPAGAFPGFPSPPAPPAAAATAVGPPLCRLAF | |
| | EDESPPAPARWPRSDPESQPLLGPGAGGAGGTG | |
| | GAGGGAPAAPPPCCAAPPPCPYLDLEPSPSDSED | |
| | SESLGGASLGGLDPWWFADFPYPYAERLGPPPGR | |
| | YWSVDKLGGWRAGSWDYLPPRSGPAAWHCRHC | |
| | ASLELLPPPRHLSCSHDGLDGGWWAPPPPPWAAG | |
| | PLPRRRARCGCPRSHPHRPRASHRTPAAAAPHHH | |
| | RHRRAAGGWDLPPPAPTSRSLEDLSSCPRAAPAR | |
| | RLTGPSRHARRCPHAAHWGPPLPTASHRRHRGG | |
| | DLGTRRGSAHFSSLESEV | |
| SEQ ID NO | RPLNVALVFSGPAYAAEAARLGPAVAAAVRSPGLD | GluN2D ATD |
| 39 | VRPVALVLNGSDPRSLVLQLCDLLSGLRVHGVVFE | |
| | DDSRAPAVAPILDFLSAQTSLPIVAVHGGAALVLTP | |
| | KEKGSTFLQLGSSTEQQLQVIFEVLEEYDWTSFVA | |
| | VTTRAPGHRAFLSYIEVLTDGSLVGWEHRGALTLD | |
| | PGAGEAVLSAQLRSVSAQIRLLFCAREEAEPVFRA | |
| | AEEAGLTGSGYVWFMVGPQLAGGGGSGAPGEPP | |
| | LLPGGAPLPAGLFAVRSAGWRDDLARRVAAGVAV | |
| | VARGAQALLRDYGFLPELGHDCRAQNRTHRGESL | |
| | HRYFMNITWDNRDYSFNEDGFLVNPSLVVISLTRD | |
| | RTWEVVGSWEQQTLRLKYPLWSR | |
| SEQ ID NO | TQHLTVATLEERPFVIVEPADPISGTCIRDSVPCRS | GluN2D S1 |
| 40 | QLNRTHSPPPDAPRPEKRCCKGFCIDILKRLAHTIG | |
| | FSYDLYLVTNGKHGKKIDGVWNGMIGEVFYQRAD | |
| | MAIGSLTINEERSEIVDFSVPFVETGISVMVAR | |
| SEQ ID NO | TVSGLSDRKFQRPQEQYPPLKFGTVPNGSTEKNIR | GluN2D S2 |
| 41 | SNYPDMHSYMVRYNQPRVEEALTQLKAGKLDAFIY | |
| | DAAVLNYMARKDEGCKLVTIGSGKVFATTGYGIALH | |
| | KGSRWKRPIDLALLQFLGDDEIEMLERLWLSGICHN | |
| SEQ ID NO | MRGAGGPRGPRGPAKMLLLLALACASPFPEEAPG | GluN2D ecd incl. |
| 42 | PGGAGGPGGGLGGARPLNVALVFSGPAYAAEAAR | signal sequence and |
| | LGPAVAAAVRSPGLDVRPVALVLNGSDPRSLVLQL | GT-linker between S1 |
| | CDLLSGLRVHGVVFEDDSRAPAVAPILDFLSAQTSL | and S2 |
| | PIVAVHGGAALVLTPKEKGSTFLQLGSSTEQQLQVI | |
| | FEVLEEYDWTSFVAVTTRAPGHRAFLSYIEVLTDGS | |
| | LVGWEHRGALTLDPGAGEAVLSAQLRSVSAQIRLL | |
| | FCAREEAEPVFRAAEEAGLTGSGYVWFMVGPQLA | |
| | GGGGSGAPGEPPLLPGGAPLPAGLFAVRSAGWRD | |
| | DLARRVAAGVAVVARGAQALLRDYGFLPELGHDC | |
| | RAQNRTHRGESLHRYFMNITWDNRDYSFNEDGFL | |
| | VNPSLVVISLTRDRTWEVVGSWEQQTLRLKYPLWS | |
| | RYGRFLQPVDDTQHLTVATLEERPFVIVEPADPISG | |
| | TCIRDSVPCRSQLNRTHSPPPDAPRPEKRCCKGFC | |
| | IDILKRLAHTIGFSYDLYLVTNGKHGKKIDGVWNGMI | |
| | GEVFYQRADMAIGSLTINEERSEIVDFSVPFVETGIS | |
| | VMVARGTTVSGLSDRKFQRPQEQYPPLKFGTVPN | |
| | GSTEKNIRSNYPDMHSYMVRYNQPRVEEALTQLKA | |
| | GKLDAFIYDAAVLNYMARKDEGCKLVTIGSGKVFAT | |
| | TGYGIALHKGSRWKRPIDLALLQFLGDDEIEMLERL | |
| | WLSGICHN | |
| SEQ ID NO | MGRVGYWTLLVLPALLVWRGPAPSAAAEKGPPAL | Human GluN2A |
| 43 | NIAVMLGHSHDVTERELRTLWGPEQAAGLPLDVNV | NM_001134407 |
| | VALLMNRTDPKSLITHVCDLMSGARIHGLVFGDDTD | |
| | QEAVAQMLDFISSHTFVPILGIHGGASMIMADKDPT | |
| | STFFQFGASIQQQATVMLKIMQDYDWHVFSLVTTIF | |
| | PGYREFISFVKTTVDNSFVGWDMQNVITLDTSFED | |
| | AKTQVQLKKIHSSVILLYCSKDEAVLILSEARSLGLT | |
| | GYDFFWIVPSLVSGNTELIPKEFPSGLISVSYDDWD | |
| | YSLEARVRDGIGILTTAASSMLEKFSYIPEAKASCYG | |
| | QMERPEVPMHTLHPFMVNVTWDGKDLSFTEEGYQ | |
| | VHPRLVVIVLNKDREWEKVGKWENHTLSLRHAVW | |
| | PRYKSFSDCEPDDNHLSIVTLEEAPFVIVEDIDPLTE | |
| | TCVRNTVPCRKFVKINNSTNEGMNVKKCCKGFCIDI | |
| | LKKLSRTVKFTYDLYLVTNGKHGKKVNNVWNGMIG | |
| | EVVYQRAVMAVGSLTINEERSEVVDFSVPFVETGIS | |
| | VMVSRSNGTVSPSAFLEPFSASVWVMMFVMLLIVS | |
| | AIAVFVFEYFSPVGYNRNLAKGKAPHGPSFTIGKAI | |
| | WLLWGLVFNNSVPVQNPKGTTSKIMVSVWAFFAVI | |
| | FLASYTANLAAFMIQEEFVDQVTGLSDKKFQRPHD | |
| | YSPPFRFGTVPNGSTERNIRNNYPYMHQYMTKFN | |
| | QKGVEDALVSLKTGKLDAFIYDAAVLNYKAGRDEG | |
| | CKLVTIGSGYIFATTGYGIALQKGSPWKRQIDLALLQ | |
| | FVGDGEMEELETLWLTGICHNEKNEVMSSQLDIDN | |
| | MAGVFYMLAAAMALSLITFIWEHLFYWKLRFCFTGV | |
| | CSDRPGLLFSISRGIYSCIHGVHIEEKKKSPDFNLTG | |
| | SQSNMLKLLRSAKNISSMSNMNSSRMDSPKRAAD | |
| | FIQRGSLIMDMVSDKGNLMYSDNRSFQGKESIFGD | |
| | NMNELQTFVANRQKDNLNNYVFQGQHPLTLNESN | |
| | PNTVEVAVSTESKANSRPRQLWKKSVDSIRQDSLS | |
| | QNPVSQRDEATAENRTHSLKSPRYLPEEMAHSDIS | |
| | ETSNRATCHREPDNSKNHKTKDNFKRSVASKYPK | |
| | DCSEVERTYLKTKSSSPRDKIYTIDGEKEPGFHLDP | |
| | PQFVENVTLPENVDFPDPYQDPSENFRKGDSTLP | |
| | MNRNPLHNEEGLSNNDQYKLYSKHFTLKDKGSPH | |
| | SETSERYRQNSTHCRSCLSNMPTYSGHFTMRSPF | |
| | KCDACLRMGNLYDIDEDQMLQETGNPATGEQVYQ | |
| | QDWAQNNALQLQKNKLRISRQHSYDNIVDKPRELD | |
| | LSRPSRSISLKDRERLLEGNFYGSLFSVPSSKLSGK | |
| | KSSLFPQGLEDSKRSKSLLPDHTSDNPFLHSHRDD | |
| | QRLVIGRCPSDPYKHSLPSQAVNDSYLRSSLRSTA | |
| | SYCSRDSRGHNDVYISEHVMPYAANKNNMYSTPR | |
| | VLNSCSNRRVYKKMPSIESDV | |
| SEQ ID NO | DNHLSIVTLEEAPFVIVEDIDPLTETCVRNTVPCRKF | GluN2A S1 |
| 44 | VKINNSTNEGMNVKKCCKGFCIDILKKLSRTVKFTY | |
| | DLYLVTNGKHGKKVNNVWNGMIGEVVYQRAVMAV | |
| | GSLTINEERSEVVDFSVPFVETGISVMVSR | |
| SEQ ID NO | QVTGLSDKKFQRPHDYSPPFRFGTVPNGSTERNIR | GluN2A S2 |
| 45 | NNYPYMHQYMTKFNQKGVEDALVSLKTGKLDAFIY | |
| | DAAVLNYKAGRDEGCKLVTIGSGYIFATTGYGIALQ | |
| | KGSPWKRQIDLALLQFVGDGEMEELETLWLTGICH | |
| | N | |
| SEQ ID NO | MGRVGYWTLLVLPALLVWRGPAPSAAAEKGPPAL | GluN2A ecd incl. |
| 46 | NIAVMLGHSHDVTERELRTLWGPEQAAGLPLDVNV | signal sequence and |
| | VALLMNRTDPKSLITHVCDLMSGARIHGLVFGDDTD | GT-linker between S1 |
| | QEAVAQMLDFISSHTFVPILGIHGGASMIMADKDPT | and S2 |
| | STFFQFGASIQQQATVMLKIMQDYDWHVFSLVTTIF | |
| | PGYREFISFVKTTVDNSFVGWDMQNVITLDTSFED | |
| | AKTQVQLKKIHSSVILLYCSKDEAVLILSEARSLGLT | |
| | GYDFFWIVPSLVSGNTELIPKEFPSGLISVSYDDWD | |
| | YSLEARVRDGIGILTTAASSMLEKFSYIPEAKASCYG | |
| | QMERPEVPMHTLHPFMVNVTWDGKDLSFTEEGYQ | |
| | VHPRLVVIVLNKDREWEKVGKWENHTLSLRHAVW | |
| | PRYKSFSDCEPDDNHLSIVTLEEAPFVIVEDIDPLTE | |
| | TCVRNTVPCRKFVKINNSTNEGMNVKKCCKGFCIDI | |
| | LKKLSRTVKFTYDLYLVTNGKHGKKVNNVWNGMIG | |
| | EVVYQRAVMAVGSLTINEERSEVVDFSVPFVETGIS | |
| | VMVSRGTQVTGLSDKKFQRPHDYSPPFRFGTVPN | |
| | GSTERNIRNNYPYMHQYMTKFNQKGVEDALVSLKT | |
| | GKLDAFIYDAAVLNYKAGRDEGCKLVTIGSGYIFAT | |
| | TGYGIALQKGSPWKRQIDLALLQFVGDGEMEELET | |
| | LWLTGICHN | |
| SEQ ID NO | MKPRAECCSPKFWLVLAVLAVSGSRARSQKSPPSI | Human GluN2B |
| 47 | GIAVILVGTSDEVAIKDAHEKDDFHHLSVVPRVELVA | NM_000834 |
| | MNETDPKSIITRICDLMSDRKIQGVVFADDTDQEAIA | |
| | QILDFISAQTLTPILGIHGGSSMIMADKDESSMFFQF | |
| | GPSIEQQASVMLNIMEEYDWYIFSIVTTYFPGYQDF | |
| | VNKIRSTIENSFVGWELEEVLLLDMSLDDGDSKIQN | |
| | QLKKLQSPIILLYCTKEEATYIFEVANSVGLTGYGYT | |
| | WIVPSLVAGDTDTVPAEFPTGLISVSYDEWDYGLP | |
| | ARVRDGIAIITTAASDMLSEHSFIPEPKSSCYNTHEK | |
| | RIYQSNMLNRYLINVTFEGRNLSFSEDGYQMHPKL | |
| | VIILLNKERKWERVGKWKDKSLQMKYYVWPRMCP | |
| | ETEEQEDDHLSIVTLEEAPFVIVESVDPLSGTCMRN | |
| | TVPCQKRIVTENKTDEEPGYIKKCCKGFCIDILKKIS | |
| | KSVKFTYDLYLVTNGKHGKKINGTWNGMIGEVVMK | |
| | RAYMAVGSLTINEERSEVVDFSVPFIETGISVMVSR | |
| | SNGTVSPSAFLEPFSADVWVMMFVMLLIVSAVAVF | |
| | VFEYFSPVGYNRCLADGREPGGPSFTIGKAIWLLW | |
| | GLVFNNSVPVQNPKGTTSKIMVSVWAFFAVIFLASY | |
| | TANLAAFMIQEEYVDQVSGLSDKKFQRPNDFSPPF | |
| | RFGTVPNGSTERNIRNNYAEMHAYMGKFNQRGVD | |
| | DALLSLKTGKLDAFIYDAAVLNYMAGRDEGCKLVTI | |
| | GSGKVFASTGYGIAIQKDSGWKRQVDLAILQLFGD | |
| | GEMEELEALWLTGICHNEKNEVMSSQLDIDNMAGV | |
| | FYMLGAAMALSLITFICEHLFYWQFRHCFMGVCSG | |
| | KPGMVFSISRGIYSCIHGVAIEERQSVMNSPTATMN | |
| | NTHSNILRLLRTAKNMANLSGVNGSPQSALDFIRRE | |
| | SSVYDISEHRRSFTHSDCKSYNNPPCEENLFSDYIS | |
| | EVERTFGNLQLKDSNVYQDHYHHHHRPHSIGSASS | |
| | IDGLYDCDNPPFTTQSRSISKKPLDIGLPSSKHSQL | |
| | SDLYGKFSFKSDRYSGHDDLIRSDVSDISTHTVTYG | |
| | NIEGNAAKRRKQQYKDSLKKRPASAKSRREFDEIE | |
| | LAYRRRPPRSPDHKRYFRDKEGLRDFYLDQFRTKE | |
| | NSPHWEHVDLTDIYKERSDDFKRDSVSGGGPCTN | |
| | RSHIKHGTGDKHGVVSGVPAPWEKNLTNVEWEDR | |
| | SGGNFCRSCPSKLHNYSTTVTGQNSGRQACIRCE | |
| | ACKKAGNLYDISEDNSLQELDQPAAPVAVTSNAST | |
| | TKYPQSPTNSKAQKKNRNKLRRQHSYDTFVDLQK | |
| | EEAALAPRSVSLKDKGRFMDGSPYAHMFEMSAGE | |
| | STFANNKSSVPTAGHHHHNNPGGGYMLSKSLYPD | |
| | RVTQNPFIPTFGDDQCLLHGSKSYFFRQPTVAGAS | |
| | KARPDFRALVTNKPVVSALHGAVPARFQKDICIGN | |
| | QSNPCVPNNKNPRAFNGSSNGHVYEKLSSIESDV | |
| SEQ ID NO | MKPRAECCSPKFWLVLAVLAVSGSRARSQKSPPSI | GluN2B ecd incl. |
| 48 | GIAVILVGTSDEVAIKDAHEKDDFHHLSVVPRVELVA | signal sequence and |
| | MNETDPKSIITRICDLMSDRKIQGVVFADDTDQEAIA | GT-linker between S1 |
| | QILDFISAQTLTPILGIHGGSSMIMADKDESSMFFQF | and S2 |
| | GPSIEQQASVMLNIMEEYDWYIFSIVTTYFPGYQDF | |
| | VNKIRSTIENSFVGWELEEVLLLDMSLDDGDSKIQN | |
| | QLKKLQSPIILLYCTKEEATYIFEVANSVGLTGYGYT | |
| | WIVPSLVAGDTDTVPAEFPTGLISVSYDEWDYGLP | |
| | ARVRDGIAIITTAASDMLSEHSFIPEPKSSCYNTHEK | |
| | RIYQSNMLNRYLINVTFEGRNLSFSEDGYQMHPKL | |
| | VIILLNKERKWERVGKWKDKSLQMKYYVWPRMCP | |
| | ETEEQEDDHLSIVTLEEAPFVIVESVDPLSGTCMRN | |
| | TVPCQKRIVTENKTDEEPGYIKKCCKGFCIDILKKIS | |
| | KSVKFTYDLYLVTNGKHGKKINGTWNGMIGEVVMK | |
| | RAYMAVGSLTINEERSEVVDFSVPFIETGISVMVSR | |
| | GTQVSGLSDKKFQRPNDFSPPFRFGTVPNGSTER | |
| | NIRNNYAEMHAYMGKFNQRGVDDALLSLKTGKLDA | |
| | FIYDAAVLNYMAGRDEGCKLVTIGSGKVFASTGYGI | |
| | AIQKDSGWKRQVDLAILQLFGDGEMEELEALWLTG | |
| | ICHN | |
| SEQ ID NO | MGGALGPALLLTSLFGAWAGLGPGQGEQGMTVAV | protein seq of fusion |
| 96 | VFSSSGPPQAQFRARLTPQSFLDLPLEIQPLTVGVN | protein #9 N2C-ATD- |
| | TTNPSSLLTQICGLLGAAHVHGIVFEDNVDTEAVAQI | FC |
| | LDFISSQTHVPILSISGGSAVVLTPKEPGSAFLQLGV | |
| | SLEQQLQVLFKVLEEYDWSAFAVITSLHPGHALFLE | |
| | GVRAVADASHVSWRLLDVVTLELGPGGPRARTQR | |
| | LLRQLDAPVFVAYCSREEAEVLFAEAAQAGLVGPG | |
| | HVWLVPNLALGSTDAPPATFPVGLISVVTESWRLSL | |
| | RQKVRDGVAILALGAHSYWRQHGTLPAPAGDCRV | |
| | HPGPVSPAREAFYRHLLNVTWEGRDFSFSPGGYL | |
| | VQPTMVVIALNRHRLWEMVGRWEHGVLYMKYPV | |
| | WPRYSASLQPVVDSRHLTVGSSTMVRSSKPTCPP | |
| | PELLGGPSVFIFPPKPKDTLMISRTPEVTCVVVDVS | |
| | QDDPEVQFTWYINNEQVRTARPPLREQQFNSTIRV | |
| | VSTLPIAHQDWLRGKEFKCKVHNKALPAPIEKTISK | |
| | ARGQPLEPKVYTMGPPREELSSRSVSLTCMINGFY | |
| | PSDISVEWEKNGKAEDNYKTTPAVLDSDGSYFLYS | |
| | KLSVPTSEWQRGDVFTCSVMHEALHNHYTQKSISR | |
| | SPGK | |
| SEQ ID NO | MGGALGPALLLTSLFGAWAGLGPGQGEQGMTVAV | Amino terminal domain |
| 97 | VFSSSGPPQAQFRARLTPQSFLDLPLEIQPLTVGVN | of GluN2C (including |
| | TTNPSSLLTQICGLLGAAHVHGIVFEDNVDTEAVAQI | signal sequence) as |
| | LDFISSQTHVPILSISGGSAVVLTPKEPGSAFLQLGV | used for example in |
| | SLEQQLQVLFKVLEEYDWSAFAVITSLHPGHALFLE | fusion protein #9 |
| | GVRAVADASHVSWRLLDVVTLELGPGGPRARTQR | |
| | LLRQLDAPVFVAYCSREEAEVLFAEAAQAGLVGPG | |
| | HVWLVPNLALGSTDAPPATFPVGLISVVTESWRLSL | |
| | RQKVRDGVAILALGAHSYWRQHGTLPAPAGDCRV | |
| | HPGPVSPAREAFYRHLLNVTWEGRDFSFSPGGYL | |
| | VQPTMVVIALNRHRLWEMVGRWEHGVLYMKYPV | |
| | WPRYSASLQPVVDSRHLTV | |

TABLE 2
Preferred nucleic acid sequences of the present invention.

| SEQ ID NO. | Amino Acid Sequence | Description |
|---|---|---|
| SEQ ID NO | ATGAGCACCATGCGCCTGCTGACGCTCGCCCTG | DNA sequence of |
| 49 | CTGTTCTCCTGCTCCGTCGCCCGTGCCGCGTGC | fusion protein #1 N1- |
| | GACCCCAAGATCGTCAACATTGGCGCGGTGCTG | ATD-Fc |
| | AGCACGCGGAAGCACGAGCAGATGTTCCGCGAG | |
| | GCCGTGAACCAGGCCAACAAGCGGCACGGCTCC | |
| | TGGAAGATTCAGCTCAATGCCACCTCCGTCACGC | |
| | ACAAGCCCAACGCCATCCAGATGGCTCTGTCGG | |
| | TGTGCGAGGACCTCATCTCCAGCCAGGTCTACG | |
| | CCATCCTAGTTAGCCATCCACCTACCCCCAACGA | |
| | CCACTTCACTCCCACCCCTGTCTCCTACACAGCC | |
| | GGCTTCTACCGCATACCCGTGCTGGGGCTGACC | |
| | ACCCGCATGTCCATCTACTCGGACAAGAGCATCC | |
| | ACCTGAGCTTCCTGCGCACCGTGCCGCCCTACT | |
| | CCCACCAGTCCAGCGTGTGGTTTGAGATGATGC | |
| | GTGTCTACAGCTGGAACCACATCATCCTGCTGGT | |
| | CAGCGACGACCACGAGGGCCGGGCGGCTCAGA | |
| | AACGCCTGGAGACGCTGCTGGAGGAGCGTGAGT | |
| | CCAAGGCAGAGAAGGTGCTGCAGTTTGACCCAG | |
| | GGACCAAGAACGTGACGGCCCTGCTGATGGAGG | |
| | CGAAAGAGCTGGAGGCCCGGGTCATCATCCTTT | |
| | CTGCCAGCGAGGACGATGCTGCCACTGTATACC | |
| | GCGCAGCCGCGATGCTGAACATGACGGGCTCCG | |
| | GGTACGTGTGGCTGGTCGGCGAGCGCGAGATCT | |
| | CGGGGAACGCCCTGCGCTACGCCCCGGACGGC | |
| | ATCCTCGGGCTGCAGCTCATCAACGGCAAGAAC | |
| | GAGTCGGCCCACATCAGCGACGCCGTAGGCGTG | |
| | GTGGCCCAGGCCGTGCACGAGCTCCTCGAGAAG | |
| | GAGAACATCACCGACCCGCCGCGGGGCTGCGT | |
| | GGGCAACACCAACATCTGGAAGACCGGGCCGCT | |
| | CTTCAAGAGAGTGCTGATGTCTTCCAAGTATGCG | |
| | GATGGGGTGACTGGTCGCGTGGAGTTCAATGAG | |
| | GATGGGGACCGGAAGTTCGCCAACTACAGCATC | |
| | ATGAACCTGCAGAACCGCAAGCTGGTGCAAGTG | |
| | GGCATCTACAATGGCACCCACGTCATCCCTAATG | |
| | ACAGGAAGATCATCTGGCCAGGCGGAGAGACAG | |
| | AGAAGCCTCGAGGGTACCAGATGTCCACCAGAC | |
| | TGAAGATTGGGTCGAGCACCATGGTTAGATCTAG | |
| | CAAGCCCACGTGCCCACCCCCTGAACTCCTGGG | |
| | GGGACCGTCTGTCTTCATCTTCCCCCCAAAACCC | |
| | AAGGACACCCTCATGATCTCACGCACCCCCGAG | |
| | GTCACATGCGTGGTGGTGGACGTGAGCCAGGAT | |
| | GACCCCGAGGTGCAGTTCACATGGTACATAAACA | |
| | ACGAGCAGGTGCGCACCGCCCGGCCGCCGCTA | |
| | CGGGAGCAGCAGTTCAACAGCACGATCCGCGTG | |
| | GTCAGCACCCTCCCCATCGCGCACCAGGACTGG | |
| | CTGAGGGGCAAGGAGTTCAAGTGCAAAGTCCAC | |
| | AACAAGGCACTCCCGGCCCCCATCGAGAAAACC | |
| | ATCTCCAAAGCCAGAGGGCAGCCCCTGGAGCCG | |
| | AAGGTCTACACCATGGGCCCTCCCCGGGAGGAG | |
| | CTGAGCAGCAGGTCGGTCAGCCTGACCTGCATG | |
| | ATCAACGGCTTCTACCCTTCCGACATCTCGGTGG | |
| | AGTGGGAGAAGAACGGGAAGGCAGAGGACAACT | |
| | ACAAGACCACGCCGGCCGTGCTGGACAGCGAC | |
| | GGCTCCTACTTCCTCTACAGCAAGCTCTCAGTGC | |
| | CCACGAGTGAGTGGCAGCGGGGCGACGTCTTCA | |
| | CCTGCTCCGTGATGCACGAGGCCTTGCACAACC | |
| | ACTACACGCAGAAGTCCATCTCCCGCTCTCCGG | |
| | GTAAATGA | |
| SEQ ID NO | ATGAGCACCATGCGCCTGCTGACGCTCGCCCTG | Amino terminal domain |
| 50 | CTGTTCTCCTGCTCCGTCGCCCGTGCCGCGTGC | (ATD) of GluN1 |
| | GACCCCAAGATCGTCAACATTGGCGCGGTGCTG | including the signal |
| | AGCACGCGGAAGCACGAGCAGATGTTCCGCGAG | sequence as in fusion |
| | GCCGTGAACCAGGCCAACAAGCGGCACGGCTCC | protein #1 |
| | TGGAAGATTCAGCTCAATGCCACCTCCGTCACGC | |
| | ACAAGCCCAACGCCATCCAGATGGCTCTGTCGG | |
| | TGTGCGAGGACCTCATCTCCAGCCAGGTCTACG | |
| | CCATCCTAGTTAGCCATCCACCTACCCCCAACGA | |
| | CCACTTCACTCCCACCCCTGTCTCCTACACAGCC | |
| | GGCTTCTACCGCATACCCGTGCTGGGGCTGACC | |
| | ACCCGCATGTCCATCTACTCGGACAAGAGCATCC | |
| | ACCTGAGCTTCCTGCGCACCGTGCCGCCCTACT | |
| | CCCACCAGTCCAGCGTGTGGTTTGAGATGATGC | |
| | GTGTCTACAGCTGGAACCACATCATCCTGCTGGT | |
| | CAGCGACGACCACGAGGGCCGGGCGGCTCAGA | |
| | AACGCCTGGAGACGCTGCTGGAGGAGCGTGAGT | |
| | CCAAGGCAGAGAAGGTGCTGCAGTTTGACCCAG | |
| | GGACCAAGAACGTGACGGCCCTGCTGATGGAGG | |
| | CGAAAGAGCTGGAGGCCCGGGTCATCATCCTTT | |
| | CTGCCAGCGAGGACGATGCTGCCACTGTATACC | |
| | GCGCAGCCGCGATGCTGAACATGACGGGCTCCG | |
| | GGTACGTGTGGCTGGTCGGCGAGCGCGAGATCT | |
| | CGGGGAACGCCCTGCGCTACGCCCCGGACGGC | |
| | ATCCTCGGGCTGCAGCTCATCAACGGCAAGAAC | |
| | GAGTCGGCCCACATCAGCGACGCCGTAGGCGTG | |
| | GTGGCCCAGGCCGTGCACGAGCTCCTCGAGAAG | |
| | GAGAACATCACCGACCCGCCGCGGGGCTGCGT | |
| | GGGCAACACCAACATCTGGAAGACCGGGCCGCT | |
| | CTTCAAGAGAGTGCTGATGTCTTCCAAGTATGCG | |
| | GATGGGGTGACTGGTCGCGTGGAGTTCAATGAG | |
| | GATGGGGACCGGAAGTTCGCCAACTACAGCATC | |
| | ATGAACCTGCAGAACCGCAAGCTGGTGCAAGTG | |
| | GGCATCTACAATGGCACCCACGTCATCCCTAATG | |
| | ACAGGAAGATCATCTGGCCAGGCGGAGAGACAG | |
| | AGAAGCCTCGAGGGTACCAGATGTCCACCAGAC | |
| | TGAAGATT | |
| SEQ ID NO | GGGTCGAGCACCATGGTTAGATCT | Linker between |
| 51 | | NMDAR domain and |
| | | rabbit Fc to be used in |
| | | fusion proteins of |
| | | NMDAR protein |
| | | constructs of the |
| | | invention |
| SEQ ID NO | AGCAAGCCCACGTGCCCACCCCCTGAACTCCTG | Rabbit Fc from Vector |
| 52 | GGGGGACCGTCTGTCTTCATCTTCCCCCCAAAAC | pFuse-rIgG-Fc1 |
| | CCAAGGACACCCTCATGATCTCACGCACCCCCG | (InvivoGen) as used in |
| | AGGTCACATGCGTGGTGGTGGACGTGAGCCAGG | fusion proteins #1-#8 |
| | ATGACCCCGAGGTGCAGTTCACATGGTACATAAA | |
| | CAACGAGCAGGTGCGCACCGCCCGGCCGCCGC | |
| | TACGGGAGCAGCAGTTCAACAGCACGATCCGCG | |
| | TGGTCAGCACCCTCCCCATCGCGCACCAGGACT | |
| | GGCTGAGGGGCAAGGAGTTCAAGTGCAAAGTCC | |
| | ACAACAAGGCACTCCCGGCCCCCATCGAGAAAA | |
| | CCATCTCCAAAGCCAGAGGGCAGCCCCTGGAGC | |
| | CGAAGGTCTACACCATGGGCCCTCCCCGGGAGG | |
| | AGCTGAGCAGCAGGTCGGTCAGCCTGACCTGCA | |
| | TGATCAACGGCTTCTACCCTTCCGACATCTCGGT | |
| | GGAGTGGGAGAAGAACGGGAAGGCAGAGGACA | |
| | ACTACAAGACCACGCCGGCCGTGCTGGACAGCG | |
| | ACGGCTCCTACTTCCTCTACAGCAAGCTCTCAGT | |
| | GCCCACGAGTGAGTGGCAGCGGGGCGACGTCT | |
| | TCACCTGCTCCGTGATGCACGAGGCCTTGCACA | |
| | ACCACTACACGCAGAAGTCCATCTCCCGCTCTCC | |
| | GGGTAAATGA | |
| SEQ ID NO | ACGCTCGCCCTGCTGTTCTCCTGCTCCGTCGCC | Amino terminal domain |
| 53 | CGTGCCGCGTGCGACCCCAAGATCGTCAACATT | of GluN1 in |
| | GGCGCGGTGCTGAGCACGCGGAAGCACGAGCA | NM_007327 with |
| | GATGTTCCGCGAGGCCGTGAACCAGGCCAACAA | domain borders as in |
| | GCGGCACGGCTCCTGGAAGATTCAGCTCAATGC | cd06379: |
| | CACCTCCGTCACGCACAAGCCCAACGCCATCCA | PBP1_iGluR_NMDA |
| | GATGGCTCTGTCGGTGTGCGAGGACCTCATCTC | NR1 (NCBI) |
| | CAGCCAGGTCTACGCCATCCTAGTTAGCCATCCA | |
| | CCTACCCCCAACGACCACTTCACTCCCACCCCTG | |
| | TCTCCTACACAGCCGGCTTCTACCGCATACCCGT | |
| | GCTGGGGCTGACCACCCGCATGTCCATCTACTC | |
| | GGACAAGAGCATCCACCTGAGCTTCCTGCGCAC | |
| | CGTGCCGCCCTACTCCCACCAGTCCAGCGTGTG | |
| | GTTTGAGATGATGCGTGTCTACAGCTGGAACCAC | |
| | ATCATCCTGCTGGTCAGCGACGACCACGAGGGC | |
| | CGGGCGGCTCAGAAACGCCTGGAGACGCTGCT | |
| | GGAGGAGCGTGAGTCCAAGGCAGAGAAGGTGCT | |
| | GCAGTTTGACCCAGGGACCAAGAACGTGACGGC | |
| | CCTGCTGATGGAGGCGAAAGAGCTGGAGGCCC | |
| | GGGTCATCATCCTTTCTGCCAGCGAGGACGATG | |
| | CTGCCACTGTATACCGCGCAGCCGCGATGCTGA | |
| | ACATGACGGGCTCCGGGTACGTGTGGCTGGTCG | |
| | GCGAGCGCGAGATCTCGGGGAACGCCCTGCGC | |
| | TACGCCCCAGACGGCATCCTCGGGCTGCAGCTC | |
| | ATCAACGGCAAGAACGAGTCGGCCCACATCAGC | |
| | GACGCCGTGGGCGTGGTGGCCCAGGCCGTGCA | |
| | CGAGCTCCTCGAGAAGGAGAACATCACCGACCC | |
| | GCCGCGGGGCTGCGTGGGCAACACCAACATCTG | |
| | GAAGACCGGGCCGCTCTTCAAGAGAGTGCTGAT | |
| | GTCTTCCAAGTATGCGGATGGGGTGACTGGTCG | |
| | CGTGGAGTTCAATGAGGATGGGGACCGGAAGTT | |
| | CGCCAACTACAGCATCATGAACCTGCAGAACCG | |
| | CAAGCTGGTGCAAGTGGGCATCTACAATGGCAC | |
| | CCACGTCATCCCTAATGACAGGAAGATCATCTGG | |
| | CCAGGC | |
| SEQ ID NO | ATGAGCACCATGCGCCTGCTGACGCTCGCCCTG | DNA seq of fusion |
| 54 | CTGTTCTCCTGCTCCGTCGCCCGTGCCGCGTGC | protein #2 N1 ecd-Fc |
| | GACCCCAAGATCGTCAACATTGGCGCGGTGCTG | |
| | AGCACGCGGAAGCACGAGCAGATGTTCCGCGAG | |
| | GCCGTGAACCAGGCCAACAAGCGGCACGGCTCC | |
| | TGGAAGATTCAGCTCAATGCCACCTCCGTCACGC | |
| | ACAAGCCCAACGCCATCCAGATGGCTCTGTCGG | |
| | TGTGCGAGGACCTCATCTCCAGCCAGGTCTACG | |
| | CCATCCTAGTTAGCCATCCACCTACCCCCAACGA | |
| | CCACTTCACTCCCACCCCTGTCTCCTACACAGCC | |
| | GGCTTCTACCGCATACCCGTGCTGGGGCTGACC | |
| | ACCCGCATGTCCATCTACTCGGACAAGAGCATCC | |
| | ACCTGAGCTTCCTGCGCACCGTGCCGCCCTACT | |
| | CCCACCAGTCCAGCGTGTGGTTTGAGATGATGC | |
| | GTGTCTACAGCTGGAACCACATCATCCTGCTGGT | |
| | CAGCGACGACCACGAGGGCCGGGCGGCTCAGA | |
| | AACGCCTGGAGACGCTGCTGGAGGAGCGTGAGT | |
| | CCAAGGCAGAGAAGGTGCTGCAGTTTGACCCAG | |
| | GGACCAAGAACGTGACGGCCCTGCTGATGGAGG | |
| | CGAAAGAGCTGGAGGCCCGGGTCATCATCCTTT | |
| | CTGCCAGCGAGGACGATGCTGCCACTGTATACC | |
| | GCGCAGCCGCGATGCTGAACATGACGGGCTCCG | |
| | GGTACGTGTGGCTGGTCGGCGAGCGCGAGATCT | |
| | CGGGGAACGCCCTGCGCTACGCCCCGGACGGC | |
| | ATCCTCGGGCTGCAGCTCATCAACGGCAAGAAC | |
| | GAGTCGGCCCACATCAGCGACGCCGTAGGCGTG | |
| | GTGGCCCAGGCCGTGCACGAGCTCCTCGAGAAG | |
| | GAGAACATCACCGACCCGCCGCGGGGCTGCGT | |
| | GGGCAACACCAACATCTGGAAGACCGGGCCGCT | |
| | CTTCAAGAGAGTGCTGATGTCTTCCAAGTATGCG | |
| | GATGGGGTGACTGGTCGCGTGGAGTTCAATGAG | |
| | GATGGGGACCGGAAGTTCGCCAACTACAGCATC | |
| | ATGAACCTGCAGAACCGCAAGCTGGTGCAAGTG | |
| | GGCATCTACAATGGCACCCACGTCATCCCTAATG | |
| | ACAGGAAGATCATCTGGCCAGGCGGAGAGACAG | |
| | AGAAGCCTCGAGGGTACCAGATGTCCACCAGAC | |
| | TGAAGATTGTGACGATCCACCAGGAGCCCTTCGT | |
| | GTACGTCAAGCCCACGCTGAGTGATGGGACATG | |
| | CAAGGAGGAGTTCACAGTCAACGGCGACCCAGT | |
| | CAAGAAGGTGATCTGCACCGGGCCCAACGACAC | |
| | GTCGCCGGGCAGCCCCCGCCACACGGTGCCTC | |
| | AGTGTTGCTACGGCTTTTGCATCGACCTGCTCAT | |
| | CAAGCTGGCACGGACCATGAACTTCACCTACGA | |
| | GGTGCACCTGGTGGCAGATGGCAAGTTCGGCAC | |
| | ACAGGAGCGGGTGAACAACAGCAACAAGAAGGA | |
| | GTGGAATGGGATGATGGGCGAGCTGCTCAGCGG | |
| | GCAGGCAGACATGATCGTGGCGCCGCTAACCAT | |
| | AAACAACGAGCGCGCGCAGTACATCGAGTTTTC | |
| | CAAGCCCTTCAAGTACCAGGGCCTGACTATTCTG | |
| | GTCAAGAAGGGCACCCGCATCACGGGCATCAAC | |
| | GACCCCCGGCTGAGGAACCCCTCGGACAAGTTT | |
| | ATCTACGCCACGGTGAAGCAGAGCTCCGTGGAT | |
| | ATCTACTTCCGGCGCCAGGTGGAGCTGAGCACC | |
| | ATGTACCGGCATATGGAGAAGCACAACTACGAG | |
| | AGTGCGGCGGAGGCCATCCAGGCCGTGAGAGA | |
| | CAACAAGCTGCATGCCTTCATCTGGGACTCGGC | |
| | GGTGCTGGAGTTCGAGGCCTCGCAGAAGTGCGA | |
| | CCTGGTGACGACTGGAGAGCTGTTTTTCCGCTC | |
| | GGGCTTCGGCATAGGCATGCGCAAAGACAGCCC | |
| | CTGGAAGCAGAACGTCTCCCTGTCCATCCTCAAG | |
| | TCCCACGAGAATGGCTTCATGGAAGACCTGGAC | |
| | AAGACGTGGGTTCGGTATCAGGAATGTGACTCG | |
| | GGGTCGAGCACCATGGTTAGATCTAGCAAGCCC | |
| | ACGTGCCCACCCCCTGAACTCCTGGGGGGACCG | |
| | TCTGTCTTCATCTTCCCCCCAAAACCCAAGGACA | |
| | CCCTCATGATCTCACGCACCCCCGAGGTCACAT | |
| | GCGTGGTGGTGGACGTGAGCCAGGATGACCCC | |
| | GAGGTGCAGTTCACATGGTACATAAACAACGAGC | |
| | AGGTGCGCACCGCCCGGCCGCCGCTACGGGAG | |
| | CAGCAGTTCAACAGCACGATCCGCGTGGTCAGC | |
| | ACCCTCCCCATCGCGCACCAGGACTGGCTGAGG | |
| | GGCAAGGAGTTCAAGTGCAAAGTCCACAACAAG | |
| | GCACTCCCGGCCCCCATCGAGAAAACCATCTCC | |
| | AAAGCCAGAGGGCAGCCCCTGGAGCCGAAGGT | |
| | CTACACCATGGGCCCTCCCCGGGAGGAGCTGAG | |
| | CAGCAGGTCGGTCAGCCTGACCTGCATGATCAA | |
| | CGGCTTCTACCCTTCCGACATCTCGGTGGAGTG | |
| | GGAGAAGAACGGGAAGGCAGAGGACAACTACAA | |
| | GACCACGCCGGCCGTGCTGGACAGCGACGGCT | |
| | CCTACTTCCTCTACAGCAAGCTCTCAGTGCCCAC | |
| | GAGTGAGTGGCAGCGGGGCGACGTCTTCACCTG | |
| | CTCCGTGATGCACGAGGCCTTGCACAACCACTA | |
| | CACGCAGAAGTCCATCTCCCGCTCTCCGGGTAA | |
| | ATGA | |
| SEQ ID NO | ATGAGCACCATGCGCCTGCTGACGCTCGCCCTG | Amino terminal domain |
| 55 | CTGTTCTCCTGCTCCGTCGCCCGTGCCGCGTGC | of GluN1 (including |
| | GACCCCAAGATCGTCAACATTGGCGCGGTGCTG | signal sequence) as |
| | AGCACGCGGAAGCACGAGCAGATGTTCCGCGAG | included in fusion |
| | GCCGTGAACCAGGCCAACAAGCGGCACGGCTCC | proteins #2, #4 |
| | TGGAAGATTCAGCTCAATGCCACCTCCGTCACGC | |
| | ACAAGCCCAACGCCATCCAGATGGCTCTGTCGG | |
| | TGTGCGAGGACCTCATCTCCAGCCAGGTCTACG | |
| | CCATCCTAGTTAGCCATCCACCTACCCCCAACGA | |
| | CCACTTCACTCCCACCCCTGTCTCCTACACAGCC | |
| | GGCTTCTACCGCATACCCGTGCTGGGGCTGACC | |
| | ACCCGCATGTCCATCTACTCGGACAAGAGCATCC | |
| | ACCTGAGCTTCCTGCGCACCGTGCCGCCCTACT | |
| | CCCACCAGTCCAGCGTGTGGTTTGAGATGATGC | |
| | GTGTCTACAGCTGGAACCACATCATCCTGCTGGT | |
| | CAGCGACGACCACGAGGGCCGGGCGGCTCAGA | |
| | AACGCCTGGAGACGCTGCTGGAGGAGCGTGAGT | |
| | CCAAGGCAGAGAAGGTGCTGCAGTTTGACCCAG | |
| | GGACCAAGAACGTGACGGCCCTGCTGATGGAGG | |
| | CGAAAGAGCTGGAGGCCCGGGTCATCATCCTTT | |
| | CTGCCAGCGAGGACGATGCTGCCACTGTATACC | |
| | GCGCAGCCGCGATGCTGAACATGACGGGCTCCG | |
| | GGTACGTGTGGCTGGTCGGCGAGCGCGAGATCT | |
| | CGGGGAACGCCCTGCGCTACGCCCCGGACGGC | |
| | ATCCTCGGGCTGCAGCTCATCAACGGCAAGAAC | |
| | GAGTCGGCCCACATCAGCGACGCCGTAGGCGTG | |
| | GTGGCCCAGGCCGTGCACGAGCTCCTCGAGAAG | |
| | GAGAACATCACCGACCCGCCGCGGGGCTGCGT | |
| | GGGCAACACCAACATCTGGAAGACCGGGCCGCT | |
| | CTTCAAGAGAGTGCTGATGTCTTCCAAGTATGCG | |
| | GATGGGGTGACTGGTCGCGTGGAGTTCAATGAG | |
| | GATGGGGACCGGAAGTTCGCCAACTACAGCATC | |
| | ATGAACCTGCAGAACCGCAAGCTGGTGCAAGTG | |
| | GGCATCTACAATGGCACCCACGTCATCCCTAATG | |
| | ACAGGAAGATCATCTGGCCAGGCGGAGAGACAG | |
| | AGAAGCCTCGAGGGTACCAGATGTCCACCAGAC | |
| | TGAAGATT | |
| SEQ ID NO | TCCACCAGACTGAAGATTGTGACGATCCACCAG | Ligand-binding domain |
| 56 | GAGCCCTTCGTGTACGTCAAGCCCACGCTGAGT | of GluN1 S1, as for |
| | GATGGGACATGCAAGGAGGAGTTCACAGTCAAC | example included in |
| | GGCGACCCAGTCAAGAAGGTGATCTGCACCGGG | fusion proteins #2, #4; |
| | CCCAACGACACGTCGCCGGGCAGCCCCCGCCA | corresponds to Ligand- |
| | CACGGTGCCTCAGTGTTGCTACGGCTTTTGCATC | binding domain of |
| | GACCTGCTCATCAAGCTGGCACGGACCATGAAC | GluN1 S1 in |
| | TTCACCTACGAGGTGCACCTGGTGGCAGATGGC | NM_007327 with |
| | AAGTTCGGCACACAGGAGCGGGTGAACAACAGC | domain borders as in |
| | AACAAGAAGGAGTGGAATGGGATGATGGGCGAG | cd13719: |
| | CTGCTCAGCGGGCAGGCAGACATGATCGTGGCG | PBP2_iGluR_NMDA |
| | CCGCTAACCATAAACAACGAGCGCGCGCAGTAC | Nr1 (NCBI) |
| | ATCGAGTTTTCCAAGCCCTTCAAGTACCAGGGCC | |
| | TGACTATTCTGGTCAAGAAG | |
| | GGCACC | Linker between S1 and |
| | | S2, as for example |
| | | included in fusion |
| | | proteins #2, #3, #4 |
| SEQ ID NO | CGCATCACGGGCATCAACGACCCCCGGCTGAGG | Ligand-binding domain |
| 57 | AACCCCTCGGACAAGTTTATCTACGCCACGGTGA | of GluN1 S2, as for |
| | AGCAGAGCTCCGTGGATATCTACTTCCGGCGCC | example included in |
| | AGGTGGAGCTGAGCACCATGTACCGGCATATGG | fusion proteins #2, #4 |
| | AGAAGCACAACTACGAGAGTGCGGCGGAGGCCA | |
| | TCCAGGCCGTGAGAGACAACAAGCTGCATGCCT | |
| | TCATCTGGGACTCGGCGGTGCTGGAGTTCGAGG | |
| | CCTCGCAGAAGTGCGACCTGGTGACGACTGGAG | |
| | AGCTGTTTTTCCGCTCGGGCTTCGGCATAGGCAT | |
| | GCGCAAAGACAGCCCCTGGAAGCAGAACGTCTC | |
| | CCTGTCCATCCTCAAGTCCCACGAGAATGGCTTC | |
| | ATGGAAGACCTGGACAAGACGTGGGTTCGGTAT | |
| | CAGGAATGTGACTCG | |
| SEQ ID NO | CGCATCACGGGCATCAACGACCCTCGGCTGAGG | Ligand-binding domain |
| 58 | AACCCCTCGGACAAGTTTATCTACGCCACGGTGA | of GluN1 S2 in |
| | AGCAGAGCTCCGTGGATATCTACTTCCGGCGCC | NM_007327 with |
| | AGGTGGAGCTGAGCACCATGTACCGGCATATGG | domain borders as in |
| | AGAAGCACAACTACGAGAGTGCGGCGGAGGCCA | cd13719: |
| | TCCAGGCCGTGAGAGACAACAAGCTGCATGCCT | PBP2_iGluR_NMDA_ |
| | TCATCTGGGACTCGGCGGTGCTGGAGTTCGAGG | Nr1 (NCBI) |
| | CCTCGCAGAAGTGCGACCTGGTGACGACTGGAG | |
| | AGCTGTTTTTCCGCTCGGGCTTCGGCATAGGCAT | |
| | GCGCAAAGACAGCCCCTGGAAGCAGAACGTCTC | |
| | CCTGTCCATCCTCAAGTCCCACGAGAATGGCTTC | |
| | ATGGAAGACCTGGACAAGACGTGGGTTCGGTAT | |
| | CAGGAATGTGAC | |
| SEQ ID NO | ATGAAGCCCAGAGCGGAGTGCTGTTCTCCCAAG | DNA seq of fusion |
| 59 | TTCTGGTTGGTGTTGGCCGTCCTGGCCGTGTCA | protein #3 N2Becd-Fc |
| | GGCAGCAGAGCTCGTTCTCAGAAGAGCCCCCCC | |
| | AGCATTGGCATTGCTGTCATCCTCGTGGGCACTT | |
| | CCGACGAGGTGGCCATCAAGGATGCCCACGAGA | |
| | AAGATGATTTCCACCATCTCTCCGTGGTACCCCG | |
| | GGTGGAACTGGTAGCCATGAATGAGACCGACCC | |
| | AAAGAGCATCATCACCCGCATCTGTGATCTCATG | |
| | TCTGACCGGAAGATCCAGGGGGTGGTGTTTGCT | |
| | GATGACACAGACCAGGAAGCCATCGCCCAGATC | |
| | CTCGATTTCATTTCAGCACAGACTCTCACCCCGA | |
| | TCCTGGGCATCCACGGGGGCTCCTCTATGATAAT | |
| | GGCAGATAAGGATGAATCCTCCATGTTCTTCCAG | |
| | TTTGGCCCATCAATTGAACAGCAAGCTTCCGTAA | |
| | TGCTCAACATCATGGAAGAATATGACTGGTACAT | |
| | CTTTTCTATCGTCACCACCTATTTCCCTGGCTACC | |
| | AGGACTTTGTAAACAAGATCCGCAGCACCATTGA | |
| | GAATAGCTTTGTGGGCTGGGAGCTAGAGGAGGT | |
| | CCTCCTACTGGACATGTCCCTGGACGATGGAGA | |
| | TTCTAAGATCCAGAATCAGCTCAAGAAACTTCAA | |
| | AGCCCCATCATTCTTCTTTACTGTACCAAGGAAG | |
| | AAGCCACCTACATCTTTGAAGTGGCCAACTCAGT | |
| | AGGGCTGACTGGCTATGGCTACACGTGGATCGT | |
| | GCCCAGTCTGGTGGCAGGGGATACAGACACAGT | |
| | GCCTGCGGAGTTCCCCACTGGGCTCATCTCTGT | |
| | ATCATATGATGAATGGGACTATGGCCTCCCCGCC | |
| | AGAGTGAGAGATGGAATTGCCATAATCACCACTG | |
| | CTGCTTCTGACATGCTGTCTGAGCACAGCTTCAT | |
| | CCCTGAGCCCAAAAGCAGTTGTTACAACACCCAC | |
| | GAGAAGAGAATCTACCAGTCCAATATGCTAAATA | |
| | GGTATCTGATCAATGTCACTTTTGAGGGGAGGAA | |
| | TTTGTCCTTCAGTGAAGATGGCTACCAGATGCAC | |
| | CCGAAACTGGTGATAATTCTTCTGAACAAGGAGA | |
| | GGAAGTGGGAAAGGGTGGGGAAGTGGAAAGAC | |
| | AAGTCCCTGCAGATGAAGTACTATGTGTGGCCCC | |
| | GAATGTGTCCAGAGACTGAAGAGCAGGAGGATG | |
| | ACCATCTGAGCATTGTGACCCTGGAGGAGGCAC | |
| | CATTTGTCATTGTGGAAAGTGTGGACCCTCTGAG | |
| | TGGAACCTGCATGAGGAACACAGTCCCCTGCCA | |
| | AAAACGCATAGTCACTGAGAATAAAACAGACGAG | |
| | GAGCCGGGTTACATCAAAAAATGCTGCAAGGGG | |
| | TTCTGTATTGACATCCTTAAGAAAATTTCTAAATC | |
| | TGTGAAGTTCACCTATGACCTTTACCTGGTTACC | |
| | AATGGCAAGCATGGGAAGAAAATCAATGGAACCT | |
| | GGAATGGTATGATTGGAGAGGTGGTCATGAAGA | |
| | GGGCCTACATGGCAGTGGGCTCACTCACCATCA | |
| | ATGAGGAACGATCGGAGGTGGTCGACTTCTCTG | |
| | TGCCCTTCATAGAGACAGGCATCAGTGTCATGGT | |
| | GTCACGCGGCACCCAGGTTTCTGGCCTGAGCGA | |
| | CAAAAAGTTCCAGAGACCTAATGACTTCTCACCC | |
| | CCTTTCCGCTTTGGGACCGTGCCCAACGGCAGC | |
| | ACAGAGAGAAATATTCGCAATAACTATGCAGAAA | |
| | TGCATGCCTACATGGGAAAGTTCAACCAGAGGG | |
| | GTGTAGATGATGCATTGCTCTCCCTGAAAACAGG | |
| | GAAACTGGATGCCTTCATCTATGATGCAGCAGTG | |
| | CTGAACTATATGGCAGGCAGAGATGAAGGCTGC | |
| | AAGCTGGTGACCATTGGCAGTGGGAAGGTCTTT | |
| | GCTTCCACTGGCTATGGCATTGCCATCCAAAAAG | |
| | ATTCTGGGTGGAAGCGCCAGGTGGACCTTGCTA | |
| | TCCTGCAGCTCTTTGGAGATGGGGAGATGGAAG | |
| | AACTGGAAGCTCTCTGGCTCACTGGCATTTGTCA | |
| | CAATGGCTCGAGCACCATGGTTAGATCTAGCAAG | |
| | CCCACGTGCCCACCCCCTGAACTCCTGGGGGGA | |
| | CCGTCTGTCTTCATCTTCCCCCCAAAACCCAAGG | |
| | ACACCCTCATGATCTCACGCACCCCCGAGGTCA | |
| | CATGCGTGGTGGTGGACGTGAGCCAGGATGACC | |
| | CCGAGGTGCAGTTCACATGGTACATAAACAACGA | |
| | GCAGGTGCGCACCGCCCGGCCGCCGCTACGGG | |
| | AGCAGCAGTTCAACAGCACGATCCGCGTGGTCA | |
| | GCACCCTCCCCATCGCGCACCAGGACTGGCTGA | |
| | GGGGCAAGGAGTTCAAGTGCAAAGTCCACAACA | |
| | AGGCACTCCCGGCCCCCATCGAGAAAACCATCT | |
| | CCAAAGCCAGAGGGCAGCCCCTGGAGCCGAAG | |
| | GTCTACACCATGGGCCCTCCCCGGGAGGAGCTG | |
| | AGCAGCAGGTCGGTCAGCCTGACCTGCATGATC | |
| | AACGGCTTCTACCCTTCCGACATCTCGGTGGAGT | |
| | GGGAGAAGAACGGGAAGGCAGAGGACAACTACA | |
| | AGACCACGCCGGCCGTGCTGGACAGCGACGGC | |
| | TCCTACTTCCTCTACAGCAAGCTCTCAGTGCCCA | |
| | CGAGTGAGTGGCAGCGGGGCGACGTCTTCACCT | |
| | GCTCCGTGATGCACGAGGCCTTGCACAACCACT | |
| | ACACGCAGAAGTCCATCTCCCGCTCTCCGGGTA | |
| | AATGA | |
| SEQ ID NO | ATGAAGCCCAGAGCGGAGTGCTGTTCTCCCAAG | Amino terminal domain |
| 60 | TTCTGGTTGGTGTTGGCCGTCCTGGCCGTGTCA | of GluN2B (including |
| | GGCAGCAGAGCTCGTTCTCAGAAGAGCCCCCCC | signal sequence), as |
| | AGCATTGGCATTGCTGTCATCCTCGTGGGCACTT | for example included |
| | CCGACGAGGTGGCCATCAAGGATGCCCACGAGA | in fusion protein #3 |
| | AAGATGATTTCCACCATCTCTCCGTGGTACCCCG | N2Becd-Fc |
| | GGTGGAACTGGTAGCCATGAATGAGACCGACCC | |
| | AAAGAGCATCATCACCCGCATCTGTGATCTCATG | |
| | TCTGACCGGAAGATCCAGGGGGTGGTGTTTGCT | |
| | GATGACACAGACCAGGAAGCCATCGCCCAGATC | |
| | CTCGATTTCATTTCAGCACAGACTCTCACCCCGA | |
| | TCCTGGGCATCCACGGGGGCTCCTCTATGATAAT | |
| | GGCAGATAAGGATGAATCCTCCATGTTCTTCCAG | |
| | TTTGGCCCATCAATTGAACAGCAAGCTTCCGTAA | |
| | TGCTCAACATCATGGAAGAATATGACTGGTACAT | |
| | CTTTTCTATCGTCACCACCTATTTCCCTGGCTACC | |
| | AGGACTTTGTAAACAAGATCCGCAGCACCATTGA | |
| | GAATAGCTTTGTGGGCTGGGAGCTAGAGGAGGT | |
| | CCTCCTACTGGACATGTCCCTGGACGATGGAGA | |
| | TTCTAAGATCCAGAATCAGCTCAAGAAACTTCAA | |
| | AGCCCCATCATTCTTCTTTACTGTACCAAGGAAG | |
| | AAGCCACCTACATCTTTGAAGTGGCCAACTCAGT | |
| | AGGGCTGACTGGCTATGGCTACACGTGGATCGT | |
| | GCCCAGTCTGGTGGCAGGGGATACAGACACAGT | |
| | GCCTGCGGAGTTCCCCACTGGGCTCATCTCTGT | |
| | ATCATATGATGAATGGGACTATGGCCTCCCCGCC | |
| | AGAGTGAGAGATGGAATTGCCATAATCACCACTG | |
| | CTGCTTCTGACATGCTGTCTGAGCACAGCTTCAT | |
| | CCCTGAGCCCAAAAGCAGTTGTTACAACACCCAC | |
| | GAGAAGAGAATCTACCAGTCCAATATGCTAAATA | |
| | GGTATCTGATCAATGTCACTTTTGAGGGGAGGAA | |
| | TTTGTCCTTCAGTGAAGATGGCTACCAGATGCAC | |
| | CCGAAACTGGTGATAATTCTTCTGAACAAGGAGA | |
| | GGAAGTGGGAAAGGGTGGGGAAGTGGAAAGAC | |
| | AAGTCCCTGCAGATGAAGTACTATGTGTGGCCCC | |
| | GAATGTGTCCAGAGACTGAAGAGCAG | |
| SEQ ID NO | GAGGATGACCATCTGAGCATTGTGACCCTGGAG | Ligand-binding domain |
| 61 | GAGGCACCATTTGTCATTGTGGAAAGTGTGGACC | of GluN2B S1, as for |
| | CTCTGAGTGGAACCTGCATGAGGAACACAGTCC | example included in |
| | CCTGCCAAAAACGCATAGTCACTGAGAATAAAAC | fusion proteins #3, #4 |
| | AGACGAGGAGCCGGGTTACATCAAAAAATGCTG | |
| | CAAGGGGTTCTGTATTGACATCCTTAAGAAAATTT | |
| | CTAAATCTGTGAAGTTCACCTATGACCTTTACCTG | |
| | GTTACCAATGGCAAGCATGGGAAGAAAATCAATG | |
| | GAACCTGGAATGGTATGATTGGAGAGGTGGTCA | |
| | TGAAGAGGGCCTACATGGCAGTGGGCTCACTCA | |
| | CCATCAATGAGGAACGATCGGAGGTGGTCGACT | |
| | TCTCTGTGCCCTTCATAGAGACAGGCATCAGTGT | |
| | CATGGTGTCACGC | |
| SEQ ID NO | AGGTTTCTGGCCTGAGCGACAAAAAGTTCCAGA | Ligand-binding domain |
| 62 | GACCTAATGACTTCTCACCCCCTTTCCGCTTTGG | of GluN2B S2, as for |
| | GACCGTGCCCAACGGCAGCACAGAGAGAAATAT | example included in |
| | TCGCAATAACTATGCAGAAATGCATGCCTACATG | fusion proteins #3, #4 |
| | GGAAAGTTCAACCAGAGGGGTGTAGATGATGCA | |
| | TTGCTCTCCCTGAAAACAGGGAAACTGGATGCCT | |
| | TCATCTATGATGCAGCAGTGCTGAACTATATGGC | |
| | AGGCAGAGATGAAGGCTGCAAGCTGGTGACCAT | |
| | TGGCAGTGGGAAGGTCTTTGCTTCCACTGGCTAT | |
| | GGCATTGCCATCCAAAAAGATTCTGGGTGGAAG | |
| | CGCCAGGTGGACCTTGCTATCCTGCAGCTCTTTG | |
| | GAGATGGGGAGATGGAAGAACTGGAAGCTCTCT | |
| | GGCTCACTGGCATTTGTCACAAT | |
| SEQ ID NO | CCCAGCATTGGCATTGCTGTCATCCTCGTGGGC | Amino terminal domain |
| 63 | ACTTCCGACGAGGTGGCCATCAAGGATGCCCAC | of GluN2B in |
| | GAGAAAGATGATTTCCACCATCTCTCCGTGGTAC | NM_000834 with |
| | CCCGGGTGGAACTGGTAGCCATGAATGAGACCG | domain borders as in |
| | ACCCAAAGAGCATCATCACCCGCATCTGTGATCT | cd06378: |
| | CATGTCTGACCGGAAGATCCAGGGGGTGGTGTT | PBP1_iGluR_NMDA |
| | TGCTGATGACACAGACCAGGAAGCCATCGCCCA | NR2 (NCBI) |
| | GATCCTCGATTTCATTTCAGCACAGACTCTCACC | |
| | CCCATCCTGGGCATCCACGGGGGCTCCTCTATG | |
| | ATAATGGCAGATAAGGATGAATCCTCCATGTTCT | |
| | TCCAGTTTGGCCCATCAATTGAACAGCAAGCTTC | |
| | CGTAATGCTCAACATCATGGAAGAATATGACTGG | |
| | TACATCTTTTCTATCGTCACCACCTATTTCCCTGG | |
| | CTACCAGGACTTTGTAAACAAGATCCGCAGCACC | |
| | ATTGAGAATAGCTTTGTGGGCTGGGAGCTAGAG | |
| | GAGGTCCTCCTACTGGACATGTCCCTGGACGAT | |
| | GGAGATTCTAAGATCCAGAATCAGCTCAAGAAAC | |
| | TTCAAAGCCCCATCATTCTTCTTTACTGTACCAAG | |
| | GAAGAAGCCACCTACATCTTTGAAGTGGCCAACT | |
| | CAGTAGGGCTGACTGGCTATGGCTACACGTGGA | |
| | TCGTGCCCAGTCTGGTGGCAGGGGATACAGACA | |
| | CAGTGCCTGCGGAGTTCCCCACTGGGCTCATCT | |
| | CTGTATCATATGATGAATGGGACTATGGCCTCCC | |
| | CGCCAGAGTGAGAGATGGAATTGCCATAATCAC | |
| | CACTGCTGCTTCTGACATGCTGTCTGAGCACAGC | |
| | TTCATCCCTGAGCCCAAAAGCAGTTGTTACAACA | |
| | CCCACGAGAAGAGAATCTACCAGTCCAATATGCT | |
| | AAATAGGTATCTGATCAATGTCACTTTTGAGGGG | |
| | AGGAATTTGTCCTTCAGTGAAGATGGCTACCAGA | |
| | TGCACCCGAAACTGGTGATAATTCTTCTGAACAA | |
| | GGAGAGGAAGTGGGAAAGGGTGGGGAAGTGGA | |
| | AAGACAAGTCCCTGCAGATGAAGTACTATGTGTG | |
| | GCCCCGA | |
| SEQ ID NO | GATGACCATCTGAGCATTGTGACCCTGGAGGAG | Ligand-binding domain |
| 64 | GCACCATTTGTCATTGTGGAAAGTGTGGACCCTC | of GluN2B S1 in |
| | TGAGTGGAACCTGCATGAGGAACACAGTCCCCT | NM_000834 with |
| | GCCAAAAACGCATAGTCACTGAGAATAAAACAGA | domain borders as in |
| | CGAGGAGCCGGGTTACATCAAAAAATGCTGCAA | cd13718: |
| | GGGGTTCTGTATTGACATCCTTAAGAAAATTTCTA | PBP2_iGluR_NMDA |
| | AATCTGTGAAGTTCACCTATGACCTTTACCTGGTT | Nr2 (NCBI) |
| | ACCAATGGCAAGCATGGGAAGAAAATCAATGGAA | |
| | CCTGGAATGGTATGATTGGAGAGGTGGTCATGA | |
| | AGAGGGCCTACATGGCAGTGGGCTCACTCACCA | |
| | TCAATGAGGAACGATCGGAGGTGGTCGACTTCT | |
| | CTGTGCCCTTCATAGAGACAGGCATCAGTGTCAT | |
| | GGTGTCACGC | |
| SEQ ID NO | CAGGTTTCTGGCCTGAGCGACAAAAAGTTCCAGA | Ligand-binding domain |
| 65 | GACCTAATGACTTCTCACCCCCTTTCCGCTTTGG | of GluN2B S2 in |
| | GACCGTGCCCAACGGCAGCACAGAGAGAAATAT | NM_000834 with |
| | TCGCAATAACTATGCAGAAATGCATGCCTACATG | domain borders as in |
| | GGAAAGTTCAACCAGAGGGGTGTAGATGATGCA | cd13718: |
| | TTGCTCTCCCTGAAAACAGGGAAACTGGATGCCT | PBP2_iGluR_NMDA_ |
| | TCATCTATGATGCAGCAGTGCTGAACTATATGGC | Nr2 (NCBI) |
| | AGGCAGAGATGAAGGCTGCAAGCTGGTGACCAT | |
| | TGGCAGTGGGAAGGTCTTTGCTTCCACTGGCTAT | |
| | GGCATTGCCATCCAAAAAGATTCTGGGTGGAAG | |
| | CGCCAGGTGGACCTTGCTATCCTGCAGCTCTTTG | |
| | GAGATGGGGAGATGGAAGAACTGGAAGCTCTCT | |
| | GGCTCACTGGCATTTGTCACAAT | |
| SEQ ID NO | ATGAGCACCATGCGCCTGCTGACGCTCGCCCTG | DNA seq of fusion |
| 66 | CTGTTCTCCTGCTCCGTCGCCCGTGCCGCGTGC | protein #4 N1 ecd- |
| | GACCCCAAGATCGTCAACATTGGCGCGGTGCTG | N2Becd-Fc |
| | AGCACGCGGAAGCACGAGCAGATGTTCCGCGAG | |
| | GCCGTGAACCAGGCCAACAAGCGGCACGGCTCC | |
| | TGGAAGATTCAGCTCAATGCCACCTCCGTCACGC | |
| | ACAAGCCCAACGCCATCCAGATGGCTCTGTCGG | |
| | TGTGCGAGGACCTCATCTCCAGCCAGGTCTACG | |
| | CCATCCTAGTTAGCCATCCACCTACCCCCAACGA | |
| | CCACTTCACTCCCACCCCTGTCTCCTACACAGCC | |
| | GGCTTCTACCGCATACCCGTGCTGGGGCTGACC | |
| | ACCCGCATGTCCATCTACTCGGACAAGAGCATCC | |
| | ACCTGAGCTTCCTGCGCACCGTGCCGCCCTACT | |
| | CCCACCAGTCCAGCGTGTGGTTTGAGATGATGC | |
| | GTGTCTACAGCTGGAACCACATCATCCTGCTGGT | |
| | CAGCGACGACCACGAGGGCCGGGCGGCTCAGA | |
| | AACGCCTGGAGACGCTGCTGGAGGAGCGTGAGT | |
| | CCAAGGCAGAGAAGGTGCTGCAGTTTGACCCAG | |
| | GGACCAAGAACGTGACGGCCCTGCTGATGGAGG | |
| | CGAAAGAGCTGGAGGCCCGGGTCATCATCCTTT | |
| | CTGCCAGCGAGGACGATGCTGCCACTGTATACC | |
| | GCGCAGCCGCGATGCTGAACATGACGGGCTCCG | |
| | GGTACGTGTGGCTGGTCGGCGAGCGCGAGATCT | |
| | CGGGGAACGCCCTGCGCTACGCCCCGGACGGC | |
| | ATCCTCGGGCTGCAGCTCATCAACGGCAAGAAC | |
| | GAGTCGGCCCACATCAGCGACGCCGTAGGCGTG | |
| | GTGGCCCAGGCCGTGCACGAGCTCCTCGAGAAG | |
| | GAGAACATCACCGACCCGCCGCGGGGCTGCGT | |
| | GGGCAACACCAACATCTGGAAGACCGGGCCGCT | |
| | CTTCAAGAGAGTGCTGATGTCTTCCAAGTATGCG | |
| | GATGGGGTGACTGGTCGCGTGGAGTTCAATGAG | |
| | GATGGGGACCGGAAGTTCGCCAACTACAGCATC | |
| | ATGAACCTGCAGAACCGCAAGCTGGTGCAAGTG | |
| | GGCATCTACAATGGCACCCACGTCATCCCTAATG | |
| | ACAGGAAGATCATCTGGCCAGGCGGAGAGACAG | |
| | AGAAGCCTCGAGGGTACCAGATGTCCACCAGAC | |
| | TGAAGATTGTGACGATCCACCAGGAGCCCTTCGT | |
| | GTACGTCAAGCCCACGCTGAGTGATGGGACATG | |
| | CAAGGAGGAGTTCACAGTCAACGGCGACCCAGT | |
| | CAAGAAGGTGATCTGCACCGGGCCCAACGACAC | |
| | GTCGCCGGGCAGCCCCCGCCACACGGTGCCTC | |
| | AGTGTTGCTACGGCTTTTGCATCGACCTGCTCAT | |
| | CAAGCTGGCACGGACCATGAACTTCACCTACGA | |
| | GGTGCACCTGGTGGCAGATGGCAAGTTCGGCAC | |
| | ACAGGAGCGGGTGAACAACAGCAACAAGAAGGA | |
| | GTGGAATGGGATGATGGGCGAGCTGCTCAGCGG | |
| | GCAGGCAGACATGATCGTGGCGCCGCTAACCAT | |
| | AAACAACGAGCGCGCGCAGTACATCGAGTTTTC | |
| | CAAGCCCTTCAAGTACCAGGGCCTGACTATTCTG | |
| | GTCAAGAAGGGCACCCGCATCACGGGCATCAAC | |
| | GACCCCCGGCTGAGGAACCCCTCGGACAAGTTT | |
| | ATCTACGCCACGGTGAAGCAGAGCTCCGTGGAT | |
| | ATCTACTTCCGGCGCCAGGTGGAGCTGAGCACC | |
| | ATGTACCGGCATATGGAGAAGCACAACTACGAG | |
| | AGTGCGGCGGAGGCCATCCAGGCCGTGAGAGA | |
| | CAACAAGCTGCATGCCTTCATCTGGGACTCGGC | |
| | GGTGCTGGAGTTCGAGGCCTCGCAGAAGTGCGA | |
| | CCTGGTGACGACTGGAGAGCTGTTTTTCCGCTC | |
| | GGGCTTCGGCATAGGCATGCGCAAAGACAGCCC | |
| | CTGGAAGCAGAACGTCTCCCTGTCCATCCTCAAG | |
| | TCCCACGAGAATGGCTTCATGGAAGACCTGGAC | |
| | AAGACGTGGGTTCGGTATCAGGAATGTGACTCG | |
| | GGGTCGACTGGTGGAGGAGGGTCTGGCGGAGG | |
| | TGGTTCCGGGGGCGGAGGATCTGGGGCCGCTT | |
| | CTAGACAGAAGAGCCCCCCCAGCATTGGCATTG | |
| | CTGTCATCCTCGTGGGCACTTCCGACGAGGTGG | |
| | CCATCAAGGATGCCCACGAGAAAGATGATTTCCA | |
| | CCATCTCTCCGTGGTACCCCGGGTGGAACTGGT | |
| | AGCCATGAATGAGACCGACCCAAAGAGCATCAT | |
| | CACCCGCATCTGTGATCTCATGTCTGACCGGAAG | |
| | ATCCAGGGGGTGGTGTTTGCTGATGACACAGAC | |
| | CAGGAAGCCATCGCCCAGATCCTCGATTTCATTT | |
| | CAGCACAGACTCTCACCCCGATCCTGGGCATCC | |
| | ACGGGGGCTCCTCTATGATAATGGCAGATAAGG | |
| | ATGAATCCTCCATGTTCTTCCAGTTTGGCCCATC | |
| | AATTGAACAGCAAGCTTCCGTAATGCTCAACATC | |
| | ATGGAAGAATATGACTGGTACATCTTTTCTATCGT | |
| | CACCACCTATTTCCCTGGCTACCAGGACTTTGTA | |
| | AACAAGATCCGCAGCACCATTGAGAATAGCTTTG | |
| | TGGGCTGGGAGCTAGAGGAGGTCCTCCTACTGG | |
| | ACATGTCCCTGGACGATGGAGATTCTAAGATCCA | |
| | GAATCAGCTCAAGAAACTTCAAAGCCCCATCATT | |
| | CTTCTTTACTGTACCAAGGAAGAAGCCACCTACA | |
| | TCTTTGAAGTGGCCAACTCAGTAGGGCTGACTG | |
| | GCTATGGCTACACGTGGATCGTGCCCAGTCTGG | |
| | TGGCAGGGGATACAGACACAGTGCCTGCGGAGT | |
| | TCCCCACTGGGCTCATCTCTGTATCATATGATGA | |
| | ATGGGACTATGGCCTCCCCGCCAGAGTGAGAGA | |
| | TGGAATTGCCATAATCACCACTGCTGCTTCTGAC | |
| | ATGCTGTCTGAGCACAGCTTCATCCCTGAGCCCA | |
| | AAAGCAGTTGTTACAACACCCACGAGAAGAGAAT | |
| | CTACCAGTCCAATATGCTAAATAGGTATCTGATC | |
| | AATGTCACTTTTGAGGGGAGGAATTTGTCCTTCA | |
| | GTGAAGATGGCTACCAGATGCACCCGAAACTGG | |
| | TGATAATTCTTCTGAACAAGGAGAGGAAGTGGGA | |
| | AAGGGTGGGGAAGTGGAAAGACAAGTCCCTGCA | |
| | GATGAAGTACTATGTGTGGCCCCGAATGTGTCCA | |
| | GAGACTGAAGAGCAGGAGGATGACCATCTGAGC | |
| | ATTGTGACCCTGGAGGAGGCACCATTTGTCATTG | |
| | TGGAAAGTGTGGACCCTCTGAGTGGAACCTGCA | |
| | TGAGGAACACAGTCCCCTGCCAAAAACGCATAGT | |
| | CACTGAGAATAAAACAGACGAGGAGCCGGGTTA | |
| | CATCAAAAAATGCTGCAAGGGGTTCTGTATTGAC | |
| | ATCCTTAAGAAAATTTCTAAATCTGTGAAGTTCAC | |
| | CTATGACCTTTACCTGGTTACCAATGGCAAGCAT | |
| | GGGAAGAAAATCAATGGAACCTGGAATGGTATGA | |
| | TTGGAGAGGTGGTCATGAAGAGGGCCTACATGG | |
| | CAGTGGGCTCACTCACCATCAATGAGGAACGAT | |
| | CGGAGGTGGTCGACTTCTCTGTGCCCTTCATAGA | |
| | GACAGGCATCAGTGTCATGGTGTCACGCGGCAC | |
| | CCAGGTTTCTGGCCTGAGCGACAAAAAGTTCCA | |
| | GAGACCTAATGACTTCTCACCCCCTTTCCGCTTT | |
| | GGGACCGTGCCCAACGGCAGCACAGAGAGAAAT | |
| | ATTCGCAATAACTATGCAGAAATGCATGCCTACA | |
| | TGGGAAAGTTCAACCAGAGGGGTGTAGATGATG | |
| | CATTGCTCTCCCTGAAAACAGGGAAACTGGATGC | |
| | CTTCATCTATGATGCAGCAGTGCTGAACTATATG | |
| | GCAGGCAGAGATGAAGGCTGCAAGCTGGTGACC | |
| | ATTGGCAGTGGGAAGGTCTTTGCTTCCACTGGCT | |
| | ATGGCATTGCCATCCAAAAAGATTCTGGGTGGAA | |
| | GCGCCAGGTGGACCTTGCTATCCTGCAGCTCTTT | |
| | GGAGATGGGGAGATGGAAGAACTGGAAGCTCTC | |
| | TGGCTCACTGGCATTTGTCACAATGGCTCGAGCA | |
| | CCATGGTTAGATCTAGCAAGCCCACGTGCCCAC | |
| | CCCCTGAACTCCTGGGGGGACCGTCTGTCTTCA | |
| | TCTTCCCCCCAAAACCCAAGGACACCCTCATGAT | |
| | CTCACGCACCCCCGAGGTCACATGCGTGGTGGT | |
| | GGACGTGAGCCAGGATGACCCCGAGGTGCAGTT | |
| | CACATGGTACATAAACAACGAGCAGGTGCGCAC | |
| | CGCCCGGCCGCCGCTACGGGAGCAGCAGTTCA | |
| | ACAGCACGATCCGCGTGGTCAGCACCCTCCCCA | |
| | TCGCGCACCAGGACTGGCTGAGGGGCAAGGAG | |
| | TTCAAGTGCAAAGTCCACAACAAGGCACTCCCG | |
| | GCCCCCATCGAGAAAACCATCTCCAAAGCCAGA | |
| | GGGCAGCCCCTGGAGCCGAAGGTCTACACCATG | |
| | GGCCCTCCCCGGGAGGAGCTGAGCAGCAGGTC | |
| | GGTCAGCCTGACCTGCATGATCAACGGCTTCTAC | |
| | CCTTCCGACATCTCGGTGGAGTGGGAGAAGAAC | |
| | GGGAAGGCAGAGGACAACTACAAGACCACGCCG | |
| | GCCGTGCTGGACAGCGACGGCTCCTACTTCCTC | |
| | TACAGCAAGCTCTCAGTGCCCACGAGTGAGTGG | |
| | CAGCGGGGCGACGTCTTCACCTGCTCCGTGATG | |
| | CACGAGGCCTTGCACAACCACTACACGCAGAAG | |
| | TCCATCTCCCGCTCTCCGGGTAAATGA | |
| SEQ ID NO | GGGTCGACTGGTGGAGGAGGGTCTGGCGGAGG | Linker between a |
| 67 | TGGTTCCGGGGGCGGAGGATCTGGGGCCGCTT | GluN1-domain and |
| | CTAGA | GluN2B-domain in a |
| | | NMDAR construct of |
| | | the invention, as for |
| | | example in fusion |
| | | protein #4 N1 ecd- |
| | | N2Becd-Fc or in fusion |
| | | proteins #7, #8 |
| SEQ ID NO | CAGAAGAGCCCCCCCAGCATTGGCATTGCTGTC | Amino terminal domain |
| 68 | ATCCTCGTGGGCACTTCCGACGAGGTGGCCATC | of GluN2B as for |
| | AAGGATGCCCACGAGAAAGATGATTTCCACCATC | example used in fusion |
| | TCTCCGTGGTACCCCGGGTGGAACTGGTAGCCA | protein #4 N1 ecd- |
| | TGAATGAGACCGACCCAAAGAGCATCATCACCC | N2Becd-Fc |
| | GCATCTGTGATCTCATGTCTGACCGGAAGATCCA | |
| | GGGGGTGGTGTTTGCTGATGACACAGACCAGGA | |
| | AGCCATCGCCCAGATCCTCGATTTCATTTCAGCA | |
| | CAGACTCTCACCCCGATCCTGGGCATCCACGGG | |
| | GGCTCCTCTATGATAATGGCAGATAAGGATGAAT | |
| | CCTCCATGTTCTTCCAGTTTGGCCCATCAATTGA | |
| | ACAGCAAGCTTCCGTAATGCTCAACATCATGGAA | |
| | GAATATGACTGGTACATCTTTTCTATCGTCACCAC | |
| | CTATTTCCCTGGCTACCAGGACTTTGTAAACAAG | |
| | ATCCGCAGCACCATTGAGAATAGCTTTGTGGGCT | |
| | GGGAGCTAGAGGAGGTCCTCCTACTGGACATGT | |
| | CCCTGGACGATGGAGATTCTAAGATCCAGAATCA | |
| | GCTCAAGAAACTTCAAAGCCCCATCATTCTTCTTT | |
| | ACTGTACCAAGGAAGAAGCCACCTACATCTTTGA | |
| | AGTGGCCAACTCAGTAGGGCTGACTGGCTATGG | |
| | CTACACGTGGATCGTGCCCAGTCTGGTGGCAGG | |
| | GGATACAGACACAGTGCCTGCGGAGTTCCCCAC | |
| | TGGGCTCATCTCTGTATCATATGATGAATGGGAC | |
| | TATGGCCTCCCCGCCAGAGTGAGAGATGGAATT | |
| | GCCATAATCACCACTGCTGCTTCTGACATGCTGT | |
| | CTGAGCACAGCTTCATCCCTGAGCCCAAAAGCA | |
| | GTTGTTACAACACCCACGAGAAGAGAATCTACCA | |
| | GTCCAATATGCTAAATAGGTATCTGATCAATGTCA | |
| | CTTTTGAGGGGAGGAATTTGTCCTTCAGTGAAGA | |
| | TGGCTACCAGATGCACCCGAAACTGGTGATAATT | |
| | CTTCTGAACAAGGAGAGGAAGTGGGAAAGGGTG | |
| | GGGAAGTGGAAAGACAAGTCCCTGCAGATGAAG | |
| | TACTATGTGTGGCCCCGAATGTGTCCAGAGACTG | |
| | AAGAGCAG | |
| SEQ ID NO | ATGGGCAGAGTGGGCTATTGGACCCTGCTGGTG | DNA seq of fusion |
| 69 | CTGCCGGCCCTTCTGGTCTGGCGCGGTCCGGC | protein #5 N2A-ATD- |
| | GCCGAGCGCGGCGGCGGAGAAGGGTCCCCCCG | Fc |
| | CGCTAAATATTGCGGTGATGCTGGGTCACAGCC | |
| | ACGACGTGACAGAGCGCGAACTTCGAACACTGT | |
| | GGGGCCCCGAGCAGGCGGCGGGGCTGCCCCTG | |
| | GACGTGAACGTGGTAGCTCTGCTGATGAACCGC | |
| | ACCGACCCCAAGAGCCTCATCACGCACGTGTGC | |
| | GACCTCATGTCCGGGGCACGCATCCACGGCCTC | |
| | GTGTTTGGGGACGACACGGACCAGGAGGCCGTA | |
| | GCCCAGATGCTGGATTTTATCTCCTCCCACACCT | |
| | TCGTCCCCATCTTGGGCATTCATGGGGGCGCAT | |
| | CTATGATCATGGCTGACAAGGATCCGACGTCTAC | |
| | CTTCTTCCAGTTTGGAGCGTCCATCCAGCAGCAA | |
| | GCCACGGTCATGCTGAAGATCATGCAGGATTATG | |
| | ACTGGCATGTCTTCTCCCTGGTGACCACTATCTT | |
| | CCCTGGCTACAGGGAATTCATCAGCTTCGTCAAG | |
| | ACCACAGTGGACAACAGCTTTGTGGGCTGGGAC | |
| | ATGCAGAATGTGATCACACTGGACACTTCCTTTG | |
| | AGGATGCAAAGACACAAGTCCAGCTGAAGAAGA | |
| | TCCACTCTTCTGTCATCTTGCTCTACTGTTCCAAA | |
| | GACGAGGCTGTTCTCATTCTGAGTGAGGCCCGC | |
| | TCCCTTGGCCTCACCGGGTATGATTTCTTCTGGA | |
| | TTGTCCCCAGCTTGGTCTCTGGGAACACGGAGC | |
| | TCATCCCAAAAGAGTTTCCATCGGGACTCATTTC | |
| | TGTCTCCTACGATGACTGGGACTACAGCCTGGA | |
| | GGCGAGAGTGAGGGACGGCATTGGCATCCTAAC | |
| | CACCGCTGCATCTTCTATGCTGGAGAAGTTCTCC | |
| | TACATCCCCGAGGCCAAGGCCAGCTGCTACGGG | |
| | CAGATGGAGAGGCCAGAGGTCCCGATGCACACC | |
| | TTGCACCCATTTATGGTCAATGTTACATGGGATG | |
| | GCAAAGACTTATCCTTCACTGAGGAAGGCTACCA | |
| | GGTGCACCCCAGGCTGGTGGTGATTGTGCTGAA | |
| | CAAAGACCGGGAATGGGAAAAGGTGGGCAAGTG | |
| | GGAGAACCATACGCTGAGCCTGAGGCACGCCGT | |
| | GTGGCCCAGGTACAAGTCCTTCTCCGACTGTGA | |
| | GCCGGATGACAACCATCTCAGCATCGGCTCGAG | |
| | CACCATGGTTAGATCTAGCAAGCCCACGTGCCC | |
| | ACCCCCTGAACTCCTGGGGGGACCGTCTGTCTT | |
| | CATCTTCCCCCCAAAACCCAAGGACACCCTCATG | |
| | ATCTCACGCACCCCCGAGGTCACATGCGTGGTG | |
| | GTGGACGTGAGCCAGGATGACCCCGAGGTGCA | |
| | GTTCACATGGTACATAAACAACGAGCAGGTGCG | |
| | CACCGCCCGGCCGCCGCTACGGGAGCAGCAGT | |
| | TCAACAGCACGATCCGCGTGGTCAGCACCCTCC | |
| | CCATCGCGCACCAGGACTGGCTGAGGGGCAAG | |
| | GAGTTCAAGTGCAAAGTCCACAACAAGGCACTCC | |
| | CGGCCCCCATCGAGAAAACCATCTCCAAAGCCA | |
| | GAGGGCAGCCCCTGGAGCCGAAGGTCTACACCA | |
| | TGGGCCCTCCCCGGGAGGAGCTGAGCAGCAGG | |
| | TCGGTCAGCCTGACCTGCATGATCAACGGCTTCT | |
| | ACCCTTCCGACATCTCGGTGGAGTGGGAGAAGA | |
| | ACGGGAAGGCAGAGGACAACTACAAGACCACGC | |
| | CGGCCGTGCTGGACAGCGACGGCTCCTACTTCC | |
| | TCTACAGCAAGCTCTCAGTGCCCACGAGTGAGT | |
| | GGCAGCGGGGCGACGTCTTCACCTGCTCCGTGA | |
| | TGCACGAGGCCTTGCACAACCACTACACGCAGA | |
| | AGTCCATCTCCCGCTCTCCGGGTAAATGA | |
| SEQ ID NO | ATGGGCAGAGTGGGCTATTGGACCCTGCTGGTG | Amino terminal domain |
| 70 | CTGCCGGCCCTTCTGGTCTGGCGCGGTCCGGC | of GluN2A (including |
| | GCCGAGCGCGGCGGCGGAGAAGGGTCCCCCCG | signal sequence) |
| | CGCTAAATATTGCGGTGATGCTGGGTCACAGCC | |
| | ACGACGTGACAGAGCGCGAACTTCGAACACTGT | |
| | GGGGCCCCGAGCAGGCGGCGGGGCTGCCCCTG | |
| | GACGTGAACGTGGTAGCTCTGCTGATGAACCGC | |
| | ACCGACCCCAAGAGCCTCATCACGCACGTGTGC | |
| | GACCTCATGTCCGGGGCACGCATCCACGGCCTC | |
| | GTGTTTGGGGACGACACGGACCAGGAGGCCGTA | |
| | GCCCAGATGCTGGATTTTATCTCCTCCCACACCT | |
| | TCGTCCCCATCTTGGGCATTCATGGGGGCGCAT | |
| | CTATGATCATGGCTGACAAGGATCCGACGTCTAC | |
| | CTTCTTCCAGTTTGGAGCGTCCATCCAGCAGCAA | |
| | GCCACGGTCATGCTGAAGATCATGCAGGATTATG | |
| | ACTGGCATGTCTTCTCCCTGGTGACCACTATCTT | |
| | CCCTGGCTACAGGGAATTCATCAGCTTCGTCAAG | |
| | ACCACAGTGGACAACAGCTTTGTGGGCTGGGAC | |
| | ATGCAGAATGTGATCACACTGGACACTTCCTTTG | |
| | AGGATGCAAAGACACAAGTCCAGCTGAAGAAGA | |
| | TCCACTCTTCTGTCATCTTGCTCTACTGTTCCAAA | |
| | GACGAGGCTGTTCTCATTCTGAGTGAGGCCCGC | |
| | TCCCTTGGCCTCACCGGGTATGATTTCTTCTGGA | |
| | TTGTCCCCAGCTTGGTCTCTGGGAACACGGAGC | |
| | TCATCCCAAAAGAGTTTCCATCGGGACTCATTTC | |
| | TGTCTCCTACGATGACTGGGACTACAGCCTGGA | |
| | GGCGAGAGTGAGGGACGGCATTGGCATCCTAAC | |
| | CACCGCTGCATCTTCTATGCTGGAGAAGTTCTCC | |
| | TACATCCCCGAGGCCAAGGCCAGCTGCTACGGG | |
| | CAGATGGAGAGGCCAGAGGTCCCGATGCACACC | |
| | TTGCACCCATTTATGGTCAATGTTACATGGGATG | |
| | GCAAAGACTTATCCTTCACTGAGGAAGGCTACCA | |
| | GGTGCACCCCAGGCTGGTGGTGATTGTGCTGAA | |
| | CAAAGACCGGGAATGGGAAAAGGTGGGCAAGTG | |
| | GGAGAACCATACGCTGAGCCTGAGGCACGCCGT | |
| | GTGGCCCAGGTACAAGTCCTTCTCCGACTGTGA | |
| | GCCGGATGACAACCATCTCAGCATC | |
| SEQ ID NO | CCCGCGCTAAATATTGCGGTGATGCTGGGTCAC | Amino terminal domain |
| 71 | AGCCACGACGTGACAGAGCGCGAACTTCGAACA | of GluN2A in |
| | CTGTGGGGCCCCGAGCAGGCGGCGGGGCTGCC | NM_001134407 with |
| | CCTGGACGTGAACGTGGTAGCTCTGCTGATGAA | domain borders as in |
| | CCGCACCGACCCCAAGAGCCTCATCACGCACGT | cd06378: |
| | GTGCGACCTCATGTCCGGGGCACGCATCCACGG | PBP1_iGluR_NMDA_ |
| | CCTCGTGTTTGGGGACGACACGGACCAGGAGGC | NR2 (NCBI) |
| | CGTAGCCCAGATGCTGGATTTTATCTCCTCCCAC | |
| | ACCTTCGTCCCCATCTTGGGCATTCATGGGGGC | |
| | GCATCTATGATCATGGCTGACAAGGATCCGACGT | |
| | CTACCTTCTTCCAGTTTGGAGCGTCCATCCAGCA | |
| | GCAAGCCACGGTCATGCTGAAGATCATGCAGGA | |
| | TTATGACTGGCATGTCTTCTCCCTGGTGACCACT | |
| | ATCTTCCCTGGCTACAGGGAATTCATCAGCTTCG | |
| | TCAAGACCACAGTGGACAACAGCTTTGTGGGCT | |
| | GGGACATGCAGAATGTGATCACACTGGACACTTC | |
| | CTTTGAGGATGCAAAGACACAAGTCCAGCTGAAG | |
| | AAGATCCACTCTTCTGTCATCTTGCTCTACTGTTC | |
| | CAAAGACGAGGCTGTTCTCATTCTGAGTGAGGC | |
| | CCGCTCCCTTGGCCTCACCGGGTATGATTTCTTC | |
| | TGGATTGTCCCCAGCTTGGTCTCTGGGAACACG | |
| | GAGCTCATCCCAAAAGAGTTTCCATCGGGACTCA | |
| | TTTCTGTCTCCTACGATGACTGGGACTACAGCCT | |
| | GGAGGCGAGAGTGAGGGACGGCATTGGCATCCT | |
| | AACCACCGCTGCATCTTCTATGCTGGAGAAGTTC | |
| | TCCTACATCCCCGAGGCCAAGGCCAGCTGCTAC | |
| | GGGCAGATGGAGAGGCCAGAGGTCCCGATGCA | |
| | CACCTTGCACCCATTTATGGTCAATGTTACATGG | |
| | GATGGCAAAGACTTATCCTTCACTGAGGAAGGCT | |
| | ACCAGGTGCACCCCAGGCTGGTGGTGATTGTGC | |
| | TGAACAAAGACCGGGAATGGGAAAAGGTGGGCA | |
| | AGTGGGAGAACCATACGCTGAGCCTGAGGCACG | |
| | CCGTGTGGCCCAGGACGCTCGCCCTGCTGTTCT | |
| | CCTGCTCCGTCGCCCGTGCCGCGTGCGACCCCA | |
| | AGATCGTCAACATTGGCGCGGTGCTGAGCACGC | |
| | GGAAGCACGAGCAGATGTTCCGCGAGGCCGTGA | |
| | ACCAGGCCAACAAGCGGCACGGCTCCTGGAAGA | |
| | TTCAGCTCAATGCCACCTCCGTCACGCACAAGCC | |
| | CAACGCCATCCAGATGGCTCTGTCGGTGTGCGA | |
| | GGACCTCATCTCCAGCCAGGTCTACGCCATCCTA | |
| | GTTAGCCATCCACCTACCCCCAACGACCACTTCA | |
| | CTCCCACCCCTGTCTCCTACACAGCCGGCTTCTA | |
| | CCGCATACCCGTGCTGGGGCTGACCACCCGCAT | |
| | GTCCATCTACTCGGACAAGAGCATCCACCTGAG | |
| | CTTCCTGCGCACCGTGCCGCCCTACTCCCACCA | |
| | GTCCAGCGTGTGGTTTGAGATGATGCGTGTCTAC | |
| | AGCTGGAACCACATCATCCTGCTGGTCAGCGAC | |
| | GACCACGAGGGCCGGGCGGCTCAGAAACGCCT | |
| | GGAGACGCTGCTGGAGGAGCGTGAGTCCAAGG | |
| | CAGAGAAGGTGCTGCAGTTTGACCCAGGGACCA | |
| | AGAACGTGACGGCCCTGCTGATGGAGGCGAAAG | |
| | AGCTGGAGGCCCGGGTCATCATCCTTTCTGCCA | |
| | GCGAGGACGATGCTGCCACTGTATACCGCGCAG | |
| | CCGCGATGCTGAACATGACGGGCTCCGGGTACG | |
| | TGTGGCTGGTCGGCGAGCGCGAGATCTCGGGG | |
| | AACGCCCTGCGCTACGCCCCAGACGGCATCCTC | |
| | GGGCTGCAGCTCATCAACGGCAAGAACGAGTCG | |
| | GCCCACATCAGCGACGCCGTGGGCGTGGTGGC | |
| | CCAGGCCGTGCACGAGCTCCTCGAGAAGGAGAA | |
| | CATCACCGACCCGCCGCGGGGCTGCGTGGGCA | |
| | ACACCAACATCTGGAAGACCGGGCCGCTCTTCA | |
| | AGAGAGTGCTGATGTCTTCCAAGTATGCGGATG | |
| | GGGTGACTGGTCGCGTGGAGTTCAATGAGGATG | |
| | GGGACCGGAAGTTCGCCAACTACAGCATCATGA | |
| | ACCTGCAGAACCGCAAGCTGGTGCAAGTGGGCA | |
| | TCTACAATGGCACCCACGTCATCCCTAATGACAG | |
| | GAAGATCATCTGGCCAGGC | |
| SEQ ID NO | ATGAAGCCCAGAGCGGAGTGCTGTTCTCCCAAG | DNA seq of fusion |
| 72 | TTCTGGTTGGTGTTGGCCGTCCTGGCCGTGTCA | protein #6 N2B-ATD- |
| | GGCAGCAGAGCTCGTTCTCAGAAGAGCCCCCCC | Fc |
| | AGCATTGGCATTGCTGTCATCCTCGTGGGCACTT | |
| | CCGACGAGGTGGCCATCAAGGATGCCCACGAGA | |
| | AAGATGATTTCCACCATCTCTCCGTGGTACCCCG | |
| | GGTGGAACTGGTAGCCATGAATGAGACCGACCC | |
| | AAAGAGCATCATCACCCGCATCTGTGATCTCATG | |
| | TCTGACCGGAAGATCCAGGGGGTGGTGTTTGCT | |
| | GATGACACAGACCAGGAAGCCATCGCCCAGATC | |
| | CTCGATTTCATTTCAGCACAGACTCTCACCCCGA | |
| | TCCTGGGCATCCACGGGGGCTCCTCTATGATAAT | |
| | GGCAGATAAGGATGAATCCTCCATGTTCTTCCAG | |
| | TTTGGCCCATCAATTGAACAGCAAGCTTCCGTAA | |
| | TGCTCAACATCATGGAAGAATATGACTGGTACAT | |
| | CTTTTCTATCGTCACCACCTATTTCCCTGGCTACC | |
| | AGGACTTTGTAAACAAGATCCGCAGCACCATTGA | |
| | GAATAGCTTTGTGGGCTGGGAGCTAGAGGAGGT | |
| | CCTCCTACTGGACATGTCCCTGGACGATGGAGA | |
| | TTCTAAGATCCAGAATCAGCTCAAGAAACTTCAA | |
| | AGCCCCATCATTCTTCTTTACTGTACCAAGGAAG | |
| | AAGCCACCTACATCTTTGAAGTGGCCAACTCAGT | |
| | AGGGCTGACTGGCTATGGCTACACGTGGATCGT | |
| | GCCCAGTCTGGTGGCAGGGGATACAGACACAGT | |
| | GCCTGCGGAGTTCCCCACTGGGCTCATCTCTGT | |
| | ATCATATGATGAATGGGACTATGGCCTCCCCGCC | |
| | AGAGTGAGAGATGGAATTGCCATAATCACCACTG | |
| | CTGCTTCTGACATGCTGTCTGAGCACAGCTTCAT | |
| | CCCTGAGCCCAAAAGCAGTTGTTACAACACCCAC | |
| | GAGAAGAGAATCTACCAGTCCAATATGCTAAATA | |
| | GGTATCTGATCAATGTCACTTTTGAGGGGAGGAA | |
| | TTTGTCCTTCAGTGAAGATGGCTACCAGATGCAC | |
| | CCGAAACTGGTGATAATTCTTCTGAACAAGGAGA | |
| | GGAAGTGGGAAAGGGTGGGGAAGTGGAAAGAC | |
| | AAGTCCCTGCAGATGAAGTACTATGTGTGGCCCC | |
| | GAATGTGTCCAGAGACTGAAGAGCAGGAGGATG | |
| | ACCATCTGAGCATTGGCTCGAGCACCATGGTTAG | |
| | ATCTAGCAAGCCCACGTGCCCACCCCCTGAACT | |
| | CCTGGGGGGACCGTCTGTCTTCATCTTCCCCCC | |
| | AAAACCCAAGGACACCCTCATGATCTCACGCACC | |
| | CCCGAGGTCACATGCGTGGTGGTGGACGTGAGC | |
| | CAGGATGACCCCGAGGTGCAGTTCACATGGTAC | |
| | ATAAACAACGAGCAGGTGCGCACCGCCCGGCCG | |
| | CCGCTACGGGAGCAGCAGTTCAACAGCACGATC | |
| | CGCGTGGTCAGCACCCTCCCCATCGCGCACCAG | |
| | GACTGGCTGAGGGGCAAGGAGTTCAAGTGCAAA | |
| | GTCCACAACAAGGCACTCCCGGCCCCCATCGAG | |
| | AAAACCATCTCCAAAGCCAGAGGGCAGCCCCTG | |
| | GAGCCGAAGGTCTACACCATGGGCCCTCCCCGG | |
| | GAGGAGCTGAGCAGCAGGTCGGTCAGCCTGACC | |
| | TGCATGATCAACGGCTTCTACCCTTCCGACATCT | |
| | CGGTGGAGTGGGAGAAGAACGGGAAGGCAGAG | |
| | GACAACTACAAGACCACGCCGGCCGTGCTGGAC | |
| | AGCGACGGCTCCTACTTCCTCTACAGCAAGCTCT | |
| | CAGTGCCCACGAGTGAGTGGCAGCGGGGCGAC | |
| | GTCTTCACCTGCTCCGTGATGCACGAGGCCTTG | |
| | CACAACCACTACACGCAGAAGTCCATCTCCCGCT | |
| | CTCCGGGTAAATGA | |
| SEQ ID NO | ATGAAGCCCAGAGCGGAGTGCTGTTCTCCCAAG | Amino terminal domain |
| 73 | TTCTGGTTGGTGTTGGCCGTCCTGGCCGTGTCA | of GluN2B (including |
| | GGCAGCAGAGCTCGTTCTCAGAAGAGCCCCCCC | signal sequence) as |
| | AGCATTGGCATTGCTGTCATCCTCGTGGGCACTT | for example used in |
| | CCGACGAGGTGGCCATCAAGGATGCCCACGAGA | fusion protein #6 |
| | AAGATGATTTCCACCATCTCTCCGTGGTACCCCG | |
| | GGTGGAACTGGTAGCCATGAATGAGACCGACCC | |
| | AAAGAGCATCATCACCCGCATCTGTGATCTCATG | |
| | TCTGACCGGAAGATCCAGGGGGTGGTGTTTGCT | |
| | GATGACACAGACCAGGAAGCCATCGCCCAGATC | |
| | CTCGATTTCATTTCAGCACAGACTCTCACCCCGA | |
| | TCCTGGGCATCCACGGGGGCTCCTCTATGATAAT | |
| | GGCAGATAAGGATGAATCCTCCATGTTCTTCCAG | |
| | TTTGGCCCATCAATTGAACAGCAAGCTTCCGTAA | |
| | TGCTCAACATCATGGAAGAATATGACTGGTACAT | |
| | CTTTTCTATCGTCACCACCTATTTCCCTGGCTACC | |
| | AGGACTTTGTAAACAAGATCCGCAGCACCATTGA | |
| | GAATAGCTTTGTGGGCTGGGAGCTAGAGGAGGT | |
| | CCTCCTACTGGACATGTCCCTGGACGATGGAGA | |
| | TTCTAAGATCCAGAATCAGCTCAAGAAACTTCAA | |
| | AGCCCCATCATTCTTCTTTACTGTACCAAGGAAG | |
| | AAGCCACCTACATCTTTGAAGTGGCCAACTCAGT | |
| | AGGGCTGACTGGCTATGGCTACACGTGGATCGT | |
| | GCCCAGTCTGGTGGCAGGGGATACAGACACAGT | |
| | GCCTGCGGAGTTCCCCACTGGGCTCATCTCTGT | |
| | ATCATATGATGAATGGGACTATGGCCTCCCCGCC | |
| | AGAGTGAGAGATGGAATTGCCATAATCACCACTG | |
| | CTGCTTCTGACATGCTGTCTGAGCACAGCTTCAT | |
| | CCCTGAGCCCAAAAGCAGTTGTTACAACACCCAC | |
| | GAGAAGAGAATCTACCAGTCCAATATGCTAAATA | |
| | GGTATCTGATCAATGTCACTTTTGAGGGGAGGAA | |
| | TTTGTCCTTCAGTGAAGATGGCTACCAGATGCAC | |
| | CCGAAACTGGTGATAATTCTTCTGAACAAGGAGA | |
| | GGAAGTGGGAAAGGGTGGGGAAGTGGAAAGAC | |
| | AAGTCCCTGCAGATGAAGTACTATGTGTGGCCCC | |
| | GAATGTGTCCAGAGACTGAAGAGCAGGAGGATG | |
| | ACCATCTGAGCATT | |
| SEQ ID NO | ATGAGCACCATGCGCCTGCTGACGCTCGCCCTG | DNA seq of fusion |
| 74 | CTGTTCTCCTGCTCCGTCGCCCGTGCCGCGTGC | protein #7 N1-ATD- |
| | GACCCCAAGATCGTCAACATTGGCGCGGTGCTG | N2A-ATD-Fc |
| | AGCACGCGGAAGCACGAGCAGATGTTCCGCGAG | |
| | GCCGTGAACCAGGCCAACAAGCGGCACGGCTCC | |
| | TGGAAGATTCAGCTCAATGCCACCTCCGTCACGC | |
| | ACAAGCCCAACGCCATCCAGATGGCTCTGTCGG | |
| | TGTGCGAGGACCTCATCTCCAGCCAGGTCTACG | |
| | CCATCCTAGTTAGCCATCCACCTACCCCCAACGA | |
| | CCACTTCACTCCCACCCCTGTCTCCTACACAGCC | |
| | GGCTTCTACCGCATACCCGTGCTGGGGCTGACC | |
| | ACCCGCATGTCCATCTACTCGGACAAGAGCATCC | |
| | ACCTGAGCTTCCTGCGCACCGTGCCGCCCTACT | |
| | CCCACCAGTCCAGCGTGTGGTTTGAGATGATGC | |
| | GTGTCTACAGCTGGAACCACATCATCCTGCTGGT | |
| | CAGCGACGACCACGAGGGCCGGGCGGCTCAGA | |
| | AACGCCTGGAGACGCTGCTGGAGGAGCGTGAGT | |
| | CCAAGGCAGAGAAGGTGCTGCAGTTTGACCCAG | |
| | GGACCAAGAACGTGACGGCCCTGCTGATGGAGG | |
| | CGAAAGAGCTGGAGGCCCGGGTCATCATCCTTT | |
| | CTGCCAGCGAGGACGATGCTGCCACTGTATACC | |
| | GCGCAGCCGCGATGCTGAACATGACGGGCTCCG | |
| | GGTACGTGTGGCTGGTCGGCGAGCGCGAGATCT | |
| | CGGGGAACGCCCTGCGCTACGCCCCGGACGGC | |
| | ATCCTCGGGCTGCAGCTCATCAACGGCAAGAAC | |
| | GAGTCGGCCCACATCAGCGACGCCGTAGGCGTG | |
| | GTGGCCCAGGCCGTGCACGAGCTCCTCGAGAAG | |
| | GAGAACATCACCGACCCGCCGCGGGGCTGCGT | |
| | GGGCAACACCAACATCTGGAAGACCGGGCCGCT | |
| | CTTCAAGAGAGTGCTGATGTCTTCCAAGTATGCG | |
| | GATGGGGTGACTGGTCGCGTGGAGTTCAATGAG | |
| | GATGGGGACCGGAAGTTCGCCAACTACAGCATC | |
| | ATGAACCTGCAGAACCGCAAGCTGGTGCAAGTG | |
| | GGCATCTACAATGGCACCCACGTCATCCCTAATG | |
| | ACAGGAAGATCATCTGGCCAGGCGGAGAGACAG | |
| | AGAAGCCTCGAGGGTACCAGATGTCCACCAGAC | |
| | TGAAGATTGGGTCGACTGGTGGAGGAGGGTCTG | |
| | GCGGAGGTGGTTCCGGGGGCGGAGGATCTGGG | |
| | GCCGCTTCTAGAGAGAAGGGTCCCCCCGCGCTA | |
| | AATATTGCGGTGATGCTGGGTCACAGCCACGAC | |
| | GTGACAGAGCGCGAACTTCGAACACTGTGGGGC | |
| | CCCGAGCAGGCGGCGGGGCTGCCCCTGGACGT | |
| | GAACGTGGTAGCTCTGCTGATGAACCGCACCGA | |
| | CCCCAAGAGCCTCATCACGCACGTGTGCGACCT | |
| | CATGTCCGGGGCACGCATCCACGGCCTCGTGTT | |
| | TGGGGACGACACGGACCAGGAGGCCGTAGCCC | |
| | AGATGCTGGATTTTATCTCCTCCCACACCTTCGT | |
| | CCCCATCTTGGGCATTCATGGGGGCGCATCTAT | |
| | GATCATGGCTGACAAGGATCCGACGTCTACCTTC | |
| | TTCCAGTTTGGAGCGTCCATCCAGCAGCAAGCC | |
| | ACGGTCATGCTGAAGATCATGCAGGATTATGACT | |
| | GGCATGTCTTCTCCCTGGTGACCACTATCTTCCC | |
| | TGGCTACAGGGAATTCATCAGCTTCGTCAAGACC | |
| | ACAGTGGACAACAGCTTTGTGGGCTGGGACATG | |
| | CAGAATGTGATCACACTGGACACTTCCTTTGAGG | |
| | ATGCAAAGACACAAGTCCAGCTGAAGAAGATCCA | |
| | CTCTTCTGTCATCTTGCTCTACTGTTCCAAAGAC | |
| | GAGGCTGTTCTCATTCTGAGTGAGGCCCGCTCC | |
| | CTTGGCCTCACCGGGTATGATTTCTTCTGGATTG | |
| | TCCCCAGCTTGGTCTCTGGGAACACGGAGCTCA | |
| | TCCCAAAAGAGTTTCCATCGGGACTCATTTCTGT | |
| | CTCCTACGATGACTGGGACTACAGCCTGGAGGC | |
| | GAGAGTGAGGGACGGCATTGGCATCCTAACCAC | |
| | CGCTGCATCTTCTATGCTGGAGAAGTTCTCCTAC | |
| | ATCCCCGAGGCCAAGGCCAGCTGCTACGGGCAG | |
| | ATGGAGAGGCCAGAGGTCCCGATGCACACCTTG | |
| | CACCCATTTATGGTCAATGTTACATGGGATGGCA | |
| | AAGACTTATCCTTCACTGAGGAAGGCTACCAGGT | |
| | GCACCCCAGGCTGGTGGTGATTGTGCTGAACAA | |
| | AGACCGGGAATGGGAAAAGGTGGGCAAGTGGG | |
| | AGAACCATACGCTGAGCCTGAGGCACGCCGTGT | |
| | GGCCCAGGTACAAGTCCTTCTCCGACTGTGAGC | |
| | CGGATGACAACCATCTCAGCATCGGCTCGAGCA | |
| | CCATGGTTAGATCTAGCAAGCCCACGTGCCCAC | |
| | CCCCTGAACTCCTGGGGGGACCGTCTGTCTTCA | |
| | TCTTCCCCCCAAAACCCAAGGACACCCTCATGAT | |
| | CTCACGCACCCCCGAGGTCACATGCGTGGTGGT | |
| | GGACGTGAGCCAGGATGACCCCGAGGTGCAGTT | |
| | CACATGGTACATAAACAACGAGCAGGTGCGCAC | |
| | CGCCCGGCCGCCGCTACGGGAGCAGCAGTTCA | |
| | ACAGCACGATCCGCGTGGTCAGCACCCTCCCCA | |
| | TCGCGCACCAGGACTGGCTGAGGGGCAAGGAG | |
| | TTCAAGTGCAAAGTCCACAACAAGGCACTCCCG | |
| | GCCCCCATCGAGAAAACCATCTCCAAAGCCAGA | |
| | GGGCAGCCCCTGGAGCCGAAGGTCTACACCATG | |
| | GGCCCTCCCCGGGAGGAGCTGAGCAGCAGGTC | |
| | GGTCAGCCTGACCTGCATGATCAACGGCTTCTAC | |
| | CCTTCCGACATCTCGGTGGAGTGGGAGAAGAAC | |
| | GGGAAGGCAGAGGACAACTACAAGACCACGCCG | |
| | GCCGTGCTGGACAGCGACGGCTCCTACTTCCTC | |
| | TACAGCAAGCTCTCAGTGCCCACGAGTGAGTGG | |
| | CAGCGGGGCGACGTCTTCACCTGCTCCGTGATG | |
| | CACGAGGCCTTGCACAACCACTACACGCAGAAG | |
| | TCCATCTCCCGCTCTCCGGGTAAATGA | |
| SEQ ID NO | ATGAGCACCATGCGCCTGCTGACGCTCGCCCTG | Amino terminal domain |
| 75 | CTGTTCTCCTGCTCCGTCGCCCGTGCCGCGTGC | of GluN1 (including |
| | GACCCCAAGATCGTCAACATTGGCGCGGTGCTG | signal sequence) as |
| | AGCACGCGGAAGCACGAGCAGATGTTCCGCGAG | for example used in |
| | GCCGTGAACCAGGCCAACAAGCGGCACGGCTCC | fusion protein #7 |
| | TGGAAGATTCAGCTCAATGCCACCTCCGTCACGC | |
| | ACAAGCCCAACGCCATCCAGATGGCTCTGTCGG | |
| | TGTGCGAGGACCTCATCTCCAGCCAGGTCTACG | |
| | CCATCCTAGTTAGCCATCCACCTACCCCCAACGA | |
| | CCACTTCACTCCCACCCCTGTCTCCTACACAGCC | |
| | GGCTTCTACCGCATACCCGTGCTGGGGCTGACC | |
| | ACCCGCATGTCCATCTACTCGGACAAGAGCATCC | |
| | ACCTGAGCTTCCTGCGCACCGTGCCGCCCTACT | |
| | CCCACCAGTCCAGCGTGTGGTTTGAGATGATGC | |
| | GTGTCTACAGCTGGAACCACATCATCCTGCTGGT | |
| | CAGCGACGACCACGAGGGCCGGGCGGCTCAGA | |
| | AACGCCTGGAGACGCTGCTGGAGGAGCGTGAGT | |
| | CCAAGGCAGAGAAGGTGCTGCAGTTTGACCCAG | |
| | GGACCAAGAACGTGACGGCCCTGCTGATGGAGG | |
| | CGAAAGAGCTGGAGGCCCGGGTCATCATCCTTT | |
| | CTGCCAGCGAGGACGATGCTGCCACTGTATACC | |
| | GCGCAGCCGCGATGCTGAACATGACGGGCTCCG | |
| | GGTACGTGTGGCTGGTCGGCGAGCGCGAGATCT | |
| | CGGGGAACGCCCTGCGCTACGCCCCGGACGGC | |
| | ATCCTCGGGCTGCAGCTCATCAACGGCAAGAAC | |
| | GAGTCGGCCCACATCAGCGACGCCGTAGGCGTG | |
| | GTGGCCCAGGCCGTGCACGAGCTCCTCGAGAAG | |
| | GAGAACATCACCGACCCGCCGCGGGGCTGCGT | |
| | GGGCAACACCAACATCTGGAAGACCGGGCCGCT | |
| | CTTCAAGAGAGTGCTGATGTCTTCCAAGTATGCG | |
| | GATGGGGTGACTGGTCGCGTGGAGTTCAATGAG | |
| | GATGGGGACCGGAAGTTCGCCAACTACAGCATC | |
| | ATGAACCTGCAGAACCGCAAGCTGGTGCAAGTG | |
| | GGCATCTACAATGGCACCCACGTCATCCCTAATG | |
| | ACAGGAAGATCATCTGGCCAGGCGGAGAGACAG | |
| | AGAAGCCTCGAGGGTACCAGATGTCCACCAGAC | |
| | TGAAGATT | |
| SEQ ID NO | GAGAAGGGTCCCCCCGCGCTAAATATTGCGGTG | Amino terminal domain |
| 76 | ATGCTGGGTCACAGCCACGACGTGACAGAGCGC | of GluN2A as for |
| | GAACTTCGAACACTGTGGGGCCCCGAGCAGGCG | example used in fusion |
| | GCGGGGCTGCCCCTGGACGTGAACGTGGTAGCT | protein #7 |
| | CTGCTGATGAACCGCACCGACCCCAAGAGCCTC | |
| | ATCACGCACGTGTGCGACCTCATGTCCGGGGCA | |
| | CGCATCCACGGCCTCGTGTTTGGGGACGACACG | |
| | GACCAGGAGGCCGTAGCCCAGATGCTGGATTTT | |
| | ATCTCCTCCCACACCTTCGTCCCCATCTTGGGCA | |
| | TTCATGGGGGCGCATCTATGATCATGGCTGACAA | |
| | GGATCCGACGTCTACCTTCTTCCAGTTTGGAGCG | |
| | TCCATCCAGCAGCAAGCCACGGTCATGCTGAAG | |
| | ATCATGCAGGATTATGACTGGCATGTCTTCTCCC | |
| | TGGTGACCACTATCTTCCCTGGCTACAGGGAATT | |
| | CATCAGCTTCGTCAAGACCACAGTGGACAACAG | |
| | CTTTGTGGGCTGGGACATGCAGAATGTGATCACA | |
| | CTGGACACTTCCTTTGAGGATGCAAAGACACAAG | |
| | TCCAGCTGAAGAAGATCCACTCTTCTGTCATCTT | |
| | GCTCTACTGTTCCAAAGACGAGGCTGTTCTCATT | |
| | CTGAGTGAGGCCCGCTCCCTTGGCCTCACCGGG | |
| | TATGATTTCTTCTGGATTGTCCCCAGCTTGGTCT | |
| | CTGGGAACACGGAGCTCATCCCAAAAGAGTTTC | |
| | CATCGGGACTCATTTCTGTCTCCTACGATGACTG | |
| | GGACTACAGCCTGGAGGCGAGAGTGAGGGACG | |
| | GCATTGGCATCCTAACCACCGCTGCATCTTCTAT | |
| | GCTGGAGAAGTTCTCCTACATCCCCGAGGCCAA | |
| | GGCCAGCTGCTACGGGCAGATGGAGAGGCCAG | |
| | AGGTCCCGATGCACACCTTGCACCCATTTATGGT | |
| | CAATGTTACATGGGATGGCAAAGACTTATCCTTC | |
| | ACTGAGGAAGGCTACCAGGTGCACCCCAGGCTG | |
| | GTGGTGATTGTGCTGAACAAAGACCGGGAATGG | |
| | GAAAAGGTGGGCAAGTGGGAGAACCATACGCTG | |
| | AGCCTGAGGCACGCCGTGTGGCCCAGGTACAAG | |
| | TCCTTCTCCGACTGTGAGCCGGATGACAACCATC | |
| | TCAGCATC | |
| SEQ ID NO | ATGAGCACCATGCGCCTGCTGACGCTCGCCCTG | DNA seq of fusion |
| 77 | CTGTTCTCCTGCTCCGTCGCCCGTGCCGCGTGC | protein #8 N1-ATD- |
| | GACCCCAAGATCGTCAACATTGGCGCGGTGCTG | N2B-ATD-Fc |
| | AGCACGCGGAAGCACGAGCAGATGTTCCGCGAG | |
| | GCCGTGAACCAGGCCAACAAGCGGCACGGCTCC | |
| | TGGAAGATTCAGCTCAATGCCACCTCCGTCACGC | |
| | ACAAGCCCAACGCCATCCAGATGGCTCTGTCGG | |
| | TGTGCGAGGACCTCATCTCCAGCCAGGTCTACG | |
| | CCATCCTAGTTAGCCATCCACCTACCCCCAACGA | |
| | CCACTTCACTCCCACCCCTGTCTCCTACACAGCC | |
| | GGCTTCTACCGCATACCCGTGCTGGGGCTGACC | |
| | ACCCGCATGTCCATCTACTCGGACAAGAGCATCC | |
| | ACCTGAGCTTCCTGCGCACCGTGCCGCCCTACT | |
| | CCCACCAGTCCAGCGTGTGGTTTGAGATGATGC | |
| | GTGTCTACAGCTGGAACCACATCATCCTGCTGGT | |
| | CAGCGACGACCACGAGGGCCGGGCGGCTCAGA | |
| | AACGCCTGGAGACGCTGCTGGAGGAGCGTGAGT | |
| | CCAAGGCAGAGAAGGTGCTGCAGTTTGACCCAG | |
| | GGACCAAGAACGTGACGGCCCTGCTGATGGAGG | |
| | CGAAAGAGCTGGAGGCCCGGGTCATCATCCTTT | |
| | CTGCCAGCGAGGACGATGCTGCCACTGTATACC | |
| | GCGCAGCCGCGATGCTGAACATGACGGGCTCCG | |
| | GGTACGTGTGGCTGGTCGGCGAGCGCGAGATCT | |
| | CGGGGAACGCCCTGCGCTACGCCCCGGACGGC | |
| | ATCCTCGGGCTGCAGCTCATCAACGGCAAGAAC | |
| | GAGTCGGCCCACATCAGCGACGCCGTAGGCGTG | |
| | GTGGCCCAGGCCGTGCACGAGCTCCTCGAGAAG | |
| | GAGAACATCACCGACCCGCCGCGGGGCTGCGT | |
| | GGGCAACACCAACATCTGGAAGACCGGGCCGCT | |
| | CTTCAAGAGAGTGCTGATGTCTTCCAAGTATGCG | |
| | GATGGGGTGACTGGTCGCGTGGAGTTCAATGAG | |
| | GATGGGGACCGGAAGTTCGCCAACTACAGCATC | |
| | ATGAACCTGCAGAACCGCAAGCTGGTGCAAGTG | |
| | GGCATCTACAATGGCACCCACGTCATCCCTAATG | |
| | ACAGGAAGATCATCTGGCCAGGCGGAGAGACAG | |
| | AGAAGCCTCGAGGGTACCAGATGTCCACCAGAC | |
| | TGAAGATTGGGTCGACTGGTGGAGGAGGGTCTG | |
| | GCGGAGGTGGTTCCGGGGGCGGAGGATCTGGG | |
| | GCCGCTTCTAGACAGAAGAGCCCCCCCAGCATT | |
| | GGCATTGCTGTCATCCTCGTGGGCACTTCCGAC | |
| | GAGGTGGCCATCAAGGATGCCCACGAGAAAGAT | |
| | GATTTCCACCATCTCTCCGTGGTACCCCGGGTG | |
| | GAACTGGTAGCCATGAATGAGACCGACCCAAAG | |
| | AGCATCATCACCCGCATCTGTGATCTCATGTCTG | |
| | ACCGGAAGATCCAGGGGGTGGTGTTTGCTGATG | |
| | ACACAGACCAGGAAGCCATCGCCCAGATCCTCG | |
| | ATTTCATTTCAGCACAGACTCTCACCCCGATCCT | |
| | GGGCATCCACGGGGGCTCCTCTATGATAATGGC | |
| | AGATAAGGATGAATCCTCCATGTTCTTCCAGTTT | |
| | GGCCCATCAATTGAACAGCAAGCTTCCGTAATGC | |
| | TCAACATCATGGAAGAATATGACTGGTACATCTTT | |
| | TCTATCGTCACCACCTATTTCCCTGGCTACCAGG | |
| | ACTTTGTAAACAAGATCCGCAGCACCATTGAGAA | |
| | TAGCTTTGTGGGCTGGGAGCTAGAGGAGGTCCT | |
| | CCTACTGGACATGTCCCTGGACGATGGAGATTCT | |
| | AAGATCCAGAATCAGCTCAAGAAACTTCAAAGCC | |
| | CCATCATTCTTCTTTACTGTACCAAGGAAGAAGC | |
| | CACCTACATCTTTGAAGTGGCCAACTCAGTAGGG | |
| | CTGACTGGCTATGGCTACACGTGGATCGTGCCC | |
| | AGTCTGGTGGCAGGGGATACAGACACAGTGCCT | |
| | GCGGAGTTCCCCACTGGGCTCATCTCTGTATCAT | |
| | ATGATGAATGGGACTATGGCCTCCCCGCCAGAG | |
| | TGAGAGATGGAATTGCCATAATCACCACTGCTGC | |
| | TTCTGACATGCTGTCTGAGCACAGCTTCATCCCT | |
| | GAGCCCAAAAGCAGTTGTTACAACACCCACGAG | |
| | AAGAGAATCTACCAGTCCAATATGCTAAATAGGT | |
| | ATCTGATCAATGTCACTTTTGAGGGGAGGAATTT | |
| | GTCCTTCAGTGAAGATGGCTACCAGATGCACCC | |
| | GAAACTGGTGATAATTCTTCTGAACAAGGAGAGG | |
| | AAGTGGGAAAGGGTGGGGAAGTGGAAAGACAAG | |
| | TCCCTGCAGATGAAGTACTATGTGTGGCCCCGAA | |
| | TGTGTCCAGAGACTGAAGAGCAGGAGGATGACC | |
| | ATCTGAGCATTGGCTCGAGCACCATGGTTAGATC | |
| | TAGCAAGCCCACGTGCCCACCCCCTGAACTCCT | |
| | GGGGGGACCGTCTGTCTTCATCTTCCCCCCAAA | |
| | ACCCAAGGACACCCTCATGATCTCACGCACCCC | |
| | CGAGGTCACATGCGTGGTGGTGGACGTGAGCCA | |
| | GGATGACCCCGAGGTGCAGTTCACATGGTACAT | |
| | AAACAACGAGCAGGTGCGCACCGCCCGGCCGC | |
| | CGCTACGGGAGCAGCAGTTCAACAGCACGATCC | |
| | GCGTGGTCAGCACCCTCCCCATCGCGCACCAGG | |
| | ACTGGCTGAGGGGCAAGGAGTTCAAGTGCAAAG | |
| | TCCACAACAAGGCACTCCCGGCCCCCATCGAGA | |
| | AAACCATCTCCAAAGCCAGAGGGCAGCCCCTGG | |
| | AGCCGAAGGTCTACACCATGGGCCCTCCCCGGG | |
| | AGGAGCTGAGCAGCAGGTCGGTCAGCCTGACCT | |
| | GCATGATCAACGGCTTCTACCCTTCCGACATCTC | |
| | GGTGGAGTGGGAGAAGAACGGGAAGGCAGAGG | |
| | ACAACTACAAGACCACGCCGGCCGTGCTGGACA | |
| | GCGACGGCTCCTACTTCCTCTACAGCAAGCTCTC | |
| | AGTGCCCACGAGTGAGTGGCAGCGGGGCGACG | |
| | TCTTCACCTGCTCCGTGATGCACGAGGCCTTGCA | |
| | CAACCACTACACGCAGAAGTCCATCTCCCGCTCT | |
| | CCGGGTAAATGA | |
| SEQ ID NO | ATGAGCACCATGCGCCTGCTGACGCTCGCCCTG | Amino terminal domain |
| 78 | CTGTTCTCCTGCTCCGTCGCCCGTGCCGCGTGC | of GluN1 (including |
| | GACCCCAAGATCGTCAACATTGGCGCGGTGCTG | signal sequence) as |
| | AGCACGCGGAAGCACGAGCAGATGTTCCGCGAG | used for example in |
| | GCCGTGAACCAGGCCAACAAGCGGCACGGCTCC | fusion protein #8 |
| | TGGAAGATTCAGCTCAATGCCACCTCCGTCACGC | |
| | ACAAGCCCAACGCCATCCAGATGGCTCTGTCGG | |
| | TGTGCGAGGACCTCATCTCCAGCCAGGTCTACG | |
| | CCATCCTAGTTAGCCATCCACCTACCCCCAACGA | |
| | CCACTTCACTCCCACCCCTGTCTCCTACACAGCC | |
| | GGCTTCTACCGCATACCCGTGCTGGGGCTGACC | |
| | ACCCGCATGTCCATCTACTCGGACAAGAGCATCC | |
| | ACCTGAGCTTCCTGCGCACCGTGCCGCCCTACT | |
| | CCCACCAGTCCAGCGTGTGGTTTGAGATGATGC | |
| | GTGTCTACAGCTGGAACCACATCATCCTGCTGGT | |
| | CAGCGACGACCACGAGGGCCGGGCGGCTCAGA | |
| | AACGCCTGGAGACGCTGCTGGAGGAGCGTGAGT | |
| | CCAAGGCAGAGAAGGTGCTGCAGTTTGACCCAG | |
| | GGACCAAGAACGTGACGGCCCTGCTGATGGAGG | |
| | CGAAAGAGCTGGAGGCCCGGGTCATCATCCTTT | |
| | CTGCCAGCGAGGACGATGCTGCCACTGTATACC | |
| | GCGCAGCCGCGATGCTGAACATGACGGGCTCCG | |
| | GGTACGTGTGGCTGGTCGGCGAGCGCGAGATCT | |
| | CGGGGAACGCCCTGCGCTACGCCCCGGACGGC | |
| | ATCCTCGGGCTGCAGCTCATCAACGGCAAGAAC | |
| | GAGTCGGCCCACATCAGCGACGCCGTAGGCGTG | |
| | GTGGCCCAGGCCGTGCACGAGCTCCTCGAGAAG | |
| | GAGAACATCACCGACCCGCCGCGGGGCTGCGT | |
| | GGGCAACACCAACATCTGGAAGACCGGGCCGCT | |
| | CTTCAAGAGAGTGCTGATGTCTTCCAAGTATGCG | |
| | GATGGGGTGACTGGTCGCGTGGAGTTCAATGAG | |
| | GATGGGGACCGGAAGTTCGCCAACTACAGCATC | |
| | ATGAACCTGCAGAACCGCAAGCTGGTGCAAGTG | |
| | GGCATCTACAATGGCACCCACGTCATCCCTAATG | |
| | ACAGGAAGATCATCTGGCCAGGCGGAGAGACAG | |
| | AGAAGCCTCGAGGGTACCAGATGTCCACCAGAC | |
| | TGAAGATT | |
| SEQ ID NO | CAGAAGAGCCCCCCCAGCATTGGCATTGCTGTC | Amino terminal domain |
| 79 | ATCCTCGTGGGCACTTCCGACGAGGTGGCCATC | of GluN2B as for |
| | AAGGATGCCCACGAGAAAGATGATTTCCACCATC | example used in fusion |
| | TCTCCGTGGTACCCCGGGTGGAACTGGTAGCCA | protein #8 |
| | TGAATGAGACCGACCCAAAGAGCATCATCACCC | |
| | GCATCTGTGATCTCATGTCTGACCGGAAGATCCA | |
| | GGGGGTGGTGTTTGCTGATGACACAGACCAGGA | |
| | AGCCATCGCCCAGATCCTCGATTTCATTTCAGCA | |
| | CAGACTCTCACCCCGATCCTGGGCATCCACGGG | |
| | GGCTCCTCTATGATAATGGCAGATAAGGATGAAT | |
| | CCTCCATGTTCTTCCAGTTTGGCCCATCAATTGA | |
| | ACAGCAAGCTTCCGTAATGCTCAACATCATGGAA | |
| | GAATATGACTGGTACATCTTTTCTATCGTCACCAC | |
| | CTATTTCCCTGGCTACCAGGACTTTGTAAACAAG | |
| | ATCCGCAGCACCATTGAGAATAGCTTTGTGGGCT | |
| | GGGAGCTAGAGGAGGTCCTCCTACTGGACATGT | |
| | CCCTGGACGATGGAGATTCTAAGATCCAGAATCA | |
| | GCTCAAGAAACTTCAAAGCCCCATCATTCTTCTTT | |
| | ACTGTACCAAGGAAGAAGCCACCTACATCTTTGA | |
| | AGTGGCCAACTCAGTAGGGCTGACTGGCTATGG | |
| | CTACACGTGGATCGTGCCCAGTCTGGTGGCAGG | |
| | GGATACAGACACAGTGCCTGCGGAGTTCCCCAC | |
| | TGGGCTCATCTCTGTATCATATGATGAATGGGAC | |
| | TATGGCCTCCCCGCCAGAGTGAGAGATGGAATT | |
| | GCCATAATCACCACTGCTGCTTCTGACATGCTGT | |
| | CTGAGCACAGCTTCATCCCTGAGCCCAAAAGCA | |
| | GTTGTTACAACACCCACGAGAAGAGAATCTACCA | |
| | GTCCAATATGCTAAATAGGTATCTGATCAATGTCA | |
| | CTTTTGAGGGGAGGAATTTGTCCTTCAGTGAAGA | |
| | TGGCTACCAGATGCACCCGAAACTGGTGATAATT | |
| | CTTCTGAACAAGGAGAGGAAGTGGGAAAGGGTG | |
| | GGGAAGTGGAAAGACAAGTCCCTGCAGATGAAG | |
| | TACTATGTGTGGCCCCGAATGTGTCCAGAGACTG | |
| | AAGAGCAGGAGGATGACCATCTGAGCATT | |
| SEQ ID NO | ATGGGTGGGGCCCTGGGGCCGGCCCTGTTGCT | Human GluN2C |
| 80 | CACCTCGCTCTTCGGTGCCTGGGCAGGGCTGGG | NM_000835 |
| | TCCGGGGCAGGGCGAGCAGGGCATGACGGTGG | |
| | CCGTGGTGTTTAGCAGCTCAGGGCCGCCCCAGG | |
| | CCCAGTTCCGTGCCCGCCTCACCCCCCAGAGCT | |
| | TCCTGGACCTACCCCTGGAGATCCAGCCGCTCA | |
| | CAGTTGGGGTCAACACCACCAACCCCAGCAGCC | |
| | TCCTCACCCAGATCTGCGGCCTCCTGGGTGCTG | |
| | CCCACGTCCACGGCATTGTCTTTGAGGACAACGT | |
| | GGACACCGAGGCGGTGGCCCAGATCCTTGACTT | |
| | CATCTCCTCCCAGACCCATGTGCCCATCCTCAGC | |
| | ATCAGCGGAGGCTCTGCTGTGGTCCTCACCCCC | |
| | AAGGAGCCGGGCTCCGCCTTCCTGCAGCTGGGC | |
| | GTGTCCCTGGAGCAGCAGCTGCAGGTGCTGTTC | |
| | AAGGTGCTGGAAGAGTACGACTGGAGCGCCTTC | |
| | GCCGTCATCACCAGCCTGCACCCGGGCCACGCG | |
| | CTCTTCCTGGAGGGCGTGCGCGCCGTCGCCGAC | |
| | GCCAGCCACGTGAGTTGGCGGCTGCTGGACGTG | |
| | GTCACGCTGGAGCTGGGCCCGGGAGGGCCGCG | |
| | CGCGCGCACGCAGCGCCTGCTGCGCCAGCTCG | |
| | ACGCGCCCGTGTTTGTGGCCTACTGCTCGCGCG | |
| | AGGAGGCCGAGGTGCTCTTCGCCGAGGCGGCG | |
| | CAGGCCGGTCTGGTGGGGCCCGGCCACGTGTG | |
| | GCTGGTGCCCAACCTGGCGCTGGGCAGCACCG | |
| | ATGCGCCCCCCGCCACCTTCCCCGTGGGCCTCA | |
| | TCAGCGTCGTCACCGAGAGCTGGCGCCTCAGCC | |
| | TGCGCCAGAAGGTGCGCGACGGCGTGGCCATTC | |
| | TGGCCCTGGGCGCCCACAGCTACTGGCGCCAG | |
| | CATGGAACCCTGCCAGCCCCGGCCGGGGACTG | |
| | CCGTGTTCACCCTGGGCCCGTCAGCCCTGCCCG | |
| | GGAGGCCTTCTACAGGCACCTACTGAATGTCAC | |
| | CTGGGAGGGCCGAGACTTCTCCTTCAGCCCTGG | |
| | TGGGTACCTGGTCCAGCCCACCATGGTGGTGAT | |
| | CGCCCTCAACCGGCACCGCCTCTGGGAGATGGT | |
| | GGGGCGCTGGGAGCATGGCGTCCTATACATGAA | |
| | GTACCCCGTGTGGCCTCGCTACAGTGCCTCTCT | |
| | GCAGCCTGTGGTGGACAGTCGGCACCTGACGGT | |
| | GGCCACGCTGGAAGAGCGGCCCTTTGTCATCGT | |
| | GGAGAGCCCTGACCCTGGCACAGGAGGCTGTGT | |
| | CCCCAACACCGTGCCCTGCCGCAGGCAGAGCAA | |
| | CCACACCTTCAGCAGCGGGGACGTGGCCCCCTA | |
| | CACCAAGCTCTGCTGTAAGGGATTCTGCATCGAC | |
| | ATCCTCAAGAAGCTGGCCAGAGTGGTCAAATTCT | |
| | CCTACGACCTGTACCTGGTGACCAACGGCAAGC | |
| | ATGGCAAGCGGGTGCGCGGCGTATGGAACGGC | |
| | ATGATTGGGGAGGTGTACTACAAGCGGGCAGAC | |
| | ATGGCCATCGGCTCCCTCACCATCAATGAGGAA | |
| | CGCTCCGAGATCGTAGACTTCTCTGTACCCTTTG | |
| | TGGAGACGGGCATCAGTGTGATGGTGGCTCGCA | |
| | GCAATGGCACCGTCTCCCCCTCGGCCTTCTTGG | |
| | AGCCATATAGCCCTGCAGTGTGGGTGATGATGTT | |
| | TGTCATGTGCCTCACTGTGGTGGCCATCACCGTC | |
| | TTCATGTTCGAGTACTTCAGCCCTGTCAGCTACA | |
| | ACCAGAACCTCACCAGAGGCAAGAAGTCCGGGG | |
| | GCCCAGCTTTCACTATCGGCAAGTCCGTGTGGC | |
| | TGCTGTGGGCGCTGGTCTTCAACAACTCAGTGC | |
| | CCATCGAGAACCCGCGGGGCACCACCAGCAAGA | |
| | TCATGGTTCTGGTCTGGGCCTTCTTTGCTGTCAT | |
| | CTTCCTCGCCAGCTACACGGCCAACCTGGCCGC | |
| | CTTCATGATCCAAGAGCAATACATCGACACTGTG | |
| | TCGGGCCTCAGTGACAAGAAGTTTCAGCGGCCT | |
| | CAAGATCAGTACCCACCTTTCCGCTTCGGCACG | |
| | GTGCCCAACGGCAGCACGGAGCGGAACATCCG | |
| | CAGTAACTACCGTGACATGCACACCCACATGGTC | |
| | AAGTTCAACCAGCGCTCGGTGGAGGACGCGCTC | |
| | ACCAGCCTCAAGATGGGGAAGCTGGATGCCTTC | |
| | ATCTATGATGCTGCTGTCCTCAACTACATGGCAG | |
| | GCAAGGACGAGGGCTGCAAGCTGGTCACCATTG | |
| | GGTCTGGCAAGGTCTTTGCTACCACTGGCTACG | |
| | GCATCGCCATGCAGAAGGACTCCCACTGGAAGC | |
| | GGGCCATAGACCTGGCGCTCTTGCAGTTCCTGG | |
| | GGGACGGAGAGACACAGAAACTGGAGACAGTGT | |
| | GGCTCTCAGGGATCTGCCAGAATGAGAAGAACG | |
| | AGGTGATGAGCAGCAAGCTGGACATCGACAACA | |
| | TGGCAGGCGTCTTCTACATGCTGCTGGTGGCCA | |
| | TGGGGCTGGCCCTGCTGGTCTTCGCCTGGGAGC | |
| | ACCTGGTCTACTGGAAGCTGCGCCACTCGGTGC | |
| | CCAACTCATCCCAGCTGGACTTCCTGCTGGCTTT | |
| | CAGCAGGGGCATCTACAGCTGCTTCAGCGGGGT | |
| | GCAGAGCCTCGCCAGCCCACCGCGGCAGGCCA | |
| | GCCCGGACCTCACGGCCAGCTCGGCCCAGGCC | |
| | AGCGTGCTCAAGATGCTGCAGGCAGCCCGCGAC | |
| | ATGGTGACCACGGCGGGCGTAAGCAGCTCCCTG | |
| | GACCGCGCCACTCGCACCATCGAGAATTGGGGT | |
| | GGCGGCCGCCGTGCGCCCCCACCGTCCCCCTG | |
| | CCCGACCCCGCGGTCTGGCCCCAGCCCATGCCT | |
| | GCCCACCCCCGACCCGCCCCCAGAGCCGAGCC | |
| | CCACGGGCTGGGGACCGCCAGACGGGGGTCGC | |
| | GCGGCGCTTGTGCGCAGGGCTCCGCAGCCCCC | |
| | GGGCCGCCCCCCGACGCCGGGGCCGCCCCTGT | |
| | CCGACGTCTCCCGAGTGTCGCGCCGCCCAGCCT | |
| | GGGAGGCGCGGTGGCCGGTGCGGACCGGGCAC | |
| | TGCGGGAGGCACCTCTCGGCCTCCGAGCGGCC | |
| | CCTGTCGCCCGCGCGCTGTCACTACAGCTCCTT | |
| | TCCTCGAGCCGACCGATCCGGCCGCCCCTTCCT | |
| | CCCGCTCTTCCCGGAGCTGGAGGACCTGCCGCT | |
| | GCTCGGTCCGGAGCAGCTGGCCCGGCGGGAGG | |
| | CCCTGCTGCACGCGGCCTGGGCCCGGGGCTCG | |
| | CGCCCGCGTCACGCTTCCCTGCCCAGCTCCGTG | |
| | GCCGAGGCCTTCGCTCGGCCCAGCTCGCTGCCC | |
| | GCTGGGTGCACCGGCCCCGCCTGCGCCCGCCC | |
| | CGACGGCCACTCGGCCTGCAGGCGCTTGGCGC | |
| | AGGCGCAGTCGATGTGCTTGCCGATCTACCGGG | |
| | AGGCCTGCCAGGAGGGCGAGCAGGCAGGGGCC | |
| | CCCGCCTGGCAGCACAGACAGCACGTCTGCCTG | |
| | CACGCCCACGCCCACCTGCCATTTTGCTGGGGG | |
| | GCTGTCTGTCCTCACCTTCCACCCTGTGCCAGCC | |
| | ACGGCTCCTGGCTCTCCGGGGCCTGGGGGCCT | |
| | CTGGGGCACAGGGGCAGGACTCTGGGGCTGGG | |
| | CACAGGCTACAGAGACAGTGGGGGACTGGACGA | |
| | GATCAGCAGGGTAGCCCGTGGGACGCAAGGCTT | |
| | CCCGGGACCCTGCACCTGGAGACGGATCTCCAG | |
| | TCTGGAGTCAGAAGTGTGA | |
| SEQ ID NO | CAGGGCATGACGGTGGCCGTGGTGTTTAGCAGC | GluN2C ATD |
| 81 | TCAGGGCCGCCCCAGGCCCAGTTCCGTGCCCG | |
| | CCTCACCCCCCAGAGCTTCCTGGACCTACCCCT | |
| | GGAGATCCAGCCGCTCACAGTTGGGGTCAACAC | |
| | CACCAACCCCAGCAGCCTCCTCACCCAGATCTG | |
| | CGGCCTCCTGGGTGCTGCCCACGTCCACGGCAT | |
| | TGTCTTTGAGGACAACGTGGACACCGAGGCGGT | |
| | GGCCCAGATCCTTGACTTCATCTCCTCCCAGACC | |
| | CATGTGCCCATCCTCAGCATCAGCGGAGGCTCT | |
| | GCTGTGGTCCTCACCCCCAAGGAGCCGGGCTCC | |
| | GCCTTCCTGCAGCTGGGCGTGTCCCTGGAGCAG | |
| | CAGCTGCAGGTGCTGTTCAAGGTGCTGGAAGAG | |
| | TACGACTGGAGCGCCTTCGCCGTCATCACCAGC | |
| | CTGCACCCGGGCCACGCGCTCTTCCTGGAGGGC | |
| | GTGCGCGCCGTCGCCGACGCCAGCCACGTGAG | |
| | TTGGCGGCTGCTGGACGTGGTCACGCTGGAGCT | |
| | GGGCCCGGGAGGGCCGCGCGCGCGCACGCAG | |
| | CGCCTGCTGCGCCAGCTCGACGCGCCCGTGTTT | |
| | GTGGCCTACTGCTCGCGCGAGGAGGCCGAGGT | |
| | GCTCTTCGCCGAGGCGGCGCAGGCCGGTCTGG | |
| | TGGGGCCCGGCCACGTGTGGCTGGTGCCCAAC | |
| | CTGGCGCTGGGCAGCACCGATGCGCCCCCCGC | |
| | CACCTTCCCCGTGGGCCTCATCAGCGTCGTCAC | |
| | CGAGAGCTGGCGCCTCAGCCTGCGCCAGAAGGT | |
| | GCGCGACGGCGTGGCCATTCTGGCCCTGGGCG | |
| | CCCACAGCTACTGGCGCCAGCATGGAACCCTGC | |
| | CAGCCCCGGCCGGGGACTGCCGTGTTCACCCTG | |
| | GGCCCGTCAGCCCTGCCCGGGAGGCCTTCTACA | |
| | GGCACCTACTGAATGTCACCTGGGAGGGCCGAG | |
| | ACTTCTCCTTCAGCCCTGGTGGGTACCTGGTCCA | |
| | GCCCACCATGGTGGTGATCGCCCTCAACCGGCA | |
| | CCGCCTCTGGGAGATGGTGGGGCGCTGGGAGC | |
| | ATGGCGTCCTATACATGAAGTACCCCGTGTGGC | |
| | CTCGC | |
| SEQ ID NO | AGTCGGCACCTGACGGTGGCCACGCTGGAAGAG | GluN2C S1 |
| 82 | CGGCCCTTTGTCATCGTGGAGAGCCCTGACCCT | |
| | GGCACAGGAGGCTGTGTCCCCAACACCGTGCCC | |
| | TGCCGCAGGCAGAGCAACCACACCTTCAGCAGC | |
| | GGGGACGTGGCCCCCTACACCAAGCTCTGCTGT | |
| | AAGGGATTCTGCATCGACATCCTCAAGAAGCTGG | |
| | CCAGAGTGGTCAAATTCTCCTACGACCTGTACCT | |
| | GGTGACCAACGGCAAGCATGGCAAGCGGGTGC | |
| | GCGGCGTATGGAACGGCATGATTGGGGAGGTGT | |
| | ACTACAAGCGGGCAGACATGGCCATCGGCTCCC | |
| | TCACCATCAATGAGGAACGCTCCGAGATCGTAGA | |
| | CTTCTCTGTACCCTTTGTGGAGACGGGCATCAGT | |
| | GTGATGGTGGCTCGC | |
| SEQ ID NO | ACTGTGTCGGGCCTCAGTGACAAGAAGTTTCAG | GluN2C S2 |
| 83 | CGGCCTCAAGATCAGTACCCACCTTTCCGCTTCG | |
| | GCACGGTGCCCAACGGCAGCACGGAGCGGAAC | |
| | ATCCGCAGTAACTACCGTGACATGCACACCCACA | |
| | TGGTCAAGTTCAACCAGCGCTCGGTGGAGGACG | |
| | CGCTCACCAGCCTCAAGATGGGGAAGCTGGATG | |
| | CCTTCATCTATGATGCTGCTGTCCTCAACTACAT | |
| | GGCAGGCAAGGACGAGGGCTGCAAGCTGGTCA | |
| | CCATTGGGTCTGGCAAGGTCTTTGCTACCACTGG | |
| | CTACGGCATCGCCATGCAGAAGGACTCCCACTG | |
| | GAAGCGGGCCATAGACCTGGCGCTCTTGCAGTT | |
| | CCTGGGGGACGGAGAGACACAGAAACTGGAGAC | |
| | AGTGTGGCTCTCAGGGATCTGCCAGAAT | |
| SEQ ID NO | ATGGGTGGGGCCCTGGGGCCGGCCCTGTTGCT | GluN2C ecd incl. |
| 84 | CACCTCGCTCTTCGGTGCCTGGGCAGGGCTGGG | signal sequence and |
| | TCCGGGGCAGGGCGAGCAGGGCATGACGGTGG | GT-linker between S1 |
| | CCGTGGTGTTTAGCAGCTCAGGGCCGCCCCAGG | and S2 |
| | CCCAGTTCCGTGCCCGCCTCACCCCCCAGAGCT | |
| | TCCTGGACCTACCCCTGGAGATCCAGCCGCTCA | |
| | CAGTTGGGGTCAACACCACCAACCCCAGCAGCC | |
| | TCCTCACCCAGATCTGCGGCCTCCTGGGTGCTG | |
| | CCCACGTCCACGGCATTGTCTTTGAGGACAACGT | |
| | GGACACCGAGGCGGTGGCCCAGATCCTTGACTT | |
| | CATCTCCTCCCAGACCCATGTGCCCATCCTCAGC | |
| | ATCAGCGGAGGCTCTGCTGTGGTCCTCACCCCC | |
| | AAGGAGCCGGGCTCCGCCTTCCTGCAGCTGGGC | |
| | GTGTCCCTGGAGCAGCAGCTGCAGGTGCTGTTC | |
| | AAGGTGCTGGAAGAGTACGACTGGAGCGCCTTC | |
| | GCCGTCATCACCAGCCTGCACCCGGGCCACGCG | |
| | CTCTTCCTGGAGGGCGTGCGCGCCGTCGCCGAC | |
| | GCCAGCCACGTGAGTTGGCGGCTGCTGGACGTG | |
| | GTCACGCTGGAGCTGGGCCCGGGAGGGCCGCG | |
| | CGCGCGCACGCAGCGCCTGCTGCGCCAGCTCG | |
| | ACGCGCCCGTGTTTGTGGCCTACTGCTCGCGCG | |
| | AGGAGGCCGAGGTGCTCTTCGCCGAGGCGGCG | |
| | CAGGCCGGTCTGGTGGGGCCCGGCCACGTGTG | |
| | GCTGGTGCCCAACCTGGCGCTGGGCAGCACCG | |
| | ATGCGCCCCCCGCCACCTTCCCCGTGGGCCTCA | |
| | TCAGCGTCGTCACCGAGAGCTGGCGCCTCAGCC | |
| | TGCGCCAGAAGGTGCGCGACGGCGTGGCCATTC | |
| | TGGCCCTGGGCGCCCACAGCTACTGGCGCCAG | |
| | CATGGAACCCTGCCAGCCCCGGCCGGGGACTG | |
| | CCGTGTTCACCCTGGGCCCGTCAGCCCTGCCCG | |
| | GGAGGCCTTCTACAGGCACCTACTGAATGTCAC | |
| | CTGGGAGGGCCGAGACTTCTCCTTCAGCCCTGG | |
| | TGGGTACCTGGTCCAGCCCACCATGGTGGTGAT | |
| | CGCCCTCAACCGGCACCGCCTCTGGGAGATGGT | |
| | GGGGCGCTGGGAGCATGGCGTCCTATACATGAA | |
| | GTACCCCGTGTGGCCTCGCTACAGTGCCTCTCT | |
| | GCAGCCTGTGGTGGACAGTCGGCACCTGACGGT | |
| | GGCCACGCTGGAAGAGCGGCCCTTTGTCATCGT | |
| | GGAGAGCCCTGACCCTGGCACAGGAGGCTGTGT | |
| | CCCCAACACCGTGCCCTGCCGCAGGCAGAGCAA | |
| | CCACACCTTCAGCAGCGGGGACGTGGCCCCCTA | |
| | CACCAAGCTCTGCTGTAAGGGATTCTGCATCGAC | |
| | ATCCTCAAGAAGCTGGCCAGAGTGGTCAAATTCT | |
| | CCTACGACCTGTACCTGGTGACCAACGGCAAGC | |
| | ATGGCAAGCGGGTGCGCGGCGTATGGAACGGC | |
| | ATGATTGGGGAGGTGTACTACAAGCGGGCAGAC | |
| | ATGGCCATCGGCTCCCTCACCATCAATGAGGAA | |
| | CGCTCCGAGATCGTAGACTTCTCTGTACCCTTTG | |
| | TGGAGACGGGCATCAGTGTGATGGTGGCTCGCg | |
| | gcaccACTGTGTCGGGCCTCAGTGACAAGAAGTTT | |
| | CAGCGGCCTCAAGATCAGTACCCACCTTTCCGCT | |
| | TCGGCACGGTGCCCAACGGCAGCACGGAGCGG | |
| | AACATCCGCAGTAACTACCGTGACATGCACACCC | |
| | ACATGGTCAAGTTCAACCAGCGCTCGGTGGAGG | |
| | ACGCGCTCACCAGCCTCAAGATGGGGAAGCTGG | |
| | ATGCCTTCATCTATGATGCTGCTGTCCTCAACTA | |
| | CATGGCAGGCAAGGACGAGGGCTGCAAGCTGGT | |
| | CACCATTGGGTCTGGCAAGGTCTTTGCTACCACT | |
| | GGCTACGGCATCGCCATGCAGAAGGACTCCCAC | |
| | TGGAAGCGGGCCATAGACCTGGCGCTCTTGCAG | |
| | TTCCTGGGGGACGGAGAGACACAGAAACTGGAG | |
| | ACAGTGTGGCTCTCAGGGATCTGCCAGAAT | |
| SEQ ID NO | ATGCGCGGCGCCGGTGGCCCCCGCGGCCCTCG | Human GluN2D |
| 85 | GGGCCCCGCTAAGATGCTGCTGCTGCTGGCGCT | U77783 (DNA) |
| | GGCCTGCGCCAGCCCGTTCCCGGAGGAGGCGC | |
| | CGGGGCCGGGCGGGGCCGGTGGGCCCGGCGG | |
| | CGGCCTCGGCGGGGCGCGGCCGCTCAACGTGG | |
| | CGCTCGTGTTCTCGGGGCCCGCGTACGCGGCC | |
| | GAGGCGGCACGCCTGGGCCCGGCCGTGGCGGC | |
| | GGCGGTGCGCAGCCCGGGCCTAGACGTGCGGC | |
| | CCGTGGCGCTGGTGCTCAACGGCTCGGACCCG | |
| | CGCAGCCTCGTGCTGCAGCTCTGCGACCTGCTG | |
| | TCGGGGTTGCGCGTGCACGGCGTGGTCTTCGAA | |
| | GACGACTCGCGCGCGCCCGCCGTCGCGCCCAT | |
| | CCTCGACTTCCTGTCGGCGCAGACCTCGCTGCC | |
| | CATCGTGGCCGTGCACGGCGGCGCCGCGCTCG | |
| | TGCTCACGCCCAAGGAGAAGGGCTCCACCTTCC | |
| | TGCAGCTGGGCTCTTCCACCGAGCAACAGCTTC | |
| | AGGTCATCTTTGAGGTGCTGGAGGAGTATGACT | |
| | GGACGTCCTTTGTAGCCGTGACCACTCGTGCCC | |
| | CTGGCCACCGGGCCTTCCTGTCCTACATTGAGG | |
| | TGCTGACTGACGGTAGTCTGGTGGGCTGGGAGC | |
| | ACCGCGGAGCGCTGACGCTGGACCCTGGGGCG | |
| | GGCGAGGCCGTGCTCAGTGCCCAGCTCCGCAGT | |
| | GTCAGCGCGCAGATCCGCCTGCTCTTCTGCGCC | |
| | CGAGAGGAGGCCGAGCCCGTGTTCCGCGCAGC | |
| | TGAGGAGGCTGGCCTCACTGGATCTGGCTACGT | |
| | CTGGTTCATGGTGGGGCCCCAGCTGGCTGGAGG | |
| | CGGGGGCTCTGGGGCCCCTGGTGAGCCCCCTC | |
| | TTCTGCCAGGAGGCGCCCCCCTGCCTGCCGGG | |
| | CTGTTTGCAGTGCGCTCGGCTGGCTGGCGGGAT | |
| | GACCTGGCTCGGCGAGTGGCAGCTGGCGTGGC | |
| | CGTAGTGGCCAGAGGTGCCCAGGCCCTGCTGC | |
| | GTGATTATGGTTTCCTTCCTGAGCTCGGCCACGA | |
| | CTGTCGCGCCCAGAACCGCACCCACCGCGGCG | |
| | AGAGTCTGCATAGGTACTTCATGAACATCACGTG | |
| | GGATAACCGGGATTACTCCTTCAATGAGGACGG | |
| | CTTCCTAGTGAACCCCTCCCTGGTGGTCATCTCC | |
| | CTCACCAGAGACAGGACGTGGGAGGTGGTGGG | |
| | CAGCTGGGAGCAGCAGACGCTCCGCCTCAAGTA | |
| | CCCGCTGTGGTCCCGCTATGGTCGCTTCCTGCA | |
| | GCCAGTGGACGACACGCAGCACCTCACGGTGGC | |
| | CACGCTGGAGGAAAGGCCGTTTGTCATCGTGGA | |
| | GCCTGCAGACCCTATCAGCGGCACCTGCATCCG | |
| | AGACTCCGTCCCCTGCCGGAGCCAGCTCAACCG | |
| | AACCCACAGCCCTCCACCGGATGCCCCCCGCCC | |
| | GGAAAAGCGCTGCTGCAAGGGTTTCTGCATCGA | |
| | CATTCTGAAGCGGCTGGCGCATACCATCGGCTT | |
| | CAGCTACGACCTCTACCTGGTCACCAATGGCAA | |
| | GCACGGAAAGAAGATCGATGGCGTCTGGAACGG | |
| | CATGATCGGGGAGGTGTTCTACCAGCGCGCAGA | |
| | CATGGCCATCGGCTCCCTCACCATCAACGAGGA | |
| | GCGCTCCGAGATCGTGGACTTCTCCGTCCCCTT | |
| | CGTGGAGACCGGCATCAGCGTCATGGTGGCGC | |
| | GCAGCAATGGCACGGTGTCCCCCTCGGCCTTCC | |
| | TCGAGCCCTACAGCCCCGCCGTGTGGGTGATGA | |
| | TGTTCGTCATGTGCCTCACTGTGGTCGCCGTCAC | |
| | TGTTTTCATCTTCGAGTACCTCAGTCCTGTTGGTT | |
| | ACAACCGCAGCCTGGCCACGGGCAAGCGCCCT | |
| | GGCGGTTCAACCTTCACCATTGGGAAATCCATCT | |
| | GGCTGCTCTGGGCCCTGGTGTTCAATAATTCGGT | |
| | GCCCGTGGAGAACCCCCGGGGAACCACCAGCA | |
| | AAATCATGGTGCTGGTGTGGGCCTTCTTCGCCGT | |
| | CATCTTCCTCGCCAGCTACACAGCCAACCTGGC | |
| | CGCCTTCATGATCCAGGAGGAGTACGTGGATAC | |
| | TGTGTCTGGGCTCAGTGACCGCAAGTTCCAGAG | |
| | GCCCCAGGAGCAGTACCCGCCCCTGAAGTTTGG | |
| | GACCGTGCCCAACGGCTCCACGGAGAAGAACAT | |
| | CCGCAGCAACTATCCCGACATGCACAGCTACAT | |
| | GGTGCGCTACAACCAGCCCCGCGTAGAGGAAGC | |
| | GCTCACTCAGCTCAAGGCAGGGAAGCTGGACGC | |
| | CTTCATCTACGATGCTGCAGTGCTCAATTACATG | |
| | GCCCGCAAGGACGAGGGCTGCAAGCTTGTCACC | |
| | ATCGGCTCCGGCAAGGTCTTCGCCACGACAGGC | |
| | TATGGCATCGCCCTGCACAAGGGCTCCCGCTGG | |
| | AAGCGGCCCATCGACCTGGCGTTGCTGCAGTTC | |
| | CTGGGGGATGATGAGATCGAGATGCTGGAGCGG | |
| | CTGTGGCTCTCTGGGATCTGCCACAATGACAAAA | |
| | TCGAGGTGATGAGCAGCAAGCTGGACATCGACA | |
| | ACATGGCGGGCGTCTTCTACATGCTCCTGGTGG | |
| | CCATGGGCCTGTCCCTGCTGGTCTTCGCCTGGG | |
| | AGCACCTGGTGTACTGGCGCCTGCGGCACTGCC | |
| | TGGGGCCCACCCACCGCATGGACTTCCTGCTGG | |
| | CCTTCTCCAGGGGCATGTACAGCTGCTGCAGCG | |
| | CTGAGGCCGCCCCACCGCCCGCCAAGCCCCCG | |
| | CCGCCGCCACAGCCCCTGCCCAGCCCCGCGTA | |
| | CCCCGCGCCGGGGCCGGCTCCCGGGCCCGCAC | |
| | CTTTCGTGCCCCGCGAGCGCGCCTCAGTGGACC | |
| | GCTGGCGCCGGACCAAGGGCGCGGGGCCGCCG | |
| | GGGGGCGCGGGCCTGGCCGACGGCTTCCACCG | |
| | CTACTACGGCCCCATCGAGCCGCAGGGCCTAGG | |
| | CCTCGGCCTGGGCGAAGCGCGCGCGGCACCGC | |
| | GGGGCGCAGCCGGGCGCCCGCTGTCCCCGCCG | |
| | GCCGCTCAGCCCCCGCAGAAGCCGCCGGCCTC | |
| | CTATTTCGCCATCGTACGCGACAAGGAGCCAGC | |
| | CGAGCCCCCCGCCGGCGCCTTCCCCGGCTTCC | |
| | CGTCGCCGCCCGCGCCCCCCGCCGCCGCGGCC | |
| | ACCGCCGTCGGGCCGCCACTCTGCCGCTTGGCC | |
| | TTCGAGGACGAGAGCCCGCCGGCGCCCGCGCG | |
| | GTGGCCGCGCTCGGACCCCGAGAGCCAACCCC | |
| | TGCTGGGGCCAGGCGCGGGCGGCGCGGGGGG | |
| | CACGGGGGGCGCAGGCGGAGGAGCCCCGGCC | |
| | GCTCCGCCCCCGTGCTGCGCCGCGCCGCCCCC | |
| | GTGCCCTTACCTCGATCTCGAGCCGTCGCCGTC | |
| | GGACTCGGAGGACTCGGAGAGCCTGGGCGGCG | |
| | CGTCGCTGGGCGGCCTGGATCCCTGGTGGTTCG | |
| | CCGACTTCCCTTACCCGTATGCCGAGCGCCTCG | |
| | GGCCGCCGCCCGGCCGCTACTGGTCGGTCGAC | |
| | AAGCTCGGGGGCTGGCGCGCCGGGAGCTGGGA | |
| | CTACCTGCCCCCGCGCAGCGGTCCGGCCGCCT | |
| | GGCACTGTCGGCACTGCGCCAGCCTGGAGCTGC | |
| | TGCCGCCGCCGCGCCATCTCAGCTGCTCGCACG | |
| | ATGGCCTGGACGGCGGCTGGTGGGCGCCACCG | |
| | CCTCCACCCTGGGCCGCCGGGCCCCTGCCCCG | |
| | ACGCCGGGCCCGCTGCGGGTGCCCGCGGTCGC | |
| | ACCCGCACCGCCCGCGGGCCTCGCACCGCACG | |
| | CCCGCCGCTGCCGCGCCCCACCACCACAGGCA | |
| | CCGGCGCGCCGCTGGGGGCTGGGACCTCCCGC | |
| | CGCCCGCGCCCACCTCGCGCTCGCTCGAGGAC | |
| | CTCAGCTCGTGCCCTCGCGCCGCCCCTGCGCGC | |
| | AGGCTTACCGGGCCCTCCCGCCACGCTCGCAGG | |
| | TGTCCGCACGCCGCGCACTGGGGGCCGCCGCT | |
| | GCCCACAGCTTCCCACCGGAGACACCGGGGCG | |
| | GGGACCTGGGCACCCGCAGGGGCTCGGCGCAC | |
| | TTCTCTAGCCTCGAGTCCGAGGTATGA | |
| SEQ ID NO | CGGCCGCTCAACGTGGCGCTCGTGTTCTCGGGG | GluN2D ATD |
| 86 | CCCGCGTACGCGGCCGAGGCGGCACGCCTGGG | |
| | CCCGGCCGTGGCGGCGGCGGTGCGCAGCCCGG | |
| | GCCTAGACGTGCGGCCCGTGGCGCTGGTGCTCA | |
| | ACGGCTCGGACCCGCGCAGCCTCGTGCTGCAG | |
| | CTCTGCGACCTGCTGTCGGGGTTGCGCGTGCAC | |
| | GGCGTGGTCTTCGAAGACGACTCGCGCGCGCCC | |
| | GCCGTCGCGCCCATCCTCGACTTCCTGTCGGCG | |
| | CAGACCTCGCTGCCCATCGTGGCCGTGCACGGC | |
| | GGCGCCGCGCTCGTGCTCACGCCCAAGGAGAA | |
| | GGGCTCCACCTTCCTGCAGCTGGGCTCTTCCAC | |
| | CGAGCAACAGCTTCAGGTCATCTTTGAGGTGCTG | |
| | GAGGAGTATGACTGGACGTCCTTTGTAGCCGTG | |
| | ACCACTCGTGCCCCTGGCCACCGGGCCTTCCTG | |
| | TCCTACATTGAGGTGCTGACTGACGGTAGTCTGG | |
| | TGGGCTGGGAGCACCGCGGAGCGCTGACGCTG | |
| | GACCCTGGGGCGGGCGAGGCCGTGCTCAGTGC | |
| | CCAGCTCCGCAGTGTCAGCGCGCAGATCCGCCT | |
| | GCTCTTCTGCGCCCGAGAGGAGGCCGAGCCCGT | |
| | GTTCCGCGCAGCTGAGGAGGCTGGCCTCACTGG | |
| | ATCTGGCTACGTCTGGTTCATGGTGGGGCCCCA | |
| | GCTGGCTGGAGGCGGGGGCTCTGGGGCCCCTG | |
| | GTGAGCCCCCTCTTCTGCCAGGAGGCGCCCCCC | |
| | TGCCTGCCGGGCTGTTTGCAGTGCGCTCGGCTG | |
| | GCTGGCGGGATGACCTGGCTCGGCGAGTGGCA | |
| | GCTGGCGTGGCCGTAGTGGCCAGAGGTGCCCA | |
| | GGCCCTGCTGCGTGATTATGGTTTCCTTCCTGAG | |
| | CTCGGCCACGACTGTCGCGCCCAGAACCGCACC | |
| | CACCGCGGCGAGAGTCTGCATAGGTACTTCATG | |
| | AACATCACGTGGGATAACCGGGATTACTCCTTCA | |
| | ATGAGGACGGCTTCCTAGTGAACCCCTCCCTGG | |
| | TGGTCATCTCCCTCACCAGAGACAGGACGTGGG | |
| | AGGTGGTGGGCAGCTGGGAGCAGCAGACGCTC | |
| | CGCCTCAAGTACCCGCTGTGGTCCCGC | |
| SEQ ID NO | ACGCAGCACCTCACGGTGGCCACGCTGGAGGAA | GluN2D S1 |
| 87 | AGGCCGTTTGTCATCGTGGAGCCTGCAGACCCT | |
| | ATCAGCGGCACCTGCATCCGAGACTCCGTCCCC | |
| | TGCCGGAGCCAGCTCAACCGAACCCACAGCCCT | |
| | CCACCGGATGCCCCCCGCCCGGAAAAGCGCTG | |
| | CTGCAAGGGTTTCTGCATCGACATTCTGAAGCGG | |
| | CTGGCGCATACCATCGGCTTCAGCTACGACCTCT | |
| | ACCTGGTCACCAATGGCAAGCACGGAAAGAAGA | |
| | TCGATGGCGTCTGGAACGGCATGATCGGGGAGG | |
| | TGTTCTACCAGCGCGCAGACATGGCCATCGGCT | |
| | CCCTCACCATCAACGAGGAGCGCTCCGAGATCG | |
| | TGGACTTCTCCGTCCCCTTCGTGGAGACCGGCA | |
| | TCAGCGTCATGGTGGCGCGC | |
| SEQ ID NO | ACTGTGTCTGGGCTCAGTGACCGCAAGTTCCAG | GluN2D S2 |
| 88 | AGGCCCCAGGAGCAGTACCCGCCCCTGAAGTTT | |
| | GGGACCGTGCCCAACGGCTCCACGGAGAAGAAC | |
| | ATCCGCAGCAACTATCCCGACATGCACAGCTACA | |
| | TGGTGCGCTACAACCAGCCCCGCGTAGAGGAAG | |
| | CGCTCACTCAGCTCAAGGCAGGGAAGCTGGACG | |
| | CCTTCATCTACGATGCTGCAGTGCTCAATTACAT | |
| | GGCCCGCAAGGACGAGGGCTGCAAGCTTGTCAC | |
| | CATCGGCTCCGGCAAGGTCTTCGCCACGACAGG | |
| | CTATGGCATCGCCCTGCACAAGGGCTCCCGCTG | |
| | GAAGCGGCCCATCGACCTGGCGTTGCTGCAGTT | |
| | CCTGGGGGATGATGAGATCGAGATGCTGGAGCG | |
| | GCTGTGGCTCTCTGGGATCTGCCACAAT | |
| SEQ ID NO | ATGCGCGGCGCCGGTGGCCCCCGCGGCCCTCG | GluN2D ecd incl. |
| 89 | GGGCCCCGCTAAGATGCTGCTGCTGCTGGCGCT | signal sequence and |
| | GGCCTGCGCCAGCCCGTTCCCGGAGGAGGCGC | GT-linker between S1 |
| | CGGGGCCGGGCGGGGCCGGTGGGCCCGGCGG | and S2 |
| | CGGCCTCGGCGGGGCGCGGCCGCTCAACGTGG | |
| | CGCTCGTGTTCTCGGGGCCCGCGTACGCGGCC | |
| | GAGGCGGCACGCCTGGGCCCGGCCGTGGCGGC | |
| | GGCGGTGCGCAGCCCGGGCCTAGACGTGCGGC | |
| | CCGTGGCGCTGGTGCTCAACGGCTCGGACCCG | |
| | CGCAGCCTCGTGCTGCAGCTCTGCGACCTGCTG | |
| | TCGGGGTTGCGCGTGCACGGCGTGGTCTTCGAA | |
| | GACGACTCGCGCGCGCCCGCCGTCGCGCCCAT | |
| | CCTCGACTTCCTGTCGGCGCAGACCTCGCTGCC | |
| | CATCGTGGCCGTGCACGGCGGCGCCGCGCTCG | |
| | TGCTCACGCCCAAGGAGAAGGGCTCCACCTTCC | |
| | TGCAGCTGGGCTCTTCCACCGAGCAACAGCTTC | |
| | AGGTCATCTTTGAGGTGCTGGAGGAGTATGACT | |
| | GGACGTCCTTTGTAGCCGTGACCACTCGTGCCC | |
| | CTGGCCACCGGGCCTTCCTGTCCTACATTGAGG | |
| | TGCTGACTGACGGTAGTCTGGTGGGCTGGGAGC | |
| | ACCGCGGAGCGCTGACGCTGGACCCTGGGGCG | |
| | GGCGAGGCCGTGCTCAGTGCCCAGCTCCGCAGT | |
| | GTCAGCGCGCAGATCCGCCTGCTCTTCTGCGCC | |
| | CGAGAGGAGGCCGAGCCCGTGTTCCGCGCAGC | |
| | TGAGGAGGCTGGCCTCACTGGATCTGGCTACGT | |
| | CTGGTTCATGGTGGGGCCCCAGCTGGCTGGAGG | |
| | CGGGGGCTCTGGGGCCCCTGGTGAGCCCCCTC | |
| | TTCTGCCAGGAGGCGCCCCCCTGCCTGCCGGG | |
| | CTGTTTGCAGTGCGCTCGGCTGGCTGGCGGGAT | |
| | GACCTGGCTCGGCGAGTGGCAGCTGGCGTGGC | |
| | CGTAGTGGCCAGAGGTGCCCAGGCCCTGCTGC | |
| | GTGATTATGGTTTCCTTCCTGAGCTCGGCCACGA | |
| | CTGTCGCGCCCAGAACCGCACCCACCGCGGCG | |
| | AGAGTCTGCATAGGTACTTCATGAACATCACGTG | |
| | GGATAACCGGGATTACTCCTTCAATGAGGACGG | |
| | CTTCCTAGTGAACCCCTCCCTGGTGGTCATCTCC | |
| | CTCACCAGAGACAGGACGTGGGAGGTGGTGGG | |
| | CAGCTGGGAGCAGCAGACGCTCCGCCTCAAGTA | |
| | CCCGCTGTGGTCCCGCTATGGTCGCTTCCTGCA | |
| | GCCAGTGGACGACACGCAGCACCTCACGGTGGC | |
| | CACGCTGGAGGAAAGGCCGTTTGTCATCGTGGA | |
| | GCCTGCAGACCCTATCAGCGGCACCTGCATCCG | |
| | AGACTCCGTCCCCTGCCGGAGCCAGCTCAACCG | |
| | AACCCACAGCCCTCCACCGGATGCCCCCCGCCC | |
| | GGAAAAGCGCTGCTGCAAGGGTTTCTGCATCGA | |
| | CATTCTGAAGCGGCTGGCGCATACCATCGGCTT | |
| | CAGCTACGACCTCTACCTGGTCACCAATGGCAA | |
| | GCACGGAAAGAAGATCGATGGCGTCTGGAACGG | |
| | CATGATCGGGGAGGTGTTCTACCAGCGCGCAGA | |
| | CATGGCCATCGGCTCCCTCACCATCAACGAGGA | |
| | GCGCTCCGAGATCGTGGACTTCTCCGTCCCCTT | |
| | CGTGGAGACCGGCATCAGCGTCATGGTGGCGC | |
| | GCGGCACCACTGTGTCTGGGCTCAGTGACCGCA | |
| | AGTTCCAGAGGCCCCAGGAGCAGTACCCGCCCC | |
| | TGAAGTTTGGGACCGTGCCCAACGGCTCCACGG | |
| | AGAAGAACATCCGCAGCAACTATCCCGACATGCA | |
| | CAGCTACATGGTGCGCTACAACCAGCCCCGCGT | |
| | AGAGGAAGCGCTCACTCAGCTCAAGGCAGGGAA | |
| | GCTGGACGCCTTCATCTACGATGCTGCAGTGCT | |
| | CAATTACATGGCCCGCAAGGACGAGGGCTGCAA | |
| | GCTTGTCACCATCGGCTCCGGCAAGGTCTTCGC | |
| | CACGACAGGCTATGGCATCGCCCTGCACAAGGG | |
| | CTCCCGCTGGAAGCGGCCCATCGACCTGGCGTT | |
| | GCTGCAGTTCCTGGGGGATGATGAGATCGAGAT | |
| | GCTGGAGCGGCTGTGGCTCTCTGGGATCTGCCA | |
| | CAAT | |
| SEQ ID NO | ATGGGCAGAGTGGGCTATTGGACCCTGCTGGTG | Human GluN2A |
| 90 | CTGCCGGCCCTTCTGGTCTGGCGCGGTCCGGC | NM_001134407 |
| | GCCGAGCGCGGCGGCGGAGAAGGGTCCCCCCG | |
| | CGCTAAATATTGCGGTGATGCTGGGTCACAGCC | |
| | ACGACGTGACAGAGCGCGAACTTCGAACACTGT | |
| | GGGGCCCCGAGCAGGCGGCGGGGCTGCCCCTG | |
| | GACGTGAACGTGGTAGCTCTGCTGATGAACCGC | |
| | ACCGACCCCAAGAGCCTCATCACGCACGTGTGC | |
| | GACCTCATGTCCGGGGCACGCATCCACGGCCTC | |
| | GTGTTTGGGGACGACACGGACCAGGAGGCCGTA | |
| | GCCCAGATGCTGGATTTTATCTCCTCCCACACCT | |
| | TCGTCCCCATCTTGGGCATTCATGGGGGCGCAT | |
| | CTATGATCATGGCTGACAAGGATCCGACGTCTAC | |
| | CTTCTTCCAGTTTGGAGCGTCCATCCAGCAGCAA | |
| | GCCACGGTCATGCTGAAGATCATGCAGGATTATG | |
| | ACTGGCATGTCTTCTCCCTGGTGACCACTATCTT | |
| | CCCTGGCTACAGGGAATTCATCAGCTTCGTCAAG | |
| | ACCACAGTGGACAACAGCTTTGTGGGCTGGGAC | |
| | ATGCAGAATGTGATCACACTGGACACTTCCTTTG | |
| | AGGATGCAAAGACACAAGTCCAGCTGAAGAAGA | |
| | TCCACTCTTCTGTCATCTTGCTCTACTGTTCCAAA | |
| | GACGAGGCTGTTCTCATTCTGAGTGAGGCCCGC | |
| | TCCCTTGGCCTCACCGGGTATGATTTCTTCTGGA | |
| | TTGTCCCCAGCTTGGTCTCTGGGAACACGGAGC | |
| | TCATCCCAAAAGAGTTTCCATCGGGACTCATTTC | |
| | TGTCTCCTACGATGACTGGGACTACAGCCTGGA | |
| | GGCGAGAGTGAGGGACGGCATTGGCATCCTAAC | |
| | CACCGCTGCATCTTCTATGCTGGAGAAGTTCTCC | |
| | TACATCCCCGAGGCCAAGGCCAGCTGCTACGGG | |
| | CAGATGGAGAGGCCAGAGGTCCCGATGCACACC | |
| | TTGCACCCATTTATGGTCAATGTTACATGGGATG | |
| | GCAAAGACTTATCCTTCACTGAGGAAGGCTACCA | |
| | GGTGCACCCCAGGCTGGTGGTGATTGTGCTGAA | |
| | CAAAGACCGGGAATGGGAAAAGGTGGGCAAGTG | |
| | GGAGAACCATACGCTGAGCCTGAGGCACGCCGT | |
| | GTGGCCCAGGTACAAGTCCTTCTCCGACTGTGA | |
| | GCCGGATGACAACCATCTCAGCATCGTCACCCT | |
| | GGAGGAGGCCCCATTCGTCATCGTGGAAGACAT | |
| | AGACCCCCTGACCGAGACGTGTGTGAGGAACAC | |
| | CGTGCCATGTCGGAAGTTCGTCAAAATCAACAAT | |
| | TCAACCAATGAGGGGATGAATGTGAAGAAATGCT | |
| | GCAAGGGGTTCTGCATTGATATTCTGAAGAAGCT | |
| | TTCCAGAACTGTGAAGTTTACTTACGACCTCTATC | |
| | TGGTGACCAATGGGAAGCATGGCAAGAAAGTTA | |
| | ACAATGTGTGGAATGGAATGATCGGTGAAGTGGT | |
| | CTATCAACGGGCAGTCATGGCAGTTGGCTCGCT | |
| | CACCATCAATGAGGAACGTTCTGAAGTGGTGGA | |
| | CTTCTCTGTGCCCTTTGTGGAAACGGGAATCAGT | |
| | GTCATGGTTTCAAGAAGTAATGGCACCGTCTCAC | |
| | CTTCTGCTTTTCTAGAACCATTCAGCGCCTCTGT | |
| | CTGGGTGATGATGTTTGTGATGCTGCTCATTGTT | |
| | TCTGCCATAGCTGTTTTTGTCTTTGAATACTTCAG | |
| | CCCTGTTGGATACAACAGAAACTTAGCCAAAGGG | |
| | AAAGCACCCCATGGGCCTTCTTTTACAATTGGAA | |
| | AAGCTATATGGCTTCTTTGGGGCCTGGTGTTCAA | |
| | TAACTCCGTGCCTGTCCAGAATCCTAAAGGGACC | |
| | ACCAGCAAGATCATGGTATCTGTATGGGCCTTCT | |
| | TCGCTGTCATATTCCTGGCTAGCTACACAGCCAA | |
| | TCTGGCTGCCTTCATGATCCAAGAGGAATTTGTG | |
| | GACCAAGTGACCGGCCTCAGTGACAAAAAGTTTC | |
| | AGAGACCTCATGACTATTCCCCACCTTTTCGATTT | |
| | GGGACAGTGCCTAATGGAAGCACGGAGAGAAAC | |
| | ATTCGGAATAACTATCCCTACATGCATCAGTACAT | |
| | GACCAAATTTAATCAGAAAGGAGTAGAGGACGCC | |
| | TTGGTCAGCCTGAAAACGGGGAAGCTGGACGCT | |
| | TTCATCTACGATGCCGCAGTCTTGAATTACAAGG | |
| | CTGGGAGGGATGAAGGCTGCAAGCTGGTGACCA | |
| | TCGGGAGTGGGTACATCTTTGCCACCACCGGTT | |
| | ATGGAATTGCCCTTCAGAAAGGCTCTCCTTGGAA | |
| | GAGGCAGATCGACCTGGCCTTGCTTCAGTTTGT | |
| | GGGTGATGGTGAGATGGAGGAGCTGGAGACCCT | |
| | GTGGCTCACTGGGATCTGCCACAACGAGAAGAA | |
| | CGAGGTGATGAGCAGCCAGCTGGACATTGACAA | |
| | CATGGCGGGCGTATTCTACATGCTGGCTGCCGC | |
| | CATGGCCCTTAGCCTCATCACCTTCATCTGGGAG | |
| | CACCTCTTCTACTGGAAGCTGCGCTTCTGTTTCA | |
| | CGGGCGTGTGCTCCGACCGGCCTGGGTTGCTCT | |
| | TCTCCATCAGCAGGGGCATCTACAGCTGCATTCA | |
| | TGGAGTGCACATTGAAGAAAAGAAGAAGTCTCCA | |
| | GACTTCAATCTGACGGGATCCCAGAGCAACATGT | |
| | TAAAACTCCTCCGGTCAGCCAAAAACATTTCCAG | |
| | CATGTCCAACATGAACTCCTCAAGAATGGACTCA | |
| | CCCAAAAGAGCTGCTGACTTCATCCAAAGAGGTT | |
| | CCCTCATCATGGACATGGTTTCAGATAAGGGGAA | |
| | TTTGATGTACTCAGACAACAGGTCCTTTCAGGGG | |
| | AAAGAGAGCATTTTTGGAGACAACATGAACGAAC | |
| | TCCAAACATTTGTGGCCAACCGGCAGAAGGATAA | |
| | CCTCAATAACTATGTATTCCAGGGACAACATCCT | |
| | CTTACTCTCAATGAGTCCAACCCTAACACGGTGG | |
| | AGGTGGCCGTGAGCACAGAATCCAAAGCGAACT | |
| | CTAGACCCCGGCAGCTGTGGAAGAAATCCGTGG | |
| | ATTCCATACGCCAGGATTCACTATCCCAGAATCC | |
| | AGTCTCCCAGAGGGATGAGGCAACAGCAGAGAA | |
| | TAGGACCCACTCCCTAAAGAGCCCTAGGTATCTT | |
| | CCAGAAGAGATGGCCCACTCTGACATTTCAGAAA | |
| | CGTCAAATCGGGCCACGTGCCACAGGGAACCTG | |
| | ACAACAGTAAGAACCACAAAACCAAGGACAACTT | |
| | TAAAAGGTCAGTGGCCTCCAAATACCCCAAGGAC | |
| | TGTAGTGAGGTCGAGCGCACCTACCTGAAAACC | |
| | AAATCAAGCTCCCCTAGAGACAAGATCTACACTA | |
| | TAGATGGTGAGAAGGAGCCTGGTTTCCACTTAGA | |
| | TCCACCCCAGTTTGTTGAAAATGTGACCCTGCCC | |
| | GAGAACGTGGACTTCCCGGACCCCTACCAGGAT | |
| | CCCAGTGAAAACTTCCGCAAGGGGGACTCCACG | |
| | CTGCCAATGAACCGGAACCCCTTGCATAATGAAG | |
| | AGGGGCTTTCCAACAACGACCAGTATAAACTCTA | |
| | CTCCAAGCACTTCACCTTGAAAGACAAGGGTTCC | |
| | CCGCACAGTGAGACCAGCGAGCGATACCGGCAG | |
| | AACTCCACGCACTGCAGAAGCTGCCTTTCCAACA | |
| | TGCCCACCTATTCAGGCCACTTCACCATGAGGTC | |
| | CCCCTTCAAGTGCGATGCCTGCCTGCGGATGGG | |
| | GAACCTCTATGACATCGATGAAGACCAGATGOTT | |
| | CAGGAGACAGGTAACCCAGCCACCGGGGAGCA | |
| | GGTCTACCAGCAGGACTGGGCACAGAACAATGC | |
| | CCTTCAATTACAAAAGAACAAGCTAAGGATTAGC | |
| | CGTCAGCATTCCTACGATAACATTGTCGACAAAC | |
| | CTAGGGAGCTAGACCTTAGCAGGCCCTCCCGGA | |
| | GCATAAGCCTCAAGGACAGGGAACGGCTTCTGG | |
| | AGGGAAATTTTTACGGCAGCCTGTTTAGTGTCCC | |
| | CTCAAGCAAACTCTCGGGGAAAAAAAGCTCCCTT | |
| | TTCCCCCAAGGTCTGGAGGACAGCAAGAGGAGC | |
| | AAGTCTCTCTTGCCAGACCACACCTCCGATAACC | |
| | CTTTCCTCCACTCCCACAGGGATGACCAACGCTT | |
| | GGTTATTGGGAGATGCCCCTCGGACCCTTACAAA | |
| | CACTCGTTGCCATCCCAGGCGGTGAATGACAGC | |
| | TATCTTCGGTCGTCCTTGAGGTCAACGGCATCGT | |
| | ACTGTTCCAGGGACAGTCGGGGCCACAATGATG | |
| | TGTATATTTCGGAGCATGTTATGCCTTATGCTGC | |
| | AAATAAGAATAATATGTACTCTACCCCCAGGGTTT | |
| | TAAATTCCTGCAGCAATAGACGCGTGTACAAGAA | |
| | AATGCCTAGTATCGAATCTGATGTTTAA | |
| SEQ ID NO | GACAACCATCTCAGCATCGTCACCCTGGAGGAG | GluN2A S1 |
| 91 | GCCCCATTCGTCATCGTGGAAGACATAGACCCC | |
| | CTGACCGAGACGTGTGTGAGGAACACCGTGCCA | |
| | TGTCGGAAGTTCGTCAAAATCAACAATTCAACCA | |
| | ATGAGGGGATGAATGTGAAGAAATGCTGCAAGG | |
| | GGTTCTGCATTGATATTCTGAAGAAGCTTTCCAG | |
| | AACTGTGAAGTTTACTTACGACCTCTATCTGGTG | |
| | ACCAATGGGAAGCATGGCAAGAAAGTTAACAATG | |
| | TGTGGAATGGAATGATCGGTGAAGTGGTCTATCA | |
| | ACGGGCAGTCATGGCAGTTGGCTCGCTCACCAT | |
| | CAATGAGGAACGTTCTGAAGTGGTGGACTTCTCT | |
| | GTGCCCTTTGTGGAAACGGGAATCAGTGTCATG | |
| | GTTTCAAGA | |
| SEQ ID NO | CAAGTGACCGGCCTCAGTGACAAAAAGTTTCAGA | GluN2A S2 |
| 92 | GACCTCATGACTATTCCCCACCTTTTCGATTTGG | |
| | GACAGTGCCTAATGGAAGCACGGAGAGAAACAT | |
| | TCGGAATAACTATCCCTACATGCATCAGTACATG | |
| | ACCAAATTTAATCAGAAAGGAGTAGAGGACGCCT | |
| | TGGTCAGCCTGAAAACGGGGAAGCTGGACGCTT | |
| | TCATCTACGATGCCGCAGTCTTGAATTACAAGGC | |
| | TGGGAGGGATGAAGGCTGCAAGCTGGTGACCAT | |
| | CGGGAGTGGGTACATCTTTGCCACCACCGGTTA | |
| | TGGAATTGCCCTTCAGAAAGGCTCTCCTTGGAAG | |
| | AGGCAGATCGACCTGGCCTTGCTTCAGTTTGTG | |
| | GGTGATGGTGAGATGGAGGAGCTGGAGACCCTG | |
| | TGGCTCACTGGGATCTGCCACAAC | |
| SEQ ID NO | ATGGGCAGAGTGGGCTATTGGACCCTGCTGGTG | GluN2A ecd incl. |
| 93 | CTGCCGGCCCTTCTGGTCTGGCGCGGTCCGGC | signal sequence and |
| | GCCGAGCGCGGCGGCGGAGAAGGGTCCCCCCG | GT-linker between S1 |
| | CGCTAAATATTGCGGTGATGCTGGGTCACAGCC | and S2 |
| | ACGACGTGACAGAGCGCGAACTTCGAACACTGT | |
| | GGGGCCCCGAGCAGGCGGCGGGGCTGCCCCTG | |
| | GACGTGAACGTGGTAGCTCTGCTGATGAACCGC | |
| | ACCGACCCCAAGAGCCTCATCACGCACGTGTGC | |
| | GACCTCATGTCCGGGGCACGCATCCACGGCCTC | |
| | GTGTTTGGGGACGACACGGACCAGGAGGCCGTA | |
| | GCCCAGATGCTGGATTTTATCTCCTCCCACACCT | |
| | TCGTCCCCATCTTGGGCATTCATGGGGGCGCAT | |
| | CTATGATCATGGCTGACAAGGATCCGACGTCTAC | |
| | CTTCTTCCAGTTTGGAGCGTCCATCCAGCAGCAA | |
| | GCCACGGTCATGCTGAAGATCATGCAGGATTATG | |
| | ACTGGCATGTCTTCTCCCTGGTGACCACTATCTT | |
| | CCCTGGCTACAGGGAATTCATCAGCTTCGTCAAG | |
| | ACCACAGTGGACAACAGCTTTGTGGGCTGGGAC | |
| | ATGCAGAATGTGATCACACTGGACACTTCCTTTG | |
| | AGGATGCAAAGACACAAGTCCAGCTGAAGAAGA | |
| | TCCACTCTTCTGTCATCTTGCTCTACTGTTCCAAA | |
| | GACGAGGCTGTTCTCATTCTGAGTGAGGCCCGC | |
| | TCCCTTGGCCTCACCGGGTATGATTTCTTCTGGA | |
| | TTGTCCCCAGCTTGGTCTCTGGGAACACGGAGC | |
| | TCATCCCAAAAGAGTTTCCATCGGGACTCATTTC | |
| | TGTCTCCTACGATGACTGGGACTACAGCCTGGA | |
| | GGCGAGAGTGAGGGACGGCATTGGCATCCTAAC | |
| | CACCGCTGCATCTTCTATGCTGGAGAAGTTCTCC | |
| | TACATCCCCGAGGCCAAGGCCAGCTGCTACGGG | |
| | CAGATGGAGAGGCCAGAGGTCCCGATGCACACC | |
| | TTGCACCCATTTATGGTCAATGTTACATGGGATG | |
| | GCAAAGACTTATCCTTCACTGAGGAAGGCTACCA | |
| | GGTGCACCCCAGGCTGGTGGTGATTGTGCTGAA | |
| | CAAAGACCGGGAATGGGAAAAGGTGGGCAAGTG | |
| | GGAGAACCATACGCTGAGCCTGAGGCACGCCGT | |
| | GTGGCCCAGGTACAAGTCCTTCTCCGACTGTGA | |
| | GCCGGATGACAACCATCTCAGCATCGTCACCCT | |
| | GGAGGAGGCCCCATTCGTCATCGTGGAAGACAT | |
| | AGACCCCCTGACCGAGACGTGTGTGAGGAACAC | |
| | CGTGCCATGTCGGAAGTTCGTCAAAATCAACAAT | |
| | TCAACCAATGAGGGGATGAATGTGAAGAAATGCT | |
| | GCAAGGGGTTCTGCATTGATATTCTGAAGAAGCT | |
| | TTCCAGAACTGTGAAGTTTACTTACGACCTCTATC | |
| | TGGTGACCAATGGGAAGCATGGCAAGAAAGTTA | |
| | ACAATGTGTGGAATGGAATGATCGGTGAAGTGGT | |
| | CTATCAACGGGCAGTCATGGCAGTTGGCTCGCT | |
| | CACCATCAATGAGGAACGTTCTGAAGTGGTGGA | |
| | CTTCTCTGTGCCCTTTGTGGAAACGGGAATCAGT | |
| | GTCATGGTTTCAAGAGGCACCCAAGTGACCGGC | |
| | CTCAGTGACAAAAAGTTTCAGAGACCTCATGACT | |
| | ATTCCCCACCTTTTCGATTTGGGACAGTGCCTAA | |
| | TGGAAGCACGGAGAGAAACATTCGGAATAACTAT | |
| | CCCTACATGCATCAGTACATGACCAAATTTAATCA | |
| | GAAAGGAGTAGAGGACGCCTTGGTCAGCCTGAA | |
| | AACGGGGAAGCTGGACGCTTTCATCTACGATGC | |
| | CGCAGTCTTGAATTACAAGGCTGGGAGGGATGA | |
| | AGGCTGCAAGCTGGTGACCATCGGGAGTGGGTA | |
| | CATCTTTGCCACCACCGGTTATGGAATTGCCCTT | |
| | CAGAAAGGCTCTCCTTGGAAGAGGCAGATCGAC | |
| | CTGGCCTTGCTTCAGTTTGTGGGTGATGGTGAGA | |
| | TGGAGGAGCTGGAGACCCTGTGGCTCACTGGGA | |
| | TCTGCCACAAC | |
| SEQ ID NO | ATGAAGCCCAGAGCGGAGTGCTGTTCTCCCAAG | Human GluN2B |
| 94 | TTCTGGTTGGTGTTGGCCGTCCTGGCCGTGTCA | NM_000834 |
| | GGCAGCAGAGCTCGTTCTCAGAAGAGCCCCCCC | |
| | AGCATTGGCATTGCTGTCATCCTCGTGGGCACTT | |
| | CCGACGAGGTGGCCATCAAGGATGCCCACGAGA | |
| | AAGATGATTTCCACCATCTCTCCGTGGTACCCCG | |
| | GGTGGAACTGGTAGCCATGAATGAGACCGACCC | |
| | AAAGAGCATCATCACCCGCATCTGTGATCTCATG | |
| | TCTGACCGGAAGATCCAGGGGGTGGTGTTTGCT | |
| | GATGACACAGACCAGGAAGCCATCGCCCAGATC | |
| | CTCGATTTCATTTCAGCACAGACTCTCACCCCCA | |
| | TCCTGGGCATCCACGGGGGCTCCTCTATGATAAT | |
| | GGCAGATAAGGATGAATCCTCCATGTTCTTCCAG | |
| | TTTGGCCCATCAATTGAACAGCAAGCTTCCGTAA | |
| | TGCTCAACATCATGGAAGAATATGACTGGTACAT | |
| | CTTTTCTATCGTCACCACCTATTTCCCTGGCTACC | |
| | AGGACTTTGTAAACAAGATCCGCAGCACCATTGA | |
| | GAATAGCTTTGTGGGCTGGGAGCTAGAGGAGGT | |
| | CCTCCTACTGGACATGTCCCTGGACGATGGAGA | |
| | TTCTAAGATCCAGAATCAGCTCAAGAAACTTCAA | |
| | AGCCCCATCATTCTTCTTTACTGTACCAAGGAAG | |
| | AAGCCACCTACATCTTTGAAGTGGCCAACTCAGT | |
| | AGGGCTGACTGGCTATGGCTACACGTGGATCGT | |
| | GCCCAGTCTGGTGGCAGGGGATACAGACACAGT | |
| | GCCTGCGGAGTTCCCCACTGGGCTCATCTCTGT | |
| | ATCATATGATGAATGGGACTATGGCCTCCCCGCC | |
| | AGAGTGAGAGATGGAATTGCCATAATCACCACTG | |
| | CTGCTTCTGACATGCTGTCTGAGCACAGCTTCAT | |
| | CCCTGAGCCCAAAAGCAGTTGTTACAACACCCAC | |
| | GAGAAGAGAATCTACCAGTCCAATATGCTAAATA | |
| | GGTATCTGATCAATGTCACTTTTGAGGGGAGGAA | |
| | TTTGTCCTTCAGTGAAGATGGCTACCAGATGCAC | |
| | CCGAAACTGGTGATAATTCTTCTGAACAAGGAGA | |
| | GGAAGTGGGAAAGGGTGGGGAAGTGGAAAGAC | |
| | AAGTCCCTGCAGATGAAGTACTATGTGTGGCCCC | |
| | GAATGTGTCCAGAGACTGAAGAGCAGGAGGATG | |
| | ACCATCTGAGCATTGTGACCCTGGAGGAGGCAC | |
| | CATTTGTCATTGTGGAAAGTGTGGACCCTCTGAG | |
| | TGGAACCTGCATGAGGAACACAGTCCCCTGCCA | |
| | AAAACGCATAGTCACTGAGAATAAAACAGACGAG | |
| | GAGCCGGGTTACATCAAAAAATGCTGCAAGGGG | |
| | TTCTGTATTGACATCCTTAAGAAAATTTCTAAATC | |
| | TGTGAAGTTCACCTATGACCTTTACCTGGTTACC | |
| | AATGGCAAGCATGGGAAGAAAATCAATGGAACCT | |
| | GGAATGGTATGATTGGAGAGGTGGTCATGAAGA | |
| | GGGCCTACATGGCAGTGGGCTCACTCACCATCA | |
| | ATGAGGAACGATCGGAGGTGGTCGACTTCTCTG | |
| | TGCCCTTCATAGAGACAGGCATCAGTGTCATGGT | |
| | GTCACGCAGCAATGGGACTGTCTCACCTTCTGC | |
| | CTTCTTAGAGCCATTCAGCGCTGACGTATGGGTG | |
| | ATGATGTTTGTGATGCTGCTCATCGTCTCAGCCG | |
| | TGGCTGTCTTTGTCTTTGAGTACTTCAGCCCTGT | |
| | GGGTTATAACAGGTGCCTCGCTGATGGCAGAGA | |
| | GCCTGGTGGACCCTCTTTCACCATCGGCAAAGC | |
| | TATTTGGTTGCTCTGGGGTCTGGTGTTTAACAAC | |
| | TCCGTACCTGTGCAGAACCCAAAGGGGACCACC | |
| | TCCAAGATCATGGTGTCAGTGTGGGCCTTCTTTG | |
| | CTGTCATCTTCCTGGCCAGCTACACTGCCAACTT | |
| | AGCTGCCTTCATGATCCAAGAGGAATATGTGGAC | |
| | CAGGTTTCTGGCCTGAGCGACAAAAAGTTCCAGA | |
| | GACCTAATGACTTCTCACCCCCTTTCCGCTTTGG | |
| | GACCGTGCCCAACGGCAGCACAGAGAGAAATAT | |
| | TCGCAATAACTATGCAGAAATGCATGCCTACATG | |
| | GGAAAGTTCAACCAGAGGGGTGTAGATGATGCA | |
| | TTGCTCTCCCTGAAAACAGGGAAACTGGATGCCT | |
| | TCATCTATGATGCAGCAGTGCTGAACTATATGGC | |
| | AGGCAGAGATGAAGGCTGCAAGCTGGTGACCAT | |
| | TGGCAGTGGGAAGGTCTTTGCTTCCACTGGCTAT | |
| | GGCATTGCCATCCAAAAAGATTCTGGGTGGAAG | |
| | CGCCAGGTGGACCTTGCTATCCTGCAGCTCTTTG | |
| | GAGATGGGGAGATGGAAGAACTGGAAGCTCTCT | |
| | GGCTCACTGGCATTTGTCACAATGAGAAGAATGA | |
| | GGTCATGAGCAGCCAGCTGGACATTGACAACAT | |
| | GGCAGGGGTCTTCTACATGTTGGGGGCGGCCAT | |
| | GGCTCTCAGCCTCATCACCTTCATCTGCGAACAC | |
| | CTTTTCTATTGGCAGTTCCGACATTGCTTTATGG | |
| | GTGTCTGTTCTGGCAAGCCTGGCATGGTCTTCTC | |
| | CATCAGCAGAGGTATCTACAGCTGCATCCATGG | |
| | GGTGGCGATCGAGGAGCGCCAGTCTGTAATGAA | |
| | CTCCCCCACCGCAACCATGAACAACACACACTCC | |
| | AACATCCTGCGCCTGCTGCGCACGGCCAAGAAC | |
| | ATGGCTAACCTGTCTGGTGTGAATGGCTCACCG | |
| | CAGAGCGCCCTGGACTTCATCCGACGGGAGTCA | |
| | TCCGTCTATGACATCTCAGAGCACCGCCGCAGC | |
| | TTCACGCATTCTGACTGCAAATCCTACAACAACC | |
| | CGCCCTGTGAGGAGAACCTCTTCAGTGACTACAT | |
| | CAGTGAGGTAGAGAGAACGTTCGGGAACCTGCA | |
| | GCTGAAGGACAGCAACGTGTACCAAGATCACTA | |
| | CCACCATCACCACCGGCCCCATAGTATTGGCAG | |
| | TGCCAGCTCCATCGATGGGCTCTACGACTGTGA | |
| | CAACCCACCCTTCACCACCCAGTCCAGGTCCATC | |
| | AGCAAGAAGCCCCTGGACATCGGCCTCCCCTCC | |
| | TCCAAGCACAGCCAGCTCAGTGACCTGTACGGC | |
| | AAATTCTCCTTCAAGAGCGACCGCTACAGTGGCC | |
| | ACGACGACTTGATCCGCTCCGATGTCTCTGACAT | |
| | CTCAACCCACACCGTCACCTATGGGAACATCGA | |
| | GGGCAATGCCGCCAAGAGGCGTAAGCAGCAATA | |
| | TAAGGACAGCCTGAAGAAGCGGCCTGCCTCGGC | |
| | CAAGTCCCGCAGGGAGTTTGACGAGATCGAGCT | |
| | GGCCTACCGTCGCCGACCGCCCCGCTCCCCTGA | |
| | CCACAAGCGCTACTTCAGGGACAAGGAAGGGCT | |
| | ACGGGACTTCTACCTGGACCAGTTCCGAACAAA | |
| | GGAGAACTCACCCCACTGGGAGCACGTAGACCT | |
| | GACCGACATCTACAAGGAGCGGAGTGATGACTT | |
| | TAAGCGCGACTCCGTCAGCGGAGGAGGGCCCT | |
| | GTACCAACAGGTCTCACATCAAGCACGGGACGG | |
| | GCGACAAACACGGCGTGGTCAGCGGGGTACCTG | |
| | CACCTTGGGAGAAGAACCTGACCAACGTGGAGT | |
| | GGGAGGACCGGTCCGGGGGCAACTTCTGCCGC | |
| | AGCTGTCCCTCCAAGCTGCACAACTACTCCACGA | |
| | CGGTGACGGGTCAGAACTCGGGCAGGCAGGCG | |
| | TGCATCCGGTGTGAGGCTTGCAAGAAAGCAGGC | |
| | AACCTGTATGACATCAGTGAGGACAACTCCCTGC | |
| | AGGAACTGGACCAGCCGGCTGCCCCAGTGGCG | |
| | GTGACGTCAAACGCCTCCACCACTAAGTACCCTC | |
| | AGAGCCCGACTAATTCCAAGGCCCAGAAGAAGA | |
| | ACCGGAACAAACTGCGCCGGCAGCACTCCTACG | |
| | ACACCTTCGTGGACCTGCAGAAGGAAGAAGCCG | |
| | CCCTGGCCCCGCGCAGCGTAAGCCTGAAAGACA | |
| | AGGGCCGATTCATGGATGGGAGCCCCTACGCCC | |
| | ACATGTTTGAGATGTCAGCTGGCGAGAGCACCTT | |
| | TGCCAACAACAAGTCCTCAGTGCCCACTGCCGG | |
| | ACATCACCACCACAACAACCCCGGCGGCGGGTA | |
| | CATGCTCAGCAAGTCGCTCTACCCTGACCGGGT | |
| | CACGCAAAACCCTTTCATCCCCACTTTTGGGGAC | |
| | GACCAGTGCTTGCTCCATGGCAGCAAATCCTACT | |
| | TCTTCAGGCAGCCCACGGTGGCGGGGGCGTCG | |
| | AAAGCCAGGCCGGACTTCCGGGCCCTTGTCACC | |
| | AACAAGCCGGTGGTCTCGGCCCTTCATGGGGCC | |
| | GTGCCAGCCCGTTTCCAGAAGGACATCTGTATAG | |
| | GGAACCAGTCCAACCCCTGTGTGCCTAACAACAA | |
| | AAACCCCAGGGCTTTCAATGGCTCCAGCAATGG | |
| | GCATGTTTATGAGAAACTTTCTAGTATTGAGTCTG | |
| | ATGTCTGA | |
| SEQ ID NO | ATGAAGCCCAGAGCGGAGTGCTGTTCTCCCAAG | GluN2B ecd incl. |
| 95 | TTCTGGTTGGTGTTGGCCGTCCTGGCCGTGTCA | signal sequence and |
| | GGCAGCAGAGCTCGTTCTCAGAAGAGCCCCCCC | GT-linker between S1 |
| | AGCATTGGCATTGCTGTCATCCTCGTGGGCACTT | and S2 |
| | CCGACGAGGTGGCCATCAAGGATGCCCACGAGA | |
| | AAGATGATTTCCACCATCTCTCCGTGGTACCCCG | |
| | GGTGGAACTGGTAGCCATGAATGAGACCGACCC | |
| | AAAGAGCATCATCACCCGCATCTGTGATCTCATG | |
| | TCTGACCGGAAGATCCAGGGGGTGGTGTTTGCT | |
| | GATGACACAGACCAGGAAGCCATCGCCCAGATC | |
| | CTCGATTTCATTTCAGCACAGACTCTCACCCCCA | |
| | TCCTGGGCATCCACGGGGGCTCCTCTATGATAAT | |
| | GGCAGATAAGGATGAATCCTCCATGTTCTTCCAG | |
| | TTTGGCCCATCAATTGAACAGCAAGCTTCCGTAA | |
| | TGCTCAACATCATGGAAGAATATGACTGGTACAT | |
| | CTTTTCTATCGTCACCACCTATTTCCCTGGCTACC | |
| | AGGACTTTGTAAACAAGATCCGCAGCACCATTGA | |
| | GAATAGCTTTGTGGGCTGGGAGCTAGAGGAGGT | |
| | CCTCCTACTGGACATGTCCCTGGACGATGGAGA | |
| | TTCTAAGATCCAGAATCAGCTCAAGAAACTTCAA | |
| | AGCCCCATCATTCTTCTTTACTGTACCAAGGAAG | |
| | AAGCCACCTACATCTTTGAAGTGGCCAACTCAGT | |
| | AGGGCTGACTGGCTATGGCTACACGTGGATCGT | |
| | GCCCAGTCTGGTGGCAGGGGATACAGACACAGT | |
| | GCCTGCGGAGTTCCCCACTGGGCTCATCTCTGT | |
| | ATCATATGATGAATGGGACTATGGCCTCCCCGCC | |
| | AGAGTGAGAGATGGAATTGCCATAATCACCACTG | |
| | CTGCTTCTGACATGCTGTCTGAGCACAGCTTCAT | |
| | CCCTGAGCCCAAAAGCAGTTGTTACAACACCCAC | |
| | GAGAAGAGAATCTACCAGTCCAATATGCTAAATA | |
| | GGTATCTGATCAATGTCACTTTTGAGGGGAGGAA | |
| | TTTGTCCTTCAGTGAAGATGGCTACCAGATGCAC | |
| | CCGAAACTGGTGATAATTCTTCTGAACAAGGAGA | |
| | GGAAGTGGGAAAGGGTGGGGAAGTGGAAAGAC | |
| | AAGTCCCTGCAGATGAAGTACTATGTGTGGCCCC | |
| | GAATGTGTCCAGAGACTGAAGAGCAGGAGGATG | |
| | ACCATCTGAGCATTGTGACCCTGGAGGAGGCAC | |
| | CATTTGTCATTGTGGAAAGTGTGGACCCTCTGAG | |
| | TGGAACCTGCATGAGGAACACAGTCCCCTGCCA | |
| | AAAACGCATAGTCACTGAGAATAAAACAGACGAG | |
| | GAGCCGGGTTACATCAAAAAATGCTGCAAGGGG | |
| | TTCTGTATTGACATCCTTAAGAAAATTTCTAAATC | |
| | TGTGAAGTTCACCTATGACCTTTACCTGGTTACC | |
| | AATGGCAAGCATGGGAAGAAAATCAATGGAACCT | |
| | GGAATGGTATGATTGGAGAGGTGGTCATGAAGA | |
| | GGGCCTACATGGCAGTGGGCTCACTCACCATCA | |
| | ATGAGGAACGATCGGAGGTGGTCGACTTCTCTG | |
| | TGCCCTTCATAGAGACAGGCATCAGTGTCATGGT | |
| | GTCACGCGGCACCCAGGTTTCTGGCCTGAGCGA | |
| | CAAAAAGTTCCAGAGACCTAATGACTTCTCACCC | |
| | CCTTTCCGCTTTGGGACCGTGCCCAACGGCAGC | |
| | ACAGAGAGAAATATTCGCAATAACTATGCAGAAA | |
| | TGCATGCCTACATGGGAAAGTTCAACCAGAGGG | |
| | GTGTAGATGATGCATTGCTCTCCCTGAAAACAGG | |
| | GAAACTGGATGCCTTCATCTATGATGCAGCAGTG | |
| | CTGAACTATATGGCAGGCAGAGATGAAGGCTGC | |
| | AAGCTGGTGACCATTGGCAGTGGGAAGGTCTTT | |
| | GCTTCCACTGGCTATGGCATTGCCATCCAAAAAG | |
| | ATTCTGGGTGGAAGCGCCAGGTGGACCTTGCTA | |
| | TCCTGCAGCTCTTTGGAGATGGGGAGATGGAAG | |
| | AACTGGAAGCTCTCTGGCTCACTGGCATTTGTCA | |
| | CAAT | |
| SEQ ID NO | ATGGGTGGCGCACTTGGCCCTGCTTTGCTGCTC | DNA seq of fusion |
| 98 | ACTAGCCTGTTTGGAGCATGGGCTGGTCTTGGG | protein #9 N2C-ATD- |
| | CCAGGTCAAGGCGAGCAAGGGATGACCGTGGC | Fc |
| | CGTGGTGTTCTCCTCAAGTGGACCACCTCAGGC | |
| | ACAGTTCCGTGCCAGACTGACACCGCAGAGCTT | |
| | CCTGGATCTCCCACTGGAGATACAGCCTCTCACT | |
| | GTGGGCGTGAACACCACCAATCCCTCATCCCTG | |
| | CTGACACAGATCTGCGGACTTCTGGGTGCAGCC | |
| | CATGTCCACGGGATCGTGTTCGAGGACAACGTG | |
| | GACACAGAAGCCGTAGCCCAGATTCTGGACTTC | |
| | ATCAGCTCACAGACCCACGTTCCCATTCTGAGCA | |
| | TTAGTGGCGGGAGCGCTGTAGTGCTCACTCCCA | |
| | AAGAGCCGGGGTCTGCATTTCTGCAGCTCGGAG | |
| | TAAGCCTTGAGCAGCAGCTCCAGGTCTTGTTCAA | |
| | GGTGCTGGAAGAGTACGACTGGTCTGCGTTTGC | |
| | CGTCATCACCTCTCTGCATCCTGGCCATGCTCTG | |
| | TTTCTGGAAGGCGTTAGGGCTGTCGCCGATGCG | |
| | TCCCACGTCAGTTGGCGGTTGCTGGATGTGGTC | |
| | ACGTTGGAGCTTGGACCTGGAGGCCCCAGGGCC | |
| | AGAACACAGCGACTGCTGCGCCAACTGGATGCT | |
| | CCCGTGTTTGTGGCCTATTGTTCCCGCGAGGAG | |
| | GCCGAGGTGCTCTTCGCCGAGGCGGCGCAGGC | |
| | CGGTCTGGTGGGGCCCGGCCACGTGTGGCTGG | |
| | TGCCCAACCTGGCGCTGGGCAGCACCGATGCGC | |
| | CCCCCGCCACCTTCCCCGTGGGCCTCATCAGCG | |
| | TCGTCACCGAGAGCTGGCGCCTCAGCCTGCGCC | |
| | AGAAGGTGCGCGACGGCGTGGCCATTCTGGCCC | |
| | TGGGCGCCCACAGCTACTGGCGCCAGCATGGAA | |
| | CCCTGCCAGCCCCGGCCGGGGACTGCCGTGTT | |
| | CACCCTGGGCCCGTCAGCCCTGCCCGGGAGGC | |
| | CTTCTACAGGCACCTACTGAATGTCACCTGGGAG | |
| | GGCCGAGACTTCTCCTTCAGCCCTGGTGGGTAC | |
| | CTGGTCCAGCCCACCATGGTGGTGATCGCCCTC | |
| | AACCGGCACCGCCTCTGGGAGATGGTGGGGCG | |
| | CTGGGAGCATGGCGTCCTATACATGAAGTACCC | |
| | CGTGTGGCCTCGCTACAGTGCCTCTCTGCAGCC | |
| | TGTGGTGGACAGTCGGCACCTGACGGTGGGCTC | |
| | GAGCACCATGGTTAGATCTAGCAAGCCCACGTG | |
| | CCCACCCCCTGAACTCCTGGGGGGACCGTCTGT | |
| | CTTCATCTTCCCCCCAAAACCCAAGGACACCCTC | |
| | ATGATCTCACGCACCCCCGAGGTCACATGCGTG | |
| | GTGGTGGACGTGAGCCAGGATGACCCCGAGGT | |
| | GCAGTTCACATGGTACATAAACAACGAGCAGGTG | |
| | CGCACCGCCCGGCCGCCGCTACGGGAGCAGCA | |
| | GTTCAACAGCACGATCCGCGTGGTCAGCACCCT | |
| | CCCCATCGCGCACCAGGACTGGCTGAGGGGCAA | |
| | GGAGTTCAAGTGCAAAGTCCACAACAAGGCACT | |
| | CCCGGCCCCCATCGAGAAAACCATCTCCAAAGC | |
| | CAGAGGGCAGCCCCTGGAGCCGAAGGTCTACAC | |
| | CATGGGCCCTCCCCGGGAGGAGCTGAGCAGCA | |
| | GGTCGGTCAGCCTGACCTGCATGATCAACGGCT | |
| | TCTACCCTTCCGACATCTCGGTGGAGTGGGAGA | |
| | AGAACGGGAAGGCAGAGGACAACTACAAGACCA | |
| | CGCCGGCCGTGCTGGACAGCGACGGCTCCTACT | |
| | TCCTCTACAGCAAGCTCTCAGTGCCCACGAGTGA | |
| | GTGGCAGCGGGGCGACGTCTTCACCTGCTCCGT | |
| | GATGCACGAGGCCTTGCACAACCACTACACGCA | |
| | GAAGTCCATCTCCCGCTCTCCGGGTAAATGA | |
| SEQ ID NO | ATGGGTGGCGCACTTGGCCCTGCTTTGCTGCTC | Amino terminal domain |
| 99 | ACTAGCCTGTTTGGAGCATGGGCTGGTCTTGGG | of GluN2C (including |
| | CCAGGTCAAGGCGAGCAAGGGATGACCGTGGC | signal sequence) as |
| | CGTGGTGTTCTCCTCAAGTGGACCACCTCAGGC | for example used in |
| | ACAGTTCCGTGCCAGACTGACACCGCAGAGCTT | fusion protein #9 |
| | CCTGGATCTCCCACTGGAGATACAGCCTCTCACT | |
| | GTGGGCGTGAACACCACCAATCCCTCATCCCTG | |
| | CTGACACAGATCTGCGGACTTCTGGGTGCAGCC | |
| | CATGTCCACGGGATCGTGTTCGAGGACAACGTG | |
| | GACACAGAAGCCGTAGCCCAGATTCTGGACTTC | |
| | ATCAGCTCACAGACCCACGTTCCCATTCTGAGCA | |
| | TTAGTGGCGGGAGCGCTGTAGTGCTCACTCCCA | |
| | AAGAGCCGGGGTCTGCATTTCTGCAGCTCGGAG | |
| | TAAGCCTTGAGCAGCAGCTCCAGGTCTTGTTCAA | |
| | GGTGCTGGAAGAGTACGACTGGTCTGCGTTTGC | |
| | CGTCATCACCTCTCTGCATCCTGGCCATGCTCTG | |
| | TTTCTGGAAGGCGTTAGGGCTGTCGCCGATGCG | |
| | TCCCACGTCAGTTGGCGGTTGCTGGATGTGGTC | |
| | ACGTTGGAGCTTGGACCTGGAGGCCCCAGGGCC | |
| | AGAACACAGCGACTGCTGCGCCAACTGGATGCT | |
| | CCCGTGTTTGTGGCCTATTGTTCCCGCGAGGAG | |
| | GCCGAGGTGCTCTTCGCCGAGGCGGCGCAGGC | |
| | CGGTCTGGTGGGGCCCGGCCACGTGTGGCTGG | |
| | TGCCCAACCTGGCGCTGGGCAGCACCGATGCGC | |
| | CCCCCGCCACCTTCCCCGTGGGCCTCATCAGCG | |
| | TCGTCACCGAGAGCTGGCGCCTCAGCCTGCGCC | |
| | AGAAGGTGCGCGACGGCGTGGCCATTCTGGCCC | |
| | TGGGCGCCCACAGCTACTGGCGCCAGCATGGAA | |
| | CCCTGCCAGCCCCGGCCGGGGACTGCCGTGTT | |
| | CACCCTGGGCCCGTCAGCCCTGCCCGGGAGGC | |
| | CTTCTACAGGCACCTACTGAATGTCACCTGGGAG | |
| | GGCCGAGACTTCTCCTTCAGCCCTGGTGGGTAC | |
| | CTGGTCCAGCCCACCATGGTGGTGATCGCCCTC | |
| | AACCGGCACCGCCTCTGGGAGATGGTGGGGCG | |
| | CTGGGAGCATGGCGTCCTATACATGAAGTACCC | |
| | CGTGTGGCCTCGCTACAGTGCCTCTCTGCAGCC | |
| | TGTGGTGGACAGTCGGCACCTGACGGTG | |

The invention further relates to functionally analogous sequences of the respective NMDAR protein constructs, domains, linkers and further elements comprised by the constructs. Protein modifications to the NMDAR protein constructs of the present invention, which may occur through substitutions in amino acid sequence, and nucleic acid sequences encoding such molecules, are also included within the scope of the invention. Substitutions as defined herein are modifications made to the amino acid sequence of the protein, whereby one or more amino acids are replaced with the same number of (different) amino acids, producing a protein which contains a different amino acid sequence than the primary protein. In some embodiments this amendment will not significantly alter the function of the protein. Like additions, substitutions may be natural or artificial. It is well known in the art that amino acid substitutions may be made without significantly altering the protein's function. This is particularly true when the modification relates to a “conservative” amino acid substitution, which is the substitution of one amino acid for another of similar properties. Such “conserved” amino acids can be natural or synthetic amino acids which because of size, charge, polarity and conformation can be substituted without significantly affecting the structure and function of the protein. Frequently, many amino acids may be substituted by conservative amino acids without deleteriously affecting the protein's function.

In general, the non-polar amino acids Gly, Ala, Val, Ile and Leu; the non-polar aromatic amino acids Phe, Trp and Tyr; the neutral polar amino acids Ser, Thr, Cys, Gin, Asn and Met; the positively charged amino acids Lys, Arg and His; the negatively charged amino acids Asp and Glu, represent groups of conservative amino acids. This list is not exhaustive. For example, it is well known that Ala, Gly, Ser and sometimes Cys can substitute for each other even though they belong to different groups.

As explained herein, in the context of the invention the NMDAR protein construct of the invention may be provided at the protein level or in the form of one or more nucleic acids encoding the respective NMDAR protein construct, which may comprise more than one protein.

Nucleic acid sequences of the invention include the nucleic acid sequences encoding NMDAR protein constructs or individual proteins that form part of a NMDAR protein construct of the invention. Protein sequences according to Table 1 and functionally analogous sequences represent preferred NMDAR protein constructs of the invention or parts thereof. Preferred nucleic acid sequence encoding NMDAR protein constructs of the invention or parts thereof are listed under Table 2.

The NMDAR protein constructs of the invention may include proteins tags that allow easy identification or binding of the provided NMDAR protein constructs through standard techniques, for example by using antibodies directed against the protein tag. A preferred protein-tag that can be encoded by a nucleic acid sequence of the invention is a V5-tag, myc-tag, HA-tag, HIS-tag or an antibody Fc-Fragment. Alternative tags may be used instead of a V5-tag. Such alternatives are well known in the art and can be selected by a skilled person.

In another aspect, the invention encompasses NMDAR protein constructs as disclosed herein as well as their use in the context of the methods disclosed herein. In particular, the invention also relates to nucleic acid molecules encoding NMDAR protein constructs of the invention, and in particular one or more nucleic acid molecules encoding such NMDAR protein constructs or parts of such constructs, selected from the group comprising:
a) one or more nucleic acid molecules comprising a nucleotide sequence which encodes an ECD of the NMDAR subunit GluN1 or a fragment thereof and an ECD of at least one of the NMDAR subunits GluN2A, GluN2B, GluN2C or GluN2D, or fragment thereof and preferably a dimerization domain and/or a capture domain;
b) one or more nucleic acid molecules which are complementary to the nucleotide sequences in accordance with a);
c) one or more nucleic acid molecules which undergo hybridization with the nucleotide sequences according to a) or b) under stringent conditions;
d) one or more nucleic acid molecules comprising a nucleotide sequence having sufficient sequence identity to be functionally analogous the nucleotide sequences according to a), b) or c);
e) one or more nucleic acid molecules which, as a consequence of the genetic code, are degenerated into nucleotide sequences according to a) through d); and
f) one or more nucleic acid molecules according the nucleotide sequences of a) through e) which are modified by deletions, additions, substitutions, translocations, inversions and/or insertions and functionally analogous to a nucleotide sequence according to a) through e)

Furthermore, the invention also relates to nucleic acid molecules encoding NMDAR protein constructs of the invention, and in particular one or more nucleic acid molecules encoding such NMDAR protein constructs or parts of such constructs, selected from the group comprising:
g) one or more nucleic acid molecules comprising a nucleotide sequence which encodes an ECD of the NMDAR subunit GluN1 or a fragment thereof and an ECD of the NMDAR subunit GluN2A or fragment thereof and/or GluN2B or fragment thereof and preferably a dimerization domain and/or a capture domain;
h) one or more nucleic acid molecules which are complementary to the nucleotide sequences in accordance with a);
i) one or more nucleic acid molecules which undergo hybridization with the nucleotide sequences according to a) or b) under stringent conditions;
j) one or more nucleic acid molecules comprising a nucleotide sequence having sufficient sequence identity to be functionally analogous the nucleotide sequences according to a), b) or c);
k) one or more nucleic acid molecules which, as a consequence of the genetic code, are degenerated into nucleotide sequences according to a) through d); and
l) one or more nucleic acid molecules according the nucleotide sequences of a) through e) which are modified by deletions, additions, substitutions, translocations, inversions and/or insertions and functionally analogous to a nucleotide sequence according to a) through e)

Accordingly, the invention encompasses nucleic acid molecules with at least 60%, preferably 70%, more preferably 80%, especially preferably 90% sequence identity to the nucleic acid molecule encoding NMDAR protein constructs of the invention or parts thereof.

Sequence variants of the claimed nucleic acids and/or proteins, for example defined by the provided % sequence identity, that maintain the said properties of the invention are also included in the scope of the invention. Such variants, which show alternative sequences, but maintain essentially the same properties, such as autoantibody-binding properties of the respective NMDAR protein construct of the invention, as the specific sequences provided are known as functional analogues, or as functionally analogous. Sequence identity relates to the percentage of identical nucleotides or amino acids when carrying out a sequence alignment, for example using software such as BLAST.

It will be appreciated by those of ordinary skill in the art that, as a result of the degeneracy of the genetic code, there are many nucleotide sequences that encode a polypeptide as described herein. Some of these polynucleotides bear minimal homology or sequence identity to the nucleotide sequence of any native gene. Nonetheless, polynucleotides that vary due to differences in codon usage are specifically contemplated by the present invention. Deletions, substitutions and other changes in sequence that fall under the described sequence identity are also encompassed in the invention.

Autoantigen and Disease Description

The invention relates to a soluble N-methyl-D-aspartate receptor (NMDAR) protein construct comprising one or more NMDAR autoantibody epitopes. As used herein, the term “NMDAR autoantibody epitopes” relates to epitopes formed by NMDAR, either by an individual subunit or epitopes that comprise residues or amino acids of more than one subunit of NMDAR. Furthermore, the conformation of the NMDAR subunits may be only stabilized by the presence of an ECD or fragments of an ECD of another subunit and certain epitopes may only be formed upon stabilization of such a conformation. In the context of the invention the NMDAR protein constructs and the epitopes formed by the constructs of the invention may be referred to as autoantigens. Binding between an autoantigen and antibody is, as such, an established phenomenon and reflects essentially the physical interaction between any given antibody and its target.

Various neurological autoimmune conditions are known to a skilled person, in which autoantibodies target typically either autoantigens of primarily the central or peripheral nervous system. Medical conditions are however also known, in which autoantibodies are directed against targets present in both the central and peripheral nervous systems. The present invention therefore envisages the use of NMDAR protein constructs of the invention in the context of diseases, in which autoantibodies predominantly target components of the central nervous system, or in which the pathogenic effect of said autoantibodies is caused by the autoantibodies targeting an autoantigen in the central nervous system.

As used herein, the “central nervous system” or CNS refers to the part of the nervous system consisting of the brain and spinal cord. The CNS is contained within the dorsal body cavity, with the brain housed in the cranial cavity and the spinal cord in the spinal canal. The CNS is divided in white and gray matter. This can also be seen macroscopically on brain tissue. The white matter consists of axons and oligodendrocytes, while the gray matter consists of neurons and unmyelinated fibers. Both tissues include a number of glial cells (although the white matter contains more), which are often referred to as supporting cells of the CNS. From and to the spinal cord are projections of the peripheral nervous system in the form of spinal nerves. The nerves connect the spinal cord to skin, joints, muscles etc. and allow for the transmission of efferent motor as well as afferent sensory signals and stimuli. This allows for voluntary and involuntary motions of muscles, as well as the perception of senses.

As used herein, the “peripheral nervous system” (PNS) consists of the nerves and ganglia outside the brain and spinal cord. The main function of the PNS is to connect the CNS to the limbs and organs, essentially serving as a relay between the brain and spinal cord and the rest of the body. Unlike the CNS, the PNS is not protected by the vertebral column and skull, or by the blood-brain barrier.

Emerging research now shows that autoantibodies do have access to the CNS (Zong et al Front Immunol. 2017; 8: 752) and that autoantibody-producing B cells are present in the CNS. Under normal conditions, immunoglobulins go through the blood brain barrier (BBB) at a low rate; a good example is immunoglobulin G (IgG). IgG concentration in the cerebrospinal fluid (CSF) is approximately 1% of the levels in the peripheral circulation. This indicates that once the autoantibodies reach the CNS they can cause disease as it has been observed in autoimmune encephalitis. In certain situations, the BBB may also become leaky because of stroke, brain trauma, hemorrhages, microangiopathy, or brain tumors, and antibody penetration might increase.

As used herein, the term “autoantibody-mediated psychiatric condition” relates to any medical condition comprising the presence of autoantibodies, preferably directed to an autoantigen primarily targeted in the central nervous system, in which psychiatric (neuropsychiatric) symptoms are also observed. A number of central nervous system disorders, including encephalitis and severe psychiatric disorders, have been demonstrated to associate with specific neuronal surface autoantibodies (NSAbs). It has become clear that specific autoantibodies targeting neuronal surface antigens and ion channels cause severe mental disturbances, i.e. lead to neuropsychiatric symptoms. A number of studies show the presence of autoantibodies in specific mental conditions such as schizophrenia and bipolar disorders. Additional disorders relate to neuropsychiatric disorders such as schizophrenia, bipolar disorder, MDD, substance-induced psychosis, Huntington's disease, Alzheimer's disease, and neuropsychiatric systemic lupus erythematosus (Zong et al, Front Immunol. 2017; 8: 752).

In some embodiments, the diseases to be treated or diagnosed by means of the present invention are an autoimmune encephalopathy or encephalomyelopathy. An “encephalopathy” is typically any disorder or disease of the brain, especially chronic degenerative conditions. Encephalopathy may refer to permanent (or degenerative) brain injury, or a reversible injury. It can be due to direct injury to the brain, or illness remote from the brain. Symptoms often include intellectual disability, irritability, agitation, delirium, confusion, somnolence, stupor, coma and psychosis. As used herein, an “autoimmune encephalopathy” refers to any brain disease with an autoimmune component including autoimmune encephalitis. As used herein, an “autoimmune encephalomyelopathy” is any disease that affects both the brain and the spinal cord with an autoimmune component.

Anti-N-methyl-D-aspartate (NMDA) receptor encephalitis is a form of encephalitis occurring often in women and is associated with antibodies against NR1 (GluN1) and/or NR2 subunits of the NMDA receptor, although primarily the NR1 subunit. Anti-NMDA receptor encephalitis was first described several years ago in multiple large studies that characterized the clinical syndrome in detail (Dalmau et al. 2008). Patients with anti-NMDAR encephalitis suffer from a severe form of encephalitis with characteristic clinical multistage features, predominantly affecting children and young women. It progresses from psychiatric symptoms, memory deficits, and epileptic seizures into a state of loss of consciousness, autonomic dysfunction, dyskinesias and hypoventilation (Dalmau et al. Lancet Neurol. 2011; 10:63-74; Prüss et al. 2010, Neurology. 75(19):1735-9; Prüss et al. 2013, Neurology. 75(19):1735-9). Hallmark of the disease are antibodies against the NR1/GluN1 subunit of the NMDAR1. This has profoundly changed the therapeutic concept in encephalitis, since NMDAR encephalitis was not recognized as a distinct subgroup of encephalitis before 2007. Therefore, it was previously considered as encephalitis of unknown etiology and was not adequately treated.

The N-methyl-D-aspartate receptor (also known as the NMDA receptor or NMDAR), is a glutamate receptor and ion channel protein found in nerve cells. The NMDA receptor is one of three types of ionotropic glutamate receptors. The other receptors are the AMPA and kainate receptors. It is activated when glutamate and glycine (or D-serine) bind to it, and when activated it allows positively charged ions to flow through the cell membrane. The NMDA receptor is very important for controlling synaptic plasticity and memory function. The receptor usually is assembled as a heteromeric complex that interacts with multiple intracellular proteins by three different subunits: NR1, NR2 and NR3. NR1 has eight different isoforms generated by alternative splicing from a single gene. There are four different NR2 subunits (A-D) and late in the 20th century NR3A and NR3B subunits have been reported. Six separate genes encode for NR2 and NR3. According to a more recent nomenclature the subunits are called GluN1, GluN2 and GluN3 instead of NR1, NR2 and NR3, respectively. The alternative variants of the subunits are identified accordingly (for example NR2A and NR2B as GluN2A and GluN2B, respectively).

Each receptor subunit has modular design. The extracellular domain contains two globular structures: an amino terminal domain (ATD, sometimes referred to as modulatory domain) and a ligand-binding domain. NR1 subunits bind the co-agonist glycine and NR2 subunits bind the neurotransmitter glutamate. The agonist-binding module links to a membrane domain, which consists of three transmembrane segments and a re-entrant loop reminiscent of the selectivity filter of potassium channels. The membrane domain contributes residues to the channel pore and is responsible for the receptor's high-unitary conductance, high-calcium permeability, and voltage-dependent magnesium block. Each subunit has an extensive cytoplasmic domain, which contain residues that can be directly modified by a series of protein kinases and protein phosphatases, as well as residues that interact with a large number of structural, adaptor, and scaffolding proteins.

The NMDAR NR1/GluN1 is a component of NMDA receptor complexes that function as heterotetrameric, ligand-gated ion channels with high calcium permeability and voltage-dependent sensitivity to magnesium. Channel activation requires binding of the neurotransmitter glutamate to the GluN2 subunit, glycine binding to the GluN1 subunit, plus membrane depolarization to eliminate channel inhibition by Mg2+. A number of protein isoforms of the NMDAR NR1 protein are known, such as, without limitation, those of the Gene Bank Accession numbers: XP_011516885.1, XP_005266130.1, XP_005266129.1, XP_005266128.1, NP 001172020.1, NP_001172019.1, NP_000823.4, NP_015566.1, NP_067544.1. Any one or more of said sequences or isoforms or functionally analogous derivatives thereof may be employed in the contest of the NMDAR protein constructs of the present invention.

With respect to GluN2/NR2, there is only a single subunit found in invertebrate organisms, four distinct isoforms of the NR2 subunit are expressed in vertebrates and are referred to with the nomenclature NR2A/GluN2A through NR2D/GluN2D (encoded by GRIN2A, GRIN2B, GRIN2C, GRIN2D). They contain the binding-site for the neurotransmitter glutamate. Unlike NR1 subunits, NR2 subunits are expressed differentially across various cell types and control the electrophysiological properties of the NMDA receptor. One particular subunit, NR2B, is mainly present in immature neurons and in extrasynaptic locations, and contains the binding-site for the selective inhibitor ifenprodil. Whereas NR2B is predominant in the early postnatal brain, the number of NR2A subunits grows, and eventually NR2A subunits outnumber NR2B. This is called the NR2B-NR2A developmental switch, and is notable because of the different kinetics each NR2 subunit lends to the receptor. For instance, greater ratios of the NR2B subunit leads to NMDA receptors which remain open longer compared to those with more NR2A.

The NMDAR has a variety of physiological roles and any dysfunction, either enhanced or decreased activity, may result in neuropsychiatric disorders, such as schizophrenia, bipolar disorder, MDD, substance-induced psychosis, Huntington's disease, Alzheimer's disease, and neuropsychiatric systemic lupus erythematosus (NPSLE). The NMDAR therefore plays a critical role in multiple psychiatric disorders including depression. In addition, a subgroup of patients with atypical dementia harbor anti-NMDAR1 antibodies, thereby qualifying as a medical condition associated with autoantibodies against the NMDAR, and removal of such NMDAR autoantibodies by unspecific removal of all antibodies resulted in clinical improvement in selected cases (Prüss et al. 2010, Neurology. 75(19):1735-9; Doss et al. 2014 Ann Clin Transl Neurol. 1(10):822-32). Furthermore, autism can occur in the children of mothers suffering from autoantibody-mediated disorders. Several studies have found a correlation between the presence of circulating maternal autoantibodies and neuronal dysfunction in the neonate (Fox-Edmiston et al, 2015, CNS Drugs, 29(9): 715-724). Specifically, maternal anti-brain autoantibodies, which may access the fetal compartment during gestation, have been identified as one risk factor for developing Autism Spectrum Disorder (ASD). The presence of NMDAR-autoantibodies may therefore lead to autism in offspring of diseased mothers, such that the present invention also represents potential treatment of such disorders and/or a prophylactic approach towards avoiding such disease in children.

As such, any medical condition, in which a contribution of NMDAR autoantibodies to pathogenesis has been described or suggested, for example due to a correlation of occurrence of NMDAR autoantibodies and disease symptoms, qualifies as a medical condition associated with NMDAR autoantibodies. Furthermore, the constructs of the invention can be used to analyze a sample of a patient suffering from a condition that has been described or suggested as a condition associated with NMDAR autoantibodies for the presence of such antibodies and can subsequently be treated by the means of the present invention.

In contrast to anti-NMDAR in autoimmune encephalitis, which mainly targets the GluN1 subunit, autoantibodies have been found that target the GluN2 subunit of NMDAR, and these were associated with depression in systemic lupus erythematosus (SLE) patients (Lapteva et al. Arthritis Rheum (2006) 54(8):2505-14).

Aspects of the In Vitro Method for the Detection of NMDAR Autoantibodies

An autoantibody is an antibody (a type of protein) manufactured by the immune system that is directed against one or more of the individual's own proteins. Many autoimmune diseases are associated with and/or caused by such autoantibodies.

The term “autoimmune disease” refers to any given disease associated with and/or caused by the presence of autoantibodies. Autoimmune diseases arise from an abnormal immune response of the body against substances and tissues normally present in the body (autoimmunity). This may be restricted to certain organs or involve a particular tissue.

As used herein, the term “sample” is a biological sample that is obtained or isolated from the patient or subject. “Sample” as used herein may, e.g., refer to a sample of bodily fluid or tissue obtained for the purpose of diagnosis, prognosis, or evaluation of a subject of interest, such as a patient. Preferably herein, the sample is a sample of a bodily fluid, such as blood, serum, plasma, cerebrospinal fluid, urine, saliva, sputum, pleural effusions, cells, a cellular extract, a tissue sample, a tissue biopsy, a stool sample and the like.

The term “individual,” “subject,” or “patient” typically refers to humans, but also to other animals including, e.g., other primates, rodents, canines, felines, equines, ovines, porcines, and the like. As used herein, the “patient” or “subject” may be a vertebrate. In the context of the present invention, the term “subject” includes both humans and animals, particularly mammals, and other organisms.

As used herein, the terms “diagnosis”, “prognosis” and “assessment of likelihood” relate to determining the probability of whether a subject with, or at risk of having and/or developing, a medical condition associated with NMDAR autoantibodies

The terms “diagnosis” and “diagnosing” include the use of the NMDAR protein construct, the method, the kit and further aspects of the invention to determine the presence or absence or likelihood of presence or absence of a medically relevant disorder in an individual. The term also includes devices, methods, and systems for assessing the level of disease activity in an individual. In some embodiments, statistical algorithms are used to diagnose a mild, moderate, severe, or fulminant form of the disorder based upon the criteria developed by Truelove et al., Br. Med. J., 12:1041-1048 (1955). In other embodiments, statistical algorithms are used to diagnose a mild to moderate, moderate to severe, or severe to fulminant form of the autoimmune disease associated with NMDAR autoantibodies.

The invention also encompasses use of the method for disease monitoring, also known as monitoring the progression or regression of the autoimmune disease and therapy monitoring. The term “monitoring” includes the use of the NMDAR constructs and the methods and other aspects of the invention disclosed herein to determine the disease state (e.g., presence or severity of the autoimmune disease) of an individual. In certain instances, the results of a statistical algorithm (e.g., a learning statistical classifier system) are compared to those results obtained for the same individual at an earlier time. In some aspects, the kits, constructs, devices, methods, and systems of the present invention can also be used to predict the progression of the autoimmune disease, e.g., by determining a likelihood for the autoimmune disease to progress either rapidly or slowly in an individual based on the presence or level of at least one marker in a sample, such as one or more kinds of NMDAR autoantibodies. The present invention can also be used to predict the regression of the autoimmune disease, e.g., by determining a likelihood for the autoimmune disease to regress either rapidly or slowly in an individual based on the presence or level of at least one marker in a sample. Therapy monitoring may also be conducted, whereby a subject is monitored for disease progression during the course of any given therapy.

In aspects of the invention, the presence or level of NMDAR autoantibodies is determined using an immunoassay or an immunohistochemical assay. A non-limiting example of an immunoassay suitable for use in the method of the present invention includes an ELISA. Examples of immunohistochemical assays suitable for use in the method of the present invention include, but are not limited to, immunofluorescence assays such as direct fluorescent antibody assays, IFA assays, anticomplement immunofluorescence assays, and avidin-biotin immunofluorescence assays. Other types of immunohistochemical assays include immunoperoxidase assays.

The term “affinity reagent” in the context of the present invention relates to an antibody, peptide, nucleic acid, small molecule, or any other molecule that specifically binds to a target molecule in order to identify, track, capture, or influence its activity. The term “capturing” refers to binding of a target molecule by an affinity reagent.

The term “secondary affinity reagent” refers to any affinity reagent according to the above definition, which is used to bind to an antigen that is already bound by another affinity reagent.

As used herein, the term “antibody” includes a population of immunoglobulin molecules, which can be polyclonal or monoclonal and of any isotype, or an immunologically active fragment of an immunoglobulin molecule. Such an immunologically active fragment contains the heavy and light chain variable regions, which make up the portion of the antibody molecule that specifically binds an antigen. For example, an immunologically active fragment of an immunoglobulin molecule known in the art as Fab, Fab′ or F(ab′)2 is included within the meaning of the term antibody. The term “monoclonal antibody” refers to antibodies that are made by identical immune cells that are all clones of a unique parent cell, in contrast to polyclonal antibodies, which are made from several different immune cells. Monoclonal antibodies can have monovalent affinity, in that they bind to the same epitope (the part of an antigen that is recognized by the antibody). Engineered bispecific monoclonal antibodies also exist, where each “arm” of the antibody is specific for a different epitope. Given almost any substance, it is possible to produce monoclonal antibodies that specifically bind to that substance; they can then serve to detect or purify that substance.

In embodiments, the invention relates to an in vitro method for the detection of NMDAR autoantibodies in a sample. In embodiments, the method is an immunoassay. Following addition of sample solution, the patients antibody included therein binds to the NMDAR protein construct. The antibody which is obtained e.g. from the serum or stool of a patient and bound to the NMDAR protein construct can subsequently detected using a label, or labelled reagent and optionally quantified. Thus, according to the invention, detection of the antibodies in this method can be effected using labelled reagents according to the well-known ELISA (Enzyme-Linked Immunosorbent Assay) technology. Labels according to the invention therefore comprise enzymes catalysing a chemical reaction which can be determined by optical means, especially by means of chromogenic substrates, chemiluminescent methods or fluorescent dyes. In another preferred embodiment the autoantibodies are detected by labelling with weakly radioactive substances in radioimmunoassays (RIA) wherein the resulting radioactivity is measured.

As examples of means for detecting the label in the method of the present invention, a variety of immunoassay techniques, including competitive and non-competitive immunoassays, can be used to determine the presence or level of one or more markers in a sample (see, e.g., Self et al., Curr. Opin. Biotechnol., 7:60-65 (1996)). The term immunoassay encompasses techniques including, without limitation, enzyme immunoassays (EIA) such as enzyme multiplied immunoassay technique (EMIT), enzyme-linked immunosorbent assay (ELISA), antigen capture ELISA, sandwich ELISA, IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-2193 (1997); Bao, J. Chromatogr. B. Biomed. Sci., 699:463-480 (1997)). Liposome immunoassays, such as flow-injection liposome immunoassays and liposome immunosensors, are also suitable for use in the present invention (see, e.g., Rongen et al., J. Immunol. Methods, 204:105-133 (1997)). In addition, nephelometry assays, in which the formation of protein/antibody complexes results in increased light scatter that is converted to a peak rate signal as a function of the marker concentration, are suitable for use in the present invention. Nephelometry assays are commercially available from Beckman Coulter (Brea, Calif.; Kit #449430) and can be performed using a Behring Nephelometer Analyzer (Fink et al., J. Clin. Chem. Clin. Biol. Chem., 27:261-276 (1989)).

The immunoassays described above are particularly useful for determining the presence or level of one or more NMDAR autoantibodies in a sample (and may be considered examples of means for detecting a label).

In another preferred embodiment of the method according to the invention the autoantibodies are detected in an immunoassay, preferably with direct or indirect coupling of one reactant to a labelling substance. This enables flexible adaptation of the method to the potentials and requirements of different laboratories and their laboratory diagnostic equipment. In one advantageous embodiment the autoimmune disease-specific antibodies are detected in an immunoassay wherein the antibodies are present dissolved in a liquid phase, preferably diluted in a conventional buffer solution well-known to those skilled in the art or in an undiluted body fluid. According to the invention, detection can also be effected using stool samples. Furthermore, the detection method of the invention may be carried out by binding the constructs of the invention to cells that express NMDAR autoantibodies on their surface. The detection of cells that bind to the constructs of the invention may occur through direct or indirect labelling of the constructs.

In another preferred embodiment of the invention, soluble or solid phase-bound NMDAR protein constructs are used to bind the antibodies. In a second reaction step, anti-human immunoglobulins can be employed, preferably selected from the group comprising anti-human IgA, anti-human IgM and/or anti-human IgG antibodies, said anti-human immunoglobulins being detectably labelled conjugates of two components which can be conjugated with any conventional labelling enzymes, especially chromogenic and/or chemiluminescent substrates, preferably with horseradish peroxidase, alkaline phosphatase. The advantage of this embodiment lies in the use of ELISA technology usually available in laboratory facilities so that detection according to the invention can be established in a cost-effective manner. In another preferred embodiment of the invention the antibody bound to an NMDAR protein construct of the invention reacts with anti-human immunoglobulins, preferably selected from the group comprising anti-human IgA, anti-human IgM and/or anti-human IgG antibodies, detectably coupled to fluorescein isothiocyanate (FITC). Much like the above-mentioned ELISA, the FITC technology represents a system that is available in many places and therefore allows smooth and low-cost establishment of the inventive detection in laboratory routine. The skilled person is aware of further standard detection technologies that can be used in the context of the method of the invention.

Specific immunological binding of the antibody to the marker of interest can be detected directly or indirectly via a label. Any given means for detecting these labels may be considered means for detecting the label according to the method of the invention. Direct labels include fluorescent or luminescent tags, metals, dyes, radionuclides, and the like, attached to the antibody. An antibody labelled with iodine-125 (125I) can be used for determining the levels of one or more markers in a sample. A chemiluminescence assay using a chemiluminescent antibody specific for the marker is suitable for sensitive, non-radioactive detection of marker levels. An antibody labelled with fluorochrome is also suitable for determining the levels of one or more markers in a sample. Examples of fluorochromes include, without limitation, DAPI, fluorescein, Hoechst 33258, R-phycocyanin, B-phycoerythrin, R-phycoerythrin, rhodamine, Texas red, and lissamine. Secondary antibodies linked to fluorochromes can be obtained commercially, e.g., goat F(ab′)2 anti-human IgG-FITC is available from Tago Immunologicals (Burlingame, Calif.). Further fluorescent labels are commonly used and known to a skilled person.

Indirect labels include various enzymes well-known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm.

A signal from the direct or indirect label can be analysed, for example, using a spectrophotometer to detect colour from a chromogenic substrate; a radiation counter to detect radiation such as a gamma counter for detection of 125I; or a fluorometer to detect fluorescence in the presence of light of a certain wavelength. For detection of enzyme-linked antibodies, a quantitative analysis of the amount of marker levels can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

Plate readers, also known as microplate readers or microplate photometers, are instruments which are used to detect biological, chemical or physical events of samples in microtiter plates. They are widely used in research, drug discovery, bioassay validation, quality control and manufacturing processes in the pharmaceutical and biotechnological industry and academic organizations. Sample reactions can be assayed, for example, without limitation, in 6-1536 well format microtiter plates. Common detection modes for microplate assays are, for example, without limitation, absorbance, fluorescence intensity, luminescence, time-resolved fluorescence, and fluorescence polarization. A camera device in the context of the present invention is a device suitable for detection of the signal of the labeled secondary affinity reagent directed against GP2. The camera device can provided as being comprised in the plate reader or individually. The person skilled in the art is familiar with such devices, which are selected based on the label of the secondary affinity reagent.

In certain embodiments, the present invention provides methods of diagnosing the autoimmune disease or clinical subtypes thereof using NMDAR protein constructs of the invention. A variety of autoimmune disease markers, such as biochemical markers, serological markers, genetic markers, or other clinical or echographic characteristics, are suitable for use and can be combined with statistical algorithms to classify a sample from an individual as an the autoimmune disease sample. Examples of further markers of autoimmune disease associated with NMDAR autoantibodies suitable for use in the present invention are known to a skilled person. One skilled in the art will know of additional markers suitable for use in the statistical algorithms of the present invention.

In another preferred embodiment of the invention the NMDAR protein constructs according to the present application are immobilized on a surface. More specifically, one or more solid phase-bound NMDAR protein constructs as disclosed herein are bound to organic, inorganic, synthetic and/or mixed polymers, preferably agarose, cellulose, silica gel, polyamides and/or polyvinyl alcohols. In the meaning of the invention, immobilization is understood to involve various methods and techniques to fix the peptides on specific carriers, e.g. according to WO 99/56126 or WO 02/26292. For example, immobilization can serve to stabilize the constructs so that their activity would not be reduced or adversely modified by biological, chemical or physical exposure, especially during storage or in single-batch use. Immobilization of the peptides allows repeated use under technical or clinical routine conditions; furthermore, a sample—preferably blood components—can be reacted with at least one of the constructs according to the invention in a continuous fashion. In particular, this can be achieved by means of various immobilization techniques, with binding of the peptides to other peptides or molecules or to a carrier proceeding in such a way that the three-dimensional structure—particularly in the active centre mediating the interaction with the autoantibodies—of the corresponding molecules, especially of said peptides, would not be changed. Advantageously, there is no loss in specificity to the autoantibodies of patients as a result of such immobilization. In the meaning of the invention, at least three basic methods can be used for immobilization:

(i) Crosslinking: in crosslinking, the peptides are fixed to one another without adversely affecting their activity. Advantageously, they are no longer soluble as a result of such crosslinking.

(ii) Binding to a carrier: binding to a carrier proceeds via adsorption, ionic binding or covalent binding, for example. Such binding may also take place inside microbial cells or liposomes or other membranous, closed or open structures. Advantageously, the peptides are not adversely affected by such fixing. For example, multiple or continuous use of carrier-bound peptides is possible with advantage in clinical diagnosis or therapy.

(iii) Inclusion: inclusion in the meaning of the invention especially proceeds in a semipermeable membrane in the form of gels, fibrils or fibres. Advantageously, encapsulated peptides are separated from the surrounding sample solution by a semipermeable membrane in such a way that interaction with the autoantibodies or fragments thereof still is possible. Various methods are available for immobilization, such as adsorption on an inert or electrically charged inorganic or organic carrier. For example, such carriers can be porous gels, aluminum oxide, bentonite, agarose, starch, nylon or polyacrylamide. Immobilization proceeds via physical binding forces, frequently involving hydrophobic interactions and ionic binding. Advantageously, such methods are easy to handle and have little influence on the conformation of the peptides. Advantageously, binding can be improved as a result of electrostatic binding forces between the charged groups of the peptides and the carrier, e.g. by using ion exchangers, particularly Sephadex.

Another method is covalent binding to carrier materials. In addition, the carriers may have reactive groups forming homopolar bonds with amino acid side chains. Suitable groups in peptides are carboxy, hydroxy and sulfide groups and especially the terminal amino groups of lysines. Aromatic groups offer the possibility of diazo coupling. The surface of microscopic porous glass particles can be activated by treatment with silanes and subsequently reacted with peptides. For example, hydroxy groups of natural polymers can be activated with bromocyanogen and subsequently coupled with peptides. Advantageously, a large number of peptides can undergo direct covalent binding with polyacrylamide resins. Inclusion in three-dimensional networks involves inclusion of the peptides in ionotropic gels or other structures well-known to those skilled in the art. More specifically, the pores of the matrix are such in nature that the peptides are retained, allowing interaction with the target molecules. In crosslinking, the peptides are converted into polymer aggregates by crosslinking with bifunctional agents. Such structures are gelatinous, easily deformable and, in particular, suitable for use in various reactors. By adding other inactive components such as gelatin in crosslinking, advantageous improvement of mechanical and binding properties is possible. In microencapsulation, the reaction volume of the peptides is restricted by means of membranes. For example, microencapsuation can be carried out in the form of an interfacial polymerization. Owing to the immobilization during microencapsulation, the peptides are made insoluble and thus reusable. In the meaning of the invention, immobilized constructs are all those peptides being in a condition that allows reuse thereof. Restricting the mobility and solubility of the peptides by chemical, biological or physical means advantageously results in lower process cost, particularly when eliminating autoantibodies from blood components.

The invention also relates to a diagnostic kit for the determination of autoimmune diseases associated with NMDAR autoantibodies, comprising one or more NMDAR protein constructs as disclosed herein. A kit for diagnosis includes all necessary analyte specific reagents required for carrying out a diagnostic test. It may also contain instructions on how to conduct the test using the provided reagents. The diagnostic kit optionally includes instructions concerning combining the contents of the kit and/or providing a formulation for the detection of an autoimmune disease associated with NMDAR autoantibodies, such as NMDAR encephalitis. For example, the instruction can be in the form of an instruction leaflet or other medium providing the user with information as to the type of method wherein the substances mentioned are to be used. Obviously, the information need not necessarily be in the form of an instruction leaflet, and the information may also be imparted via the Internet, for example. To a patient, one advantageous effect of such a kit is, for instance, that he or she, without directly addressing a physician, can determine the actual state of a disease even during a journey and optionally adapt diet and activities accordingly.

Aspects of the Blood Treatment Devices and Immobilization of the NMDAR Protein Constructs in the Device

The invention also relates to a blood treatment device configured to remove NMDAR autoantibodies from the blood or blood plasma of a person in need thereof in an extracorporeal blood circuit, wherein the device comprises a matrix having one or more NMDAR protein constructs of the invention immobilized thereon.

The “matrix” as used herein thus refers to a material inside the blood treatment device that provides an internal material or surface through or over which blood or plasma is passed. The matrix as used in the context of the present invention preferably comprises a support to which the NMDAR protein construct is bound. The support therefore serves as a carrier for the NMDAR protein constructs, even though it may fulfil other functions.

The “support” as used herein refers to the portion of the matrix which serves as the “substrate” or “support material” to which the constructs according to the invention are bound. Such support or support material is sometimes also referred to as “adsorption material” or “adsorber”, as used in an “adsorption column” or “column” or “adsorption cartridge”. A suitable support according to the present invention should be uniform, hydrophilic, mechanically and chemically stable over the relevant pH range and temperature with no or a negligible leaching of the NMDAR protein constructs during use, have good flow characteristics for whole blood and/or blood plasma, and provides a large surface area for NMDAR protein construct attachment.

The support can for example be a resin, a membrane or a non-woven material. A “non-woven” material refers to a material which is broadly defined as a sheet, fabric or web structure bonded together by entangling fiber or filaments (and by perforating films) mechanically, thermally, or chemically but not by weaving or knitting. A “resin” refers to an insoluble material which can take the form of gels or gel beads or microporous beads, or a sponge. Such resins can be natural or bio-polymers, synthetic polymers and inorganic materials. Agarose, dextrose and cellulose beads are commonly employed natural supports. Synthetic polymeric or organic supports are mostly based on acrylamide, polystyrene and polymethacrylate derivatives, whereas, porous silica and glass are some frequently used inorganic supports.

According to one embodiment of the invention, the resin is composed of polymers selected from the group consisting of alginate, chitosan, chitin, collagen, carrageenan, gelatin, cellulose, starch, pectin and sepharose; inorganic materials selected from the group consisting of zeolites, ceramics, celite, silica, glass, activated carbon and char-coal; or synthetic polymers selected from the group consisting of polyethylene (PE), polyoxymethylene (POM), polypropylene (PP), polyvinylchloride (PVC), polyvinyl acetate (PVA), polyvinylidene chloride (PVDC), polystyrene (PS), polytetrafluoroethylene (PTFE), polyacrylate (PAA), polymethyl methacrylate (PMMA), polyacrylamide, polyglycidyl methac-rylate (PGMA), acrylonitrile butadiene styrene (ABS), polyacrylonitrile (PAN), polyester, polycarbonate, polyethylene terephthalate (PET), polyamide, polyaramide, polyethylene glycol (PEG), polyvinylpyrrolidone (PVP), polysulfone (PS), polyethersulfone (PES), polyarylethersulfone (PEAS), ethylene vinyl acetate (EVA), ethylene vinyl alcohol (EVOH), polyamideimide, polyaryletherketone (PAEK), polybutadiene (PBD), polybutylene (PB), polybutylene terephthalate (PBT), polycaprolactone (PCL), polyhydroxyalkanoate, polyether ether ketone (PEEK), polyether ketone ketone (PEKK), polyether imide (PEI), polyimide, polylactic acid (PLA), polymethyl pentene (PMP), poly(p-phenylene ether) (PPE), polyurethane (PU), styrene acrylonitrile (SAN), polybutenoic acid, poly(4-allyl-benzoic acid), poly(glycidyl acrylate), polyglycidyl methacrylate (PGMA), acrylonitrile butadiene styrene (ABS), polydivinylbenzene (PDVB), poly(allyl glycidyl ether), poly(vinyl glycidyl ether), poly(vinyl glycidyl urethane), polyallylamine, pol-yvinylamine, copolymers of said polymers and any of these polymers modified by introduction of functional groups.

Various known methods can be used to immobilize the NMDAR protein constructs to the support and/or matrix according to the invention. Such immobilization preferably is specific or selective in that it immobilizes the NMDAR protein constructs whereas other proteins and components present in blood or blood plasma or a sample thereof (in vitro) are not immobilized to a significant degree.

The “immobilizing” of an NMDAR protein construct to the support for providing a matrix which can be used in a device according to the invention refers to a non-covalent or covalent interaction that holds two molecules together. According to one embodiment of the invention, the expression refers to a covalent interaction, i.e. to covalently bound NMDAR protein constructs. Non-covalent interactions include, but are not limited to, hydrogen bonding, ionic interactions among charged groups, van der Waals interactions, and hydrophobic interactions among non-polar groups. One or more of these interactions can mediate the binding of two molecules to each other. Binding may otherwise be specific or selective, or unspecific.

According to one embodiment, the NMDAR protein construct comprises affinity tags for immobilizing it on the support. Affinity tags can be used for purifying the protein during production and/or for immobilizing them on the support of the matrix of the present invention. Affinity tags can be short polypeptide sequences or whole proteins, co-expressed as fusion partners with the NMDAR protein constructs. Different types of affinity tags are well known in the art, wherein polyhistidine or Hiss-tags, C-myc-tags and FLAG-tags are especially well described and are options for binding the constructs according to the invention to the support material. The noncovalent linkage of biotin to strepavidin or avidin can also be used to immobilize the NMDAR protein construct to a support. In embodiments, binding is mediated through a Fc fragment that forms part of certain constructs of the invention.

According to another embodiment of the invention, the NMDAR protein constructs are covalently attached to the support as further detailed below and/or as described the prior art. Covalent coupling generally includes either covalent non-site directed attachment of the protein or site-directed attachment of the protein. The support which forms the basis for the generation of a matrix must provide or facilitate chemical activation, thus allowing the chemical coupling of the construct. Many coupling methods for immobilizing proteins, such as NMDAR protein constructs, are well known in the art.

For example, the activation chemistry should be stable over a wide range of pH, buffer conditions and temperature resulting in negligible leaching of the constructs. The coupling method should avoid improper orientation, multisite attachment or steric hindrance of the constructs. The construct density per volume of matrix can be optimized to promote target accessibility and reaction.

The covalent coupling can be carried out via common functional groups, including amines, alcohols, carboxylic acids, aldehydes and epoxy groups. Carbodiimide compounds can be used to activate carboxylic groups of proteins for direct conjugation to the primary amines on the support surface via amide bonds. The most commonly used carbodiimides are the water-soluble EDC (1-ethyl-3-(-3-dimethylaminopropyl) carbodiimide) for aqueous crosslinking and the water-insoluble DCC (N′,N′-dicyclohexyl carbodiimide) for non-aqueous organic synthesis methods.

Alternatively, the supports may carry specific functional groups for coupling a linker and/or protein construct thereto. For example, functionalized resins are commercially available and known to a person with skill in the art. A wide range of coupling chemistries, involving primary amines, sulfhydryls, aldehydes, hydroxyls and carboxylic acids are available in said commercial supports. Examples for commercially available activated resins are CarboLink Coupling resin, Profinity™ Epoxide resin, Affi-Gel 10 and 15, Epoxy-activated Sepharose™ 6B, Tresyl chloride-activated agarose, and Purolite® Lifetech™ methacrylate polymers functionalized with epoxy groups.

According to one embodiment of the invention, the support material should be porous, wherein the pore size is in the range of from 10 to 200 nm. According to another embodiment of the invention, the support takes the form of beads. According to yet another embodiment, the support according to the invention comprises magnetic beads. Magnetic beads are prepared by entrapping magnetite within agarose or other polymeric material, on which the NMDAR protein construct according to the invention is immobilized.

According to another embodiment of the present invention the support is a membrane. Membranes as components of affinity matrices have been used in protein purification, due to their simplicity, ease of handling, reduced surface area and lower diffusion limitations compared to gels, resins and beads. The membranes can take the physical form of a hollow fiber or, alternatively, of a flat sheet membrane. According to one embodiment, the support comprises a hemodialysis hollow fiber membrane dialyzer, wherein the filter is a hemodialyzer.

The hollow fiber or flat sheet membranes for use as supports in a device according to the invention may be composed of cellulose, cellulose ester (cellulose acetate and cellulose triacetate), poly(methylmethacrylate)(PMMA), polyamide (PA), other nitrogen-containing polymers (polybenzimidazole, polyacrylonitrile (PAN), polyglycidyl methacrylate (PGMA), polyvinylpyrrolidone (PVP), polysulfone (PS), polyethersulfone (PES) or polyarylethersulfone (PAES). A hollow fiber membrane which can advantageously be utilized for providing a device according to the invention preferably has an inner diameter in the range of 100 to 500 μm. According to another embodiment of the invention, specifically when the membrane support is a hemodialysis membrane as described above, the hollow fiber membranes are additionally or alternatively functionalized with an NMDAR protein construct according to the invention on the lumen side of the fibers where they can directly interact with the target metabolite in the blood or blood plasma which perfuses the lumen of the hollow fiber membrane. The construct may also or alternatively be immobilized to the outside of the membrane.

Methods of Extracorporeal Blood Treatment

The invention includes devices which are configured to be located in an extracorporeal blood circuit through which the blood of a patient passes and which comprises means for transporting blood from the patients vascular system to a blood treatment device at a defined flow rate and then returning the treated blood back to the patient, and wherein the device is further configured to reduce levels of NMDAR autoantibodies in the blood.

According to the invention, the expression “extracorporeal blood purification” refers preferably to the process of removing substances from body fluids through their clearance from flowing blood in a diverted circuit outside the patient's body (extracorporeal). Said substances may include endogenous toxins (i.e., uremic toxins), exogenous poisons (i.e., ethylene glycol or fungal toxin), administered drugs, viruses, bacteria, antibodies, metabolites and proteins (i.e., IMHA, myasthenia gravis), abnormal cells (i.e., leukemia), and excessive water. Therapeutic procedures include hemodialysis, including intermittent hemodialysis (HD, HDF, HF) and continuous renal replacement therapy (CRRT); hemoperfusion; plasma exchange and therapeutic apheresis. Such methods are known to a skilled person and the device of the invention can be incorporated accordingly.

The expression “blood” as used herein refers to whole blood which contains all components of the blood of an organism, including red cells, white cells, and platelets suspended in plasma. The expression “blood plasma” refers to the fluid, composed of about 92% water, 7% proteins such as albumin, gamma globulin, fibrinogen, complement factors, clotting factors, and 1% mineral salts, sugars, fats, electrolytes, hormones and vitamins which forms part of whole blood but no longer contains red and white cells and platelets. In the context of the present invention, the expression “blood plasma” or “plasma” refers to specific fractions of the above defined blood plasma in its standard meaning, such as, for example, blood serum.

According to one aspect, blood flow rates in an extracorporeal blood purification circuit are between 20 ml and 700 ml/min. Typical dialysate flow rates in an extracorporeal circuit comprising a hemodialyzer for the treatment of renal failure, either in addition to the blood treatment device according to the invention or in cases where the hemodialyzer in addition is configured to bind NMDAR autoantibodies, is in the range of between 0.5 l/h and 800 ml/min.

In therapeutic apheresis whole blood can be treated or blood is separated into its component fractions, for example by centrifugation or by means of a plasma membrane or filter, and the fraction containing the solute which shall be removed, is specifically treated prior to return to the patient.

The present invention provides for an apheresis treatment in which whole blood or plasma (containing the target proteins) is removed from the patient's flowing blood and, after having been contacted with a device or matrix according to the invention is returned to the patient. Typical blood or plasma flow rates in an extracorporeal circuit wherein the blood treatment device is perfused with whole blood or plasma is in the range of between 30 ml/min and 200 ml/min, or 7 ml/min and 50 ml/min respectively.

According to one aspect, the extracorporeal blood circuit according to the invention is configured to perform hemodialysis. In this case, the device according to the invention is, for example, a hemodialyzer which additionally has been configured to immobilize/bind NMDAR autoantibodies according to the invention. The circuit can be operated in different treatment modes depending on the medical need, including hemodialysis, hemodiafiltration, hemofiltration mode.

Unless defined otherwise, all technical and scientific terms used herein have the same meaning as commonly understood by one of ordinary skill in the art to which this invention belongs. Also, all publications, patent applications, patents and other references mentioned herein are incorporated by reference in their entirety.

### FIGURES

The invention is further described by the following figures. These are not intended to limit the scope of the invention, but represent preferred embodiments of aspects of the invention provided for greater illustration of the invention described herein.

### BRIEF DESCRIPTION OF THE FIGURES

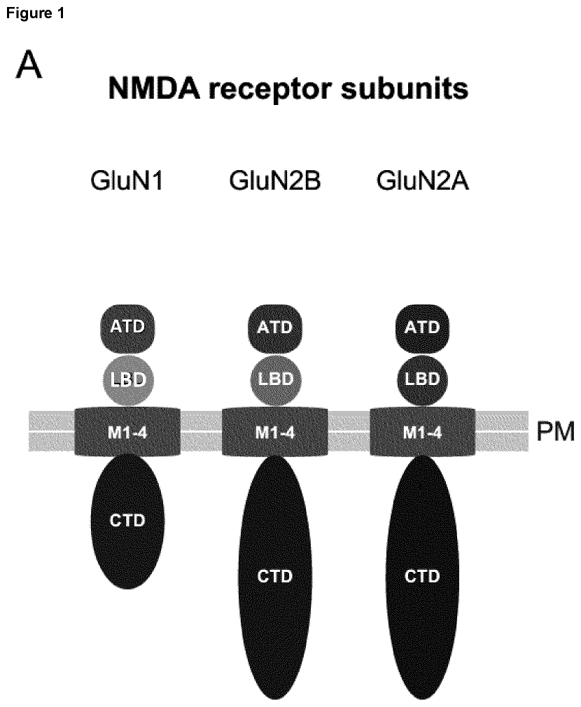

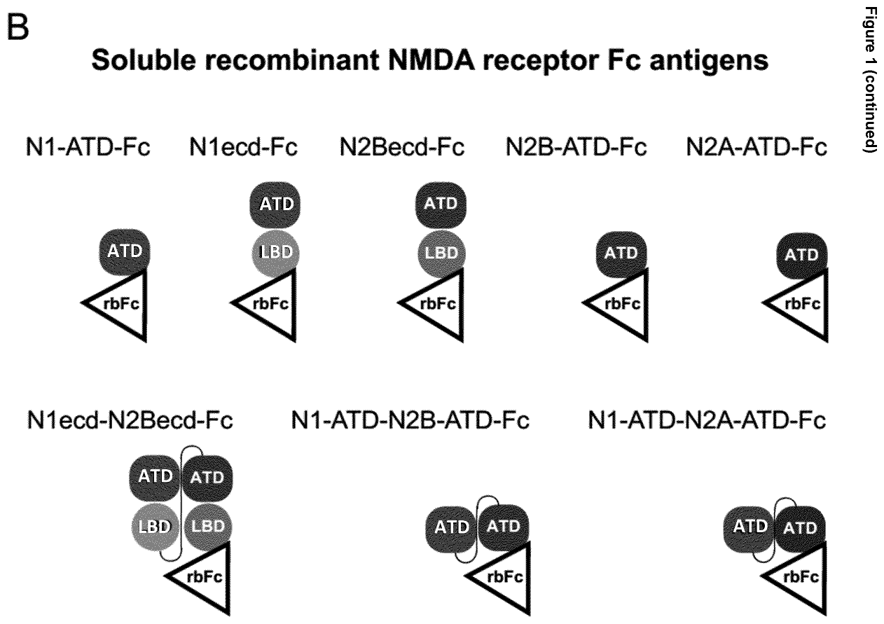

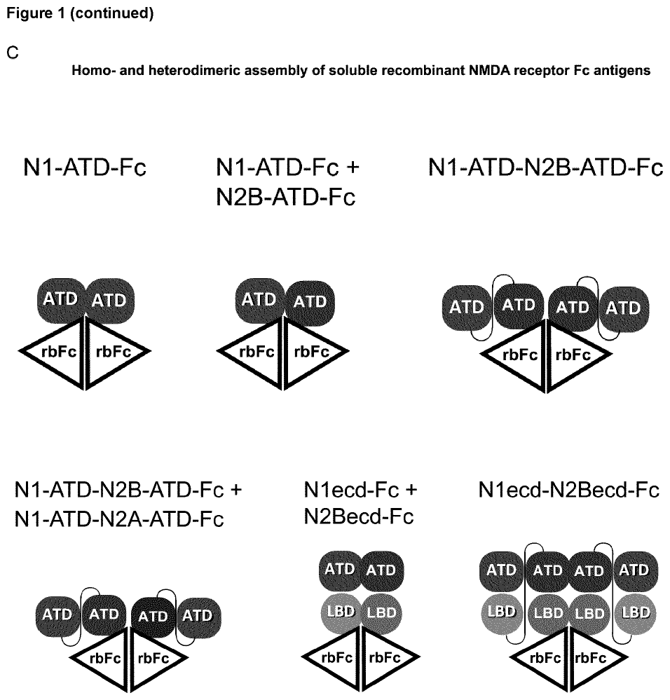

FIG. 1: Scheme of soluble recombinant NMDA receptor Fc (srNR-Fc) antigens/fusion proteins. In the context of the invention, srNR-Fc antigen and srNR-Fc fusion protein are used interchangeably.

FIG. 2: Soluble recombinant NMDA receptor Fc fusion proteins are recognized by a recombinant human GluN1 autoantibody.

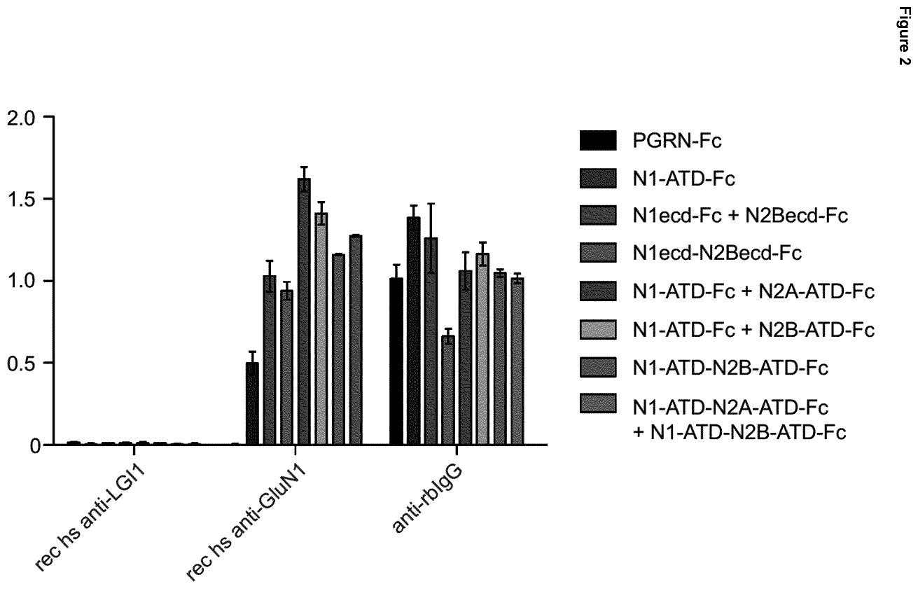

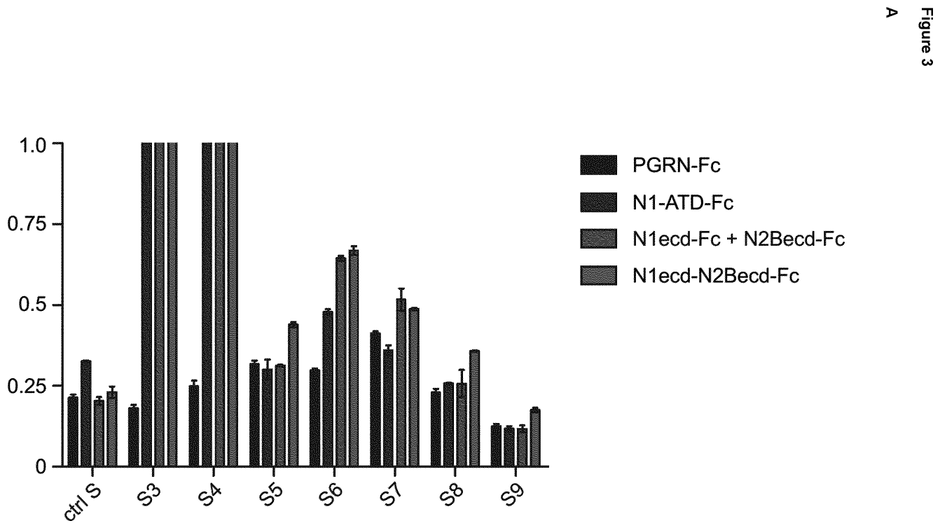

FIG. 3: Soluble recombinant NMDA receptor Fc fusion proteins detect NMDA receptor autoantibodies in patients' sera.

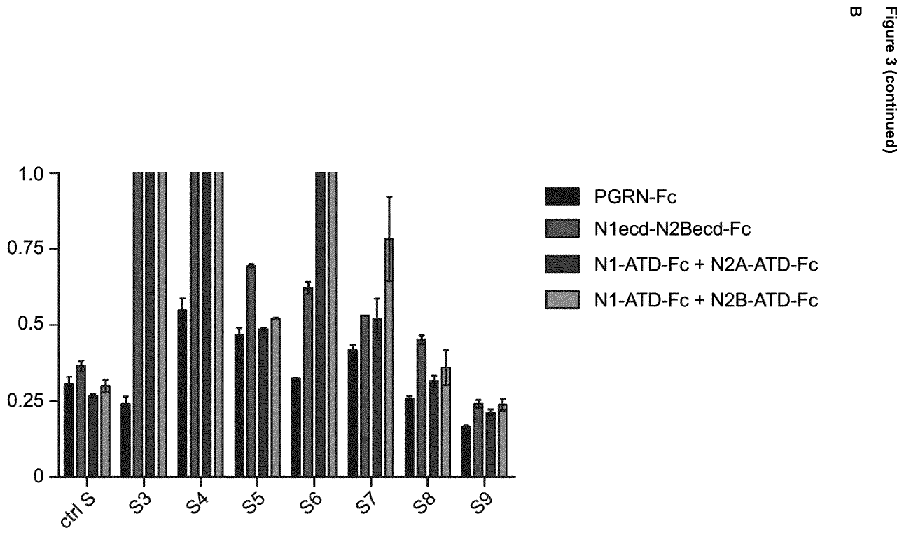

FIG. 4: Autoantibody detection by soluble recombinant NMDA receptor Fc fusion proteins in sera with known anti-NMDA receptor titers.

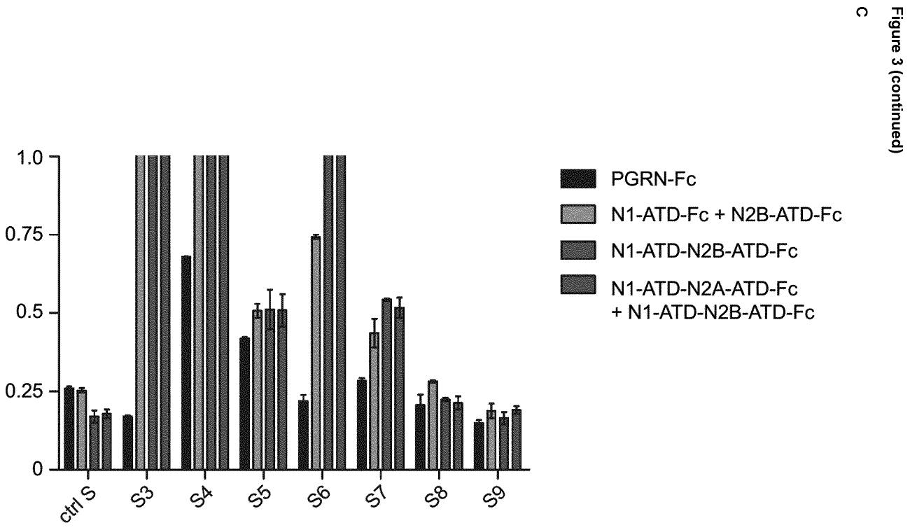

FIG. 5: ELISA screen for LGI1 antibodies using a soluble recombinant NMDA receptor Fc fusion protein (fusion protein #1 as disclosed herein) as a control.

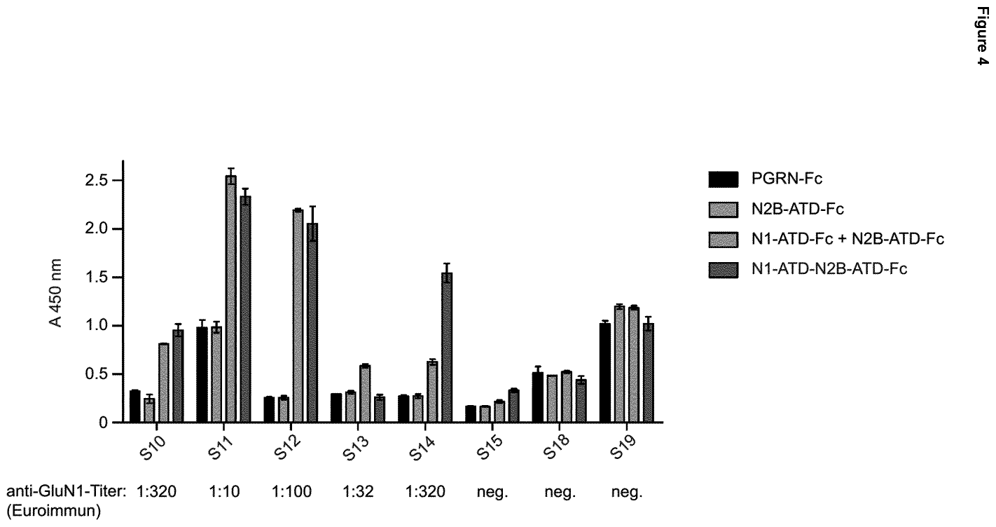

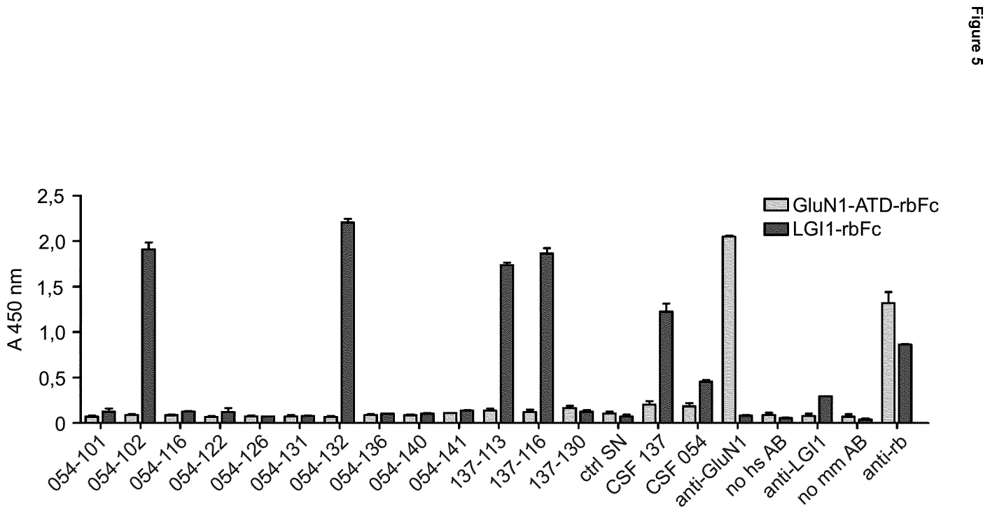

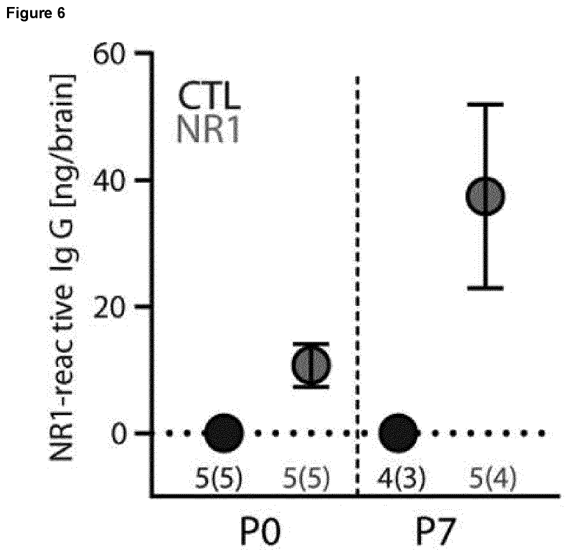

FIG. 6: ELISA quantification of recombinant human NR1(GluN1) AB in mouse brain extracts using a soluble recombinant NMDA receptor Fc fusion protein according to fusion protein #1 disclosed herein.

FIG. 7: Soluble NMDA receptor Fc protein constructs of the invention detect heterodimer-selective recombinant human NMDA receptor autoantibodies.

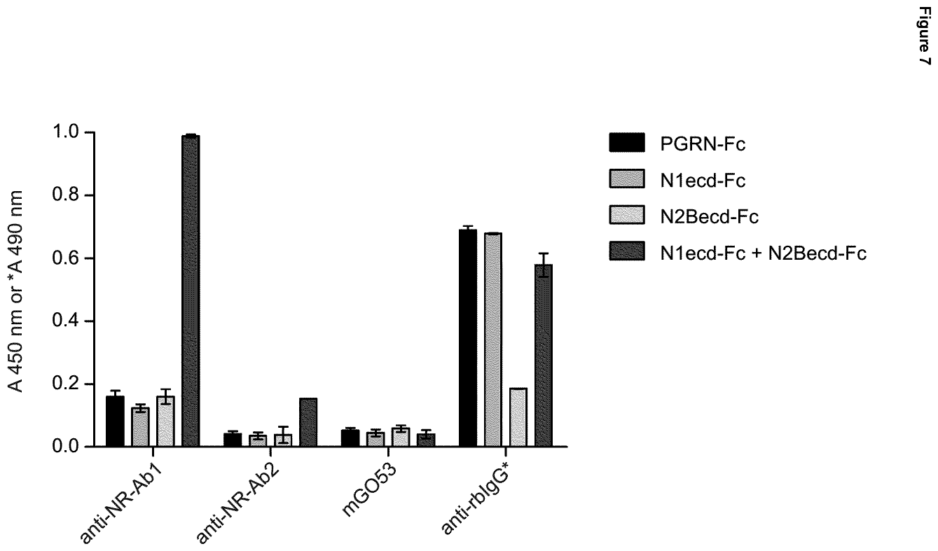

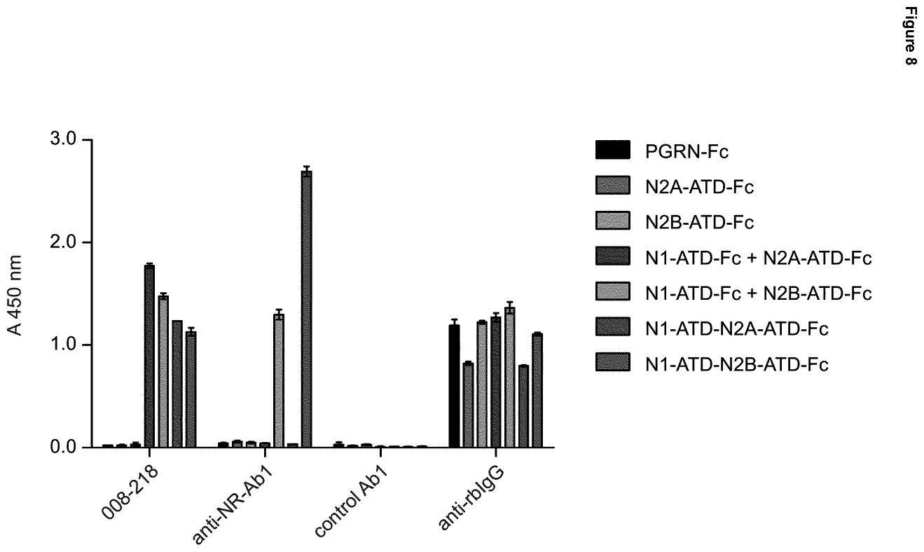

FIG. 8: Soluble NMDA receptor Fc protein constructs of the invention detect subtype-selective recombinant human NMDA receptor autoantibodies.

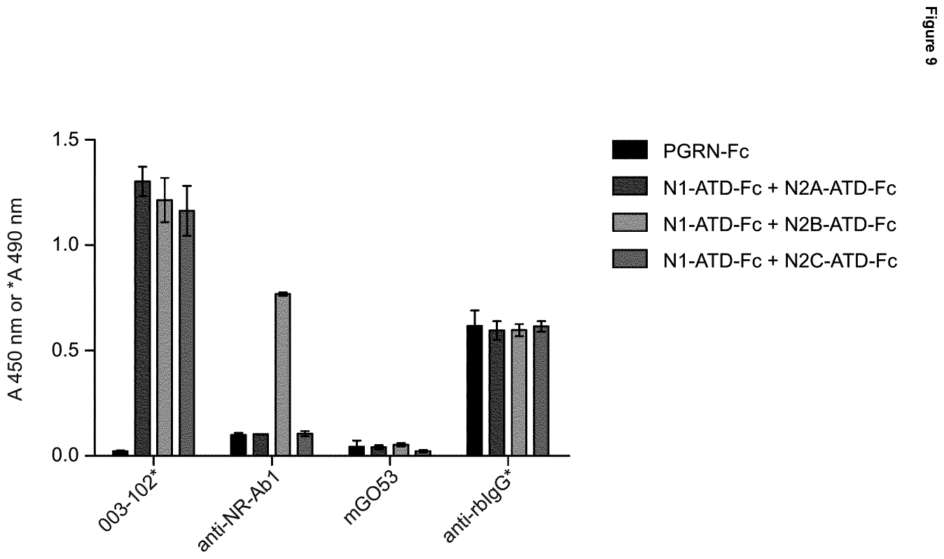

FIG. 9: Soluble NMDA receptor Fc protein constructs of the invention reveal the subtype selectivity of recombinant human NMDA receptor autoantibodies.

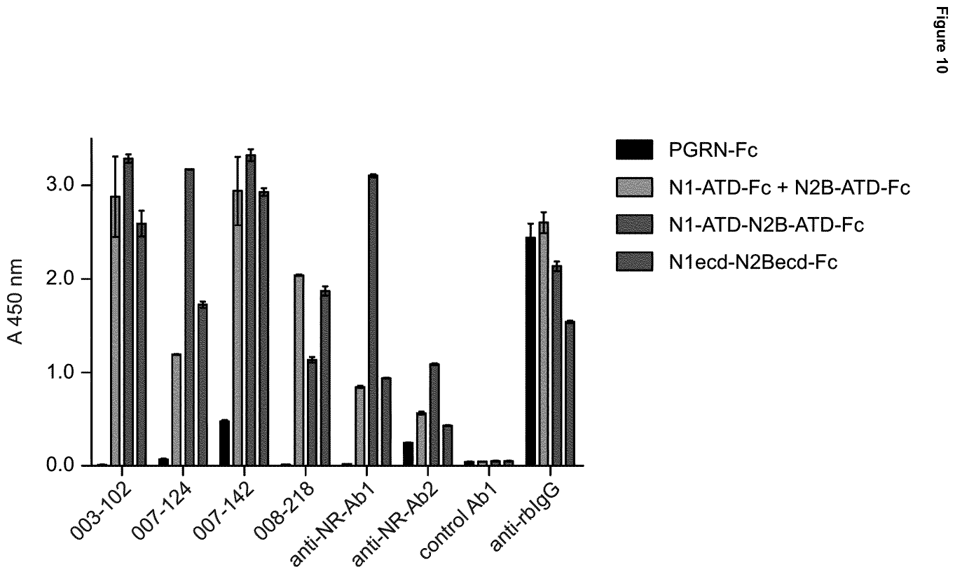

FIG. 10: Detection of recombinant human NMDA receptor autoantibodies by three soluble NMDA receptor Fc protein constructs of the invention.

### DETAILED DESCRIPTION OF THE FIGURES

FIG. 1. A. Simplified representation of NMDA receptor subunits encompassing amino terminal domain (ATD), ligand-binding domain (LBD), plasma membrane (PM)-spanning/associated segments (M1-4) and intracellular carboxyterminal domain (CTD). For details please see Paoletti et al. (2013) Nature Rev Neurosci 14, 383ff. B. Extracellular domains of NMDA receptor subunits depicted in A fused to rabbit Fc (rbFc, black triangle represents a single polypeptide containing CH2 and CH3) yield soluble antigens. The black line represents a linker between domains derived from different subunits (for details see Table 3). C. The soluble antigens are expected to form homo- or heteromeric dimers through rbFc upon (co)expression (a selection of the possible combination is shown).

FIG. 2. Cell culture supernatants of HEK293 cells expressing and secreting the designated fusion proteins (Table 3) were captured on a 96-well plate through an anti-rabbit IgG antibody. Single recombinant (rec) human (hu) autoantibodies to LGI1 or GluN1 (Kreye et al., 2016) derived from patients' CSF cells were applied at 1 μg/ml and detected using a horseradish peroxidase (HRP)-conjugated anti-human IgG antibody; captured Fc fusion proteins were detected directly using an HRP-conjugated anti-rabbit (rb) IgG antibody at 0.016 μg/ml. ELISA signals are shown as mean±SD of two wells in a single experiment after subtraction of the signal generated by the anti-human IgG antibody alone. PGRN, progranulin. Fc, constant region of rabbit IgG1 heavy chain.

FIG. 3. Cell culture supernatants of HEK293 cells expressing and secreting the designated fusion proteins (Table 3) were captured on a 96-well plate through an anti-rabbit Fc antibody. Sera derived from seven NMDAR encephalitis patients (S3-9) and a control serum (ctrl S) were applied at a 1:200 dilution and human IgG detected using Biotin-conjugated anti-human IgG and HRP-conjugated streptavidin. Panels A to C represent separate experiments. ELISA signals are shown as mean±SD of two wells in a single experiment after subtraction of the signal caused by the combination of Biotin-conjugated anti-human IgG and HRP-conjugated streptavidin alone. PGRN, progranulin. Fc, constant region of rabbit IgG1 heavy chain.

FIG. 4. Cell culture supernatants of HEK293 cells expressing and secreting the designated fusion proteins were captured on a 96-well plate through an anti-rabbit Fc antibody. Sera derived from five NMDAR encephalitis patients (S10-14) and three control sera (S15, S18, S19) were applied at a 1:100 dilution and human IgG detected using Biotin-conjugated anti-human IgG and HRP-conjugated streptavidin. ELISA signals are shown as mean±SD of two wells in a single experiment after subtraction of the signal caused by the combination of Biotin-conjugated anti-human IgG and HRP-conjugated streptavidin alone. NMDAR antibody titers of the sera determined by the Euroimmun cell-based assay are given below for comparison. PGRN, progranulin. Fc, constant region of rabbit IgG1 heavy chain.

FIG. 5. To screen for LGI1 reactivity, cell culture supernatants of HEK293T cells expressing CSF cell-derived antibody cDNAs were applied to LGI1-Fc and to NMDA receptor subunit GluN1-ATD-Fc captured on an ELISA plate. Example assay shows results for 13 supernatants as well as controls including CSF samples (diluted 1:5), human recombinant anti-GluN1, mouse anti-LGI1 and secondary antibodies to human, mouse and rabbit IgG alone. Signals are shown as mean±SD of two wells.

FIG. 6. NR1 (GluN1) specific human IgG was found in neonatal mouse whole brain extracts via ELISA only following prenatal NR1 antibody injections with increasing concentrations between PO and P7. ELISA quantification of NR1-specific human AB in mouse brain extracts revealed increasing levels of brain-bound IgG from PO (mean=10.7 ng) to P7 (mean=37.4 ng) in the NR1 group.

FIG. 7. Cell culture supernatants of HEK293 cells expressing and secreting the designated fusion proteins (Table 3) were captured on a 96-well plate through an anti-rabbit IgG antibody. Single recombinant human NMDA receptor autoantibodies (anti-NR-Ab1 and 2) derived from patients' CSF cells or a control antibody (mGO53) were applied at 4.0 μg/ml (anti-NR-Ab1), 3.1 μg/ml (anti-NR-Ab2) and 5.9 μg/ml (mGO53) and were detected using a horseradish peroxidase (HRP)-conjugated anti-human IgG antibody; captured Fc fusion proteins were detected directly using an HRP-conjugated anti-rabbit (rb) IgG antibody at 0.016 μg/ml. ELISA signals are shown as mean±SD of two wells in a single experiment after subtraction of the signal generated by the anti-human IgG antibody alone. A 490 nm values are shown for anti-rabbit IgG. PGRN, progranulin. Fc, constant region of rabbit IgG1 heavy chain.

FIG. 8. Cell culture supernatants of HEK293 cells expressing and secreting the designated fusion proteins (Table 3) were captured on a 96-well plate through an anti-rabbit IgG antibody. Single recombinant human NMDA receptor autoantibodies derived from patients' CSF cells (Kreye et al., 2016 or unpublished) or a recombinant human control antibody (control Ab1) were applied at 1 μg/ml and were detected using a horseradish peroxidase (HRP)-conjugated anti-human IgG antibody; captured Fc fusion proteins were detected directly using an HRP-conjugated anti-rabbit (rb) IgG antibody at 0.016 μg/ml. ELISA signals are shown as mean±SD of two wells in a single experiment after subtraction of the signal generated by the anti-human IgG antibody alone. PGRN, progranulin. Fc, constant region of rabbit IgG1 heavy chain.

FIG. 9. Cell culture supernatants of HEK293 cells expressing and secreting the designated fusion proteins (Table 3) were captured on a 96-well plate through an anti-rabbit IgG antibody. Single recombinant human NMDA receptor autoantibodies derived from patients' CSF cells (Kreye et al., 2016 or unpublished) or a control antibody (mGO53) were applied at 0.09 μg/ml (003-102) or 1.8 μg/ml (anti-NR-Ab1, mGO53) and were detected using a horseradish peroxidase (HRP)-conjugated anti-human IgG antibody; captured Fc fusion proteins were detected directly using an HRP-conjugated anti-rabbit (rb) IgG antibody at 0.016 μg/ml. ELISA signals are shown as mean±SD of two wells in a single experiment after subtraction of the signal generated by the anti-human IgG antibody alone. A 490 nm values are shown for 003-102 and for anti-rabbit IgG. PGRN, progranulin. Fc, constant region of rabbit IgG1 heavy chain.

FIG. 10. Cell culture supernatants of HEK293 cells expressing and secreting the designated fusion proteins (Table 3) were captured on a 96-well plate through an anti-rabbit IgG antibody. Single recombinant human NMDA receptor autoantibodies derived from patients' CSF cells (Kreye et al., 2016 or unpublished) were applied at 0.01 μg/ml (003-102), 1 μg/ml (008-218, anti-NR-Ab1) or 10 μg/ml (all others) and were detected using a horseradish peroxidase (HRP)-conjugated anti-human IgG antibody; captured Fc fusion proteins were detected directly using an HRP-conjugated anti-rabbit (rb) IgG antibody at 0.016 μg/ml. ELISA signals are shown as mean±SD of two wells in a single experiment after subtraction of the signal generated by the anti-human IgG antibody alone. PGRN, progranulin. Fc, constant region of rabbit IgG1 heavy chain.

Concerning the FIGS. 2-10, in each of the figures the order of the tested constructs in the legend from top to bottom corresponds to the order of the bars from left to right for the respective condition.

### Examples

The invention is further described by the following examples. These are not intended to limit the scope of the invention but represent preferred embodiments of aspects of the invention provided for greater illustration of the invention described herein.

Technical Question

Is it possible to generate recombinant soluble fusion proteins for labelling, detection and isolation of NMDA receptor autoantibodies against NMDA receptors of different subunit compositions within the serum and CSF of patients?

### Solution

The amino terminal domain (ATD) of the NMDA receptor subunit GluN1, with or without additional extracellular domains of GluN1, alone or combined with extracellular domains of the NMDA receptor subunits GluN2A or GluN2B were fused to the constant region of rabbit IgG1 heavy chain (rbFc).

Fc mediated dimerization of expressed proteins may lead to epitopes highly similar to native NMDA receptors and may lend unprecedented stability to the fusion proteins. These soluble recombinant NMDA receptor Fc (srNR-Fc) fusion proteins/antigens are able to detect NMDA receptor autoantibodies against different NMDA receptor subunit compositions present in the serum of NMDAR encephalitis patients and therefore represent the core subject matter of this invention.

The ELISA method used to detect NMDA receptor autoantibodies with srNR-Fc fusion proteins/antigens may be used as a companion diagnostic.

### Detailed Examples

### Example 1: Exemplary Protein Constructs of the Invention Generated for Experiments

We have generated constructs encompassing extracellular parts of GluN1 and GluN2 subunits (FIGS. 1A and 1B, Table 1), expressed them in HEK293 cells and isolated cell culture supernatants containing the secreted Fc fusion proteins three days later.

Constructs #1, 2, 3, 5, 6 and 9 (Table 3) are Fc fusion proteins of either GluN1 or GluN2 domains, whereas in constructs #4, 7 and 8 domains of both GluN1 and GluN2B, separated by an artificial linker, are fused to Fc in a single molecule. The Fc domain will likely lead to dimerization of all the fusion proteins, leading to GluN1/GluN2 heterodimers upon co-expression of construct #1 or 2 with #5, 6, 9 or 3, respectively, or dimers of GluN1/GluN2 heterodimers upon expression of constructs #4, 7 and/or 8 (FIG. 1C).

TABLE 3
Soluble recombinant NMDA receptor rbFc fusion proteins that are comprised
by and/or represent NMDAR protein constructs of the invention.

| | | | NMDA receptor part: amino acids in |
|---|---|---|---|
| | | | GenBank entry |
| | | Fusion Protein | No of amino acids in NMDA receptor |
| # | Designation | Composition | part/whole protein |
| 1 | N1-ATD-Fc | hsGluN1-ATD-rbFc | 1-400 (NM_007327) |
| | | | 400/631 |
| 2 | N1ecd-Fc | hsGluN1-ATD-S1-GT- | 1-544-GT-663-800 (NM_007327) |
| | | S2-rbFc | 684/915 |
| 3 | N2Becd-Fc | hsGluN2B-ATD-S1-GT- | 1-540-GT-662-803 (NM_000834) |
| | | S2-rbFc | 684/915 |
| 4 | N1ecd-N2Becd- | hsGluN1-ATD-S1-GT- | 1-544-GT-663-800 (NM_007327)-GST- |
| | Fc | S2-linker-hsGluN2B- | 3xGGGGS-GAA-SR- |
| | | ATD-S1-GT-S2-rbFc | 29-540-GT-662-803 (NM_000834) |
| | | | 1363/1594 |
| 5 | N2A-ATD-Fc | hsGluN2A-ATD-rbFc | 1-408 (NM_001134407) |
| | | | 408/639 |
| 6 | N2B-ATD-Fc | hsGluN2B-ATD-rbFc | 1-408 (NM_000834) |
| | | | 408/639a |
| 7 | N1-ATD-N2A- | hsGluN1-ATD-linker- | 1-400(NM_007327)-GST-3xGGGGS-GAA-SR- |
| | ATD-Fc | hsGluN2A-ATD-rbFc | 28-408 (NM_001134407) |
| | | | 804/1035 |
| 8 | N1-ATD-N2B- | hsGluN1-ATD-linker- | 1-400(NM_007327)-GST-3xGGGGS-GAA-SR- |
| | ATD-Fc | hsGluN2B-ATD-rbFc | 29-408(NM_000834) |
| | | | 803/1034 |
| 9 | N2C-ATD-Fc | hsGluN2C-ATD-rbFc | 1-405 (NM_000835) |
| | | | 405/636a |

hs, human.
rb, rabbit.
Ecd, extracellular domain.
ATD, amino terminal domain.
No, number.
Fc, constant region of rabbit IgG1 heavy chain.

We established an ELISA to test the ability of srNR-Fc proteins to detect NMDA receptor antibodies in the serum of patients. Briefly, srNR-Fc proteins in cell culture supernatants were captured on 96-well plates via anti-rabbit Fc or anti-rabbit IgG antibodies. NMDAR encephalitis patient sera or human monoclonal antibodies were applied and bound antibody detected using either Biotin-conjugated anti-human IgG and horseradish peroxidase (HRP)-conjugated streptavidin or HRP-conjugated anti-human IgG antibody, and the HRP substrate ultraTMB.

### Example 2: Soluble NMDA Receptor rbFc Fusion Proteins are Recognized by a Recombinant Human GluN1 Autoantibody

FIG. 2 shows that all srNR-Fc antigens tested, but not a control antigen (Progranulin-Fc) were recognized by a recombinant human anti-NMDA receptor antibody. In contrast, none of the antigens was recognized by a recombinant human anti-LGI1 antibody. An anti-rabbit IgG antibody detecting the rbFc portion of the fusion proteins was used to confirm that all rbFc fusion proteins were successfully expressed and immobilized on the ELISA plate.

### Example 3: Soluble NMDA Receptor rbFc Fusion Proteins Detect NMDA Receptor Autoantibodies in Patients' Sera

FIG. 3 summarizes results of three ELISA experiments with a set of human sera from NMDAR encephalitis patients on the different srNR-Fc antigens. The data reveals that GluN1-ATD-Fc as well as antigens containing additional extracellular regions of GluN1 or GluN2 subunits yielded signals as in most of the NMDAR encephalitis patient sera, compared to Progranulin as a control. Three of the sera strongly reacted with the antigens; the other four sera reacted only with selected srNR-Fc antigens and yielded lower signals. Sera were diluted 1:200 suggesting that the srNR-Fc antigens allow high sensitivity NMDA receptor autoantibody detection. Addition of regions of GluN2 subunits improved the sensitivity of the detection. A subset of NMDAR autoantibodies may recognize GluN1 only in the presence of an assembled GluN2 domain. The antigen yielding the highest signal differed from serum to serum. For example, antigen containing N1ecd-N2Becd was superior in detecting antibodies in serum 5, whereas antigens containing GluN1-ATD and GluN2B-ATD worked better in the detection of antibodies in serum 7.

### Example 4: Autoantibody Detection by Soluble NMDA Receptor rbFc Fusion Proteins in Sera with Known Anti-NMDA Receptor Titers

To test how the ELISA based on srNR-Fc proteins compares to the clinical standard assay, we went on to measure sera with a known titer from the Euroimmun CBA (FIG. 4). All sera that had scored positive at Euroimmun (S10-14) yielded a positive signal with at least one of the antigens tested as compared to Progranulin and GluN2B-ATD as controls, while the sera that scored negative in the Euroimmun CBA (S15, S18, S19) did not. These data indicate that the srNR-Fcs containing GluN1-ATD specifically detect NMDAR autoantibodies in sera.

We used two srNR-Fc combinations expressing the ATDs of GluN1 and GluN2B in this assay (N1-ATD-Fc+N2B-ATD-Fc and N1-ATD-N2B-ATD-Fc). They contain the same amino acids of the NMDAR (Table 1), but in one case the antigen is reconstituted from two separate proteins, while the other construct contains the ATDs connected by an artificial linker as a single protein. These two antigens gave comparable signals with sera S10-S12, but differed considerably in sera S13 and S14. Antigen N1-ATD-Fc+N2B-ATD-Fc yielded small, comparable signals in S13 and S14. In contrast, N1-ATD-N2B-ATD-Fc did not detect any NMDAR antibody signal in S13 and a strong signal in S14. The molecular make-up of N1-ATD-N2B-ATD-Fc may have prevented access of the NMDAR autoantibodies present in S13 to their epitope. This finding emphasizes that several srNR-Fc combinations should be tested to detect as many antibodies as possible.

### Example 5: Soluble NMDA Receptor rbFc Fusion Proteins Detect Subtype-Selective Recombinant Human NMDA Receptor Autoantibodies

Some autoantibodies were detected by soluble NMDA receptor Fc antigens encompassing the extracellular domains of two different NMDA receptor subunits, but not by soluble NMDA receptor Fc antigens containing the extracellular domains of a single NMDA receptor subunit (FIG. 7). These NMDA receptor antibodies are not detected by assays based on GluN1-expressing cells.

The soluble NMDA receptor Fc antigens were used to determine if a specific subunit combination is targeted by recombinant human NMDA receptor autoantibodies. NMDA receptor autoantibody 008-218 was detected by soluble NMDA receptor Fc antigens either containing the ATDs of GluN1 and GluN2A or containing the ATDs of GluN1 and GluN2B with a comparable efficiency. However, anti-NR-Ab1 was detected by soluble NMDA receptor Fc antigens containing the ATDs of GluN1 and GluN2B, but not by soluble NMDA receptor Fc antigens containing the ATDs of GluN1 and GluN2A (FIG. 8). Furthermore, this antibody was not detected by an additional soluble NMDA receptor Fc antigen containing the ATDs of GluN1 and GluN2C (FIG. 9). The soluble NMDA receptor Fc antigens therefore classify anti-NR-Ab1 as a GluN1/GluN2B-subtype-selective antibody.

The soluble recombinant NMDA receptor Fc antigen N1-ATD-N2B-ATD-Fc yielded higher signals than the assembled antigen N1-ATD-Fc+N2B-ATD-Fc with the GluN1/GluN2B-subtype-selective antibody anti-NR-Ab1 (FIG. 8), suggesting that the specific molecular make-up of the Fc fusion protein N1-ATD-N2B-ATD-Fc, with the ATDs of GluN1 and GluN2B being part of a single protein, provides an advantage in detecting subtype-selective antibodies.

### Example 6: Detection of Recombinant Human NMDA Receptor Autoantibodies by Three Soluble NMDA Receptor rbFc Fusion Proteins

In a test of three soluble recombinant NMDA receptor Fc antigens, N1-ATD-N2B-ATD-Fc yielded the highest signals with several of the examined human recombinant NMDA receptor autoantibodies, while N1-ATD-Fc+N2B-ATD-Fc yielded a comparable or higher signal for others (FIG. 10). These data indicate a differential antibody-specific sensitivity of the soluble recombinant NMDA receptor Fc antigens and complement the findings in sera described in Example 4.

Discussion of the Examples

The results provided here serve as a proof of concept. We conclude (1) that soluble fusion proteins containing the amino terminal domain of GluN1 and rabbit Fc heterologously expressed in and secreted from HEK293 cells are able to bind NMDA receptor autoantibodies in patients' serum and (2) that efficient detection of NMDA receptor autoantibodies by soluble antigens benefits from the incorporation of extracellular domains of GluN2. Furthermore, use of the different srNR-Fc antigens may allow classifying patients' anti-NMDA receptor immune response.

Detection of autoreactivity against select NMDA receptor subtypes may enable a differential diagnosis in anti-NMDA receptor encephalitis and in other medical conditions associated with antibodies to the NMDA receptor.

### Further Examples of Experimental Applications of Constructs of the Invention

ELISA Screen for Recombinant LGI1 Antibodies Using an NMDA Receptor Fusion Protein as a Control.

For generating the mammalian expression constructs used in this experiment the cDNAs for amino acids 1-558 of human LGI1 (NM_005097.3) and for amino acids 1-400 of human GluN1 (NM_007327) were inserted into pFuse-rIgG-Fc1 (InvivoGen). The resulting plasmids encode hsLGI1 or the amino terminal domain (ATD) of hsGluN1 fused to the Fc region of rabbit IgG (amino acids SKP-PGK) linked by amino acids GSSTMVRS. The chimeric constructs LRR1-EPTP2 and LRR2-EPTP1 encode rabbit Fc fusions of amino acids 1-223 of LGI1 and amino acids 218-545 of LGI2 or amino acids 1-217 of LGI2 and amino acids 224-557 of LGI1, respectively.

Antibody binding to LGI1-Fc and to NMDA receptor subunit GluN1-ATD-Fc was compared in an ELISA. 96-well high-binding microplates (Greiner #655061) coated with donkey anti-rabbit IgG (10 μg/mL, Dianova, #711-005-152) were blocked and incubated with cell culture supernatants of HEK293 cells that expressed Fc fusion proteins. Cell culture supernatants containing monoclonal antibodies, CSF samples or purified antibodies and horseradish peroxidase (HRP)-conjugated donkey-anti-human IgG (1:5,000, Dianova, #709-035-149) were sequentially applied. After thorough washing, HRP activity was measured using 1-Step Ultra TMB-ELISA substrate (Thermo Fisher). The presence of immobilized antigens was confirmed by incubation with HRP-conjugated F(ab′)2 donkey-anti-rabbit IgG (1:50,000, Dianova, #711-036-152). Human recombinant anti-GluN1 antibody 003-102 (Kreye J, Wenke N K, Chayka M, et al. Human cerebrospinal fluid monoclonal N-methyl-D-aspartate receptor autoantibodies are sufficient for encephalitis pathogenesis. Brain 2016; 139:2641-2652) was used at 10 ng/ml. The results are displayed in FIG. 5.

ELISA Quantification of Recombinant Human NR1(GluN1) AB in Mouse Brain Extracts Using an NMDA Receptor Fusion Protein.

Concentration of recombinant human NR1 AB #003-102 in brain extracts was determined in 96-well plates coated overnight at 4° C. with donkey-anti-rabbit IgG (20 μg/mL, Dianova, #711-005-152). After blocking with 2% BSA in PBS/0.05% Tween-20 (PBS/T) at RT, cell culture supernatants of HEK293 cells that expressed the amino terminal domain (amino acids 1-400) of human NR1 (GluN1) fused to rabbit Fc were applied. Mouse brain extracts were diluted 1:25/1:100 in 0.4% BSA-PBS/T and added in duplicates. Plates were washed with PBS/T and incubated with horseradish peroxidase (HRP)-conjugated donkey-anti-human IgG (1:5,000, Dianova, #709-035-149). After washing, HRP activity was measured using 1-Step Ultra TMB-ELISA substrate (Thermo Fisher). The concentrations of #003-102 in the extracts were deduced from a calibration curve generated with purified #003-102. The results are displayed in FIG. 6.

### REFERENCES

Dalmau, J., Geis, C., and Graus, F. (2017). Autoantibodies to Synaptic Receptors and Neuronal Cell Surface Proteins in Autoimmune Diseases of the Central Nervous System. Physiol Rev 97, 839-887.
Dalmau, J., Gleichman, A. J., Hughes, E. G., Rossi, J. E., Peng, X., Lai, M., Dessain, S. K., Rosenfeld, M. R., Balice-Gordon, R., and Lynch, D. R. (2008). Anti-NMDA-receptor encephalitis: case series and analysis of the effects of antibodies. Lancet Neurol 7, 1091-1098.
Dalmau, J., and Graus, F. (2018). Antibody-Mediated Encephalitis. N Engl J Med 378, 840-851.
Gable, M. S., Sheriff, H., Dalmau, J., Tilley, D. H., and Glaser, C. A. (2012). The frequency of autoimmune N-methyl-D-aspartate receptor encephalitis surpasses that of individual viral etiologies in young individuals enrolled in the California Encephalitis Project. Clin Infect Dis 54, 899-904.
Gleichman, A. J., Spruce, L. A., Dalmau, J., Seeholzer, S. H., and Lynch, D. R. (2012). Anti-NMDA receptor encephalitis antibody binding is dependent on amino acid identity of a small region within the GluN1 amino terminal domain. J Neurosci 32, 11082-11094.
Granerod, J., Ambrose, H. E., Davies, N. W., Clewley, J. P., Walsh, A. L., Morgan, D., Cunningham, R., Zuckerman, M., Mutton, K. J., Solomon, T., et al. (2010). Causes of encephalitis and differences in their clinical presentations in England: a multicentre, population-based prospective study. Lancet Infect Dis 10, 835-844.
Hughes, E. G., Peng, X., Gleichman, A. J., Lai, M., Zhou, L., Tsou, R., Parsons, T. D., Lynch, D. R., Dalmau, J., and Balice-Gordon, R. J. (2010). Cellular and synaptic mechanisms of anti-NMDA receptor encephalitis. J Neurosci 30, 5866-5875.
Jezequel, J., Johansson, E. M., Dupuis, J. P., Rogemond, V., Grea, H., Kellermayer, B., Hamdani, N., Le Guen, E., Rabu, C., Lepleux, M., et al. (2017a). Dynamic disorganization of synaptic NMDA receptors triggered by autoantibodies from psychotic patients. Nat Commun 8, 1791.
Jezequel, J., Rogemond, V., Pollak, T., Lepleux, M., Jacobson, L., Grea, H., Iyegbe, C., Kahn, R., McGuire, P., Vincent, A., et al. (2017b). Cell- and Single Molecule-Based Methods to Detect Anti-N-Methyl-D-Aspartate Receptor Autoantibodies in Patients With First-Episode Psychosis From the OPTiMiSE Project. Biol Psychiatry 82, 766-772.
Jones, B. E., Tovar, K. E., Goehring, A., Okada, N.J., Gouaux, E., and Westbrook, G. L. (2018). Anti-NMDA receptor encephalitis in mice induced by active immunization with conformationally-stabilized holoreceptors. bioRxiv, doi: http://dx.doi.org/10.1101/467902.
Kim, T. J., Lee, S. T., Moon, J., Sunwoo, J. S., Byun, J. I., Lim, J. A., Shin, Y. W., Jun, J. S., Lee, H. S., Lee, W. J., et al. (2017). Anti-LGI1 encephalitis is associated with unique HLA subtypes. Ann Neurol 81, 183-192.
Kreye, J., Wenke, N. K., Chayka, M., Leubner, J., Murugan, R., Maier, N., Jurek, B., Ly, L. T., Brandl, D., Rost, B. R., et al. (2016). Human cerebrospinal fluid monoclonal N-methyl-D-aspartate receptor autoantibodies are sufficient for encephalitis pathogenesis. Brain 139, 2641-2652.
Ladepeche, L., Planaguma, J., Thakur, S., Suarez, I., Hara, M., Borbely, J. S., Sandoval, A., Laparra-Cuervo, L., Dalmau, J., and Lakadamyali, M. (2018). NMDA Receptor Autoantibodies in Autoimmune Encephalitis Cause a Subunit-Specific Nanoscale Redistribution of NMDA Receptors. Cell Rep 23, 3759-3768.
Lee, C. H., and Gouaux, E. (2011). Amino terminal domains of the NMDA receptor are organized as local heterodimers. PLoS One 6, e19180.
Lee, C. H., Lu, W., Michel, J. C., Goehring, A., Du, J., Song, X., and Gouaux, E. (2014). NMDA receptor structures reveal subunit arrangement and pore architecture. Nature 511, 191-197.
Malviya, M., Barman, S., Golombeck, K. S., Planaguma, J., Mannara, F., Strutz-Seebohm, N., Wrzos, C., Demir, F., Baksmeier, C., Steckel, J., et al. (2017). NMDAR encephalitis: passive transfer from man to mouse by a recombinant antibody. Ann Clin Transl Neurol 4, 768-783.
Mueller, S. H., Farber, A., Pruss, H., Melzer, N., Golombeck, K. S., Kumpfel, T., Thaler, F., Elisak, M., Lewerenz, J., Kaufmann, M., et al. (2018). Genetic predisposition in anti-LGI1 and anti-NMDA receptor encephalitis. Ann Neurol 83, 863-869.
Paoletti, P., Bellone, C., and Zhou, Q. (2013). NMDA receptor subunit diversity: impact on receptor properties, synaptic plasticity and disease. Nat Rev Neurosci 14, 383-400.
Sharma, R., Al-Saleem, F. H., Puligedda, R. D., Rattelle, A., Lynch, D. R., and Dessain, S. K. (2018). Membrane-bound and soluble forms of an NMDA receptor extracellular domain retain epitopes targeted in auto-immune encephalitis. BMC Biotechnol 18, 41.
Tajima, N., Karakas, E., Grant, T., Simorowski, N., Diaz-Avalos, R., Grigorieff, N., and Furukawa, H. (2016). Activation of NMDA receptors and the mechanism of inhibition by ifenprodil. Nature 534, 63-68.
Wenke, N. K., Kreye, J., Andrzejak, E., van Casteren, A., Leubner, J., Murgueitio, M. S., Reincke, S. M., Secker, C., Schmidl, L., Geis, C., Ackermann, F., Nikolaus, M., Garner, C. C., Wardemann, H., Wolber, G. and Prüss H. (2019). N-methyl-D-aspartate receptor dysfunction by unmutated human antibodies against the NR1 subunit. Ann Neurol 85, 771-776.
Zhu, S., Stein, R. A., Yoshioka, C., Lee, C. H., Goehring, A., McHaourab, H. S., and Gouaux, E. (2016). Mechanism of NMDA Receptor Inhibition and Activation. Cell 165, 704-714.

1. An N-methyl-D-aspartate receptor (NMDAR) protein construct comprising one or more NMDAR autoantibody epitopes, wherein the construct comprises an extracellular domain (ECD) of the NMDAR subunit GluN1 or a fragment thereof and an ECD of at least one of the NMDAR subunits GluN2A, GluN2B, GluN2C or GluN2D, or fragment thereof, wherein the protein construct lacks a NMDAR transmembrane domain.

2. (canceled)

3. The NMDAR protein construct according to claim 1, wherein the construct comprises a dimerization domain and/or a capture domain.

4. The NMDAR protein construct according to claim 3, wherein the dimerization domain is the capture domain, preferably formed by an antibody Fc-fragment.

5. The NMDAR protein construct according to claim 1, wherein the ECD of GluN1 or fragment thereof comprises or consists of the amino terminal domain (ATD) of GluN1 or a fragment thereof, and/or wherein the ECD of at least one of the NMDAR subunits GluN2A, GluN2B, GluN2C or GluN2D, or fragment thereof comprises or consists of the ATD of at least one of the NMDAR subunits GluN2A, GluN2B, GluN2C or GluN2D, or fragment thereof, respectively.

6. The NMDAR protein construct according to claim 1, wherein the ECD of GluN1 and the ECD of at least one of the NMDAR subunits GluN2A, GluN2B, GluN2C or GluN2D, or fragment thereof, are covalently linked, preferably as a fusion protein.

7. The NMDAR protein construct according to claim 1, wherein the construct is a protein dimer of non-covalently bound monomers, wherein the construct can be a homodimer or a heterodimer.

8. The NMDAR protein construct according to claim 7, wherein the construct is a heterodimer formed from the ECD of GluN1 or fragment thereof (as one monomer) and the ECD of at least one of the NMDAR subunits GluN2A, GluN2B, GluN2C or GluN2D, or fragment thereof (as one monomer).

9. An in vitro method for the detection of NMDAR autoantibodies in a sample, the method comprising,
a. providing a sample suspected of comprising NMDAR autoantibodies,
b. providing a NMDA protein construct according to claim 1 comprising a capture domain as a capture molecule,
c. contacting said sample with said NMDAR protein construct, thereby binding NMDAR autoantibodies from said sample to said NMDAR protein construct, and
d. determining the presence of bound NMDAR autoantibodies.

10. The method according to claim 9, wherein the NMDAR autoantibodies in said sample are present in solution or on a cell-membrane.

11. The method according to claim 9, wherein the method is carried out with multiple and different of the NMDAR protein constructs.

12. The method according to claim 9, wherein the method is applied for the diagnosis, prognosis, disease monitoring, patient stratification and/or therapy monitoring of a medical condition associated with autoantibodies against the NMDAR, preferably anti-NMDAR encephalitis, and the sample suspected of comprising NMDAR autoantibodies is a sample of a human subject exhibiting symptoms of having said medical disorder.

13. The method according to claim 9, wherein the method is applied for therapy guidance of a subject suspected of having and/or developing a medical condition associated with NMDAR autoantibodies, the method comprising selecting one or more corresponding NMDAR protein construct(s) for subsequent treatment of said subject.

14. A kit for the diagnosis of an autoimmune disease associated with NMDAR autoantibodies in a subject by detection of NMDAR autoantibodies, comprising:
a. an NMDAR protein construct according to claim 1 or an NMDAR protein construct according to claim 1 immobilized on a solid surface, and a labelled secondary affinity reagent directed to human NMDAR autoantibodies and a detector for detecting the signal emitted from said label, or
b. a labelled NMDAR protein construct according to claim 1.

15. A blood treatment device configured to remove NMDAR autoantibodies from the blood or blood plasma of a person in need thereof in an extracorporeal blood circuit, wherein the device comprises a matrix having one or more NMDAR protein constructs according to claim 1 immobilized thereon.

16. The method according to claim 11, further comprising additionally determining against which NMDAR protein construct of said multiple constructs the NMDAR autoantibodies bind.

17. The method according to claim 11, further comprising additionally determining against which NMDAR protein construct of said multiple constructs the NMDAR autoantibodies bind in the largest amounts and/or most efficiently bind.

18. The kit according to claim 14, wherein the disease associated with NMDAR autoantibodies is-NMDAR encephalitis.
